(19) United States
(12) Patent Application Publication
(10) Pub. No.: US 2024/0294513 A1
(43) Pub. Date: Sep. 5, 2024

(54) PYRANONE COMPOUNDS USEFUL TO MODULATE OMA1 PROTEASE

(71) Applicant: 712 North Inc., Oakland, CA (US)

(72) Inventor: Marcel Victor Alavi Khorassani Moghadam, Berkeley, CA (US)

(73) Assignee: 712 North Inc., Oakland, CA (US)

(21) Appl. No.: 18/642,913

(22) Filed: Apr. 23, 2024

Related U.S. Application Data
(62) Division of application No. 17/156,237, filed on Jan. 22, 2021, now Pat. No. 11,993,590.
Application No. 17/156,237 is a continuation-in-part of application No. 16/022,481, filed on Jun. 28, 2018, now Pat. No. 10,906,931.
Application No. 16/022,481 is a continuation of application No. PCT/US2017/064195, filed on Dec. 1, 2017.
(60) Provisional application No. 62/581,723, filed on Nov. 5, 2017.
(60) Provisional application No. 62/429,846, filed on Dec. 4, 2016.
(60) Provisional application No. 62/481,392, filed on Apr. 4, 2017.
(60) Provisional application No. 63/041,292, filed on Jun. 19, 2020.

(51) Int. Cl. C07D 405/12; A61K 9/00; A61P 25/28; A61P 35/00

(52) U.S. Cl. CPC C07D 405/12; A61K 9/0043; A61K 9/0073; A61P 25/28; A61P 35/00

(57) ABSTRACT

Means and methods for therapeutic intervention of mitochondrial disorders or diseases, and in particular to a method for the treatment, prevention and/or amelioration of a disorder or disease correlated with mitochondrial stress or dysfunction, a mitochondrial disorder or disease, or a disorder or disease characterized by OPA1 alterations. Thereby, a pharmaceutically active amount of a compound capable of modulating the activity of OMA1 and/or an oligomeric complex of OMA1 is administered to a patient in need of medical intervention. Methods of screening for a compound capable of modulating the activity of OMA1 and/or an oligomeric complex comprising OMA1 are performed. Methods for determining the susceptibility for, predisposition for, or the presence of such a disorder or disease and whether a person in need will benefit from the therapeutic intervention, i.e., personalized medicine are also performed.

### CROSS-REFERENCE TO RELATED APPLICATIONS

This application is a Divisional of U.S. application Ser. No. 17/156,237 filed on Jan. 22, 2021, which is incorporated herein by reference in its entirety.

U.S. application Ser. No. 17/156,237 is a Continuation-in-Part of U.S. application Ser. No. 16/022,481 filed on Jun. 28, 2018, now U.S. Pat. No. 10,906,931 issued Feb. 2, 2021, which is incorporated herein by reference in its entirety. U.S. application Ser. No. 16/022,481 is a Continuation of PCT International Application No. PCT/US2017/064195 with an international filing date of Dec. 1, 2017, which is incorporated by reference in its entirety.

PCT International Application No. PCT/US2017/064,195 claims the benefit under 35 U.S.C. § 119(c) of U.S. Provisional Application No. 62/581,723 filed on Nov. 5, 2017, U.S. Provisional Application No. 62/481,392 filed on Apr. 4, 2017, and U.S. Provisional Application No. 62/429,846 filed on Dec. 4, 2016, each of which is incorporated by reference in its entirety.

U.S. application Ser. No. 17/156,237 claims also the benefit under 35 U.S.C. § 119(c) of U.S. Provisional Application No. 63/041,292 filed on Jun. 19, 2020, which is incorporated herein by reference in its entirety as well.

The foregoing applications, and all documents cited therein or during their prosecution (“applications' cited documents”) and all documents cited or referenced in the applications' cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. Citation or identification of any document in this application is not an admission that such document is available as prior art to the present invention.

### STATEMENT OF FEDERALLY SPONSORED RESEARCH AND DEVELOPMENT

This invention was made with government support under Grant No. 1R43AG063642-01 awarded by the National Institutes of Health. The government has certain rights in the invention.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One xml file, size 158,336 bytes, named “Sequence_Listing_ST26,” created on Apr. 16, 2024.

### FIELD OF THE INVENTION

The present invention relates to compounds disclosed herein as having OMA1 and/or OPA1 modulatory properties, pharmaceutical compositions comprising these compounds, chemical processes for preparation of these compounds, and their use as pharmacological tools or in the treatment of diseases connected to OMA1 and/or OPA1 in cells, animals and in particular humans. More particularly, the present disclosure provides novel pyranone derivates and methods of use of such compounds.

### BACKGROUND

Mitochondria are essential for cells; their proper function is an absolute requirement for cell survival. Mitochondria form large networks of dynamic interconnected tubules that are maintained by balanced fission and fusion events. Morphological alterations of mitochondria and the mitochondrial network have been reported in human disorders. Impairment of mitochondrial fusion or fission is causative of various neurodegenerative diseases such as Charcot-Marie-Tooth disease type 2A and 4A, familial Parkinson's disease, Alzheimer's disease, Autosomal Dominant Optic Atrophy (ADOA) and other optic neuropathies (Alexander et al. 2000; Delettre et al. 2000; Carelli et al. 2002; Sadun 2002; Carelli et al. 2004; Zuchner et al. 2004; Niemann et al. 2005; Kong et al. 2009; Lenaers et al. 2009; Osborne 2010; Lec et al. 2011; Chrysostomou et al. 2013; Maresca et al. 2013; Burte et al. 2015; Salminen et al. 2015; Bose and Beal 2016; Voigt et al. 2016; Zhang et al. 2016). There is strong evidence in particular for a causal relationship between mitochondrial dysfunction and Parkinson's disease (Schapira et al. 1990; Keeney et al. 2006; Parker et al. 2008; Santos et al. 2015; Dolle et al. 2016). On the other hand, dysfunctional mitochondria have been recognized for many years in brains from deceased patients with Alzheimer's disease as well (Parker et al. 1990; Smith et al. 1996; Gibson et al. 1998; Maurer et al. 2000; Butterfield et al. 2001; Devi et al. 2006). Cumulative evidence also exists for mitochondrial fusion/fission being necessary for normal cardiac function (Dorn 2013; Piquereau et al. 2013; Burke et al. 2015; Marin-Garcia and Akhmedov 2016; Ong et al. 2017). Another example for a mitochondrial disorder is cancer. Research over the past century or so has generated a complex and rich body of knowledge revealing cancer to be a disease correlated to mitochondrial dysfunction (Alirol and Martinou 2006; Frezza and Gottlieb 2009; Hanahan and Weinberg 2011; Wallace 2012; Vyas et al. 2016).

OPA1 is a mitochondrial pro-fusion protein with two functions in mitochondrial inner membrane fusion/cristae remodeling and cytochrome C release/cell death (Alavi and Fuhrmann 2013). Proteolytic cleavage of long OPA1 isoforms (OPA1L) produces short OPA1 isoforms (OPA1S). OPA1's dual functions in mitochondrial inner membrane remodeling and cytochrome C release (Olichon et al. 2003; Frezza et al. 2006) are regulated by the ratio of OPA1L-to-OPA1S (Song et al. 2007). OPA1 forms a complex together with IMMT at the inner membrane termed MICOS complex to regulate mitochondrial cristae morphology (Barrera et al. 2016; Glytsou et al. 2016; Hessenberger et al. 2017). This complex is associated with the mitochondrial outer membrane by binding to SAMM50 (Koob et al. 2015; Ott et al. 2015). OPA1 interacts with the apoptotic machinery at the mitochondrial outer membrane through the interaction with BNIP3 (Landes et al. 2010). OPA1L cleavage and OPA1S can promote mitochondrial fragmentation, cytochrome C release and correlates with cell death (Olichon et al. 2003; Duvezin-Caubet et al. 2006; Ishihara et al. 2006; Griparic et al. 2007; Song et al. 2007; Merkwirth et al. 2008; Ehses et al. 2009; Head et al. 2009). Several proteases are directly or indirectly involved in this conversion, such as YMEIL1, PARL, HTRA2, and OMA1, which plays the key role during stress-induced OPA1 cleavage (Cipolat et al. 2006; Duvezin-Caubet et al. 2006; Ishihara et al. 2006; Griparic et al. 2007; Song et al. 2007; Ehses et al. 2009; Head et al. 2009).

OMA1 is a zinc metallo-endopeptidase located in the mitochondrial inner membrane (Kaser et al. 2003). Heterozygous mutations in conserved OMA1 residues have been reported in several patients afflicted with familial and sporadic forms of amyotrophic lateral sclerosis (ALS) (Daoud et al. 2011). OMA1 is activated upon cellular stress events, such as increased reactive oxygen species or loss of mitochondrial function (Richter et al. 2015; Bohovych et al. 2016; Rainbolt et al. 2016). In the case of chronic or high stress, there is prolonged OMA1 activation, which in turn triggers the release of cytochrome C, ultimately leading to cell death (Jiang et al. 2014). OMA1 and YMEIL1, which proteolytically cleaves OPA1 at the S2 cleavage site, are organized and regulated in the inner mitochondrial membrane by PHB and PHB2 (Wai et al. 2016).

Cells have an innate regulatory feedback loop to counterbalance stress-induced OPA1 cleavage by OMA1 (Alavi and Fuhrmann 2013). Hypoxic conditions can cause decreased mitochondrial membrane potential, which activates OMA1 (Richter et al. 2015; Bohovych et al. 2016; Rainbolt et al. 2016). HIGD1A is a mitochondrial protein upregulated under hypoxic conditions by HIF1α (Ameri et al. 2015; Ameri and Maltepe 2015). HIGD1A can bind to the SI cleavage site thereby protecting OPA1 from proteolytic cleavage by OMA1 (An et al. 2011; An et al. 2013), while HIGD1A translocates to the nucleus in cells undergoing apoptosis (Ameri et al. 2013).

### SUMMARY

There remains an unmet medical need for therapeutic intervention because at present we still have no method for the treatment, prevention and/or amelioration of a disorder or disease correlated with mitochondrial dysfunction, for a mitochondrial disorder or disease; or for a disorder or disease characterized by OPA1 alterations

The present disclosure relates to means and methods for therapeutic intervention of mitochondrial disorders or diseases, in particular to a method for the treatment, prevention and/or amelioration of a disorder or disease correlated with mitochondrial stress or dysfunction, a mitochondrial disorder or disease, or a disorder or disease characterized by OPA1 alterations. Thereby, a pharmaceutically active amount of a compound capable of modulating the activity of OMA1 and/or an oligomeric complex comprising OMA1 is administered to a patient in need of medical intervention. The present invention also relates to a method of screening for a compound capable of modulating the activity of OMA1 and/or an oligomeric complex comprising OMA1. The present invention further relates to a method for determining the susceptibility for, predisposition for, or the presence of such a disorder or disease and whether a person in need will benefit from the therapeutic intervention, i.e., personalized medicine.

We identified compounds disclosed herein which are known in the arts as HIV-1 protease inhibitors. These compounds, as shown in the provided examples, surprisingly and quite unexpectedly activated the OMA1 protease. The disclosed compounds had also a dose-dependent antagonistic effect on OPA1. Low doses resulted in OPA1 cleavage. However, higher doses prevented OPA1 cleavage even under conditions that would otherwise result in OPA1 cleavage.

The OMA1 protease has a number of disease implications and is supported by epidemiological and genetic data from humans and animal disease models. And yet, there are still until now no OMA1 modulators disclosed in the arts although many may have tried without much success to identify such molecules.

The present invention solves this problem by providing OMA1 modulators. Such compounds may be useful for the treatment of cancer and other disorders and diseases for which OMA1 activation and/or OPA1 cleavage is desired. The compounds can be used in medical therapy, alone or in combination, for example to treat cancer, angiogenesis, cardiovascular disease, neurological disease, eye disease, inflammation, autoimmune disease, and for regulating contraception, and other conditions that are connected to OMA1 and/or OPA1.

Clinical and/or pathological examples for a disorder or disease correlated with mitochondrial dysfunction, for a mitochondrial disorder or disease; or for a disorder or disease characterized by OPA1 alterations and hence, intended to be therapeutically intervened in context of this invention, are given in the non-exhaustive table below.
Ageing; in particular pathological and/or pre-mature aging
Age-related Macular Degeneration (AMD)
Alzheimer's disease
Amyotrophic lateral sclerosis (ALS)
Apoptosis
Ataxia
Autism
Autosomal Dominant Optic Atrophy (ADOA)
Barth syndrome, (familial)
Bipolar disorder
Cancer (e.g. renal cell and colorectal carcinoma, early liver, prostate, breast, bladder, primary lung, head and neck tumors, astrocytomas, adenocarcinomas in Barrett's esophagus)
Cardiomyopathy
Charcot-Marie-Tooth disease (e.g., Charcot-Marie-Tooth disease type 2a and type 4a)
Congenital lactic acidosis
Crohn disease
Deafness
Diabetes
Diabetic sensory neuropathy
Encephalomyopathy
Endotoxemia
External ophthalmoplegia (e.g. PEO)
Eye diseases
Friedreich's ataxia
Glaucoma
Heart disease
Hepatopathy (e.g. defects in SCO1)
Hepato-cerebral form of mtDNA depletion syndrome
Hereditary sensory neuropathy
Hereditary spastic paraplegia
Infantile encephalopathy
Infantile myopathy
Infectious diseases
Inflammatory diseases
Ischemia-reperfusion injury/Hypoxic damage/Oxidative damage
Kearns-Sayre syndrome
Lactic acidosis
Leber's hereditary opticus neuropathy (LHON)
Leigh's syndrome
Leukodystrophy
Metabolic disorders (e.g. defective glucose and fatty acid metabolism)
Mitochondrial neurogastrointestinal-encephalomyopathy
Mohr-Tranebjaerg-syndrome
Motor neuron disorders
mtDNA depletion syndrome
Multiple Sclerosis (MS)
Myoclonus epilepsy and ragged-red fibers syndrome (MERRF)
Myopathy
Myopathy encephalopathy lactic acidosis and stroke-like episodes (MELAS)
Myositis
Neurodegenerative disorders
Non-alcoholic fatty liver disease
Obesity
Ocular myopathy
Optic neuropathy
Optic atrophy type 1
Optic atrophy types 2 to 11
Paraganglioma (e.g. defects in complex II/SDH)
Parkinson's disease
Pearson's syndrome
Respiratory chain disorder
Rhabdomyolysis
Schizophrenia
Sideroblastic anemia
Stroke
Tubulopathy (e.g. defects in BCS1L)
Viral and bacterial infections
Wolf-Hirschhorn syndrome
Wolfram syndrome

However, the disorders or diseases to be medically intervened in context of this invention are not strictly construed to the clinical and/or pathological situations described above.

The technical problem underlying the present invention is the provision of suitable means and methods for therapeutic intervention against mitochondrial dysfunction and diseases or disorders related thereto. Further, means and methods for determining the susceptibility for, predisposition for, and/or the presence of such a disease or disorder are of need.

The solution to the above technical problem is achieved by providing the embodiments characterized in the claims.

The present invention solves the above identified technical problem since, as documented herein below and in the appended examples, it was found that:
i) a disorder or disease correlated with mitochondrial dysfunction, or a mitochondrial disorder or disease, or a disorder or disease characterized by OPA1 alterations also correlated with changes in the activity of OMA1 and/or an oligomeric complex comprising OMA1 and/or HIDG1A and/or OPA1 and/or BNIP3 and/or YMEIL1 and/or PHB and/or SAMM50 and/or IMMT and/or PHB2 or a variant thereof.
ii) measurements of the changes of OMA1 and/or an oligomeric complex comprising OMA1 and/or HIDG1A and/or OPA1 and/or BNIP3 and/or YMEIL1 and/or PHB and/or SAMM50 and/or IMMT and/or PHB2 or a variant thereof informed selection of suitable interventions for patients with such a disorder or disease and in need of medical intervention.
iii) administering a pharmaceutically active amount of a compound capable of adjusting said changes of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof ameliorated, prevented and/or treated said disorder or disease.

In this context, it is evident that measuring the activity and/or the gene expression levels and/or the protein levels of OMA1 and/or an oligomeric complex comprising OMA1 and/or HIDG1A and/or OPA1 and/or BNIP3 and/or YMEIL1 and/or PHB and/or SAMM50 and/or IMMT and/or PHB2 or a variant thereof can be utilized for determining the susceptibility for, predisposition for or the presence of a disorder or disease correlated with mitochondrial dysfunction or characterized by OPA1 alterations, as well as for selecting the appropriate medical interventions. The term biomarker(s) may be utilized mutatis mutandis to describe the result from such measurements.

It is of note that the present invention is particularly useful in the treatment, prevention and/or amelioration of a disease or disorder described herein before any clinical and/or pathological symptoms are diagnosed or determined or can be diagnosed or determined by the attending physician. Thereby, prior to the herein disclosed medical interventions, particular advantage can and shall also be taken of the means and methods disclosed herein for determining the susceptibility for, predisposition for or the presence of a corresponding disorder or disease.

In an additional main aspect, the present invention relates to a method of screening for a compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof comprising the steps of
(a) contacting OPA1 with OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof in the presence of said compound to be screened for under conditions allowing OPA1 processing to occur; and
(b) evaluating whether OPA1 processing is altered compared to a control, wherein OPA1 and OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof are contacted in the absence of said compound to be screened for under conditions allowing OPA1 processing to occur (herein referred to as “control sample”).

The herein disclosed method of screening may further comprise the step of determining the extent of OPA1 processing in the test sample and in the control sample and/or the step of comparing the corresponding results from the test sample with those of the control sample. Thereby, if the extent of OPA1 processing in the test sample differs from that of the control sample, the compound to be screened for is considered to be a modulator of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof, i.e. a “compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof in accordance with the present invention.

If the extend of OPA1 processing in the test sample exceeds that of the control sample, the compound screened is considered to be an “agonist” of said oligomeric complex in accordance with the present invention. If the extend of OPA1 processing in the test sample falls short of that of the control sample, the compound screened is considered to be an “antagonist” of said oligomeric complex in accordance with the present invention.

The terms “agonist” and “antagonist” are known in the arts and it is to be understood that both agonists as well as antagonists capable of modulating the activity of OMA1 and/or an oligomeric complex comprising OMA1 and/or HIDG1A and/or OPA1 and/or BNIP3 and/or YMEIL1 and/or PHB and/or SAMM50 and/or IMMT and/or PHB2 or a variant thereof can provide desirable effects for a patient in need of medical intervention.

A person skilled in the art is, based on the teaching provided herein, readily in a position to select the appropriate medical intervention, i.e., agonist or antagonist, for a patient in need of such intervention based on the evaluation of said biomarker(s). Suitable means and methods for therapeutic intervention and selection of these interventions based on the disclosed biomarker(s) may be referred to as personalized medicine and/or precision medicine.

According to the present invention, methods for the treatment, prevention and/or amelioration of
(i) a disorder or disease correlated with mitochondrial dysfunction, or a mitochondrial disorder or disease; or
(ii) a disorder or disease characterized by OPA1 alterations, comprise the administration to a patient in need of medical intervention a pharmaceutically active amount of a compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof.

According to the present invention, methods of screening for a compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof comprise the steps of
(a) contacting OPA1 with said OMA1 and/or oligomeric complex comprising OMA1 and/or HIGD1A and/or OPA1 and/or BNIP3 and/or YMEIL1 and/or PHB and/or PHB2 and/or SAMM50 and/or IMMT or a variant thereof in the presence of said compound to be screened for under conditions allowing OPA processing to occur; and
(b) evaluating whether OPA1 processing is altered compared to a control, where OPA1 and OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof are contacted in the absence of said compound to be screened for under conditions allowing OPA1 processing to occur.

According to the present invention, methods for determining the susceptibility for, predisposition for or the presence of
(i) a disorder or disease correlated with mitochondrial dysfunction or a mitochondrial disorder or disease; or
(ii) a disorder or disease characterized by OPA1 alterations, comprise the steps of
(a) obtaining a sample from the subject and measuring the activity of OMA1 and/or YMEIL1 or a combination thereof in the sample, and/or measuring the gene expression levels of OMA1, HIGD1A, OPA1, BNIP3, YMEIL1, PHB, SAMM50, IMMT and/or PHB2 or a combination thereof in the sample, and/or measuring the protein levels of OMA1, HIGD1A, OPA1, BNIP3, YMEIL1, PHB, SAMM50, IMMT and/or PHB2 or a combination thereof in the sample;
(b) comparing the increase and/or decrease of measured activity and/or gene expression levels and/or protein levels of OMA1 and/or HIGD1A and/or OPA1 and/or BNIP3 and/or YMEIL1 and/or PHB and/or SAMM50 and/or IMMT and/or PHB2 or a combination thereof in the sample compared to a reference;
(c) integrating the results of these measurements through combination of 3 or more genes selected from the group of OMA1, HIGD1A, OPA1, BNIP3, YMEIL1, PHB, SAMM50, IMMT and PHB2.

According to the present invention, compounds are capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof for the treatment, prevention and/or amelioration of:
(i) a disorder or disease correlated with mitochondrial dysfunction or a mitochondrial disorder or disease; or
(ii) a disorder or disease characterized by OPA1 alterations, wherein said oligomeric complex is defined as in claim 1, said compound is defined as in claim 1, said disorder or disease is defined as in claim 3 and/or said OPA1 alterations, wherein said altered OPA1 processing is characterized by an altered (decrease of a) certain amount of at least one large isoform of OPA1, an altered (increase of a) certain amount of at least one small isoform of OPA1 and/or an altered (decrease of a) certain ratio of at least one large versus at least one small isoform of OPA1 compared to a control/standard.

According to the present invention, methods of treating a disease or disorder in a patient comprise administering to a patient in need of such treatment a therapeutically effective amount of a compound according to the present invention.

According to the present invention, pharmaceutical compositions comprise a compound according to the present invention and a pharmaceutically acceptable excipient.

According to the present invention, methods of treating a disease or disorder in a patient comprise administering to a patient in need of such treatment a therapeutically effective amount of the pharmaceutical composition according to the present invention.

According to the present invention, a combination two or more methods according to the present invention result in a medical intervention individualized for one or more patients and that may be referred to as personalized medicine and/or precision medicine.

### BRIEF DESCRIPTION OF THE DRAWINGS

The drawings described herein are for illustration purposes only. The drawings are not intended to limit the scope of the present disclosure.

FIG. 1 shows an Illustration of the 5 human OPA1 isoforms resolved by SDS-PAGE/Western-blot.

FIG. 2. (A) OMA1 gene expression levels in the hippocampus (AD: n=8; C: n=9) from post-mortem samples of patients with Alzheimer's disease (AD) and control subjects (C). (B) OMA1 gene expression levels in the temporal cortex (AD: n=13; C: n=16) from post-mortem samples of patients with Alzheimer's disease (AD) and control subjects (C). (C) OPA1 gene expression levels in the hippocampus (AD: n=8; C: n=9) from post-mortem samples of patients with Alzheimer's disease (AD) and control subjects (C). (D) OPA1 gene expression levels in the temporal cortex (AD: n=13; C: n=16) from post-mortem samples of patients with Alzheimer's disease (AD) and control subjects (C). (E) BNIP3 gene expression levels in the hippocampus (AD: n=8; C: n=9) from post-mortem samples of patients with Alzheimer's disease (AD) and control subjects (C). (F) BNIP3 gene expression levels in the temporal cortex (AD: n=13; C: n=16) from post-mortem samples of patients with Alzheimer's disease (AD) and control subjects (C).

FIG. 3. (A) SAMM50 gene expression levels in the hippocampus (AD: n=8; C: n=9) from post-mortem samples of patients with Alzheimer's disease (AD) and control subjects (C). (B) SAMM50 gene expression levels in the frontal cortex (AD: n=16; C: n=17) from post-mortem samples of patients with Alzheimer's disease (AD) and control subjects (C). (C) IMMT gene expression levels in the hippocampus (AD: n=8; C: n=9) from post-mortem samples of patients with Alzheimer's disease (AD) and control subjects (C). (D) IMMT gene expression levels in the frontal cortex (AD: n=16; C: n=17) from post-mortem samples of patients with Alzheimer's disease (AD) and control subjects (C). (E) HIGD1A gene expression levels in the hippocampus (AD: n=8; C: n=9) from post-mortem samples of patients with Alzheimer's disease (AD) and control subjects (C). (F) HIGD1A gene expression levels in the frontal cortex (AD: n=16; C: n=17) from post-mortem samples of patients with Alzheimer's disease (AD) and control subjects (C).

FIG. 4. (A) shows expression levels of ONA1 in human hepatocytes for Huh7.5.1 cells not exposed and exposed to Ribavirin (CAS #36791-04-5). (B) shows expression levels of ONA1 in human hepatocytes for Huh7.5.1 cells not exposed and exposed to Ribavirin (CAS #36791-04-5).

FIG. 5(A) shows OMA1 gene expression levels in post mortem samples of the substantia nigra from a cohort of healthy subjects (C; n=9) and patients with Parkinson's disease (PD; n=16) collected at Rochester, Minn. (GEO accession number: GDS2821). (B) shows OPA1 gene expression levels in post mortem samples of the substantia nigra from a cohort of healthy subjects (C; n=9) and patients with Parkinson's disease (PD; n=16) collected at Rochester, Minn. (GEO accession number: GDS2821). (C) shows HIGD1A gene expression levels in post mortem samples of the substantia nigra from a cohort of healthy subjects (C; n=9) and patients with Parkinson's disease (PD; n=16) collected at Rochester, Minn. (GEO accession number: GDS2821). (D) shows IMMT gene expression levels in postmortem samples of the substantia nigra from a cohort of healthy subjects (C; n=9) and patients with Parkinson's disease (PD; n=16) collected at Rochester, Minn. (GEO accession number: GDS2821). (E) shows SAMM50 gene expression levels in post mortem samples of the substantia nigra from a cohort of healthy subjects (C; n=9) and patients with Parkinson's disease (PD; n=16) collected at Rochester, Minn. (GEO accession number: GDS2821). (F) shows BNIP3 gene expression levels in post mortem samples of the substantia nigra from a cohort of healthy subjects (C; n-9) and patients with Parkinson's disease (PD; n=16) collected at Rochester, Minn. (GEO accession number: GDS2821).

FIG. 6(A) shows OPA1 gene expression levels in post mortem samples of the substantia nigra from an independent cohort of healthy subjects (C; n=15) and patients with Parkinson's disease (PD; n=11) collected at Syracuse, N.Y. (GEO accession number: GSE20292). (B) shows HIGD1A gene expression levels in postmortem samples of the substantia nigra from an independent cohort of healthy subjects (C; n=15) and patients with Parkinson's disease (PD; n=11) collected at Syracuse, N.Y. (GEO accession number: GSE20292). (C) shows BNIP3 gene expression levels in post mortem samples of the substantia nigra from an independent cohort of healthy subjects (C; n=15) and patients with Parkinson's disease (PD; n=11) collected at Syracuse, N.Y. (GEO accession number: GSE20292). (D) shows IMMT gene expression levels in post mortem samples of the substantia nigra from an independent cohort of healthy subjects (C; n=15) and patients with Parkinson's disease (PD; n=11) collected at Syracuse, N.Y. (GEO accession number: GSE20292). (E) shows YMEIL1 gene expression levels in post mortem samples of the substantia nigra from an independent cohort of healthy subjects (C; n=15) and patients with Parkinson's disease (PD; n=11) collected at Syracuse, N.Y. (GEO accession number: GSE20292). (F) shows SAMM50 gene expression levels in post mortem samples of the substantia nigra from an independent cohort of healthy subjects (C; n=15) and patients with Parkinson's disease (PD; n=11) collected at Syracuse, N.Y. (GEO accession number: GSE20292).

FIG. 7. (A) shows OPA1 gene expression levels in the prefrontal cortex from healthy subjects (C; n=15) and patients with Parkinson's disease (PD; n=11) from Syracuse, N.Y. (GEO accession number: GSE20168). (B) shows HIGD1A gene expression levels in the prefrontal cortex from healthy subjects (C; n=15) and patients with Parkinson's disease (PD; n=11) from Syracuse, N.Y. (GEO accession number: GSE20168). (C) shows BNIP3 gene expression levels in the prefrontal cortex from healthy subjects (C; n=15) and patients with Parkinson's disease (PD; n=11) from Syracuse, N.Y. (GEO accession number: GSE20168). (D) shows IMMT gene expression levels in the prefrontal cortex from healthy subjects (C; n=15) and patients with Parkinson's disease (PD; n=11) from Syracuse, N.Y. (GEO accession number: GSE20168). (E) shows YMEIL1 gene expression levels in the prefrontal cortex from healthy subjects (C; n=15) and patients with Parkinson's disease (PD; n=11) from Syracuse, N.Y. (GEO accession number: GSE20168). (F) shows PAB gene expression levels in the prefrontal cortex from healthy subjects (C; n=15) and patients with Parkinson's disease (PD; n=11) from Syracuse, N.Y. (GEO accession number: GSE20168).

FIG. 8 shows data ranges for OPA1, OMA1, HIGD1A, BNIP3, YMEIL1, PHB, PHB2, SAMM50 and IMMT gene expression levels in tissue samples from patients with breast cancer (top) and lung cancer (bottom).

FIG. 9 shows data ranges for OPA1, OMA1, HIGD1A, BNIP3, YMEIL1, PHB, PHB2, SAMM50 and IMMT gene expression levels in tissue samples from patients with gastric cancer (top) and ovarian cancer (bottom).

FIG. 10 shows Kaplan-Meier curves showing the overall survival of 1764 patients with breast cancer (average; dark grey, solid line; GEO accession numbers: E-MTAB-365, GSE12276, GSE16391, GSE16446, GSE17907, GSE19615, GSE20685, GSE20711, GSE21653, GSE42568, GSE9195) stratified by a proprietary 3-gene signature based on OMA1, HIGD1A and BNIP3 expression levels (i.e., OMA1: high, HIGD1A: high, BNIP3: low).

FIG. 11 shows Kaplan-Meier curves showing the overall survival of 1145 patients with lung cancer (average; dark grey, solid line; GEO accession numbers: GSE19188, GSE3141, GSE50081, GSE37745, GSE29013, GSE30219, GSE31210) stratified by the proprietary 3-gene signature (i.e., OMA1: high, HIGD1A: high, BNIP3: low).

FIG. 12 shows Kaplan-Meier curves showing the overall survival of 631 patients with gastric cancer (average; dark grey, solid line; GEO accession numbers: GSE22377, GSE15459, GSE51105, GSE62254, GSE62254) stratified by the proprietary 3-gene signature (i.e., OMA1: high, HIGD1A: high, BNIP3: low).

FIG. 13 shows Kaplan-Meier curves showing the overall survival of 1764 patients with breast cancer (average; dark grey, solid line; GEO accession numbers: E-MTAB-365, GSE12276, GSE16391, GSE16446, GSE17907, GSE19615, GSE20685, GSE20711, GSE21653, GSE42568, GSE9195) stratified by a proprietary 6-gene signature based on OMA1, HIGD1A, OPA1, BNIP3, YMEIL1 and IMMT expression levels.

FIG. 14 shows Kaplan-Meier curves showing the overall survival of 1145 patients with lung cancer (average; dark grey, solid line; GEO accession numbers: GSE19188, GSE3141, GSE50081, GSE37745, GSE29013, GSE30219, GSE31210) stratified by a proprietary 6-gene signature based on OMA1, HIGD1A, YMEIL1, PHB, SAMM50 and PHB2 expression levels.

FIG. 15 shows Kaplan-Meier curves showing the overall survival of 631 patients with gastric cancer (average; dark grey, solid line; GEO accession numbers: GSE22377, GSE15459, GSE51105, GSE62254, GSE62254) stratified by a proprietary 7-gene signature based on OMA1, HIGD1A, YMEIL1, PHB, SAMM50 and PHB2 expression levels.

FIG. 16 shows Kaplan-Meier curves showing the overall survival of 1435 patients with ovarian cancer (average; dark grey, solid line; GEO accession numbers: GSE51373, GSE9891, GSE15622, GSE26712, GSE26193, GSE63885, GSE65986, GSE30161, GSE14764, TCGA) stratified by a proprietary 5-gene signature based on OPA1, BNIP3, YMEIL1, IMMT, SAMM50 and PHB expression levels.

FIG. 17 shows the cleavage of OPA isoforms following exposure of HEK293T cells to thiophan.

FIG. 18 shows the cleavage of OPA isoforms following exposure of HEK293T cells to phenanthroline.

FIG. 19 shows the cleavage of OPA isoforms following exposure of HEK293T cells to SB-3CT.

FIG. 20 shows a list of different drugs and compounds that can modify OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof.

FIG. 21 shows OMA1 gene expression levels of KMH2 cells following exposure to glucosamine.

FIG. 22(A) shows OMA1 gene expression levels of SUM139 cells following exposure to micro-RNA miR-203 (NCBI Reference Sequence: NR_029620.1). (B) shows OMA1 gene expression levels of SUM139 cells following exposure to micro-RNA miR-203 (NCBI Reference Sequence: NR_029620.1).

FIG. 23 shows OMA1 gene expression levels of RS4.11 cells following exposure to GSK-3 inhibitor SB216763.

FIG. 24(A) shows OMA1 gene expression levels in monocyte derived macrophages following exposure to small cytokine CXCL4. (B) shows OMA1 gene expression levels in monocyte derived macrophages following exposure to small cytokine CXCL4.

FIG. 25 shows OMA1 gene expression levels in rat brain following exposure to isoflurane.

FIG. 26(A) shows OPA1 gene expression levels in tobacco smokers and non-smokers. (B) shows PHB gene expression levels in tobacco smokers and non-smokers. (C) shows YMEIL1 gene expression levels in tobacco smokers and non-smokers. (D) shows BNIP3 gene expression levels in tobacco smokers and non-smokers. (E) shows PHB2 gene expression levels in tobacco smokers and non-smokers. (F) shows OMA1 gene expression levels in tobacco smokers and non-smokers.

FIG. 27. Tipranavir can prevent CCCP-induced OPA1 cleavage. An OPA1-Western blot shows Hek293T cells incubated with 100 M tipranavir (TPV) in minimal essential media for 1 hour did not hydrolyze the large isoforms of the OPA1 protein upon exposure to 3 μM CCCP for 30 minutes.

FIG. 28. Tipranavir shows dose-dependent antagonistic effects on OMA1/CCCP-induced OPA1 cleavage. An OPA1-Western blot shows Hek293T cells, which were cultured in minimal essential media for 1 hour with the indicated tipranavir (TPV) concentrations. OMA1 was activated with 3 μM CCCP for 30 minutes.

FIG. 29. Tipranavir can activate OMA1 at concentrations above 20 μM. An OPA1-Western blot shows Hek293T cells, which were cultured in minimal essential media for 1 hour with the indicated tipranavir (TPV) concentrations. OMA1 was then activated with 4 μM CCCP for 30 minutes in the indicated samples.

FIG. 30. Tipranavir can prevent CCCP-induced OPA1 cleavage and sorafenib-induced OPA1 cleavage. An OPA1-Western blot shows Hek293T cells incubated with 0 μM, 50 μM or 100 μM tipranavir (TPV) in minimal essential media for 1 hour. OMA1 was then activated with either 3 μM CCCP or 30 μM sorafenib (SFB) for 30 minutes in the indicated samples.

FIG. 31. Tipranavir can reduce OMA1 protein levels. An OMA1-Western blot shows Hek293T cells incubated with 50 μM or 100 μM tipranavir (TPV) in minimal essential media for 1 hour.

### DETAILED DESCRIPTION

The terms “personalized medicine” and “precision medicine” are known in the arts and involve, inter alia, the use of molecular markers that characterize a patient's disease to direct the medical care the patient receives.

The terms “agonist” and “antagonist” are known in the arts. If the extend of OPA1 processing in the test sample exceeds that of the control sample, the compound screened is considered to be an “agonist” of said oligomeric complex in accordance with the present invention. If the extend of OPA1 processing in the test sample falls short of that of the control sample, the compound screened is considered to be an “antagonist” of said oligomeric complex in accordance with the present invention.

The term “conditions allowing OPA1 processing to occur” means that OPA1, i.e., one or more of its splice forms, can be proteolytically cleaved to form one or more of the OPA1 isoforms, whenever an agent/compound capable to cleave OPA1, i.e., capable to trigger OPA1 processing, is present. In other words, said “conditions” are such that said agent/compound capable to cleave OPA1 is active.

In general, the term “oligomeric complex comprising OMA1 and/or HIGD1A and/or OPA1 and/or BNIP3 and/or YMEIL1 and/or PHB and/or PHB2 and/or SAMM50 and/or IMMT or a variant thereof” as described and defined in context of this invention (also referred to herein as “oligomeric complex”) refers to a certain kind of protease, OMA1 and any interacting protein.

It is of note that at least one subunit comprised in the herein defined oligomeric complex must be proteolytically active regardless whether the remaining subunits are. Otherwise said oligomeric complex would not be proteolytically active. Irrespective whether active or not, all subunits, however, must be assembly competent with respect to said oligomeric complex.

From the above, it is evident that the herein described “oligomeric complex” can be a homo-oligomeric complex or a hetero-oligomeric complex.

The meaning of the terms “OMA1” and “HIGD1A” and “BNIP3” and “OPA1” and “YME1L1” and “PHB” and “SAMM50” and “IMMT” and “PHB2” is well known in the art and is, if not explicitly prescribed differentially, used accordingly in context of the present invention. In context of this invention, these terms are likewise used to refer to the corresponding nucleotide sequences (e.g. the genes) as well as to the corresponding polypeptides (e.g. the polypeptides encoded by said genes).

In a specific embodiment of this invention, the oligomeric complex as defined and described herein comprises a polypeptide selected from the group consisting of:
(a) a polypeptide comprising an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
(b) a polypeptide comprising an amino acid sequence encoded by a nucleic acid molecule as depicted in SEQ ID NO 1; 3; 5; 7; 9; 11; 13; 15; 17; 19; 21; 23; 25; 27; 31; 33; 35; 37; 39; 41; 43 or 45.
(c) a polypeptide comprising an amino acid sequence encoded by a nucleic acid molecule encoding an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
(d) a polypeptide comprising an amino acid sequence being homologous to the polypeptide of any one of (a) to (c);
(c) a polypeptide comprising an amino acid sequence encoded by a nucleic acid molecule being homologous to the nucleic acid molecule as defined in any one of (b) to (c);
(f) a polypeptide comprising an amino acid sequence encoded by a nucleic acid molecule hybridizing (under stringent conditions) to the complement stand of a nucleic acid molecule as defined in any one of (b) to (c); and
(g) fragment of a polypeptide of any one of (a) to (f).

The polypeptides as defined in (d) to (g) and the nucleic acid molecule as defined in (c) to (g) are, for example, “variants” in accordance with the present invention.

“Homologous” or “homology” as used in context of this invention, for example, means at least 50%, at least 60%, at least 70%, at least 80%, at least 85%, at least 90%, at least 95%, at least 98% or at least 99% identical on the level of the amino acid or nucleic acid sequence. Thereby, the higher values of percentage are preferred.

It is of note that the meaning of the terms “nucleic acid molecule”, “nucleic acid sequence” or “nucleotide sequence”, and the like, as used herein are well known in the art and, for example, comprise DNA (e.g. cDNA or gDNA) and RNA (e.g. mRNA or siRNA).

The term “variant(s)” of the subunits comprised in the “oligomeric complex” is also intended to encompass “a fragment” of said subunits (or of the mentioned variants thereof). Thereby, the term “fragment” means amino acid stretches of at least 50, at least 100, at least 150, at least 200, at least 300, at least 500 or at least 700 amino acids of the “subunits” defined herein, or nucleotide stretches of at least 150, at least 300, at least 450, at least 600, at least 900, at least 1500 or at least 2100 nucleotides of the corresponding nucleic acid sequences defined herein.

In context of the present invention the meaning of the mentioned term “variant(s)” also encompasses conservative amino acid exchanges and further known modifications.

The meanings of terms like “OPA1”, “OPA1 alterations”, “OPA1 processing”, “proteolytic cleavage of OPA1”, “large/small OPA1 isoforms”, and the like, are known in the art (Duvezin-Caubet et al. 2006; Ishihara et al. 2006) and can also be deduced from PCT/EP2007/004466 (claiming priority to U.S. 60/801,484) and PCT/EP2008/005400 (claiming priority to U.S. Ser. No. 12/667,329). Moreover, the corresponding definitions given herein-below, apply here mutatis mutandis.

As mentioned above, a “compound” to be employed, i.e., to be administered, in context of this invention can be any compound “capable of (specifically) modulating the activity, function and/or expression of OMA1 and/or an oligomeric complex comprising OMA1 and/or HIDG1A and/or OPA1 and/or BNIP3 and/or YMEIL1 and/or PHB and/or PHB2 and/or SAMM50 and/or IMMT or a variant thereof”.

In one specific embodiment, such a “compound” is intended to be a compound screened for by the corresponding method of screening of this invention.

Generally, it is intended herein that a “compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof” as employed herein is or comprises an agonist or antagonist of the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof.

The definitions of the term “activity” given herein-above apply here, mutatis mutandis.

In a specific embodiment of this invention an “agonist” or “antagonist” is a molecule compound selected from the group consisting of:
(a) a binding molecule that (specifically) binds to/interacts with OMA1 and/or an oligomeric complex comprising OMA1 and/or HIDG1A and/or OPA1 and/or BNIP3 and/or YMEIL1 and/or PHB and/or PHB2 and/or SAMM50 and/or IMMT or a variant thereof as defined herein or (specifically) binds to/interacts with a nucleic acid molecule encoding (a subunit of) OMA1 and/or an oligomeric complex comprising OMA1 and/or HIDG1A and/or OPA1 and/or BNIP3 and/or YMEIL1 and/or PHB and/or PHB2 and/or SAMM50 and/or IMMT or a variant thereof as defined herein;
(b) a nucleic acid molecule capable of specifically introducing an insertion of a heterologous sequence or a mutation into a nucleic acid molecule encoding (a subunit of) OMA1 and/or an oligomeric complex comprising OMA1 and/or HIDG1A and/or OPA1 and/or BNIP3 and/or YMEIL1 and/or PHB and/or PHB2 and/or SAMM50 and/or IMMT or a variant thereof as defined herein via in vivo mutagenesis;
(c) a nucleic acid molecule capable of specifically reducing the expression of mRNA encoding (a subunit of) OMA1 and/or an oligomeric complex comprising OMA1 and/or HIDG1A and/or OPA1 and/or BNIP3 and/or YMEIL1 and/or PHB and/or PHB2 and/or SAMM50 and/or IMMT or a variant thereof as defined herein by cosuppression; and
(d) a low molecular weight compound or a small molecule, for example being capable of inhibiting the activity of OMA1 and/or an oligomeric complex comprising OMA1 and/or HIDG1A and/or OPA1 and/or BNIP3 and/or YMEIL1 and/or PHB and/or PHB2 and/or SAMM50 and/or IMMT or a variant thereof as defined herein.

Non-limiting examples of a binding molecule as employed in context of this invention are is selected form the group consisting of antibodies, affybodies, trinectins, anticalins, aptamers, PNA, DNA or RNA, and the like.

Based on prior art literature, the person skilled in the art is familiar with obtaining specific binding molecules that may be useful in the context of the present invention. These molecules are directed and bind/interact specifically to or specifically label the oligomeric complex as defined herein or nucleotide sequences encoding a subunit thereof.

For example, such binding molecules may, inter alia, be selected from the group consisting of:
(a) an antibody that specifically binds to the polypeptide or the nucleic acid molecule as defined herein-above or to (a subunit of) OMA1 and/or an oligomeric complex comprising OMA1 and/or HIDG1A and/or OPA1 and/or BNIP3 and/or YMEIL1 and/or PHB and/or PHB2 and/or SAMM50 and/or IMMT or a variant thereof as defined herein;
(b) an antisense nucleotide sequence that specifically hybridizes to the nucleic acid molecule as defined herein-above;
(c) a siRNA that specifically interacts with the nucleic acid molecule as defined herein-above;
(d) an aptamer that specifically binds to the polypeptide or the nucleic acid molecule as defined herein-above or to (a subunit of) OMA1 and/or an oligomeric complex comprising OMA1 and/or HIDG1A and/or OPA1 and/or BNIP3 and/or YMEIL1 and/or PHB and/or PHB2 and/or SAMM50 and/or IMMT or a variant thereof as defined herein; and
(e) a ribozyme that specifically interacts with the nucleic acid molecule as defined herein-above.

A binding molecule (for example an antibody) to be employed in context of this invention may, for example, (specifically) bind to a particular epitope of the herein defined (subunit(s) of) OMA1 and/or an oligomeric complex comprising OMA1 and/or HIDG1A and/or OPA1 and/or BNIP3 and/or YMEIL1 and/or PHB and/or PHB2 and/or SAMM50 and/or IMMT or a variant thereof. Preferably, this particular epitope is essential for the activity of said complex, like, for example, an epitope comprising the active center of said complex. Moreover, such an epitope may, for example, comprise the consensus amino acid sequence of the metal binding site.

In this context, it is to be understood that the person skilled in the art is, based on the teaching provided herein, readily in a position to deduce (further) amino acid stretches/peptides being specific for (a particular subunit of) the oligomeric complex defined herein and therefore, representing an “epitope” as employed herein.

The antibody useful as a binding molecule in context of the present invention (commonly known as therapeutic antibody) can be, for example, polyclonal or monoclonal. The term “antibody” also comprises derivatives or fragments thereof which still retain the binding specificity. Techniques for the production of antibodies are well known in the art and described, e.g. in Harlow and Lane “Antibodies, A Laboratory Manual”, CSH Press, Cold Spring Harbor, 1988. These antibodies can be used as particular binding molecules defined herein. Surface plasmon resonance as employed in the BIAcore system can be used to increase the efficiency of phage antibodies which bind to an epitope of the polypeptide/complex employed in this invention. Accordingly, also phage antibodies can be used in context of this invention.

The present invention furthermore includes the use of chimeric, single chain and humanized antibodies, as well as antibody fragments, like, inter alia, Fab fragments. Antibody fragments or derivatives further comprise F(ab′)2, Fv or scFv fragments. Accordingly, in context of the present invention, the term “antibody molecule” relates to full immunoglobulin molecules as well as to parts of such immunoglobulin molecules. Furthermore, the term relates, as discussed above, to modified and/or altered antibody molecules, like chimeric and humanized antibodies. The term also relates to monoclonal or polyclonal antibodies as well as to recombinantly or synthetically generated/synthesized antibodies. The term also relates to intact antibodies as well as to antibody fragments thereof, like, separated light and heavy chains, Fab, Fab/c, Fv, Fab′, F(ab′)2. The term “antibody molecule” also comprises bifunctional antibodies, trifunctional antibodies and antibody constructs, like single chain Fvs (scFv) or antibody-fusion proteins.

Non-limiting examples of a low molecular weight compound or a small molecule to be employed as “agonist” or “antagonists” herein are any protease inhibitors or metal chelators, such as EDTA (CAS #60-00-4), phenanthroline (CAS #66-71-7), Deferiprone (CAS #30652-11-0), Deferasirox (CAS #201530-41-8) capable of inhibiting, preferably specifically inhibiting, the activity of the oligomeric complex described herein.

Particularly, metalloprotease inhibitors (like phenanthroline, DCI, and the like) are intended to be employed as low molecular weight compound or a small molecule in context of the present invention.

A further low molecular weight compound or a small molecule as employed in context of this invention may, for example, be a nucleotide analog, such as, for example, ATPγS, and the like.

As mentioned, in one particular embodiment, the “agonist” or “antagonist” to be employed is a nucleic acid molecule that leads to a reduction or depletion of the activity of the oligomeric complex defined herein via in vivo mutagenesis. Thereby, without being bound by theory, an insertion of a heterologous sequence or a mutation into a nucleotide sequence encoding a subunit of said complex, leads to a reduction of the amount of said subunit and hence, to a reduced expression of the intact complex. Generally, methods of “in vivo mutagenesis” (also known as “chimeroplasty”) are known in the art. In such methods, a hybrid RNA/DNA oligonucleotide (chimeroplast) is introduced into cells (WO 95/15972; Kren, Hepatology 25 (21997), 1462-1468; Cole-Stauss, Science 273 (1996), 1386-1389). Without being bound by theory, a part of the DNA component of the RNA/DNA oligonucleotide is thereby homologous to a nucleotide sequence occurring endogenously in the cell and encoding a corresponding protein, but displays a mutation or comprises a heterologous part which lies within the homologous region. Due to base pairing of the regions of the RNA/DNA oligonucleotide which are homologous to the endogenous sequence with these sequences, followed by homologous recombination, the mutation or the heterologous part contained in the DNA component of the oligonucleotide can be introduced into the cell genome. This leads to a reduction of the activity, i.e., expression, of the gene, into which the heterologous part or the mutation has been introduced.

In view of the above, it is clear that the nucleic acid molecule causing in vivo mutagenesis may comprise a heterologous sequence or a sequence carrying a mutation flanked by parts of a nucleotide sequence encoding a subunit of the oligomeric complex defined herein.

In a further particular embodiment of the invention, the “agonist” or “antagonist” to be employed is a nucleic acid molecule that leads to a reduction or depletion of the activity of the oligomeric complex defined herein by a cosuppression effect. “Cosuppression effect” means that the synthesis of a nucleotide sequence, particularly of an RNA, in a living cell reduces the expression of a gene being homologous to said nucleotide sequence. The general principle of cosuppression and corresponding methods are well known to the person skilled in the art and are described, for example, in Pal-Bhadra (Cell 90, 1997), 479-490) and Birchler (Nature Genetics 21 (1999), 148-149). In a particular embodiment, the nucleic acid molecule causing a cosuppression effect comprises a nucleotide sequence encoding a subunit of the oligomeric complex defined herein or a fragment of said nucleotide sequence.

In another specific embodiment of this invention an “agonist” is a molecule selected from the group consisting of:
(a) a polypeptide as defined herein above, for example a subunit of the herein defined oligomeric complex or said oligomeric complex itself, or a nucleotide sequence comprising a nucleic acid molecule as defined herein above, for example a nucleic acid molecule encoding a subunit of the herein defined oligomeric complex;
(b) a low molecular weight compound or a small molecule, for example being capable of enhancing the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined herein; and
(c) a binding molecule as defined herein, wherein said binding molecule is agonistic with respect to the activity of the oligomeric complex as defined and described herein (for example an agonistic antibody or agonistic aptamer).

Particularly, a low molecular weight compound or a small molecule as employed in context of this invention may be a compound/molecule having a molecular weight of less than about 2500 g/mol, preferably less than about 1500 g/mol, more preferably less than about 1000 g/mol and most preferably less than about 500 g/mol.

The skilled person is readily in the position to find out whether a certain binding molecule as defined herein is an agonist (for example an agonistic antibody or agonistic aptamer) or an antagonist (for example an antisense nucleotide sequence, siRNA or ribozyme).

Based on the findings provided herein, it is envisaged in one embodiment of this invention that particular such oligomeric complexes are administered, the subunit composition of which varies dependent on the tissue affected by the disease or disorder to be addressed, i.e., dependent on cell type specific mitochondrial defects. In other words, based on the teaching provided herein, the subunit composition of the oligomeric complex may be adjusted to a particular tissue affected by a disorder or disease described herein.

It is envisaged herein that the compound to be administered in accordance with this invention may, optionally, comprise a pharmaceutically acceptable carrier and/or diluent.

Examples of suitable pharmaceutically acceptable carriers, excipients and/or diluents are well known in the art and include phosphate buffered saline solutions, water, emulsions, such as oil/water emulsions, various types of wetting agents, sterile solutions etc. Compositions comprising such carriers can be formulated by well-known conventional methods. The resulting pharmaceutical compositions can be administered to the subject at a suitable dose, i.e., a dose leading to a pharmaceutically active amount of the compound to be employed/used herein at its desired site of effect.

Administration of the compound to be administered in accordance with the present invention may be effected by different ways, e.g., by intravenous, intraperitoneal, subcutaneous, intramuscular, topical, intradermal, intranasal or intrabronchial administration (for example as effected by inhalation) or by direct administration (for example injection) into a particular tissue or organ.

The dosage regimen of the compound to be administered in accordance with this invention will be determined by the attending physician and clinical factors. As it is well known in the medical arts, dosages for any one patient depends upon many factors, including the patient's size, body surface area, age, the particular compound to be administered, sex, time and route of administration, general health, and other drugs being administered concurrently. A person skilled in the art is aware of and is able to test the relevant doses, the compounds to be used in terms of the present invention are to be administered in.

In the context of the invention, it is of note that a preferred subject/patient in the context of the present invention is a mammalian subject/patient, more preferably a primate subject/patient, most preferably a human being, preferably in need of medical intervention, either in form of treatment, prevention and/or amelioration.

In a particular embodiment, the method for medical intervention provided, and hence the corresponding compound to be administered, are envisaged to be employed in context of gene therapy. This is particularly envisaged, when the “compound” as employed herein is or comprises a nucleic acid molecule or is encoded by a nucleic acid molecule. For example, such corresponding nucleic acid molecule may then be employed in form of an insert comprised in a vector, particularly in an expression vector. Such (expression) vector may particularly be a vector suitable for gene therapy approaches (for example a viral (expression) vector).

Gene therapy, which is based on introducing therapeutic genes into cells by ex-vivo or in-vivo techniques is one of the most important applications of gene transfer. Suitable vectors, methods or gene-delivering systems for in-vitro or in-vivo gene therapy are described in the literature and are known to the person skilled in the art.

In another particular embodiment, the method for medical intervention provided, and hence the corresponding compound to be administered, are envisaged to be employed in context of gene silencing through RNAi (RNA-interference) by use of short interfering RNA (siRNA) or any other approach suitable to suppress gene expression and/or protein levels by at least 5%, 10%, 20%, 30%, 40%, 50%, 60%, 70% 80%, 90% 95%, or 99%.

The CRISPR/Cas9 systems has emerged as a powerful tool for genomic modification and regulation of gene expression in mammalian cells. In other aspects, the invention is envisioned to also provide compositions and methods for targeted regulation of endogenous genes encoding OMA1 and/or an oligomeric complex comprising OMA1 and/or HIDG1A and/or OPA1 and/or BNIP3 and/or YMEIL1 and/or PHB and/or PHB2 and/or SAMM50 and/or IMMT or a variant thereof as defined herein by use of the CRISPR-Cas reagents. Like the compositions and methods for genome editing, the compositions and methods for genome modulation by transcription regulation utilize the highly targeted DNA binding activity of artificial CRISPR-Cas components, where targeting of a modified Cas9 protein to a promoter region of interest can result in either transcriptional activation or transcriptional repression. The invention also provides for multiplex gene regulation, where more than one gene can be simultaneously targeted for transcriptional modulation (either up regulation or down regulation).

The nucleic acid molecules and vectors may be designed for direct introduction or for introduction via liposomes, viral vectors (e.g. adenoviral, retroviral), electroporation, ballistic (e.g. gene gun) or other delivery systems into the cell. Additionally, a baculoviral system can be used as eukaryotic expression system for the above-defined nucleic acid molecules. The introduction and gene therapeutic approach should, preferably, lead to the expression of a functional “compound” in accordance with this invention (for example an antisense or siRNA construct), whereby said expressed “compound” is particularly useful in the treatment, amelioration and/or prevention of the diseases or disorders defined herein.

The term “vector” as used herein particularly refers to plasmids, cosmids, viruses, bacteriophages and other vectors commonly used in genetic engineering. In a preferred embodiment, the vectors of the invention are suitable for the transformation of cells, like fungal cells, cells of microorganisms such as yeast or bacterial cells or animal cells. As mentioned, in a particularly preferred embodiment such vectors are suitable for use in gene therapy.

In one aspect of the invention, the vector to be employed is suitable for stable transformation of an organism, and hence is an expression vector. Generally, expression vectors have been widely described in the literature. As a rule, they may not only contain a selection marker gene and a replication-origin ensuring replication in the host selected, but also a promoter, for example a promoter as defined herein, and in most cases a termination signal for transcription. Between the promoter and the termination signal there is in general at least one restriction site or a polylinker which enables the insertion of a nucleotide sequence desired to be expressed.

Examples of vectors suitable to comprise the nucleic acid molecule(s) as employed in context of the present invention are known in the art.

As mentioned, the meanings of terms like “OPA1 alterations”, “OPA1 processing” and “proteolytic cleavage of OPA1” are known in the art and can also be deduced from PCT/EP2007/004466 and PCT/EP2008/005400. These known definitions apply in context of this invention, if not explicitly defined otherwise.

In view of this, “OPA1 alterations” as defined herein is intended to be characterized by a certain amount of at least one large isoform of OPA1, a certain amount of at least one small isoform of OPA1 and/or a certain ratio of at least one large versus at least one small isoform of OPA1. Thereby, the OPA1 isoforms are formed by proteolytic cleavage of OPA1, i.e., of one or more of the OPA1 spliceforms. Usually, in mammalian cells, “OPA1 processing” usually occurs to a relatively moderate extent, referred to herein as “normal OPA1 processing” or simply “OPA1 processing”. In difference to this, “altered OPA1 processing” or “OPA1 alterations” as defined herein is intended to be characterized by an altered amount of at least one large and/or at least one small isoform of OPA1 and/or an altered ratio of at least one large versus at least one small isoform of OPA1 (due to an altered proteolytic cleavage of OPA1) as compared to a control/standard. “Control/standard” in this context means a physiological condition, where “normal OPA1 processing” or simply “OPA1 processing” occurs (For example in healthy living cells, like the HEK293T cells employed herein).

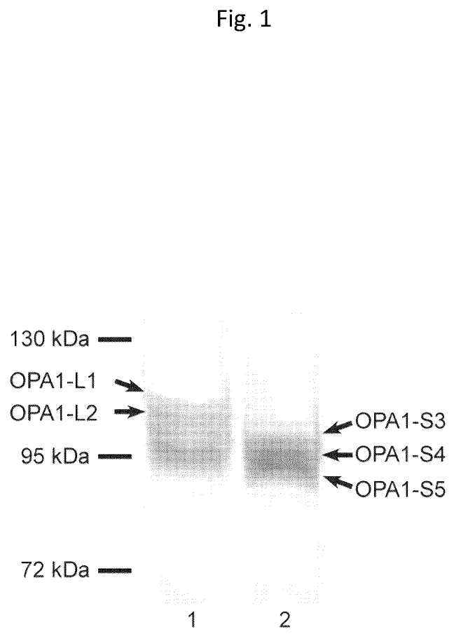

Large isoform(s) of OPA1 as defined herein have an apparent molecular weight of more than about 95 kD and small isoform(s) as defined herein have an apparent molecular weight of less than about 95 kD, when said molecular weights being determined by SDS-PAGE analysis, as disclosed herein and described in the appended drawings (FIG. 1).

It is evident for the person skilled in the art that also other SDS gels and means (in particular Western-Blot analysis and the like) are useful and envisaged in context of the present invention. It is of note that the herein given value of 91 kD is, accordingly, an illustrative example and the person skilled in the art can also use other means to deduce the identity, amount and/or ratio of the herein described OPA1 isoforms (e.g. the presence or absence of said OPA1-isoforms) in a given sample to be analyzed. For example, said large OPA1 isoforms have an apparent molecular weight of more than about 95 kD or, preferably, of more than about 99 kD and the small OPA1 isoforms have an apparent molecular weight of less than about 95 kD or, preferably, of more than about 99 kD, when said molecular weights being determined by peptide analysis, e.g. mass spectrometry.

In context of the present invention, “OPA” or “OPA1” means the optic atrophy 1 protein/gene, in particular OPA1 of human origin. Yet, in certain embodiments it is also envisaged that OPA1 of other organisms, e.g. of mouse, rat, pig, dog, bovine species or fruit fly, be assessed in context of this invention. The nucleotide and amino acid sequences of human OPA1, particularly of the eight spliceforms of OPA1, are given in the appended sequence listing and examples.

The same applies for “OMA1”, “HIGD1A”, “YMEIL1”, “SAMM50”, “IMMT”, “PHB”, “PHB2”, and “BNIP3”, the nucleotide and amino acid sequences of human OMA1, HIGD1A, YMEIL1, SAMM50, IMMT, PHB, PHB2, and BNIP3 are given in the appended

### SEQUENCE LISTING

It is of note that the nucleotide and amino acid sequences of OPA1, OMA1, HIGD1A, YMEIL1, SAMM50, IMMT, PHB, PHB2, and BNIP3 given herein below are not limiting. Accordingly, the terms “OPA1”, “OMA1”, “HIGD1A”, “YMEIL1”, “SAMM50”, “IMMT”, “PHB”, “PHB2”, and “BNIP3” also encompasses OPA1, OMA1, HIGD1A, YMEIL1, SAMM50, IMMT, PHB, PHB2, and BNIP3 proteins/genes having amino acid or nucleotide sequences being derivatives of those given sequences.

In terms of the present invention the term “derivatives” or “derivatives thereof” or “variants” refers to amino acid or nucleotide sequences being homologous to the amino acid or nucleotide sequences shown herein, e.g. those of human OPA1, and/or amino acid or nucleotide sequences as shown herein, e.g. those of human OPA1, but having a particular (conservative) amino acid exchanged. For instance, in context of the present invention, “homologous” means that amino acid or nucleotide sequences have identities of at least 50%, 60%, 70%, 80%, 90%, 95%, 98% or 99% to the sequences shown herein, e.g. those of human OPA1, wherein the higher identity values are preferred upon the lower ones.

As shown herein and in the arts, upon drug-induced apoptosis, processing of OPA1 and mitochondrial fragmentation precedes cytochrome c release. When the mitochondrial membrane potential is dissipated, processing of OPA1 and fragmentation of mitochondria can be observed.

Yet, based on the teaching provided in context of this invention, mitochondrial dysfunction (or a corresponding mitochondrial disease or disorder) is not merely correlated with decrease of any one of OPA1 isoforms, but with a decrease of particularly the large isoforms, e.g. OPA1-LI (as defined herein) and OPA1-L2 (as defined herein), accompanied by an increase of the small isoforms, e.g. OPA1-S3 (as defined herein), OPA1-S4 (as defined herein), and OPA1-S5 (as defined herein).

In context of the present invention, the term “about”, with respect to certain given molecular weight values, means +/−3 kD, preferably +/−2 kD, more preferably +/−1 kD, more preferably +/−0.5 kD and most preferably +/−0.1 kD. Moreover, in context of the present invention, it is envisaged that the term “less than about xx kD”, for example “less than about 91 kD”, “less than about 95 kD” or “less than about 99 kD”, also comprises molecular weight values being equal to xx kD, for example equal to 91, 95 kD or 99 kD.

It is evident for the person skilled in the art that certain given molecular weight values may vary, dependent on the preparational/experimental conditions employed, or, for example with respect to mass spectrometry, dependent on the information content resulting from the preparational/experimental method employed or dependent on an employed modification of the proteins/peptides to be analyzed due to a specific preparational/experimental procedure. It is, for example, known in the art that proteins/peptides to be analyzed via mass spectrometry can be modified, i.e., their theoretical molecular weight can be increased (e.g. by certain chemical modifications) or decreased (e.g. by using a certain protease) by a certain value. It is therefore of note in context of the present invention that the molecular weight values given for certain OPA1 isoforms can change, dependent on the particular preparational/experimental conditions employed during the corresponding mass spectrometry experiment (or other methods for determining molecular weights). The skilled person is readily in the position to deduce whether certain changes/differences of given molecular weight values result from the particular preparational/experimental method employed or form a specific composition of the protein/peptide analyzed.

In context of the present invention, the term “isoform” of OPA1 means a certain form of the OPA1 protein. Without bound by theory, an OPA1 isoform derives from (a protein encoded by) any one of spliceforms 1 to 8 of OPA1, e.g. by posttranslational processing (e.g. proteolytical processing). Without bound by theory, said posttranslational processing (e.g. proteolytical processing) leads to a shortened N-terminus of OPA1, particularly of the spliceforms thereof, wherein the C-terminus remains complete. The “isoforms” of OPA1 to be scrutinized in context of the present invention are described herein in more detail. Accordingly, the term “corresponding” in context of OPA1 isoforms and OPA1 spliceforms, e.g. in the term “an OPA1 isoform having an apparent molecular weight calculated from amino acid sequences of the corresponding spliceform”, means that the respective OPA1 isoform can be related to or may be derived from said OPA1 spliceform. These spliceforms are also described herein below.

In context of the present invention, the term “spliceform” or “splice variant” of OPA1 means a form of OPA1 that emerges by alternative splicing of the primary transcript transcribed from the OPA1 gene. It is envisaged herein, that the term “spliceform” either refers to the mature transcript generated by alternative splicing, but also refers to the corresponding protein which has been translated from said mature transcript. Accordingly, the term “isoform being derived from (corresponding) spliceform” means that an OPA1 isoform originates from a protein that has been translated from a mature (alternatively spliced) transcript of the OPA1 gene. Thereby, posttranslational processing (e.g. proteolytical processing) of said protein that has been translated from a mature (alternatively spliced) transcript of the OPA1 gene may occur. However, an OPA1 isoform may also directly originate from said protein, without further posttranslational processing. In such specific case, said protein then is said OPA1 isoform.

At present, 8 spliceforms of OPA1 are known in the art, which emerge by alternative splicing of exon 4, exon 4b and/or exon 5. The corresponding amino acid sequences of these 8 spliceforms are given in SEQ ID NO: 2, 4, 6, 8, 10, 12, 14 and 16. Their corresponding nucleotide acid sequences are given in SEQ ID NO: 1, 3, 5, 7, 9, 11, 13 and 15.

Dependent whether exon 4, exon 4b and/or exon 5 is comprised, the OPA1 spliceforms can be defined by specific amino acid sequences, e.g. by one of the following amino acid sequences:

EYKWIVPDIVWEIDEYIDFGHKLVSEVIGASDLLLLL (SEQ ID NO: 60) corresponds to the amino acid sequences from exon 3 to exon 4b (lack of exon 4) and is comprised in spliceforms 3 and 6.

EYKWIVPDIVWEIDEYIDFGSPEETAFRATDRGSESDKHFRK (SEQ ID NO: 61) corresponds to the amino acid sequences from exon 3 to exon 5 (lack of exon 4 and 4b) and is comprised in spliceforms 2 and 4.

EKIRKALPNSEDLVKLAPDFDKIVESLSLLKDFFTSGSPEETAFRATDRGSESD KHFRK (SEQ ID NO: 62) corresponds to the amino acid sequences from exon 4 to exon 5 (lack of exon 4b) and is comprised in spliceforms 1 and 7.

GSPEETAFRATDRGSESDKHFRKVSDKEKIDQLQEELLHTQLKYQRILERLEK ENKELRK (SEQ ID NO: 63) corresponds to the amino acid sequences from exon 5 to exon 6 (lack of exon 5b) and is comprised in spliceforms 1, 2, 3 and 5.

Other amino acid sequences specific for a certain OPA1 spliceform can be derived from the amino acid sequences of the OPA1 spliceforms given herein below.

Since an OPA1 isoform to be employed in context of the present invention may be derive from one particular OPA1 spliceform, the above-mentioned amino acid sequences defining the different OPA1 spliceforms may also be used to determine the identity, amount and/or ratio (e.g. the presence or absence) of a given OPA1 isoform as defined herein. For example, since the present invention provides evidence that OPA1-LI be derived from spliceform 7 and OPA1-L2 be derived from spliceform 1, OPA1-LI may, e.g. be characterized in that it comprises the amino acid sequence

EKIRKALPNSEDLVKLAPDFDKIVESLSLLKDFFTSGSPEETAFRATDRGSESDKHFRK (SEQ ID NO: 62) and in that it not comprises the amino acid sequence.

GSPEETAFRATDRGSESDKHFRKVSDKEKIDQLQEELLHTQLKYQRILERLEKENKEL RK (SEQ ID NO: 63) and OPA1-L2 may, e.g. be characterized in that it comprises the amino acid sequence

EKIRKALPNSEDLVKLAPDFDKIVESLSLLKDFFTSGSPEETAFRATDRGSESDKHFRK (SEQ ID NO: 62) and

GSPEETAFRATDRGSESDKHFRKVSDKEKIDQLQEELLHTQLKYQRILERLEKENKEL RK (SEQ ID NO: 63).

However, since the OPA1 isoforms to be employed in context of the present invention may derive from the OPA1 spliceforms by (proteolytical) processing, not the complete amino acid sequences as given above, but fragments or derivatives thereof, may be used to determine a certain OPA1 isoform.

The meaning of the term “Mass spectrometry” (MS) is, and corresponding methods, are known in the art. Particularly useful “mass spectrometry” methods to be employed in context of the present invention are MALDI-MS or LC-MS/MS. Further “mass spectrometry” methods are known in the art and can easily be adapted to the specific needs of the present invention by a person skilled in the art.

The term “molecular weights being determined by mass spectrometry” means that the apparent molecular weight of a certain OPA1 isoform is determined by performing mass spectrometry analysis on said OPA1 isoform and using the results of said mass spectrometry analysis to calculate said apparent molecular weight of said certain OPA1 isoform on the basis of the amino acid sequence of OPA1. Since eight alternative spliceforms exist of OPA1, having different amino acid sequences, the result of said calculation may vary, dependent on the spliceform, the amino acid sequence of which is used for said calculation.

It is of note that the so determined theoretical molecular weight may be further increased by the presence of a few further N-terminally located amino acid residues present in the (proteolytically) processed mature OPA1 isoform. The person skilled in the art is readily in the position to determine said slightly increased molecular weight, by taking advantage of the teaching of the present invention.

In context of the present invention, the large isoforms of OPA1 may comprise two isoforms (e.g. OPA1-LI and OPA1-L2) and the small isoforms of OPA1 may comprise three isoforms (e.g., OPA1-S3, OPA1-S4 and OPA1-S5). However, it is also envisaged that further, possibly existing isoforms may be assigned as large or small isoforms in context of the present invention. For instance, it is evident for a skilled person that, e.g., single bands of an SDS-PAGE/Western-blot as exemplified herein, may represent not only one, but several different isoforms and/or that further isoforms, larger or smaller than the particular isoforms defined herein may be present. For example, particularly the band corresponding to OPA1-S4 as defined herein may correspond to a further OPA1 isoform. Again, the gist of the present invention is based on the fact the determination of “small” versus “large” isoforms is illustrative for mitochondrial dysfunction and corresponding related disorders/diseases. Therefore, further, possibly existing isoforms may, e.g., be detectable by alternative comparable methods known in the art and may also be taken into consideration in the herein provided methods and means. For example, such methods may be SDS-PAGEs taking advantage of gels having very low polyacrylamide concentrations (e.g. 1%, 2%, 3% or 4%) and/or Western-blots taking advantage of radionuclide labelling, e.g. radionucleotide labelling of (secondary) antibodies used in said Western-blots, or other labelling approaches known in the art, e.g. other very sensitive labelling approaches being suitable for the detection of proteins being present in low amount(s)/concentration(s). Moreover, such methods may be a two-dimensional gelelectrophoresis methods. These and other alternative methods for detecting isoforms of certain proteins/genes, like OPA1, are known in the art. It is envisaged that such alternative methods may also be employed in context of the present invention.

However, it is preferred that each single band as evident from the SDS-PAGE analysis as employed and exemplified herein represents one single OPA1 isoform. Accordingly, in one embodiment of the present invention, the two large OPA1 isoforms as defined herein (e.g. OPA1-LI and OPA1-L2) are represented by two single bands, and the three small OPA1 isoforms as defined herein (e.g. OPA1-S3, OPA1-S4 and OPA1-S5) are represented by three single bands occurring in an SDS-PAGE, e.g. an SDS-PAGE as exemplified herein (FIG. 1).

FIG. 1 shows an illustration of the 5 human OPA1 isoforms resolved by SDS-PAGE/Western-blot. Human HEK293T cells were harvested, solubilized in RIPA buffer, and separated utilizing an 8% Tris-Glycine Gel (Novex, ThermoFisher Scientific, CA). The large bands of OPA1 comprise two isoforms, OPA1-LI and OPA1-L2, and the small bands of OPA1 comprise three isoforms, OPA1-S3, OPA1-S4 and OPA1-S5 (lane 1). Under conditions allowing OPA1 processing to occur, large OPA isoforms are proteolytically cleaved and convert into one or more of the small OPA1 isoforms. OPA1 processing was evoked in this particular non-limiting example by uncoupling of the oxidative phosphorylation through addition of 10 μMCCCP for 30 minutes at 37º C (lane 2).

In context of the present invention, the two large OPA1 isoforms are indicated by numbers 1 and 2, namely 1 for the largest and 2 for the second largest OPA1 isoform. The three small OPA1 isoforms are indicated by numbers 3, 4 and 5, namely 3 for the largest of the three small isoforms, 4 for the second largest of the three small isoforms and 5 for the smallest isoform. The numbering of the OPA1 isoforms to be employed in context of the present invention is also given in FIG. 1. In accordance thereto the OPA1 isoforms as employed in context of the present invention are termed as follows: OPA1-L/11, L/1-OPA1 #1, OPA1 #1 or L/11-OPA1 for the largest OPA1 isoform. OPA1-L/12, L/1-OPA1 #2, OPA1 #2 or L/12-OPA1 for the second largest OPA1 isoform. Large isoform(s) in context of the present invention is (are), e.g., OPA1-LI and/or OPA1-L2. OPA1-S/s3, S/s-OPA1 #3, OPA1 #3 or S/s3-OPA1 for the largest of the three small OPA1 isoforms. OPA1-S/s4, S/s-OPA1 #4, OPA1 #4 or S/s4-OPA1 for the second largest of the three small isoforms. OPA1-S/s5, S/s-OPA1 #5, OPA1 #5 or S/s5-OPA1 for the smallest OPA1 isoform. Accordingly, small isoform(s) in context of the present invention is (arc), e.g., OPA1-S3, OPA1-S4 and/or OPA1-S5.

It is of note that the specific numbering is indicative for the specific OPA1 isoform, and that the additional terming, like “1” for large; “s” for small or “OPA”, “OPA1” or “OPA1 #” for OPA1 may slightly vary. However, the abbreviations “L” or “1” indicate large isoforms and “S” or “s” indicate small isoforms of OPA1.

In view of the teaching provided herein, also in the appended examples, the OPA1 isoforms employed in context of the present invention are defined as follows:

In one aspect of the present invention, the term “OPA1 isoform” means a protein encoded by the OPA1 gene, but particularly be derived from at least one of the different spliceforms of OPA1 (e.g. from at least one of spliceforms 1 to 8), e.g. by posttranslational (e.g. proteolytical) processing, wherein said proteins are distinguishable by their molecular weight and/or a certain amino acid sequence. From the above, it is, inter alia, evident that an “OPA1 isoform” as employed in context of the present invention comprises an amino acid stretch which unambiguously characterize it as a polypeptide/protein derived from OPA1. In this context, “derived from OPA1” particularly means encoded by the OPA1 gene and/or generated from OPA1 by the herein described and defined OPA1 processing. Thus, an “OPA1 isoform” as employed can particularly be characterized by a certain amino acid stretch of any one of SEQ ID NO: 2, 4, 6, 8, 10, 12, 14 or 16 or by a certain amino acid stretch encoded by any one of SEQ ID NO: 1, 3, 5, 7, 9, 11, 13 or 15.

In context of the present invention, the term “molecular weight” may, inter alia, refer to the apparent molecular weight. Said apparent molecular weight can be determined by methods known in the art. E.g., said apparent molecular weight can be determined by SDS-PAGE, and, accordingly, also from Western-blots, or can be calculated from the amino acid sequence of OPA1, particularly from the amino acid sequence(s) of the corresponding spliceform(s) by taking advantage of mass spectrometry methods. Examples of the determination of the OPA1 isoforms by using these techniques are given in the appended examples.

As already mentioned above, in context of the present invention, certain given molecular weight values are apparent molecular weight values. It is envisaged, that the certain molecular weight values given herein may slightly vary, e.g. with respect to the molecular weight of the protein present in vivo. Said variation may by in the range of 5 kD, 4 kD, 3 kD, 2 kD, 1 kD, 0.5 kD, 0.4 kD, 0.3 kD, 0.2 kD or 0.1 kD, whereby the smaller variations are preferred over the larger variations. The definitions given for the term “about” with respect to molecular weight values herein above, apply here, mutatis mutandis.

In context of the present invention, large isoforms comprise an isoform having an apparent molecular weight of about 97 kD (96.8 kD) (defined as OPA1-L1) or an isoform having an apparent molecular weight of about 92 kD (92.3 kD) (defined as OPA1-L2), said molecular weights being determined by SDS-PAGE analysis. Moreover, in context of the present invention large isoforms comprise an isoform having an apparent molecular weight of about 104 kD (104.0 kD) or, preferably, of about 105 kD (105.1 kD) (defined as OPA1-L1) or an isoform having an apparent molecular weight of about 99 kD (99.2 kD) or, preferably, of about 100 kD (100.0 kD) (defined as OPA1-L2), said molecular weights being determined by mass spectrometry.

In context of the present invention, small isoforms comprise an isoform having an apparent molecular weight of about 88 kD (88.1 kD) (defined as OPA1-S3), an isoform having an apparent molecular weight of about 84 kD (84.4 kD) (defined as OPA1-S4) or an isoform having an apparent molecular weight of about 81 kD (80.9 kD) (defined as OPA1-S5), said molecular weights being determined by SDS-PAGE analysis. Moreover, in context of the present invention small isoforms comprise an isoform having an apparent molecular weight of about 92 kD (91.8 kD) or, preferably, of about 96 kD (95.9 kD) (defined as OPA1-S3), an isoform having an apparent molecular weight of about 89 kD (89.2 kD) or, preferably, of about 92 kD (91.8 kD) (defined as OPA1-S4) or an isoform having an apparent molecular weight of about 87 kD (86.8 kD) or, preferably, of about 87 kD (86.8 kD) (defined as OPA1-S5), said molecular weights being determined by mass spectrometry.

It is of note that the molecular weight values of the OPA1 isoforms scrutinized herein are given as averaged values corresponding to the molecular weight values of different isoform bands within an SDS-PAGE/Western-blot.

The term “derivatives” or “derivatives thereof” as well as “homologous” as defined herein above, also apply, mutatis mutandis, in context of the peptides shown above, e.g. the peptides comprised in the OPA1 isoforms or the peptides that characterize the OPA1 spliceforms. Moreover, the term “derivatives” or “derivatives thereof” also refers to (a) fragment(s), e.g. a fragment of the peptides shown above, e.g. the peptides comprised in the OPA1 isoforms or the peptides that characterize the OPA1 spliceforms. The term “fragment(s)” means amino acid stretches of at least 3, 4, 5, 6, 7, 8, 9, 10, 11, 12, 13, 14, 15, 20, 30, 50, 100 or 150 amino acids. Also, amino acid stretches having other numbers of amino acids are envisaged.

In terms of the present invention the term “derivatives” or “derivatives thereof” also comprises homologies as well as conservative amino acid exchanges and further known modifications.

In a non-limiting example, it is envisaged in context of the present invention that the identity, amount and/or ratio of the large OPA1 isoforms as defined herein, namely OPA1-L1 and OPA1-L2, can be determined via specific detection of any amino acid stretch of the large OPA1 isoforms lying in N-terminal direction to the amino acid stretches corresponding to the N-terminal amino acids of the “most N-terminal peptide(s)” defined herein of the small OPA1 isoform(s), alternatively and preferred lying in N-terminal direction to the amino acid stretches corresponding to the N-terminal amino acid of the small OPA1 isoforms. In analogy to the above, said amino acid stretch to be detected may be any epitope-bearing portion, or, e.g. any other portion to which a binding molecule as defined herein can bind and said detection may be a detection method as defined and exemplified herein, e.g. a detection method taking advantage of corresponding OPA1 antibodies as defined and exemplified herein, or a detection method taking advantage of other corresponding OPA1 binding molecules as defined herein.

In another particular embodiment of this invention, it is envisaged to distinguish between various types of mitochondrial dysfunction(s)/disease(s). In particular, it is envisaged to differentiate between mitochondrial dysfunction(s)/disease(s) dependent on depletion of mitochondrial DNA and other types of mitochondrial dysfunction(s)/disease(s). Moreover, a quantitative measure of mitochondrial dysfunction and the employment of a corresponding adapted medical intervention is also envisaged.

In context of the present invention, it is intended that the identity, amount or ratio of large and/or small isoforms of OPA1 is determined by optical, spectrophotometric and/or densitometric measurements or analysis. Such determination methods are well known in the art. A particular choice of such methods is described in the appended examples. For instance, such methods comprise the SDS-PAGE analysis, Western blots, ELISA, RIA, CLIA, IRMA and/or EIA. These and further methods are known in the art and are, e.g., described in “Cell Biology: Laboratory manual 3rd edition” (2005, J. Celis, editor. Academic Press, New York).

It is also intended that the identity, amount or ratio of large and/or small isoforms of OPA1 is determined by peptide analysis. Again, such peptide analysis methods are well known in the art. For example, such peptide analysis methods comprise mass spectrometry methods, like MALDI-MS or LC-MS/MS. The use of these particular mass spectrometry methods is described in the appended examples.

As detailed herein one gist of the present invention is based on the finding that a determined reduction of large OPA1 isoforms as described herein (OPA1-LI and/or —L2) and/or a determined increase of small OPA1 isoforms as described herein (OPA1-S3, OPA1-S4 and/or OPA1-S5) is indicative for the presence of or the susceptibility to a mitochondrial disease/disorder/dysfunction.

Ratios between large and small OPA1 isoforms can be deduced from the activity of OMA1 and/or an oligomeric complex comprising OMA1 and/or HIDG1A and/or OPA1 and/or BNIP3 and/or YMEIL1 and/or PHB and/or PHB2 and/or SAMM50 and/or IMMT or a variant thereof as defined herein.

It is understood that the ratios to be determined in context of the present invention may also differ from the ones exemplified above. As already mentioned above, examples that, in a non-limiting manner, describe the evaluation of such ratios are given herein below.

Inter alia, in context of the present invention, the term “ratio” or “density ratio”, inter alia, refers to a comparison of density values of bands corresponding to OPA1 isoforms, as, e.g., derived from an SDS-PAGE/Western-blot. Methods how such density values can be obtained are known in the art and exemplified in the appended non-limiting examples.

It is to be understood that not only the comparison of small (OPA1-S3, —S4 and/or —S5) versus large isoforms of OPA1 (OPA1-LI and/or —L2) or small or large versus other or all OPA1 isoforms derived from an individual patient sample or sample to be tested is of relevance with respect to a certain disorder or disease, but that also a comparison to a healthy control or a corresponding standard is of relevance and can, in accordance with the teachings provided herein, be obtained. This applies, mutatis mutandis, for all methods provided herein.

The person skilled in the art is readily in a position to determine the ratio of individual (or more) OPA1 isoforms as described herein by methods known in the art, like for example densitometric, spectrophotometric, luminescent, autoradiographic or fluorescent quantification methods. Also in this context, methods comprising tests with specific anti-OPA1 isoform antibodies (also specific antibodies against individual OPA1-isoforms as provided herein) are useful. Accordingly, methods, like Western-blot analysis or ELISA/RIA-tests may be employed to determine the OPA1 isoform ratio(s). Corresponding non-limiting examples are illustrated in the appended experimental part.

In context of the present invention, the amount or ratio of large and/or small isoforms of OPA1 can be, inter alia, derived from measurements of the enzymatic activity and/or the gene expression levels and/or the protein levels of OMA1 and/or an oligomeric complex comprising OMA1 and/or HIDG1A and/or OPA1 and/or BNIP3 and/or YMEIL1 and/or PHB and/or PHB2 and/or SAMM50 and/or IMMT or a variant thereof. In an embodiment, these different measurements can be integrated to determine the subject's prognosis based on said integration, wherein depending on the disorder or disease an increase and/or decrease of any of the aforementioned activities and/or levels indicates the subject has a favorable or unfavorable susceptibility for, predisposition for or the presence of the disorder or disease and therefore requires and/or responds to a treatment, prevention and/or amelioration of the disorder or disease. The term “biomarker” refers to such a prognostic method and is well known to the person skilled in the art.

As used herein, the term “subject” and “patient” are used interchangeably and refer to both human and non-human animals. The term “non-human animals” of the disclosure includes all vertebrates, e.g., mammals and non-mammals, such as nonhuman primates, sheep, dog, cat, horse, cow, chickens, rodents, amphibians, reptiles, and the like. Preferably, the subject is a human patient.

As used herein the term, “integration” refers to providing a probability-based analysis of how a particular subject will develop a disorder or disease and/or how a particular subject will require or respond to a treatment. The prediction of responsiveness is not a guarantee or absolute, only a statistically probable indication of the responsiveness of the subject.

The prediction of responsiveness to a therapy may indicate that the subject is likely to be responsive to a therapy or alternatively may indicate that the subject is not likely to be responsive to a therapy. Alternatively, the prediction, may indicate that a method for the treatment, prevention and/or amelioration of a disorder or disease correlated with mitochondrial dysfunction or a mitochondrial disorder or disease, or a disorder or disease characterized by OPA1 alterations regime may be counter-productive and lead to a worse result for the subject than if no therapy was used or a placebo was used. Responsiveness includes but is not limited to, any measure of a likelihood of clinical benefit. For example, clinical benefits include an increase in overall survival, an increase in progression free survival, an increase in time to progression, increased response, decreased symptoms, or other quality of live benefits.

In one embodiment, the method includes determining the expression levels of the proteins or the RNA transcripts for the biomarkers in a sample from a patient with cancer or any other disorder or disease. Biomarker expression in some instances may be normalized against the expression levels of all proteins or RNA transcripts in the sample, or against a reference set of proteins or RNA transcripts in the sample. The level of expression of the biomarkers is indicative of the prognosis for the subject or predictive of the effectiveness of a particular treatment.

The methods of the present disclosure can also be used to assist in selecting appropriate courses of treatment and to identify patients that would benefit; from a particular course of therapy. Thus, the expression of the particular biomarkers described herein provides insight into which treatment regimens will be most effective for the patient. This information can be used, to generate treatment plans for the patient to prolong survival and minimize side effects or therapy related toxicity.

In some embodiments described herein, prognostic performance of the biomarkers and/or other clinical parameters was assessed by determining the threshold of OMA1 activation and/or the activation threshold of an oligomeric complex comprising OMA1 and/or HIDG1A and/or OPA1 and/or BNIP3 and/or YMEIL1 and/or PHB and/or PHB2 and/or SAMM50 and/or IMMT or a variant thereof upon induction of inner membrane permeabilization through addition of mitochondrial uncouplers, such as carbonyl cyanide m-chlorophenyl hydrazine (CCCP; CAS #555-60-2).

It is to be understood that any binding molecule capable of lowering or increasing the threshold levels is an agonist or antagonist in the context of the present invention and therefore administration of such a binding molecule to a patient in need of medical intervention is a method of treatment, prevention and/or amelioration of a disorder or disease correlated with mitochondrial dysfunction or a mitochondrial disorder or disease; or a disorder or disease characterized by OPA1 alterations.

“Sample” is intended to include a sampling of cells, tissues, or bodily fluids in which expression of a biomarker can be detected. Examples of such samples include, but are not limited to, biopsies, cerebrospinal fluid, blood, lymph, urine, saliva, or any other bodily secretion or derivative thereof. Blood can include whole blood, plasma (citrate, EDTA, heparin), serum, or any derivative of blood. Samples may be obtained from a patient by a variety of techniques available to those skilled in the art. Methods for collecting various samples are well known in the art.

Any methods available in the art for detecting expression of biomarkers are encompassed herein. The expression of a biomarker of the invention can be detected on a nucleic acid level (e.g., as an RNA transcript) or a protein level. By “detecting or determining expression” is intended determining the quantity or presence of a protein or its RNA transcript for any combination of at least two genes/proteins from the list compromising OMA1, HIGD1A, OPA1, BNIP3, YMEIL1, PHB, SAMM50, IMMT or PHB2. Thus, “detecting expression” encompasses instances where a biomarker is determined not to be expressed, not to be detectably expressed, expressed at a low level, expressed at a normal level, or overexpressed.

Methods suitable for detecting or determining the expression levels of biomarkers are known to those of skill in the art and include, but are not limited to, ELISA, immunofluorescence, FACS analysis, Western blot, magnetic Immunoassays, and both antibody-based micro arrays and non-antibody-based microarrays. Methods for detecting expression of the biomarkers described herein are not limited to protein expression. Gene expression profiling including methods based on hybridization analysis of polynucleotides, methods based on sequencing of polynucleotides, immunohistochemistry methods, and proteomics-based methods may also be used. Alternatively, antibodies may be employed that can recognize specific duplexes, including DNA duplexes, RNA duplexes, and DNA-RNA hybrid duplexes, or DNA-protein duplexes. Representative methods for sequencing-based gene expression analysis include Serial Analysis of Gene Expression (SAGE) and gene expression analysis by massively parallel signature sequencing.

The term “probe” refers to any molecule that is capable of selectively binding to a specifically intended target biomolecule, for example, a nucleotide transcript or a protein encoded by or corresponding to a biomarker. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

### Aspects of the Invention

Aspect 1. A method for the treatment, prevention and/or amelioration of
(i) a disorder or disease correlated with mitochondrial dysfunction, or a mitochondrial disorder or disease; or
(ii) a disorder or disease characterized by OPA1 alterations, wherein said method comprises the administration to a patient in need of medical intervention a pharmaceutically active amount of a compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof.

Aspect 2. The method of aspect 1, wherein OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof comprises a polypeptide selected from the group consisting of:
(a) a polypeptide comprising an amino acid sequence encoded by a nucleic acid molecule as depicted in SEQ ID NO 1; 3; 5; 7; 9; 11; 13; 15; 17; 19; 21; 23; 25; 27; 31; 33; 35; 37; 39; 41; 43 or 45.
(b) a polypeptide comprising an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
(c) a polypeptide comprising an amino acid sequence encoded by a nucleic acid molecule encoding an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
(d) a polypeptide comprising an amino acid sequence having at least 50% sequence identity to the polypeptide of any one of (a) to (c);
(e) a polypeptide comprising an amino acid sequence encoded by a nucleic acid molecule having at least 50% sequence identity to the nucleic acid molecule as defined in any one of (a) to (c);
(f) a polypeptide comprising an amino acid sequence encoded by a nucleic acid molecule hybridizing under stringent conditions to the complement stand of a nucleic acid molecule as defined in any one of (a) to (c); and
(g) fragment of a polypeptide of any one of (a) to (f).

Aspect 3. The method of aspect 1, wherein OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof is a homo-oligomeric complex or a hetero-oligomeric complex.

Aspect 4. The method of aspect 1, wherein OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof comprises a polypeptide selected from the group consisting of:
(a) a polypeptide comprising an amino acid sequence encoded by a nucleic acid molecule as depicted in SEQ ID NO 1; 3; 5; 7; 9; 11; 13; 15; 17; 19; 21; 23; 25; 27; 31; 33; 35; 37; 39; 41; 43 or 45.
(b) a polypeptide comprising an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
(c) a polypeptide comprising an amino acid sequence encoded by a nucleic acid molecule encoding an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
(d) a polypeptide comprising an amino acid sequence having at least 50% sequence identity to the polypeptide of any one of (a) to (c);
(e) a polypeptide comprising an amino acid sequence encoded by a nucleic acid molecule having at least 50% sequence identity to the nucleic acid molecule as defined in any one of (a) to (c);
(f) a polypeptide comprising an amino acid sequence encoded by a nucleic acid molecule hybridizing under stringent conditions to the complement stand of a nucleic acid molecule as defined in any one of (a) to (c); and
(g) fragment of a polypeptide of any one of (a) to (f).

Aspect 5. The method of aspect 1, wherein OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof is a homo-oligomeric complex or a hetero-oligomeric complex.

Aspect 6. The method of aspect 5, wherein said hetero-oligomeric complex comprises AFG311 and/or AFG312 and/or paraplegin and/or HIGD1a and/or OPA1 and/or BNIP3 and/or YMEIL1 and/or PHB and/or PHB2 or a variant thereof.

Aspect 7. The method of any one of aspects 1 to 6, wherein said compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is a compound obtained by a method comprising the steps of:
(a) contacting OPA1 with OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof in the presence of said compound to be screened for under conditions allowing OPA1 processing to occur; and
(b) evaluating whether OPA1 processing is altered compared to a control, where OPA1 and OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof are contacted in the absence of said compound to be screened for under conditions allowing OPA1 processing to occur.

Aspect 8. The method of any one of aspects 1 to 6, wherein said compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is or comprises an agonist or antagonist of the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof.

Aspect 9. The method of any one of aspects 1 to 6, wherein said compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is or comprises an agonist or antagonist of the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof, wherein said agonist or antagonist is a molecule selected from the group consisting of:
(a) a binding molecule that binds to/interacts with OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1 or binds to/interacts with a nucleic acid molecule encoding (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1;
(b) a nucleic acid molecule capable of introducing an insertion of a heterologous sequence or a mutation into a nucleic acid molecule encoding (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1 via in vivo mutagenesis;
(c) a nucleic acid molecule capable of reducing the expression of mRNA encoding (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1 by co-suppression; and
(d) a low molecular weight compound or a small molecule.

Aspect 10. The method of aspect 9, wherein said binding molecule is selected form the group consisting of antibodies, affybodies, trinectins, anticalins, aptamers, PNA, DNA or RNA.

Aspect 11. The method of any one of aspects 1 to 6, wherein said compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is or comprises an agonist or antagonist of the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof, wherein said agonist or antagonist is a molecule selected from the group consisting of:

(A) a binding molecule that binds to/interacts with OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1 or binds to/interacts with a nucleic acid molecule encoding (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1;
(B) a nucleic acid molecule capable of introducing an insertion of a heterologous sequence or a mutation into a nucleic acid molecule encoding (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1 via in vivo mutagenesis;
(C) a nucleic acid molecule capable of reducing the expression of mRNA encoding (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1 by co-suppression; and
(D) a low molecular weight compound or a small molecule, wherein said binding molecule is selected from the group consisting of:
   (i) an antibody that binds to the polypeptide or the nucleic acid molecule selected from the group consisting of:
      (a) a polypeptide comprising an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
      (b) a polypeptide comprising an amino acid sequence encoded by a nucleic acid molecule as depicted in SEQ ID NO 1; 3; 5; 7; 9; 11; 13; 15; 17; 19; 21; 23; 25; 27; 31; 33; 35; 37; 39; 41; 43 or 45.
      (c) a polypeptide comprising an amino acid sequence encoded by a nucleic acid molecule encoding an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
      (d) a polypeptide comprising an amino acid sequence having at least 50% sequence identity to the polypeptide of any one of (a) to (c);
      (e) a polypeptide comprising an amino acid sequence encoded by a nucleic acid molecule having at least 50% sequence identity to the nucleic acid molecule as defined in any one of (b) to (c);
      (f) a polypeptide comprising an amino acid sequence encoded by a nucleic acid molecule hybridizing under stringent conditions to the complement stand of a nucleic acid molecule as defined in any one of (b) to (c); and
      (g) fragment of a polypeptide of any one of (b) to (f).
      or to (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1;
   (ii) an antisense nucleotide sequence that hybridizes to the nucleic acid molecule as defined in (i);
   (iii) a siRNA that interacts with the nucleic acid molecule as defined in (i);
   (iv) an aptamer that binds to the polypeptide or the nucleic acid molecule as defined in (i) or to (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1; and
   (v) ribozyme that interacts with the nucleic acid molecule as defined in (i).

Aspect 12. The method of any one of aspects 1 to 6, wherein said compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is or comprises an agonist or antagonist of the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof, wherein said agonist or antagonist is a molecule selected from the group consisting of:
(A) a polypeptide as defined in (a)-(g) or a nucleotide sequence comprising a nucleic acid molecule as defined in (a)-(g);
   (a) a polypeptide comprising an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
   (b) a polypeptide comprising an amino acid sequence encoded by a nucleic acid molecule as depicted in SEQ ID NO 1; 3; 5; 7; 9; 11; 13; 15; 17; 19; 21; 23; 25; 27; 31; 33; 35; 37; 39; 41; 43 or 45.
   (c) a polypeptide comprising an amino acid sequence encoded by a nucleic acid molecule encoding an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
   (d) a polypeptide comprising an amino acid sequence having at least 50% sequence identity to the polypeptide of any one of (a) to (c);
   (e) a polypeptide comprising an amino acid sequence encoded by a nucleic acid molecule having at least 50% sequence identity to the nucleic acid molecule as defined in any one of (b) to (c);
   (f) a polypeptide comprising an amino acid sequence encoded by a nucleic acid molecule hybridizing under stringent conditions to the complement stand of a nucleic acid molecule as defined in any one of (b) to (c); and
   (g) fragment of a polypeptide of any one of (a) to (f),
(B) a binding molecule as defined in any one of claims 9, 10, and 11 (a) and (d) being an agonistic binding molecule; and
(C) a low molecular weight compound or a small molecule.

Aspect 13. The method of any one of aspects 1 to 6, wherein said compound is selected from thiorphan, phenanthroline, ARP100, glucosamine, micro-RNA miR-203. SB2K763, cytokine CXCL4, and isoflurane.

Aspect 14. The method of aspects 1 to 6, wherein said compound is SB-3CT.

Aspect 15. The method of any one of aspects 1 to 14, wherein said activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is a protease activity.

Aspect 16. The method of any one of aspects 1 to 14, wherein said activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is an AAA protease activity.

Aspect 17. The method of any one of aspects 1 to 14, wherein said activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is proteolytic cleavage of OPA1.

Aspect 18. The method of aspect 17, wherein said proteolytic cleavage of OPA1 leads to OPA processing.

Aspect 19. The method of any one of aspects 1 to 18, wherein said altered OPA1 processing is characterized by an altered (decrease of a) certain amount of at least one large isoform of OPA1, an altered (increase of a) certain amount of at least one small isoform of OPA1 and/or an altered (decrease of a) certain ratio of at least one large versus at least one small isoform of OPA1 compared to a control/standard.

Aspect 20. The method of aspect 19, wherein said at least one small isoform of OPA1 is OPA1-S3, OPA1-S4 and/or OPA1-S5.

Aspect 21. The method of any one of aspec201 to 18, wherein said disorder or disease is selected from the group consisting of premature ageing, cardiomyopathy, a respiratory chain disorder, mtDNA depletion syndrome, myoclonus epilepsy, ragged-red fibers syndrome (MERRF), myopathy encephalopathy lactic acidosis, stroke-like episodes (MELAS) and optic atrophy, glaucoma, optic neuropathy, Parkinson's Disease, Alzheimer's Disease or any other form of neurodegenerative disease or aging-related disease, such as cancer or diseases related to ischemia.

Aspect 22. A method of screening for a compound capable of modulating the activity of OMA1 and/or an oligomeric complex comprising OMA1 and/or HIGD1A and/or

OPA1 and/or BNIP3 and/or YMEIL1 and/or PHB and/or PHB2 and/or SAMM50 and/or

IMMT or a variant thereof comprising the steps of
(a) contacting OPA1 with said OMA1 and/or oligomeric complex comprising OMA1 and/or HIGD1A and/or OPA1 and/or BNIP3 and/or YMEIL1 and/or PHB and/or PHB2 and/or SAMM50 and/or IMMT or a variant thereof in the presence of said compound to be screened for under conditions allowing OPA1 processing to occur; and
(b) evaluating whether OPA1 processing is altered compared to a control, where OPA1 and OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof are contacted in the absence of said compound to be screened for under conditions allowing OPA1 processing to occur.

Aspect 23. The method of aspect 22, wherein OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof comprises a polypeptide selected from the group consisting of:
(a) a polypeptide comprising an amino acid sequence encoded by a nucleic acid molecule as depicted in SEQ ID NO 1; 3; 5; 7; 9; 11; 13; 15; 17; 19; 21; 23; 25; 27; 31; 33; 35; 37; 39; 41; 43 or 45.
(b) a polypeptide comprising an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
(c) a polypeptide comprising an amino acid sequence encoded by a nucleic acid molecule encoding an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
(d) a polypeptide comprising an amino acid sequence having at least 50% sequence identity to the polypeptide of any one of (a) to (c);
(e) a polypeptide comprising an amino acid sequence encoded by a nucleic acid molecule having at least 50% sequence identity to the nucleic acid molecule as defined in any one of (a) to (c);
(f) a polypeptide comprising an amino acid sequence encoded by a nucleic acid molecule hybridizing under stringent conditions to the complement stand of a nucleic acid molecule as defined in any one of (a) to (c); and
(g) fragment of a polypeptide of any one of (a) to (f).

Aspect 24. The method of aspect 22, wherein OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof is a homo-oligomeric complex or a hetero-oligomeric complex.

Aspect 25. The method of aspect 22, wherein OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof comprises a polypeptide selected from the group consisting of:
(a) a polypeptide comprising an amino acid sequence encoded by a nucleic acid molecule as depicted in SEQ ID NO 1; 3; 5; 7; 9; 11; 13; 15; 17; 19; 21; 23; 25; 27; 31; 33; 35; 37; 39; 41; 43 or 45.
(b) a polypeptide comprising an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
(c) a polypeptide comprising an amino acid sequence encoded by a nucleic acid molecule encoding an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
(d) a polypeptide comprising an amino acid sequence having at least 50% sequence identity to the polypeptide of any one of (a) to (c);
(e) a polypeptide comprising an amino acid sequence encoded by a nucleic acid molecule having at least 50% sequence identity to the nucleic acid molecule as defined in any one of (a) to (c);
(f) a polypeptide comprising an amino acid sequence encoded by a nucleic acid molecule hybridizing under stringent conditions to the complement stand of a nucleic acid molecule as defined in any one of (a) to (c); and
(g) fragment of a polypeptide of any one of (a) to (f).

Aspect 26. The method of aspect 22, wherein OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof is a homo-oligomeric complex or a hetero-oligomeric complex.

Aspect 27. The method of aspect 26, wherein said hetero-oligomeric complex comprises AFG311 and/or AFG312 and/or paraplegin and/or HIGD1a and/or OPA1 and/or BNIP3 and/or YMEIL1 and/or PHB and/or PHB2 or a variant thereof.

Aspect 28. The method of any one of aspects 22 to 27, wherein said compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is a compound obtained by a method comprising the steps of:
(a) contacting OPA1 with OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof in the presence of said compound to be screened for under conditions allowing OPA1 processing to occur; and
(b) evaluating whether OPA1 processing is altered compared to a control, where OPA1 and OMA1 and/or said oligomeric complex comprising OMA1 and/or HIGD1A and/or OPA1 and/or BNIP3 and/or YMEIL1 and/or PHB and/or PHB2 and/or SAMM50 and/or IMMT or a variant thereof are contacted in the absence of said compound to be screened for under conditions allowing OPA1 processing to occur.

Aspect 29. The method of any one of aspects 22 to 27, wherein said compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is or comprises an agonist or antagonist of the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof.

Aspect 30. The method of any one of aspects 20 to 25, wherein said compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is or comprises an agonist or antagonist of the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof, wherein said agonist or antagonist is a molecule selected from the group consisting of:
(a) a binding molecule that binds to/interacts with OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1 or binds to/interacts with a nucleic acid molecule encoding (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1;
(b) a nucleic acid molecule capable of introducing an insertion of a heterologous sequence or a mutation into a nucleic acid molecule encoding (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1 via in vivo mutagenesis;
(c) a nucleic acid molecule capable of reducing the expression of mRNA encoding (a subunit of) OMA1 and/or the oligomeric complex comprising OMA1 and/or

HIGD1A and/or OPA1 and/or BNIP3 and/or YMEIL1 and/or PHB and/or PHB2 and/or SAMM50 and/or IMMT or a variant thereof as defined in claim 1 by co-suppression; and

(d) a low molecular weight compound or a small molecule.

Aspect 31. The method of aspect 30, wherein said binding molecule is selected form the group consisting of antibodies, affybodies, trinectins, anticalins, aptamers, PNA, DNA or RNA.

Aspect 32. The method of any one of aspects 20 to 25, wherein said compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is or comprises an agonist or antagonist of the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof, wherein said agonist or antagonist is a molecule selected from the group consisting of:
(A) a binding molecule that binds to/interacts with OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1 or binds to/interacts with a nucleic acid molecule encoding (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1;
(B) a nucleic acid molecule capable of introducing an insertion of a heterologous sequence or a mutation into a nucleic acid molecule encoding (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1 via in vivo mutagenesis;
(C) a nucleic acid molecule capable of reducing the expression of mRNA encoding (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1 by co-suppression; and
(D) a low molecular weight compound or a small molecule, wherein said binding molecule is selected from the group consisting of:
   (i) an antibody that binds to the polypeptide or the nucleic acid molecule selected from the group consisting of:
      (a) a polypeptide comprising an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
      (b) a polypeptide comprising an amino acid sequence encoded by a nucleic acid molecule as depicted in SEQ ID NO 1; 3; 5; 7; 9; 11; 13; 15; 17; 19; 21; 23; 25; 27; 31; 33; 35; 37; 39; 41; 43 or 45.
      (c) a polypeptide comprising an amino acid sequence encoded by a nucleic acid molecule encoding an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
      (d) a polypeptide comprising an amino acid sequence having at least 50% sequence identity to the polypeptide of any one of (a) to (c);
      (e) a polypeptide comprising an amino acid sequence encoded by a nucleic acid molecule having at least 50% sequence identity to the nucleic acid molecule as defined in any one of (b) to (c);
      (f) a polypeptide comprising an amino acid sequence encoded by a nucleic acid molecule hybridizing under stringent conditions to the complement stand of a nucleic acid molecule as defined in any one of (b) to (c); and
      (g) fragment of a polypeptide of any one of (b) to (f).
      or to (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1;
   (ii) an antisense nucleotide sequence that hybridizes to the nucleic acid molecule as defined in (i);
   (iii) a siRNA that interacts with the nucleic acid molecule as defined in (i);
   (iv) an aptamer that binds to the polypeptide or the nucleic acid molecule as defined in (i) or to (a subunit of) OMA1 and/or the oligomeric complex comprising OMA1 and/or HIGD1A and/or OPA1 and/or BNIP3 and/or YMEIL1 and/or PHB and/or PHB2 and/or SAMM50 and/or IMMT or a variant thereof as defined in claim 1; and
   (v) ribozyme that interacts with the nucleic acid molecule as defined in (i).

Aspect 33. The method of any one of aspects 22 to 27, wherein said compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is or comprises an agonist or antagonist of the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof, wherein said agonist or antagonist is a molecule selected from the group consisting of:
(A) a polypeptide as defined in (a)-(g) or a nucleotide sequence comprising a nucleic acid molecule as defined in (a)-(g);
   (a) a polypeptide comprising an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
   (b) a polypeptide comprising an amino acid sequence encoded by a nucleic acid molecule as depicted in SEQ ID NO 1; 3; 5; 7; 9; 11; 13; 15; 17; 19; 21; 23; 25; 27; 31; 33; 35; 37; 39; 41; 43 or 45.
   (c) a polypeptide comprising an amino acid sequence encoded by a nucleic acid molecule encoding an amino acid sequence as depicted in SEQ ID NO 2; 4; 6; 8; 10; 12; 14; 16; 18; 20; 22; 24; 26; 28; 30; 32; 34; 36; 38; 40; 42; 44 or 46.
   (d) a polypeptide comprising an amino acid sequence having at least 50% sequence identity to the polypeptide of any one of (a) to (c);
   (e) a polypeptide comprising an amino acid sequence encoded by a nucleic acid molecule having at least 50% sequence identity to the nucleic acid molecule as defined in any one of (b) to (c);
   (f) a polypeptide comprising an amino acid sequence encoded by a nucleic acid molecule hybridizing under stringent conditions to the complement stand of a nucleic acid molecule as defined in any one of (b) to (c); and
   (g) fragment of a polypeptide of any one of (a) to (f),
(B) a binding molecule as defined in any one of claims 9, 10, and 11 (a) and (d) being an agonistic binding molecule; and
(C) a low molecular weight compound or a small molecule.

Aspect 34. The method of any one of aspects 22 to 33, wherein said activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is a protease activity.

Aspect 35. The method of any one of aspects 22 to 33, wherein said activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is an AAA protease activity.

Aspect 36. The method of any one of aspects 22 to 33, wherein said activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is proteolytic cleavage of OPA1.

Aspect 37. The method of claim 36, wherein said proteolytic cleavage of OPA1 leads to OPA1 processing.

Aspect 38. The method of any one of aspects 22 to 37, wherein said OPA1 processing is characterized by (a decrease of) a certain amount of at least one large isoform of OPA1, (an increase of) a certain amount of at least one small isoform of OPA1 and/or (a decrease of) a certain ratio of at least one large versus at least one small isoform of OPA1 (compared to a control/standard).

Aspect 39. The method of aspect 38, whereby a large isoform of OPA1 has an apparent molecular weight of more than about 91 kD and whereby a small isoform of OPA1 has an apparent molecular weight of less than about 91 kD, said molecular weights being determined by SDS-PAGE analysis; and/or whereby a large isoform of OPA1 has an apparent molecular weight of more than about 95 kD and whereby a small isoform of OPA1 has an apparent molecular weight of less than about 95 kD, said molecular weights being determined by mass spectrometry.

Aspect 40. The method of aspect 39, wherein said SDS-PAGE is a 10% SDS-PAGE.

Aspect 41. The method of any one of aspects 39 to 40, wherein said mass spectrometry is MALDI-MS or LC-MS/MS.

Aspect 42. The method of any one of aspects 39 to 41, wherein said at least one large isoform of OPA1 is OPA1-LI and/or OPA1-L2.

Aspect 43. The method of aspect 38, wherein said at least one large isoform of OPA1 comprises two isoforms (OPA1-LI and OPA1-L2) and/or wherein said at least one small isoform of OPA1 comprises three isoforms (OPA1-S3, OPA1-S4 and OPA1-S5).

Aspect 44. The method of aspect 43, wherein said at least one large isoform of OPA1 comprises an isoform having an apparent molecular weight of about 97 kD (OPA1-L1) or an isoform having an apparent molecular weight of about 92 kD (OPA1-L2), said molecular weights being determined by SDS-PAGE analysis.

Aspect 45. The method of aspect 43, wherein said at least one small isoform of OPA1 comprises an isoform having an apparent molecular weight of about 88 kD (OPA1-S3), an isoform having an apparent molecular weight of about 84 kD (OPA1-S4) or an isoform having an apparent molecular weight of about 81 kD (OPA1-S5), said molecular weights being determined by SDS-PAGE analysis.

Aspect 46. The method of aspect 43, wherein said at least one large isoform of OPA1 comprises an isoform having an apparent molecular weight of about 104 kD (OPA1-L1) or an isoform having an apparent molecular weight of about 99 kD (OPA1-L2), said molecular weights being determined by mass spectrometry.

Aspect 47. The method of aspect 43, wherein said at least one small isoform of OPA1 comprises an isoform having an apparent molecular weight of about 92 kD (OPA1-S3), an isoform having an apparent molecular weight of about 89 kD (OPA1-S4) or an isoform having an apparent molecular weight of about 87 kD (OPA1-S5), said molecular weights being determined by mass spectrometry.

Aspect 48. The method of aspect 43, wherein,
said OPA1-LI has an apparent molecular weight of about 97 kD,
said OPA1-L2 has an apparent molecular weight of about 92 kD,
said OPA1-S3 has an apparent molecular weight of about 88 kD,
said OPA1-S4 has an apparent molecular weight of about 84 kD, and/or
said OPA1-S5 has an apparent molecular weight of about 81 kD,
said molecular weights being determined by SDS-PAGE analysis; or wherein
said OPA1-LI has an apparent molecular weight of about 104 kD,
said OPA1-L2 has an apparent molecular weight of about 99 kD,
said OPA1-S3 has an apparent molecular weight of about 92 kD,
said OPA1-S4 has an apparent molecular weight of about 89 kD, and/or
said OPA1-S5 has an apparent molecular weight of about 87 kD,
said molecular weights being determined by mass spectrometry.

Aspect 49. The method of aspect 43, wherein,
(a) OPA1-LI and OPA1-L2 are characterized by comprising amino acid stretches or amino acid peptides comprising one or more of the following sequences:

(SEQ ID NO: 47)
YLILGSAVGGGYTAK;
(SEQ ID NO: 48)
TFDQWK;
(SEQ ID NO: 49)
DMIPDLSEYK;
(SEQ ID NO: 50)
WIVPDIVWEIDEYIDFEK;
(SEQ ID NO: 51)
LAPDFDK;
(SEQ ID NO: 52)
IVESLSLLK;
(SEQ ID NO: 53)
ALPNSEDLVK;
(SEQ ID NO: 54)
DFFTSGSPEETAFR;
(SEQ ID NO: 55)
TRLLKLRYLILGS;
and
(SEQ ID NO: 56)
FWPARLATRLLKLRYLILGS;

or derivatives thereof;
(b) OPA1-S3 is characterized by comprising amino acid stretches or amino acid peptides comprising one or more of the following sequences:

(SEQ ID NO: 52)
IVESLSLLK;
(SEQ ID NO: 54)
DFFTSGSPEETAFR;
(SEQ ID NO: 57)
GLLGELILLQQQIQEHEEEAR;
(SEQ ID NO: 58)
AAGQYSTSYAQQK;
and
(SEQ ID NO: 59)
IDQLQEELLHTQLK;

or derivatives thereof;
(c) OPA1-S4 is characterized by comprising amino acid stretches or amino acid peptides comprising one or more of the following sequences:

(SEQ ID NO: 57)
GLLGELILLQQQIQEHEEEAR;
(SEQ ID NO: 58)
AAGQYSTSYAQQK;
and
(SEQ ID NO: 59)
IDQLQEELLHTQLK;

or derivatives thereof; and/or
(d) OPA1-S5 is characterized by comprising amino acid stretches or amino acid peptides comprising the following sequence:

(SEQ ID NO: 59)
IDQLQEELLHTQLK;

or derivatives thereof.

Aspect 50. The method of aspect 43, wherein
(a) OPA1-L2 is characterized by not comprising amino acid stretches or amino acid peptides comprising one or more of the following sequences:

(SEQ ID NO: 57)
GLLGELILLQQQIQEHEEEAR;
and
(SEQ ID NO: 58)
AAGQYSTSYAQQK;

or derivatives thereof; and/or
(b) OPA1-S3 is characterized by not comprising amino acid stretches or amino acid peptides comprising one or more of the following sequences:

(SEQ ID NO: 47)
YLILGSAVGGGYTAK
(SEQ ID NO: 48)
TFDQWK;
(SEQ ID NO: 49)
DMIPDLSEYK;
(SEQ ID NO: 50)
WIVPDIVWEIDEYIDFEK;
(SEQ ID NO: 51)
LAPDFDK;
(SEQ ID NO: 52)
IVESLSLLK;
(SEQ ID NO: 53)
ALPNSEDLVK;
(SEQ ID NO: 55)
TRLLKLRYLILGS;
and
(SEQ ID NO: 56)
FWPARLATRLLKLRYLILGS;

or derivatives thereof;
(c) OPA1-S4 is characterized by not comprising amino acid stretches or amino acid peptides comprising one or more of the following sequences:

(SEQ ID NO: 47)
YLILGSAVGGGYTAK;
(SEQ ID NO: 48)
TFDQWK;
(SEQ ID NO: 49)
DMIPDLSEYK;
(SEQ ID NO: 50)
WIVPDIVWEIDEYIDFEK;
(SEQ ID NO: 51)
LAPDFDK;
(SEQ ID NO: 52)
IVESLSLLK;
(SEQ ID NO: 53)
ALPNSEDLVK;
(SEQ ID NO: 54)
DFFTSGSPEETAFR;
(SEQ ID NO: 55)
TRLLKLRYLILGS;
and
(SEQ ID NO: 56)
FWPARLATRLLKLRYLILGS;

or derivatives thereof; and/or
(d) OPA1-S5 is characterized by not comprising amino acid stretches or amino acid peptides comprising one or more of the following sequences:

(SEQ ID NO: 47)
YLILGSAVGGGYTAK;
(SEQ ID NO: 48)
TFDQWK;
(SEQ ID NO: 49)
DMIPDLSEYK;
(SEQ ID NO: 50)
WIVPDIVWEIDEYIDFEK;
(SEQ ID NO: 51)
LAPDFDK;
(SEQ ID NO: 52)
IVESLSLLK;
(SEQ ID NO: 53)
ALPNSEDLVK;
(SEQ ID NO: 54)
DFFTSGSPEETAFR;
(SEQ ID NO: 55)
TRLLKLRYLILGS;
and
(SEQ ID NO: 57)
GLLGELILLQQQIQEHEEEAR;
and
(SEQ ID NO: 58)
AAGQYSTSYAQQK;

or derivatives thereof.

Aspect 51. A method for determining the susceptibility for, predisposition for or the presence of
(i) a disorder or disease correlated with mitochondrial dysfunction or a mitochondrial disorder or disease; or
(ii) a disorder or disease characterized by OPA1 alterations, wherein said method comprises the steps of
(a) obtaining a sample from the subject and measuring the activity of OMA1 and/or YMEIL1 or a combination thereof in the sample, and/or measuring the gene expression levels of OMA1, HIGD1A, OPA1, BNIP3, YMEIL1, PHB, SAMM50, IMMT and/or PHB2 or a combination thereof in the sample, and/or measuring the protein levels of OMA1, HIGD1A, OPA1, BNIP3, YMEIL1, PHB, SAMM50, IMMT and/or PHB2 or a combination thereof in the sample;
(b) comparing the increase and/or decrease of measured activity and/or gene expression levels and/or protein levels of OMA1 and/or HIGD1A and/or OPA1 and/or BNIP3 and/or YMEIL1 and/or PHB and/or SAMM50 and/or IMMT and/or PHB2 or a combination thereof in the sample compared to a reference;
(c) integrating the results of these measurements through combination of 3 or more genes selected from the group of OMA1, HIGD1A, OPA1, BNIP3, YMEIL1, PHB, SAMM50, IMMT and PHB2.

Aspect 52. The method of aspect 50, wherein said activity of OMA1 and/or an oligomeric complex comprising OMA1 and/or HIGD1A and/or OPA1 and/or BNIP3 and/or YME1L1 and/or PHB and/or PHB2 and/or SAMM50 and/or IMMT or a variant thereof is a protease activity.

Aspect 53. The method of aspect 50, wherein said activity of OMA1 and/or an oligomeric complex comprising OMA1 and/or HIGD1A and/or OPA1 and/or BNIP3 and/or YME1L1 and/or PHB and/or PHB2 and/or SAMM50 and/or IMMT or a variant thereof is an AAA protease activity.

Aspect 54. The method of aspect 50, wherein said activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof is proteolytic cleavage of OPA1.

Aspect 55. The method of aspect 54, wherein said proteolytic cleavage of OPA1 leads to OPA1 processing.

Aspect 56. The method of any one of aspects 50 to 55, wherein said altered OPA1 processing is characterized by an altered (decrease of a) certain amount of at least one large isoform of OPA1, an altered (increase of a) certain amount of at least one small isoform of OPA1 and/or an altered (decrease of a) certain ratio of at least one large versus at least one small isoform of OPA1 compared to a control/standard.

Aspect 57. The method of aspect 56, wherein said at least one small isoform of OPA1 is OPA1-S3, OPA1-S4 and/or OPA1-S5.

Aspect 58. The method of any one of aspects 50 to 57, wherein said disorder or disease is selected from the group consisting of premature ageing, cardiomyopathy, a respiratory chain disorder, mtDNA depletion syndrome, myoclonus epilepsy, ragged-red fibers syndrome (MERRF), myopathy encephalopathy lactic acidosis, stroke-like episodes (MELAS) and optic atrophy, glaucoma, optic neuropathy, Parkinson's Disease, Alzheimer's Disease or any other form of neurodegenerative disease or aging-related disease, such as cancer or diseases related to ischemia.

Aspect 59. The method of any one of aspects 50 to 58, wherein said method is utilized for determining whether a patient in need for medical intervention will benefit from administration of a pharmaceutically active amount of a compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof.

Aspect 60. The method of any one of aspects 50 to 59, wherein said method is a biomarker for the predisposition of a disorder or disease, a biomarker for a disorder or disease or a biomarker for evaluating efficacy of a method for treatment, prevention and/or amelioration of a disorder or disease.

Aspect 61. The method of aspect 60, wherein said biomarker is informative for selecting medical interventions and/or therapies, monitoring medical interventions and/or therapies or predicting outcomes of medical interventions and/or therapies.

Aspect 62. The method of any one of aspects 50 to 61, wherein said method compromises the use of a kit comprising reagents for the quantification of the expression levels of OMA1 and/or HIGD1A and/or OPA1 and/or BNIP3 and/or YMEIL1 and/or PHB and/or SAMM50 and/or IMMT and/or PHB2 or a functionally equivalent variant thereof or of any combination of these molecules.

Aspect 63. The method of any one of aspects 50 to 61, wherein said method compromises the use of a kit comprising reagents for the quantification of the levels of OMA1 and/or HIGD1A and/or OPA1 and/or BNIP3 and/or YMEIL1 and/or PHB and/or SAMM50 and/or IMMT and/or PHB2 or a functionally equivalent variant thereof or of any combination of these molecules, wherein such quantification is performed by means of Western blot, immunohistochemistry or ELISA.

Aspect 64. A compound capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof for the treatment, prevention and/or amelioration of
(i) a disorder or disease correlated with mitochondrial dysfunction or a mitochondrial disorder or disease; or
(ii) a disorder or disease characterized by OPA1 alterations, wherein the compound is capable of modulating the activity of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof, and wherein said altered OPA1 processing is characterized by an altered (decrease of a) certain amount of at least one large isoform of OPA1, an altered (increase of a) certain amount of at least one small isoform of OPA1 and/or an altered (decrease of a) certain ratio of at least one large versus at least one small isoform of OPA1 compared to a control/standard,

Aspect 65. A method of treating a disease or disorder in a patient comprising administering to a patient in need of such treatment a therapeutically effective amount of the compound of aspect 65.

Aspect 66. A pharmaceutical composition comprising the compound of aspect 65 and a pharmaceutically acceptable excipient.

Aspect 67. A method of treating a disease or disorder in a patient comprising administering to a patient in need of such treatment a therapeutically effective amount of the pharmaceutical composition of aspect 66.

Aspect 68. A combination of two or more methods according to aspects 1, 22, and 51 that result in a medical intervention individualized for one or more patients and that may be referred to as personalized medicine and/or precision medicine.

Mitochondria are dynamic organelles forming networks of interconnected tubules, which maintain their homeostasis by constantly fusing and dividing. Fragmented mitochondria thereby are more susceptible to apoptotic cell death.

OPA1 is an essential fusion protein, which is deactivated by the OMA1 protease under conditions of cell stress. OPA1 occurs in two forms, large L-OPA1 isoforms and small S-OPA1 isoforms. S-OPA1 is derived from L-OMA1 by proteolytic cleavage by the OMA1 protease and the YMEIL1 protease, which is also known as i-AAA protease. OMA1 cleaves OPA1 and thereby generates S-OPA1 under conditions broadly defined as cell-stress, whereby OMA1 activation facilitates outer membrane permeabilization and cytochrome c release resulting in apoptotic cell death.

We identified compounds disclosed herein which are known in the arts as HIV-1 protease inhibitors. These compounds, as shown in the example provided below and in Appendix 1, surprisingly and quite unexpectedly activated the OMA1 protease. The disclosed compounds had also a dose-dependent antagonistic effect on OPA1. Low doses resulted in OPA1 cleavage. However, higher doses prevented OPA1 cleavage even under conditions that would otherwise result in OPA1 cleavage.

The OMA1 protease has a number of disease implications and is supported by epidemiological and genetic data from humans and animal disease models. And yet, there are still until now no OMA1 modulators disclosed in the arts although many have tried without much success to identify such molecules.

The meaning of the terms “OMA1” and “OPA1” and “YMEIL1” are well known in the art and is, if not explicitly prescribed differentially, used accordingly in context of the present invention. In context of this invention, these terms are likewise used to refer to the corresponding nucleotide sequences (e.g., the genes) as well as to the corresponding polypeptides (e.g., the polypeptides encoded by said genes). It is to be understood that these terms are defined as broad as possible and shall include all natural and non-natural variants thereof from any and every species.

As shown herein and in the arts, mitochondrial dysfunction (or a corresponding mitochondrial disease or disorder) is not merely correlated with decrease of any one of OPA1 isoforms, but with a decrease of particularly the large isoforms. Referring to FIG. 1, the large isoforms of OPA1 (L-OPA1) may comprise two isoforms and the small isoforms of OPA1 (S-OPA1) may comprise three isoforms. However, it is also within the context of the present invention that additional, possibly existing OPA1 isoforms may be assigned as large or small isoforms.

In this context, it is to be understood that the OPA1 isoforms serve as proxy for protease activity, in particular for OMA1 protease activity. Therefore, the present invention is not limited to the modulation of OPA1 isoforms but encompasses also any and every other proxy for OMA1 activity, including other OMA1 substrates, such as DELEI, PGAM5, or PINK1. A person skilled in the art is also readily in a position to deduce (further) amino acid stretches/peptides that are (artificial) OMA1 substrates (see, for example, U.S. Ser. No. 16/101,134.)

Conversely, compounds of present disclosure may modulate the ratio of OPA1 isoforms by direct or indirect interaction with the OMA1 protease, for example by interacting with a protein complex comprising OMA1, or by interacting with other proteases, which may cleave OPA1 and/or OMA1, such as the i-AAA protease, or by interacting with OMA1-regulating enzymes, such as the m-AAA protease or prohibitin (see also Alavi M. V. “OMA1—An integral membrane protease” Biochim Biophys Acta Proteins Proteom. 2020 Oct 29:140558.)

Clinical and/or pathological examples for diseases linked to OPA1 modulation in the context of the present invention are given in the list above.

However, these disorders or diseases are mere examples to be covered by this invention which is not strictly construed to the clinical and/or pathological situations described above.

Cancer is defined broadly and can refer to any and all forms of malignancy.

The singular forms “a”, “an”, and “the” as used herein and in the claims include plural reference unless the context dictates otherwise. For example, “a cell” means as well a plurality of cells, and so forth. The term “and/or” as used in the present specification and in the claims implies that the phrases before and after this term are to be considered either as alternatives or in combination.

The terms “agent”, “reagent”, “modulator”, “compound”, “molecule” and the like refer to any substance, chemical, composition or extract that have a positive or negative biological effect on a cell, tissue, body fluid, or within the context of any biological system, or any assay system.

The compounds of the present invention can be in any suitable form, without limitation. Forms suitable for oral use, include, but are not limited to, tablets, troches, lozenges, aqueous or oily suspensions, dispersible powders or granules, emulsions, hard or soft capsules, solutions, syrups and elixirs. Compositions intended for oral use may be prepared according to any method known to the art for the manufacture of pharmaceutical compositions.

The compounds can be formulated with other ingredients, e.g., “pharmaceutically acceptable carriers” or “excipients” to indicate they are combined with the active drug and can be administered safely to a subject for therapeutic purposes. These include, but are not limited to, antioxidants, preservatives, dyes, tablet-coating compositions, plasticizers, inert carriers, excipients, polymers, coating materials, osmotic barriers, devices and agents which slow or retard solubility, etc.

The compositions intended for oral use may be prepared according to any suitable method known to the art for the manufacture of pharmaceutical compositions. Such compositions may contain one or more agents selected from the group consisting of diluents, sweetening agents, flavoring agents, coloring agents and preserving agents in order to provide palatable preparations.

Non-toxic pharmaceutically acceptable excipients that are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; and binding agents, for example magnesium stearate, stearic acid or talc.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions containing the active materials in admixture with excipients suitable for the manufacture of aqueous suspensions may also be used. Such excipients are suspending agents, for example sodium carboxymethylcellulose, methylcellulose, hydroxypropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecacthylene oxycetanol, or condensation products of ethylene oxide with partial esters derived from fatty acids and hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

The compositions intended for oral use may contain lecithin as an emulsifier and solubilizing agent to achieve a high loading of the disclosed compounds. The amount of active ingredient in the emulsion formulations of the present invention may vary or be adjusted widely depending on the intended route of administration, ages of patients being treated, the severity of the retroviral infection and the required concentration. In preparing the emulsion formulations, the amount of active ingredient is not particularly restricted up to 200 mg/ml (20% by weight) for maintaining high oral bioavailability, but preferably ranges from about 1% to about 20%, and more preferably from about 2% to about 15%, by weight of the total composition.

The emulsion formulations may be administered orally, or parenterally at a dosage to obtain and maintain a concentration, that is, an amount, or blood-level of the active ingredient in the patients, which will be therapeutically effective. Generally, such therapeutically effective amount of active ingredient ranges from about 0.1 to about 300 mg/kg of body weight per day. Also, it is to be understood that the initial dosage administered may be increased beyond the above upper level in order to rapidly achieve the desired blood-level or the initial dosage may be smaller than the optimum and the daily dosage may be progressively increased during the course of treatment depending on the particular situation. If desired, the daily dose may also be divided into multiple doses for administration, e.g., two or more times per day.

The term “oil component” refers to monoglyceride, diglyceride, triglyceride, or a mixture comprising at least two members of these three glycerides. Preferably, the oil component is triglyceride, a mixture of diglyceride and monoglyceride in a ratio of from about 9:1 to about 1:9 by weight, a mixture of diglyceride and triglyceride in a ratio of from about 9:1 to about 1:9 by weight, or a mixture of monoglyceride, diglyceride and triglyceride in a ratio of from about 1 to about 8 parts of diglyceride per 10 parts of the mixture, about 1 to 5 parts of monoglyceride per 10 parts of the mixture, and about 1 to about 8 parts of triglyceride per 10 parts of the mixture.

More preferably, the oil component is triglyceride, a mixture of diglyceride and monoglyceride in a ratio of about 8:2 (diglyceride:monoglyceride) by weight, a mixture of diglyceride and triglyceride in a ratio of about 2:8 (triglyceride:diglyceride) by weight.

Diglyceride (hereinafter DGO) of the present invention refers to a long chain fatty acid ester of glycerol having structural formula HOCH₂—CH(O₂CR)—CH₂(O₂CR) or (RCO₂)CH₂—CH(OH)—CH₂(O₂CR), wherein R is mono-unsaturated or di-unsaturated alkyl group having fifteen to twenty-one carbon atoms. The preferred diglyceride is diolein (R is mono-unsaturated alkyl group with seventeen carbon atoms), dilinoleate (R is di-unsaturated alkyl group with seventeen carbon atoms), or a mixture of diolein and dilinoleate. The most preferred diglyceride is diolein.

Monoglyceride (hereinafter GMO) of the present invention refers to a long chain fatty acid ester of glycerol having structural formula HOCH₂—CH(OH)—CH₂(O₂CR) or HOCH₂—CH(O₂CR)—CH₂OH, wherein R is a mono-unsaturated or di-unsaturated alkyl group having fifteen to twenty-one carbon atoms. The preferred monoglyceride is monoolein (R is mono-unsaturated alkyl group with seventeen carbon atoms), monolinoleate (R is di-unsaturated alkyl group with seventeen carbon atoms), or a mixture of monoolein and monolinoleate. The most preferred monoglyceride is monoolein.

Triglyceride (hereinafter MCT) of the present invention refers to a medium chain fatty acid ester of glycerol having structural formula R¹OCH₂—CH(O₂CR₂)—CH₂(O₂CR¹), wherein R1 is a saturated alkyl group having five to eleven carbon atoms. The preferred triglyceride is under brand names Miglyol 810, Miglyol 812, Miglyol 829, etc. which refer to different grades of fractionated and purified coconut oil consisting mainly of medium chain triglycerides.

In preparing the emulsion of the present invention, the amount of the oil component is not particularly restricted, but preferably ranges from about 5% to about 40%, more preferably from about 10% to about 30%, and most preferably from about 10% to about 20%, by weight of the total composition.

An oil component which comprises the mixture of diglyceride, monoglyceride or triglyceride may be prepared by mixing individual oil in appropriate proportion or by partial hydrolysis of triglyceride, or transesterification reaction of triglycerides or diglycerides with glycerol.

All of the glycerides of the present invention are known and can be obtained by conventional methods from a broad spectrum of water-immiscible material, such as soybean oil, avocado oil, squalene oil, sesame oil, olive oil, canola oil, corn oil, rapeseed oil, safflower oil, sunflower oil, coconut oil or mixtures thereof.

The term “lecithin” used herein refers monoaminomonophospholipid, a group of substances having structure formula FACOO—CH₂—CH(OOCFA)—CH₂—OPO₄—NR″₃, wherein FA is a fatty acid, R″ is an alkyl radical. Lecithin also refers to esters of oleic, steric, palmitic, or other fatty acids with glycerolphosphoric acid and choline. Lecithin may be, for instance, yolk lecithin and soybean lecithin, or synthetic nontoxic didecanoyl phosphatidycholine, dilauroyl phosphatidycholine, dimyristoyl phosphatidycholine, dipalmitoyl phosphatidycholine or a mixture thereof. Therefore, lecithin is not restricted to specific ones so far as they have compositions comprising the foregoing element. Lecithin is used as an emulsifying agent in the present invention. It also operates as a solubilizing agent.

In preparing the lipid emulsion of the present invention, the amount of lecithin to be used is not particularly restricted, but preferably ranges from about 0.5% to about 20%, more preferably from about 1% to about 10% and most preferably from about 2% to about 5%, by weight of the total composition.

Solvents of the present invention refer to propylene glycol, polyethylene glycol, glycerol, triacetin, dimethyl isosorbide, glycofurol, propylene carbonate, ethanol, water, dimethyl acetamide or a mixture thereof. The preferred solvent is propylene glycol, ethanol, water or a mixture thereof. The most preferred solvent is water.

If desired, the compositions may further comprise conventional pharmaceutical additives such as coloring agents, flavoring agents, thickening agents, stabilizers such as sodium deoxycholate, anti-oxidants such as BHT or vitamin E, and preserving agents such as methyl paraben, or propyl paraben.

The compositions may be prepared in a conventional manner, for example, (1) preparing an oil phase by dissolving an active ingredient in a mixture of an oil component, solvent(s), lecithin and optional water-immiscible excipients by heat, (2) preparing an aqueous phase by dissolving optional excipients in water, (3) combining the oil phase and the aqueous phase using a high energy homogenizer to achieve a submicron lipid emulsion.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example, sweetening, flavoring and coloring agents, may also be present.

The compounds may also be in the form of non-aqueous liquid formulations, e.g., oily suspensions which may be formulated by suspending the active ingredients in a vegetable oil, for example arachis oil, olive oil, sesame oil or peanut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The compounds may also be administered in the form of suppositories for rectal or vaginal administration of the drug. These compositions can be prepared by mixing the drug with a suitable non-irritating excipient which is solid at ordinary temperatures but liquid at the rectal temperature or vaginal temperature and will therefore melt in the rectum or vagina to release the drug. Such materials include cocoa butter and polyethylene glycols.

The compounds of the invention may also be administered transdermally using methods known to those skilled in the art (see, for example: Chien; “Transdermal Controlled Systemic Medications”; Marcel Dekker, Inc.; 1987. Lipp et al. WO94/04157 3 Mar. 94). For example, a solution or suspension of a compound of formula XII in a suitable volatile solvent optionally containing penetration enhancing agents can be combined with additional additives known to those skilled in the art, such as matrix materials and bactericides. After sterilization, the resulting mixture can be formulated following known procedures into dosage forms. In addition, on treatment with emulsifying agents and water, a solution or suspension of a compound of formula XII may be formulated into a lotion or salve.

Suitable solvents for processing transdermal delivery systems are known to those skilled in the art, and include lower alcohols such as ethanol or isopropyl alcohol, lower ketones such as acetone, lower carboxylic acid esters such as ethyl acetate, polar ethers such as tetrahydrofuran, lower hydrocarbons such as hexane, cyclohexane or benzene, or halogenated hydrocarbons such as dichloromethane, chloroform, trichlorotrifluoroethane, or trichlorofluoroethane. Suitable solvents may also include mixtures of one or more materials selected from lower alcohols, lower ketones, lower carboxylic acid esters, polar ethers, lower hydrocarbons, halogenated hydrocarbons.

Suitable penetration enhancing materials for transdermal delivery system are known to those skilled in the art, and include, for example, monohydroxy or polyhydroxy alcohols such as ethanol, propylene glycol or benzyl alcohol, saturated or unsaturated C8-C18 fatty alcohols such as lauryl alcohol or cetyl alcohol, saturated or unsaturated C8-C18 fatty acids such as stearic acid, saturated or unsaturated fatty esters with up to 24 carbons such as methyl, ethyl, propyl, isopropyl, n-butyl, sec-butyl, isobutyl, tertbutyl or monoglycerin esters of acetic acid, caproic acid, lauric acid, myristinic acid, stearic acid, or palmitic acid, or diesters of saturated or unsaturated dicarboxylic acids with a total of up to 24 carbons such as diisopropyl adipate, diisobutyl adipate, diisopropyl sebacate, diisopropyl maleate, or diisopropyl fumarate. Additional penetration enhancing materials include phosphatidyl derivatives such as lecithin or cephalin, terpenes, amides, ketones, ureas and their derivatives, and ethers such as dimethyl isosorbid and diethyleneglycol monoethyl ether. Suitable penetration enhancing formulations may also include mixtures of one or more materials selected from monohydroxy or polyhydroxy alcohols, saturated or unsaturated C8-C18 fatty alcohols, saturated or unsaturated C8-C18 fatty acids, saturated or unsaturated fatty esters with up to 24 carbons, diesters of saturated or unsaturated dicarboxylic acids with a total of up to 24 carbons, phosphatidyl derivatives, terpenes, amides, ketones, ureas and their derivatives, and ethers.

Suitable binding materials for transdermal delivery systems are known to those skilled in the art and include polyacrylates, silicones, polyurethanes, block polymers, styrenebutadiene copolymers, and natural and synthetic rubbers. Cellulose ethers, derivatized polyethylenes, and silicates may also be used as matrix components. Additional additives, such as viscous resins or oils may be added to increase the viscosity of the matrix.

Compositions comprising the compounds can also be formulated for controlled release, where release of the active ingredient is regulated or modulated to achieve a desired rate of delivery into the systemic circulation. A controlled release formulation can be pulsed, delayed, extended, slow, steady, immediate, rapid, fast, etc. It can comprise one or more release formulations, e.g., extended- and immediate-release components. Extended delivery systems can be utilized to achieve a dosing internal of once every 24 hours, once every 12 hours, once every 8 hours, once every 6 hours, etc. The dosage form/delivery system can be a tablet or a capsule suited for extended release, but a sustained release liquid or suspension can also be used. A controlled release pharmaceutical formulation can be produced which maintains the release of, and or peak blood plasma levels of a compound of the aspect 1 or 11 of the present invention.

Compounds of the invention may also be administered to the nasal mucosa thereby delivering the agent through the olfactory epithelium into or along olfactory neurons and the olfactory neural pathway to the brain of a mammal or preferentially a human subject. Compounds of the invention may also be administered to a central nervous system of a mammal by administering a composition comprising an effective amount of the compound to a tissue of the mammal innervated by the trigeminal nerve, the olfactory nerve, or a combination thereof, wherein the compound is absorbed through the tissue and transported into the central nervous system of the mammal in an amount effective to provide a diagnostic, protective, or therapeutic effect on a cell of the central nervous system.

Compounds of the invention may be combined with a pharmaceutically-acceptable carrier, or a transfer component, or a combination thereof; the composition containing the compound in an amount effective for treating or preventing a brain disease or disorder in the mammal. Suitable pharmaceutically-acceptable carrier are known in the arts. The composition can include, for example, any pharmaceutically acceptable additive, carrier, or adjuvant that is suitable for administering a compound to tissue innervated by the olfactory and/or trigeminal nerves. Preferably, the pharmaceutical composition can be employed in diagnosis, prevention, or treatment of a disease, disorder, or injury of the CNS, brain, and/or spinal cord. Preferably, the composition includes a compound in combination with a pharmaceutical carrier, additive, and/or adjuvant that can promote the transfer of the compound within or through tissue innervated by the olfactory and/or trigeminal nerves. Alternatively, the compound may be combined with substances that may assist in transporting the compounds to sites of malignancy or nerve cell damage.

The composition typically contains a pharmaceutically acceptable carrier mixed with the compound and other components in the pharmaceutical composition. By “pharmaceutically acceptable carrier” is intended a carrier that is conventionally used in the art to facilitate the storage, administration, and/or the healing effect of the disclosed compounds. A carrier may also reduce any undesirable side effects of the compound. A suitable carrier should be stable, i.e., incapable of reacting with other ingredients in the formulation. It should not produce significant local or systemic adverse effect in recipients at the dosages and concentrations employed for treatment. Such carriers are generally known in the art.

Suitable carriers for this invention include those conventionally used for large stable macromolecules such as albumin, gelatin, collagen, polysaccharide, monosaccharides, polyvinylpyrrolidone, polylactic acid, polyglycolic acid, polymeric amino acids, fixed oils, ethyl oleate, liposomes, glucose, sucrose, lactose, mannose, dextrose, dextran, cellulose, mannitol, sorbitol, polyethylene glycol (PEG), and the like.

Water, saline, aqueous dextrose, and glycols are preferred liquid carriers, particularly (when isotonic) for solutions. The carrier can be selected from various oils, including those of petroleum, animal, vegetable or synthetic origin, for example, peanut oil, soybean oil, mineral oil, sesame oil, and the like. Suitable pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like. The compositions can be subjected to conventional pharmaceutical expedients, such as sterilization, and can contain conventional pharmaceutical additives, such as preservatives, stabilizing compounds, wetting, or emulsifying agents, salts for adjusting osmotic pressure, buffers, and the like.

A composition formulated for intranasal delivery may optionally comprise an odorant. An odorant agent is combined with the compounds of present invention to provide an odoriferous sensation, and/or to encourage inhalation of the intranasal preparation to enhance delivery of the active compound to the olfactory neuroepithelium. The odoriferous sensation provided by the odorant agent may be pleasant, obnoxious, or otherwise malodorous. The odorant receptor neurons are localized to the olfactory epithelium that, in humans, occupies only a few square centimeters in the upper part of the nasal cavity. The cilia of the olfactory neuronal dendrites which contain the receptors are fairly long (about 30-200 μm). A 10-30 μm layer of mucus envelops the cilia that the odorant agent must penetrate to reach the receptors. See Snyder et al. (1998) J Biol. Chem. 263:13972-13974. Use of a lipophilic odorant agent having moderate to high affinity for odorant binding protein (OBP) is preferred. OBP has an affinity for small lipophilic molecules found in nasal secretions and may act as a carrier to enhance the transport of a lipophilic odorant substance and compounds to the olfactory receptor neurons. It is also preferred that an odorant agent is capable of associating with lipophilic additives such as liposomes and micelles within the preparation to further enhance delivery of the compounds by means of OBP to the olfactory neuroepithelium. OBP may also bind directly to lipophilic agents to enhance transport of the compounds to olfactory neural receptors.

Suitable odorants having a high affinity for OBP include terpenoids such as citralva and citronellol, aldehydes such as amyl cinnamaldehyde and hexyl cinnamaldehyde, esters such as octyl isovalerate, jasmines such as CIS-jasmine and jasmal, and musk 89. Other suitable odorant agents include those which may be capable of stimulating odorant-sensitive enzymes such as aderrylate cyclase and guanylate cyclase, or which may be capable of modifying ion channels within the olfactory system to enhance absorption of the compound.

Other acceptable components in the composition include, but are not limited to, pharmaceutically acceptable agents that modify isotonicity, including water, salts, sugars, polyols, amino acids and buffers, such as, phosphate, citrate, succinate, acetate, and other organic acids or their salts. Typically, the pharmaceutically acceptable carrier also includes one or more stabilizers, reducing agents, anti-oxidants and/or anti-oxidant chelating agents. The use of buffers, stabilizers, reducing agents, anti-oxidants and chelating agents in the preparation of protein-based compositions, particularly pharmaceutical compositions, is well known in the art. See Wang et al. (1980) J. Parent. Drug Assn., 34(6):452-462; Wang et al. (1988) J. Parent. Sci. and Tech. 42:S4-S26 (Supplement); Lachman, et al. (1968) Drug and Cosmetic Industry, 102(1): 36-38, 40 and 146-148; Akers, M.J. (1988) J. Parent. Sci. and Tech., 36(5):222-228; and Colowick et al. Methods in Enzymology, Vol. XXV, p. 185-188.

Suitable buffers include acetate, adipate, benzoate, citrate, lactate, maleate, phosphate, tartarate, borate, tri(hydroxymethyl aminomethane), succinate, glycine, histidine, the salts of various amino acids, or the like, or combinations thereof. See Wang (1980) supra at page 455. Suitable salts and isotonicifiers include sodium chloride, dextrose, mannitol, sucrose, trehalose, or the like. Where the carrier is a liquid, it is preferred that the carrier is hypotonic or isotonic with oral, conjunctival or dermal fluids and have a pH within the range of 4.5-8.5. Where the carrier is in powdered form, it is preferred that the carrier is also within an acceptable non-toxic pH range.

Suitable reducing agents, which maintain the reduction of reduced cysteines, include dithiothreitol (DTT also known as Cleland's reagent) or dithioerythritol at 0.01% to 0.1% weight/weight; acetylcysteine or cysteine at 0.1% to 0.5% (pH 2-3); and thioglycerol at 0.1% to 0.5% (pH 3.5 to 7.0) and glutathione. See Akers (1988) supra at pages 225 to 226. Suitable antioxidants include sodium bisulfite, sodium sulfite, sodium metabisulfite, sodium thiosulfate, sodium formaldehyde sulfoxylate, and ascorbic acid. See Akers (1988) supra at pages 225. Suitable chelating agents, which chelate trace metals to prevent the trace metal catalyzed oxidation of reduced cysteines, include citrate, tartarate, ethylenediaminetetraacetic acid (EDTA) in its disodium, tetrasodium, and calcium disodium salts, and diethylenetriamine pentaacetic acid (DTPA). Sec, e.g., Wang (1980) supra at pages 457-458 and 460-461, and Akers (1988) supra at pages 224-227.

The composition can include one or more preservatives such as phenol, cresol, p-aminobenzoic acid, BDSA, sorbitrate, chlorhexidine, benzalkonium chloride, or the like. Suitable stabilizers include carbohydrates such as trehalose or glycerol. The composition can include a stabilizer such as one or more of microcrystalline cellulose, magnesium stearate, mannitol, sucrose to stabilize, for example, the physical form of the composition; and one or more of glycine, arginine, hydrolyzed collagen, or protease inhibitors to stabilize, for example, the chemical structure of the composition. Suitable suspending additives include carboxymethyl cellulose, hydroxypropyl methylcellulose, hyaluronic acid, alginate, chondroitin sulfate, dextran, maltodextrin, dextran sulfate, or the like. The composition can include an emulsifier such as polysorbate 20, polysorbate 80, pluronic, triolein, soybean oil, lecithins, squalene and squalanes, sorbitan treioleate, or the like. The composition can include an antimicrobial such as phenylethyl alcohol, phenol, cresol, benzalkonium chloride, phenoxyethanol, chlorhexidine, thimerosol, or the like. Suitable thickeners include natural polysaccharides such as mannans, arabinans, alginate, hyaluronic acid, dextrose, or the like; and synthetic ones like the PEG hydrogels of low molecular weight and aforementioned suspending compounds.

The composition can include an adjuvant such as cetyl trimethyl ammonium bromide, BDSA, cholate, deoxycholate, polysorbate 20 and 80, fusidic acid, or the like, and a cationic lipid. Suitable sugars include glycerol, threose, glucose, galactose, mannitol, and sorbitol. A suitable protein is human serum albumin.

Preferred compositions include one or more of a solubility enhancing additive, preferably a cyclodextrin; a hydrophilic additive, preferably a monosaccharide or oligosaccharide; an absorption promoting additive, preferably a cholate, a deoxycholate, a fusidic acid, or a chitosan; a cationic surfactant, preferably a cetyl trimethyl ammonium bromide; a viscosity enhancing additive, preferably to promote residence time of the composition at the site of administration, preferably a carboxymethyl cellulose, a maltodextrin, an alginic acid, a hyaluronic acid, or a chondroitin sulfate; or a sustained release matrix, preferably a polyanhydride, a polyorthoester, a hydrogel, a particulate slow release depo system, preferably a polylactide co-glycolides (PLG), a depo foam, a starch microsphere, or a cellulose derived buccal system; a lipid-based carrier, preferably an emulsion, a liposome, a niosomes, or a micelles. The composition can include a bilayer destabilizing additive, preferably a phosphatidyl ethanolamine; a fusogenic additive, preferably a cholesterol hemisuccinate.

Other preferred compositions for sublingual administration including, for example, a bioadhesive to retain the compound sublingually; a spray, paint, or swab applied to the tongue; retaining a slow dissolving pill or lozenge under the tongue; or the like. Other preferred compositions for transdermal administration include a bioadhesive to retain the compound on or in the skin; a spray, paint, cosmetic, or swab applied to the skin; or the like.

These lists of carriers and additives is by no means complete and a worker skilled in the art can choose excipients from the GRAS (generally regarded as safe) list of chemicals allowed in the pharmaceutical preparations and those that are currently allowed in topical and parenteral formulations.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

From the foregoing description, one skilled in the art can easily ascertain the essential characteristics of this invention and, without departing from the spirit and scope thereof, can make various changes and modifications of the invention to adapt it to various usages and conditions.

Without further elaboration, it is believed that one skilled in the art can, using the preceding description and the information provided in the examples below, practice the present invention to its fullest extent.

### EXAMPLES

### Example 1

In view of the teaching provided herein, it is envisioned that measurements of OMA1, HIGD1A, OPA1, BNIP3, YMEIL1, PHB, SAMM50, IMMT and/or PHB2 gene expression levels are utilized for determining the susceptibility for, predisposition for, or the presence of a disorder or disease correlated with mitochondrial dysfunction, or a mitochondrial disorder or disease; or a disorder or disease characterized by OPA1 alterations.

A non-limiting example for such a disorder or disease is Alzheimer's disease. It is known in the arts that mitochondrial damage and dysfunction are early features of

Alzheimer's disease and other neurodegenerative diseases. Briefly, the two neuropathological hall marks of Alzheimer's disease are extracellular amyloid-ß deposits and intracellular tau accumulation causing plaques and neurofibrillary tangles, respectively (Taylor et al. 2002). However, dysfunctional mitochondria also have been recognized for many years in brains from deceased patients with Alzheimer's disease suggesting a direct connection between mitochondrial homeostasis and Alzheimer's disease. (Parker et al. 1990; Smith et al. 1996; Gibson et al. 1998; Maurer et al. 2000; Butterfield et al. 2001; Devi et al. 2006). Different genetic mouse models (Aliev et al. 2003; Li et al. 2004; Lustbader et al. 2004; Reddy et al. 2004; Caspersen et al. 2005; Manczak et al. 2006; Eckert et al. 2008; Yao et al. 2009) and cellular models for Alzheimer's disease showed mitochondrial impairments as well (Butterfield et al. 2001; Cardoso et al. 2004; Diana et al. 2008; Eckert et al. 2008; Schmidt et al. 2008; Wang et al. 2008). Amyloid-ß overexpression in cell culture studies decreased levels of DRP1 and OPA1, two key regulators of the dynamic mitochondrial network (Wang et al. 2008). Subsequent studies revealed that both amyloid-ß and tau can interact with DRP1 and OPA1 leading to altered OPA1 processing, impaired mitochondrial dynamics and function (Wang et al. 2008; Manczak et al. 2011; Manczak and Reddy 2012; Shields et al. 2015; Akhtar et al. 2016; Yang et al. 2017). Moreover, neuronal damage in Amyloid-ß and tau mouse models could be delayed by blocking DRP1-mediated mitochondrial fission or OMA1 knock-out (Merkwirth et al. 2008; Yan et al. 2015; Kandimalla et al. 2016; Korwitz et al. 2016; Manczak et al. 2016; Back et al. 2017; Reddy et al. 2017).

We mined all gene expression data available through the NCBI GEO database (Edgar et al. 2002) for studies comparing human brain samples from patients with and without Alzheimer's disease. We obtained the data set from the Hisayama study (GEO accession number: GDS4758), which examined 88 autopsy samples from Hisayama residents obtained between 15 Dec. 2008 and 24 Feb. 2011 (Hokama et al. 2014). We also obtained the data set from a large-scale transcriptional analysis of postmortem brain samples from deceased patients with late-onset Alzheimer's disease provided by two tissue centers (Alzheimer's Disease Center, Oregon Health and Sciences University, and Human Brain and Spinal Fluid Resource Center; GEO accession number: GSE29378) (Miller et al. 2013). We then analyzed the gene expression levels of OMA1, OPA1, HIGD1A, BNIP3, SAMM50, IMMT, YMEIL1, PHB and PHB2 in the different brain regions of patients with and without Alzheimer's disease. We calculated the differential expression with respect to region, disease, and sex. Differences were considered statistically significant for P-values of ≤0.05 using a Student's T-test.

We also mined the data repositories for FDA-approved drugs that modify the gene expression levels of OMA1, OPA1, HIGD1A, BNIP3, SAMM50, IMMT, YMEIL1, PHB or

PHB2. In addition, we searched for interventions that would modify the gene expression levels of OMA1, OPA1, HIGD1A, BNIP3, SAMM50, IMMT, YMEIL1, PHB or PHB2. We identified Ribavirin (CAS #: 36791-04-5) as OMA1 antagonist in a study of human hepatocytes (Huh7.5.1 cells; GEO accession number: GSE23031) (Thomas et al. 2011). Ribavirin is approved by the regulatory authorities for the use as antiviral for patients with hepatitis C.

In context of the present invention, it was, inter alia, found that OMA1, OPA1, BNIP3, SAMM50, IMMT and HIGD1A gene expression levels are differentially regulated in Alzheimer's disease brains in a sex-specific manner. Therefore, susceptibility and/or presence of Alzheimer's disease as well as treatment selection can be predicted by integrating these sex-specific expression profiles.

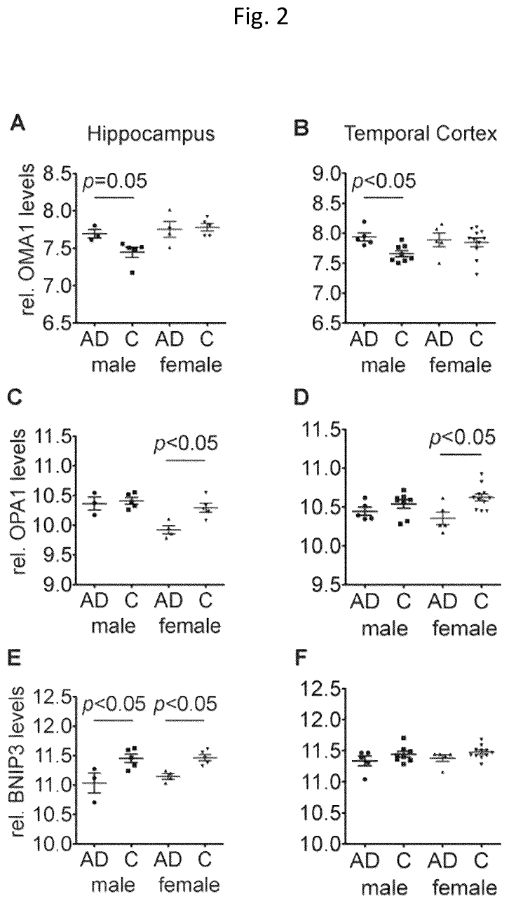

In one particular embodiment, we found significant gene expression changes in postmortem brain tissue from patients pathologically diagnosed as having Alzheimer's disease. OMA1 expression was significantly reduced in the hippocampus and temporal cortex of male individuals without Alzheimer's disease (FIGS. 2A and 2B), while OPA1 was significantly reduced in the hippocampus and temporal cortex of female Alzheimer's disease patients (FIG. 2C and FIG. 2D). BNIP3 levels were significantly reduced in the hippocampus of both male and female Alzheimer's disease patients (FIG. 2E), but not in the temporal cortex (FIG. 2F).

FIGS. 2A-2F show gene expression levels in the hippocampus (AD: n=8; C: n=9) and temporal cortex (AD: n=13; C: n=16) from post-mortem samples of patients with Alzheimer's disease (AD) and control subjects (C). OMA1 expression levels were significantly increased in males with Alzheimer's disease in the hippocampus by 3.3% (FIG. 2A) and the temporal cortex by 3.7% (FIG. 2B). Females appeared to have overall elevated OMA1 levels compared to males and there was no difference between Alzheimer's samples and controls. OPA1 levels were significantly reduced in the hippocampus of female patients with Alzheimer's by 3.7% (FIG. 2C) and the temporal cortex by 2.5% (FIG. 2C and FIG. 2D). BNIP3 levels were significantly reduced in the hippocampus from both male and female Alzheimer's patients by 3.7% and 2.8%, respectively (FIG. 2E), while there were no differences in the temporal cortex (FIG. 2F).

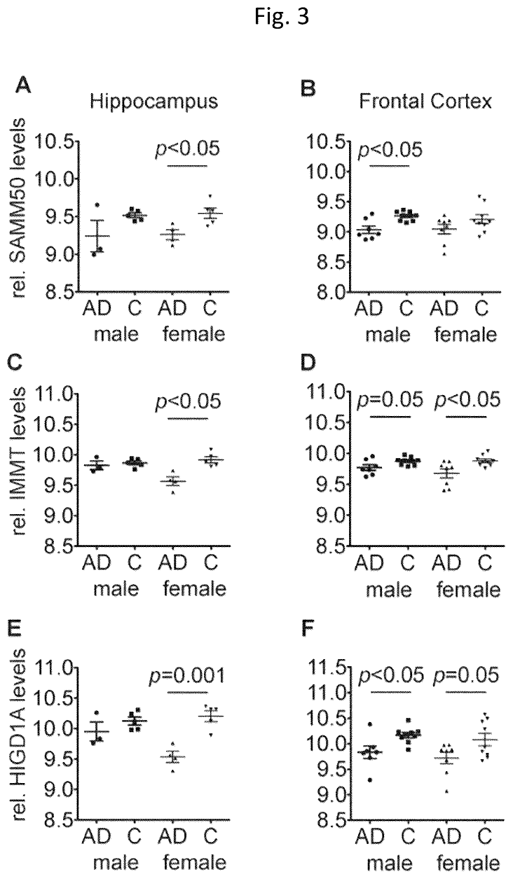

Expression of SAMM50 was significantly reduced in the hippocampus of female Alzheimer's disease patients (FIG. 3A) and the frontal cortex of male Alzheimer's disease patients (FIG. 3B). IMMT levels were significantly reduced in the hippocampus of female Alzheimer's disease patients (FIG. 3C) and in the frontal cortex of both male and female Alzheimer's disease patients (FIG. 3D). Expression of the endogenous OMA1 antagonist HIGD1A was also significantly reduced in the hippocampus of female Alzheimer's disease patients (FIG. 3E) and the frontal cortex of both male and female Alzheimer's disease patients (FIG. 3F).

FIGS. 3A-3F show gene expression levels in the hippocampus (AD: n=8; C: n=9) and frontal cortex (AD: n=16; C: n=17) from post-mortem samples of patients with Alzheimer's disease (AD) and control subjects C. SAMM50 expression levels appeared to be reduced in patients with Alzheimer's disease. They were significantly reduced by 3.0% in the hippocampus from Alzheimer's females (FIG. 3A) and by 2.4% in the frontal cortex from Alzheimer's males (FIG. 3B). IMMT levels were significantly reduced by 3.5% in the hippocampus from female subjects with Alzheimer's disease (FIG. 3C), while in the frontal cortex both males and females had significantly reduced IMMT levels by 2.0% and 1.0%, respectively (FIG. 3D). HIGD1A levels were significantly reduced by 6.6% in the hippocampus of female Alzheimer's patients (FIG. 3E), and in the frontal cortex of both male and female Alzheimer's patients by 3.3% and 3.6%, respectively (FIG. 3F).

In another embodiment, we found gene expression changes in the hippocampus from deceased late-onset Alzheimer's patients acquired at the Oregon Health and Sciences University (GEO accession number: GSE29378) (Miller et al. 2013). Again, OMA1 expression was significantly reduced in the hippocampus of non-Alzheimer's disease males (88.4%+15.1% S.D., p=0.05). We also found a trend for reduced HIGD1A levels in Alzheimer's brains at Oregon (86.3%±16.3% S.D., p-0.07), which agrees with the above data.

The specific changes in the gene expression of the genes OMA1, OPA1, BNIP3, SAMM50, IMMT and HIGD1A depends on the sex of a subject and the brain region. The specific expression profile and/or gene signature of these sex-dependent changes therefore represent a biomarker according to the definitions given above and is envisioned to be utilized, inter alia, for differentiation of Alzheimer's disease from other mitochondrial disorders or diseases, such as Parkinson's disease.

In view of the teaching provided herein, the means and methods of the present disclosure can also be used to inform selection and/or assist in selecting appropriate courses of treatment and/or medical interventions for patients in need of such interventions.

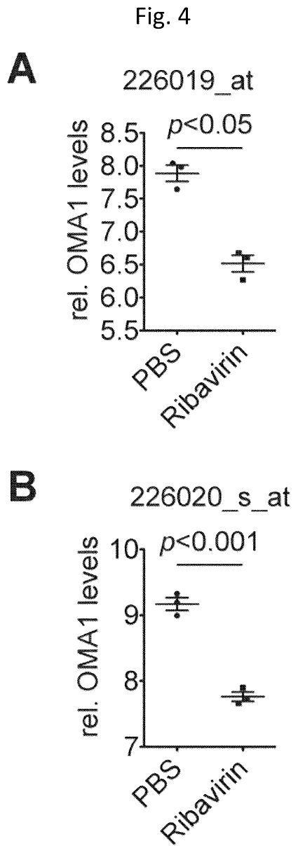

In one particular embodiment it was, inter alia, found that Ribavirin (CAS #: 36791-04-5) significantly decreases the gene expression levels of OMA1 by 15% to 17% (FIG. 4; Student's T-test: p≤0.05). Human hepatocytes (Huh7.5.1 cells) were cultured with or without 100 μg/mL Ribavirin (Thomas et al. 2011). Cells without Ribavirin (FIG. 4; PBS) expressed OMA1 at 7.89 (±0.21 S.D.; Spot ID: 226019_at) and 9.17 (±0.17 S.D. (FIG. 4A); Spot ID: 226020_s_at), while Ribavirin-treated cells had significantly reduced OMA1 levels at 6.52 (±0.22 S.D.; Spot ID: 226019_at) and 7.76 (±0.12 S.D.; Spot ID: 226020_s_at) (FIG. 4B), respectively.

Referring to FIGS. 4A and 4B, the antiviral Ribavirin (CAS #36791-04-5) represents an antagonist of OMA1. Ribavirin can significantly decrease the expression levels of OMA1 in human hepatocytes by 15% to 17%. Huh7.5.1 cells were cultured with either the addition of PBS or 100 μg/mL Ribavirin for 24 hours, after which mRNA was isolated and analyzed by gene expression microarrays. Vehicle treated cells (PBS) expressed OMA1 at 7.89 (±0.21 S.D.; Spot ID: 226019_at) and 9.17 (±0.17 S.D.; Spot ID: 226020_s_at) (FIG. 4A), while Ribavirin significantly reduced OMA1 levels to 6.52 (±0.22 S.D.; Spot ID: 226019_at) and 7.76 (±0.12 S.D.; Spot ID: 226020_s_at) (FIG. 4B), respectively.

Ribavirin is known in the arts as an antiviral agent that also possesses immunosuppressant activity. Neurotrophic activity of Ribavirin and analogs thereof are also known in the arts (WO 00/30656). However, the finding that Ribavirin can decrease OMA1 levels was unexpected and is non-obvious to a person skilled in the arts.

In context of the present invention Ribavirin thus represents an antagonist of OMA1. Accordingly, Ribavirin represents a medical intervention in particular for the treatment, of patients with Alzheimer's disease. Apovir is a combination drug of Ribavirin and Pleconaril that has been tested in a clinical study on patients with Alzheimer's disease (EudraCT number: 2013-002126-23) to investigate its effect on disease progression as assessed by the Alzheimer's Disease Assessment Scale-Cognitive Subscale [ADAS-cog]. Patients that received 600 mg Apovir per day (n=18) showed an improvement of −1.963 (±4.398 S.D.) points on the ADAS-cog. Subscale after 9 months, compared to placebo-treated patients (n=31), which showed worsening of 1.817 (±8.623 S.D.) points. Although these changes did not achieve statistical significance (P=0.1809), these results validate our method for the treatment of a mitochondrial disease, on the basis of Alzheimer's disease as an example for such a disease.

### Example 2

Another non-limiting example for a disease correlated with altered OPA1 processing is Parkinson's disease for which there is strong evidence for mitochondrial dysfunction (Schapira et al. 1990; Keeney et al. 2006; Parker et al. 2008; Santos et al. 2015; Dolle et al. 2016). Briefly, familial forms of Parkinson's disease are associated with several proteins that directly impact mitochondrial fission and fusion. For example, a-synuclein can localize to mitochondria, and mitochondria associated ER membranes (Imaizumi et al. 2012; Nakamura 2013; Guardia-Laguarta et al. 2014; Ghio et al. 2016). Overexpression of mutant a-synuclein increased mitophagy in cortical neurons along with mitochondrial fragmentation and neuronal cell death by promoting OPA1 cleavage (Guardia-Laguarta et al. 2014). The adverse effects of mutant a-synuclein could be rescued in part by inhibiting mitophagy (Choubey et al. 2011; Nakamura et al. 2011). Pink1, Parkin and DJ-1 form an E3 ubiquitin-ligase complex on the mitochondrial surface that initiates mitophagy by ubiquitinating, among other proteins, DRP1 (Yang et al. 2008; Lutz et al. 2009; Wang et al. 2011). Mutations in any of the genes result in frustrated clearance and accumulation of damaged mitochondria leading to neuronal loss in the substantia nigra and other parts of the brain (Mukherjee et al. 2015). LRRK2 also resides on the mitochondrial outer membrane where it affects mitochondrial network dynamics through interactions with DRP1 and OPA1 (Stafa ct al. 2014). As is the case for Alzheimer's disease, preventing mitochondrial fission through DRP1 inhibition also can attenuate neurotoxicity in animal models of Parkinson's disease (Rappold et al. 2014).

We mined all gene expression data available through the NCBI GEO database (Edgar et al. 2002) for studies comparing human brain samples from patients with and without Parkinson's disease. We obtained gene expression data from 16 patients with Parkinson's disease for which post mortem brain specimens were investigated. These patients were enrolled prospectively at the Department of Neurology of the Mayo Clinic in Rochester, MN, from June 1996 through May 2004 (GEO accession number: GDS2821) (Maraganore et al. 2005; Lesnick et al. 2007). We also obtained an independent second data set from postmortem brain tissue of 11 subjects diagnosed with neuropathologically confirmed Parkinson's disease. The tissue blocks were acquired from the University of Maryland Brain and Tissue Bank, the New York Brain Bank at Columbia University, the Human Brain and Spinal Fluid Resource Center at the West Los Angeles VA Medical Center, McLean Hospital of Harvard University, and the Miami Brain and Tissue Bank (GEO accession number: GSE20168) (Zhang et al. 2005). We then analyzed the gene expression levels of OMA1, OPA1, HIGD1A, BNIP3, SAMM50, IMMT, YMEIL1, PHB and PHB2 in the different brain regions of patients with and without Parkinson's disease. We calculated the differential expression with respect to region, disease, and sex. Differences were considered statistically significant for P-values of ≤0.05 using a Student's T-test.

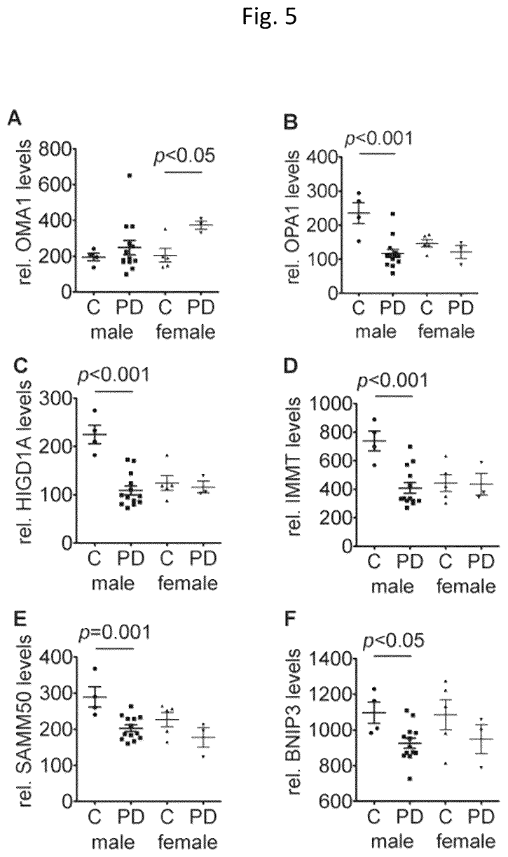

In one particular embodiment, we found significant gene expression changes in samples of the substantia nigra from postmortem brain tissue from patients with Parkinson's disease (GEO accession number: GDS2821). OMA1 gene expression levels were significantly increased in samples of the substantia nigra from female Parkinson's patients (FIG. 5A). OPA1 gene expression levels, on the other hand, were significantly decreased in Parkinson's samples from male subjects (FIG. 5B). Females had overall lower OPA1 expression levels and there was no significant difference between non-PD and PD samples (FIG. 5B). HIGD1A levels were also significantly decreased in males with Parkinson's, while females had overall lower HIGD1A levels and did not show any significant differences (FIG. 5C). IMMT levels were significantly decreased in male Parkinson's samples to about the levels of healthy females and females with Parkinson's (FIG. 5D). SAMM50 was decreased in Parkinson's samples; this difference was statistically significant for males, while females showed lower overall levels (FIG. 5E). PHB2 levels appeared to be reduced in Parkinson's samples, which was statistically significant for male subjects (FIG. 5F).

FIGS. 5A-5F show gene expression levels in post mortem samples of the substantia nigra from a cohort of healthy subjects (C; n=9) and patients with Parkinson's disease (PD; n=16) collected at Rochester, MN (GEO accession number: GDS2821). OMA1 genc expression levels appeared to be increased in Parkinson's samples; they were significantly increased by 27.6% in samples from female Parkinson's patients (FIG. 5A). OPA1 gene expression levels were significantly decreased by 50.3% in samples from male Parkinson's subjects (FIG. 5B). Females had overall lower OPA1 expression levels in the substantia nigra than males and there was no significant difference between C and PD samples (5B). HIGD1A levels were significantly decreased by 51.5% in males with Parkinson's (FIG. 5C). Again, females had overall lower HIGD1A levels and did not show any significant differences. IMMT levels were also significantly decreased in males with Parkinson's by 44.6%, while females had overall lower IMMT levels and no significant changes (FIG. 5D). SAMM50 levels appeared to be decreased in Parkinson's samples; this difference was statistically significant for males (29.9% reduction), while females again showed lower overall levels (FIG. 5E). PHB2 levels appeared to be reduced in Parkinson's samples as well, which was statistically significant for males (FIG. 5F; 15.5% reduction).

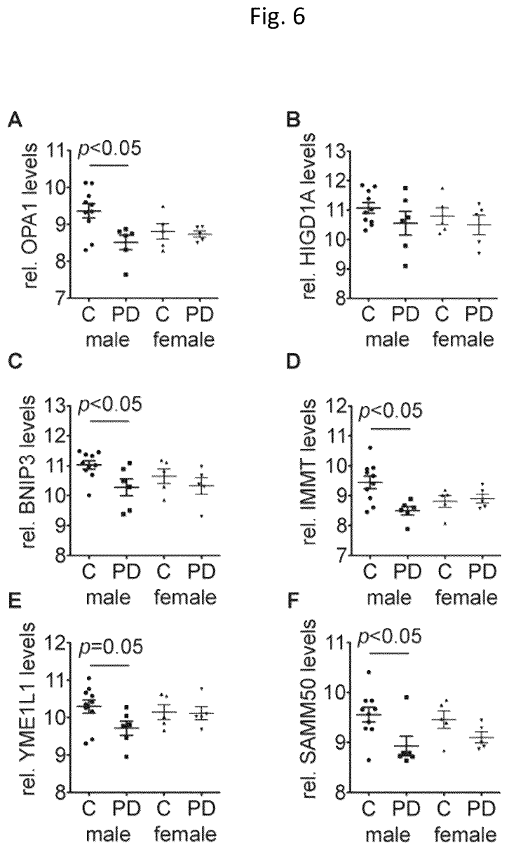

In another embodiment, we found significant gene expression changes in an independent set of samples from the substantia nigra of postmortem brain tissue from patients with Parkinson's disease (GEO accession number: GSE20292). In agreement with the data obtained from the first study, OPA1 gene expression levels were significantly decreased in Parkinson's samples from male subjects (FIG. 6A). Females had overall lower OPA1 expression levels and there was no significant difference between non-PD and PD samples (FIG. 6A). HIGD1A levels were overall lower in Parkinson's samples but did not reach statistical significance (FIG. 6B). BNIP3 gene expression levels were significantly reduced in male Parkinson's samples and females appear to have overall lower BNIP3 expression levels and there was no significant difference (FIG. 6C). Also, IMMT levels were significantly decreased in males with Parkinson's, while females had overall lower levels and did not show any significant differences (FIG. 6D). YMEIL1 levels showed a small but significant decrease in male Parkinson's samples (FIG. 6E). SAMM50 was decreased in Parkinson's samples and this difference was statistically significant for males (FIG. 6F).

FIGS. 6A-6F show gene expression levels in post mortem samples of the substantia nigra from an independent cohort of healthy subjects (C; n=15) and patients with Parkinson's disease (PD; n=11) collected at Syracuse, NY (GEO accession number: GSE20292). OPA1 gene expression levels were significantly decreased by 9.1% in Parkinson's samples from male subjects (FIG. 6A). HIGD1A levels were overall lower in Parkinson's samples but did not reach statistical significance (FIG. 6B). BNIP3 gene expression levels were significantly reduced by 6.8% in male Parkinson's samples (FIG. 6C). Females appear to have overall lower BNIP3 expression levels and there was no significant difference. IMMT levels were significantly decreased by 10.1% in males with Parkinson's, while females had overall lower levels and again did not show any significant differences (FIG. 6D). YMEIL1 levels were significantly reduced by 5.6% in male Parkinson's samples (FIG. 6E). Overall SAMM50 levels were decreased in Parkinson's samples; males had a significant reduction by 6.6% while the difference between female samples did not reach statistical significance (FIG. 6F).

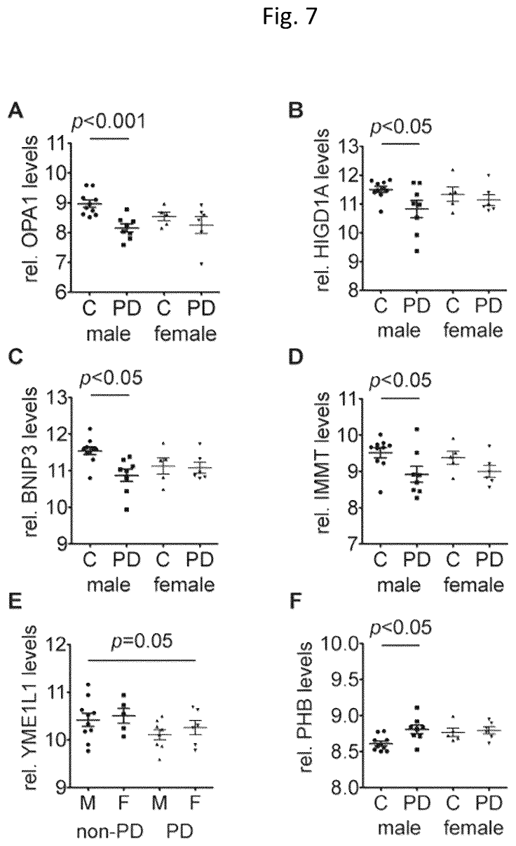

In yet another embodiment, we found significant gene expression changes in post mortem samples of the prefrontal cortex from patients with Parkinson's disease (GEO accession number: GSE20168). OPA1 gene expression levels were also significantly decreased in the prefrontal cortex of male patients with Parkinson's (FIG. 7A). HIGD1A levels were significantly lower in male Parkinson's samples (FIG. 7B). BNIP3 gene expression levels were significantly reduced in male Parkinson's samples (FIG. 7C). IMMT levels appeared to be lower in Parkinson's samples and there was a statistically significant reduction in male samples (FIG. 7D). YMEIL1 levels showed a small but significant reduction in Parkinson's samples when male and female samples were analyzed together (FIG. 7E). Females tended to have lower overall expression levels and did not show any significant changes for these genes. PHB showed a small but significant increase in the prefrontal cortex of males with Parkinson's (FIG. 7F).

FIGS. 7A-7F show gene expression levels in the prefrontal cortex from healthy subjects (C; n=15) and patients with Parkinson's disease (PD; n=11) from Syracuse, NY (GEO accession number: GSE20168). OPA1 gene expression levels were significantly decreased by 9.1% in Parkinson's samples from male subjects (FIG. 7A). HIGD1A levels were significantly reduced by 5.9% in male Parkinson's samples (FIG. 7B). BNIP3 gene expression levels were significantly reduced by 5.8% in male Parkinson's samples (FIG. 7C). IMMT levels appeared to be lower in Parkinson's samples and there was a statistically significant reduction by 6.2% in male samples (FIG. 7D). There was a 2.6% decrease of YME1L1 levels, in Parkinson's samples, which was significant when male and female samples were analyzed together (FIG. 7E). PHB was significantly increased in the prefrontal cortex from male Parkinson's patients by 2.3% (FIG. 7F).

We have demonstrated above that Parkinson's disease correlates with specific changes in the gene expression levels of OMA1, OPA1, HIGD1A, BNIP3, SAMM50, IMMT, YMEIL1, PHB and PHB2. These changes depend on the sex of a subject and the brain region. In context of the present invention, it is envisioned that measuring and integrating these gene expression changes can be utilized, inter alia, for determining the susceptibility for, predisposition for, or the presence of Parkinson's disease. These measurements also can be utilized for differentiation of Parkinson's disease from other disorders or diseases correlated with mitochondrial dysfunction, such as Alzheimer's disease. Moreover, this biomarker is envisioned to guide treatment selection.

### Example 3

Yet another non-limiting example for a mitochondrial disorder or disease is cancer. Research over the past century or so has generated a complex and rich body of knowledge revealing cancer to be a disease correlated to mitochondrial dysfunction (Alirol and Martinou 2006; Frezza and Gottlieb 2009; Hanahan and Weinberg 2011; Wallace 2012; Vyas et al. 2016). Uncontrolled cell proliferation represents the essence of neoplastic disease and entails adjustments of energy metabolism in order to fuel cell growth and division. Aerobic glycolysis is an anomalous characteristic of cancer cell energy metabolism referred to as “Warburg effect”: even in the presence of oxygen, cancer cells largely adopt glycolysis (Warburg 1956). The existence of this metabolic switch in cancer cells has been substantiated and other mitochondrial changes have been described in the ensuing decades (Alirol and Martinou 2006; Frezza and Gottlieb 2009; Hanahan and Weinberg 2011; Wallace 2012; Vyas et al. 2016). It is also known in the arts that changes in energy metabolism are tightly correlation with alterations in mitochondrial morphology (Hackenbrock 1966; Jakobs ct al. 2003; Cogliati et al. 2013). Changes in OPA1 levels and OPA1 processing have been found in various cancers, accordingly (Zhao et al. 2013; Kong et al. 2014; Faccenda et al. 2017). These mitochondrial alterations might not be the primary cause for cancer. And yet, they are functionally important for the development and progression of many forms of human cancer. Therefore, mitochondrial dynamics can adjust chemoresistance in cancer (Kong et al. 2015) and regulate tumorigenesis, metastatic spread and overall survival of cancer cells.

We obtained gene-expression data paired with survival data for different tumor samples through public data repositories and conducted meta studies on 1764 patients with breast cancer (GEO accession numbers: E-MTAB-365, GSE12276, GSE16391, GSE16446, GSE17907, GSE19615, GSE20685, GSE20711, GSE21653, GSE42568, GSE9195), on 1145 patients with lung cancer (GEO accession numbers: GSE19188, GSE3141, GSE50081, GSE37745, GSE29013, GSE30219, GSE31210), on 631 patients with gastric cancer (GEO accession numbers: GSE22377, GSE15459, GSE51105, GSE62254, GSE62254), and on 1435 patients with ovarian cancer (GEO accession numbers: GSE51373, GSE9891,

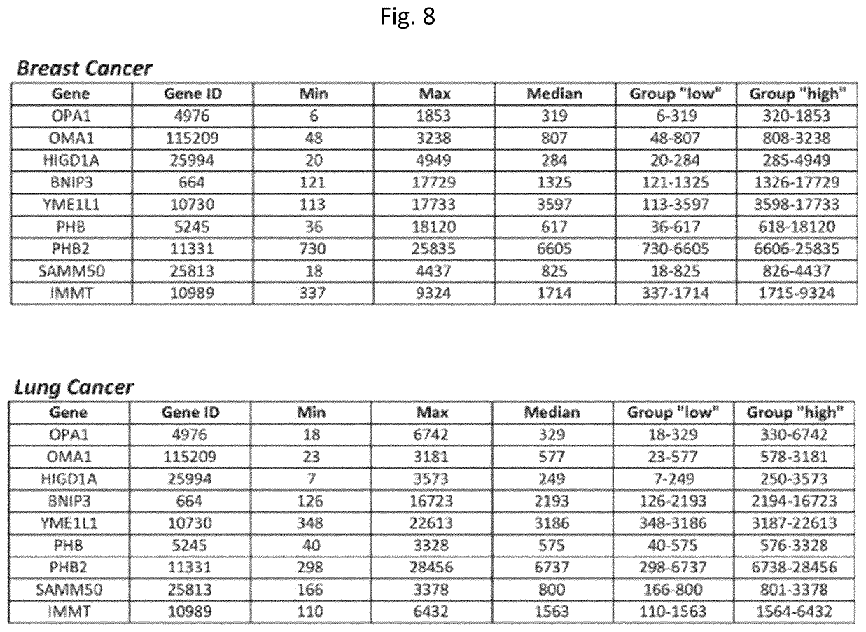

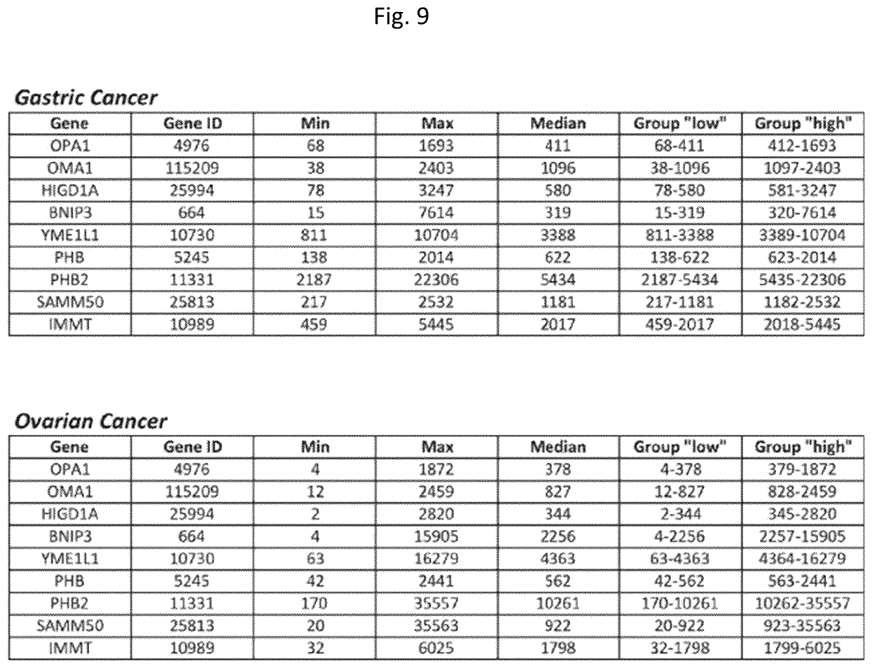

GSE15622, GSE26712, GSE26193, GSE63885, GSE65986, GSE30161, GSE14764, TCGA). We analyzed different gene signatures for their predictive value on patient survival. To this end we calculated the median gene-expression levels for OMA1, HIGD1A, OPA1, BNIP3, YMEIL1, PHB, SAMM50, IMMT and/or PHB2 in the different datasets and defined two groups of patients, depending on whether a subject's expression levels were below (group “low”) or above the median gene expression levels (group “high”). The minimum, maximum and median gene expression levels for OPA1, OMA1, HIGD1A, BNIP3, YMEIL1, PHB, PHB2, SAMM50 and IMMT for each of the 4 investigated cancer types (i.e., breast cancer, lung cancer, gastric cancer, ovarian cancer) are given in the tables depicted in FIG. 8 and FIG. 9, respectively. Also, the data range for each group, “low” or “high”, for each gene is depicted in FIG. 8 and FIG. 9. Based on these classifications we tested the predictive value of different combinations of at least 3 genes. We termed these groups of 3 or more combinations of gene expression groups “gene signatures” (e.g., OMA1: high, HIGD1A: high, BNIP3: low). We tested whether patients with particular gene signatures had an increased chance of overall survival using a Log-rank Test. Differences were considered statistically significant for P-values of ≤0.05.

FIG. 8 shows data ranges for OPA1, OMA1, HIGD1A, BNIP3, YMEIL1, PHB, PHB2, SAMM50 and IMMT gene expression levels in tissue samples from patients with breast cancer (top) and lung cancer (bottom). The median expression levels and the data range for the classification of patients, depending on whether a subject's expression levels were below (group “low”) or above the median gene expression levels (group “high”) are given as well.

FIG. 9 shows data ranges for OPA1, OMA1, HIGD1A, BNIP3, YMEIL1, PHB, PHB2, SAMM50 and IMMT gene expression levels in tissue samples from patients with gastric cancer (top) and ovarian cancer (bottom). The median expression levels and the data range for the classification of patients, depending on whether a subject's expression levels were below (group “low”) or above the median gene expression levels (group “high”) are given as well.

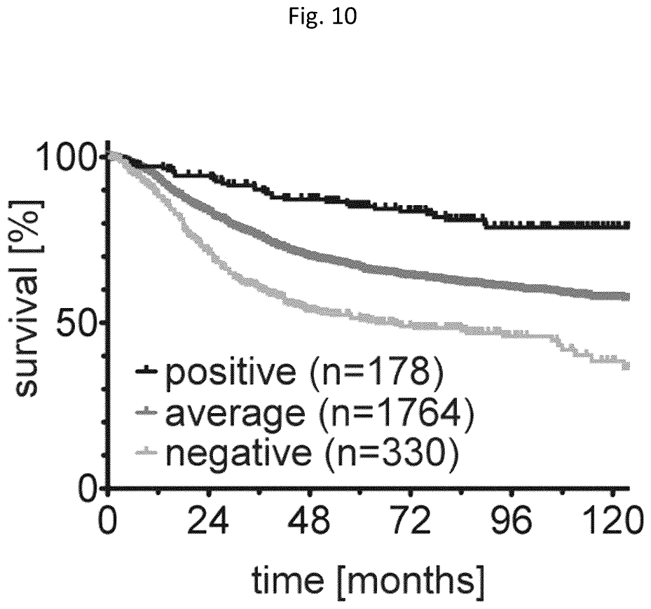

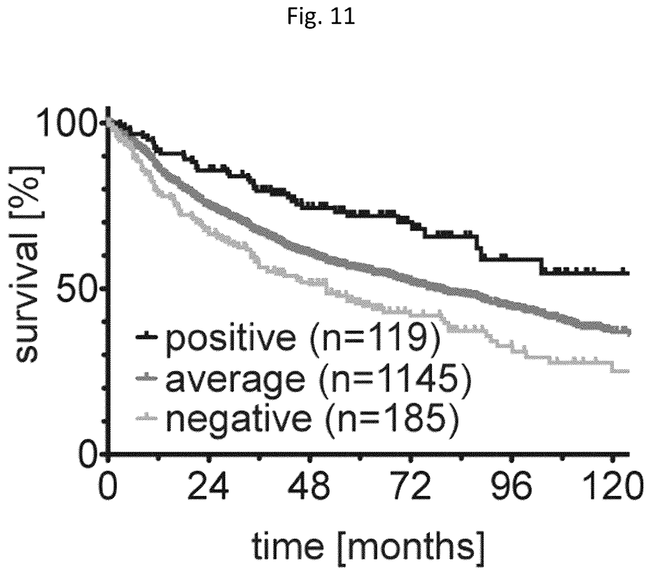

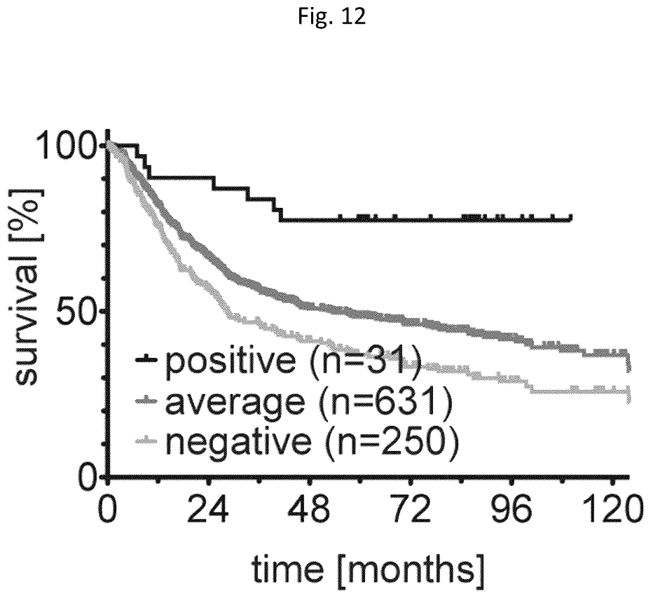

In one particular embodiment we have invented a 3-gene expression signature particularly useful for the prognosis of survival of patients with cancer. This 3-gene signature comprises OMA1, HIGD1A and BNIP3, wherein OMA1 and HIGD1A expression levels are elevated and BNIP3 expression levels are reduced compared to the median expression levels. This particular 3-gene signature can stratify patients with breast cancer based on whether the expression levels of OMA1, HIGD1A and BNIP3 matched (positive; i.e., OMA1: high; HIGD1A: high; BNIP3 low) or did not match (negative; i.e., OMA1: low; HIGD1A: low; BNIP3: high) the signature (FIG. 10). The median survival of patients with a negative gene signature was only 68 months, while patients with a positive signature had a significantly higher chance of survival (FIG. 10; Log-rank Test: p<0.0001; Chi square=65.60; df=2). This particular 3-gene signature has also been proven useful to stratify patients with lung cancer (FIG. 11) and gastric cancer (FIG. 12). Patients with lung cancer survived on average 78 months, while subjects with a negative signature survived only 52 months and subjects with a positive signature had significantly better chances of survival (FIG. 11; Log-rank Test: p<0.0001; Chi square=21.77; df=2). On the other hand, patients with gastric cancer had on average 53 months to survive, while subjects with a negative signature survived only 28 months and subjects with a positive signature had significantly better chances of survival (FIG. 12; Log-rank Test: p<0.0001; Chi square=24.12; df=2).

FIG. 10 shows Kaplan-Meier curves showing the overall survival of 1764 patients with breast cancer (average; dark grey, solid line; GEO accession numbers: E-MTAB-365, GSE12276, GSE16391, GSE16446, GSE17907, GSE19615, GSE20685, GSE20711, GSE21653, GSE42568, GSE9195) stratified by a proprietary 3-gene signature based on OMA1, HIGD1A and BNIP3 expression levels (i.e., OMA1: high, HIGD1A: high, BNIP3: low). Patients whose signature matched all 3 genes (positive; black, solid line) had a 100% chance of survival, while patients whose signature was inverted (i.e., OMA1: low, HIGD1A: low, BNIP3: high; negative; light grey, dotted line) had only a 60% chance with a median survival of 42 months (Log-rank Test: p<0.0001; Chi square=65.60; df=2).

FIG. 11 shows Kaplan-Meier curves showing the overall survival of 1145 patients with lung cancer (average; dark grey, solid line; GEO accession numbers: GSE19188, GSE3141, GSE50081, GSE37745, GSE29013, GSE30219, GSE31210) stratified by the proprietary 3-gene signature (i.e., OMA1: high, HIGD1A: high, BNIP3: low). Patients whose signature matched all 3 genes (positive; black, solid line) had an 82% chance of survival, while patients whose signature was inverted (i.e., OMA1: low, HIGD1A: low, BNIP3: high; negative; light grey, dotted line) had only a 55% chance with a median survival of 25 months (Log-rank Test: p<0.0001; Chi square=21.77; df=2).

FIG. 12 shows Kaplan-Meier curves showing the overall survival of 631 patients with gastric cancer (average; dark grey, solid line; GEO accession numbers: GSE22377, GSE15459, GSE51105, GSE62254, GSE62254) stratified by the proprietary 3-gene signature (i.e., OMA1: high, HIGD1A: high, BNIP3: low). Patients whose signature matched all 3 genes (positive; black, solid line) had an 84% chance of survival, while patients whose signature was inverted (i.e., OMA1: low, HIGD1A: low, BNIP3: high; negative; light grey, dotted line) had only a 53% chance with a median survival of 25 months (Log-rank Test: p<0.0001; Chi square=24.12; df=2).

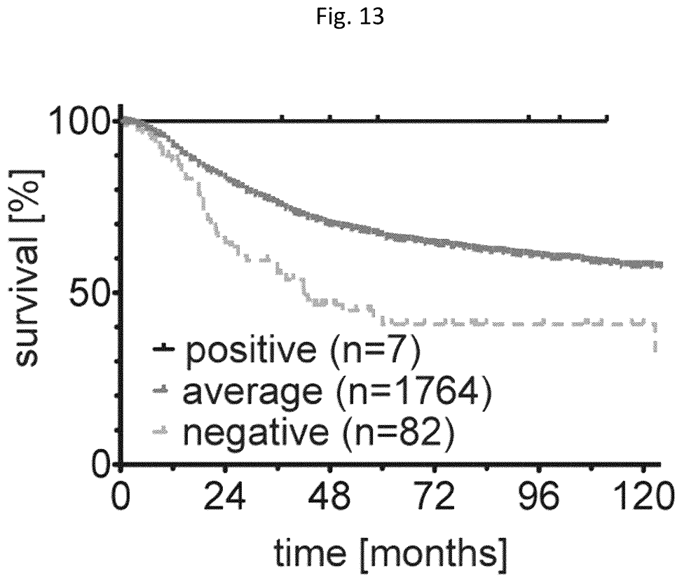

In another particular embodiment we have invented a more refined 6-gene expression signature particularly useful for the prognosis of survival of patients with breast cancer (FIG. 13). This 6-gene signature comprises OMA1, HIGD1A, BNIP3, OPA1, YMEIL1 and IMMT, wherein OMA1 and HIGD1A expression levels are elevated and BNIP3, OPA1, YMEIL1 and IMMT expression levels are reduced compared to the median expression levels (i.e., OMA1: high, HIGD1A: high, BNIP3: low, OPA1: low, YMEIL1: low, IMMT: low). In a meta-study of 1764 patients with breast cancer, we were able to identify patients with 100% chance of survival provided that all 6 genes matched the 6-gene signature (FIG. 13, positive). Subjects whose gene expression profile did not match the 6-gene signature (negative; i.e., OMA1: low, HIGD1A: low, BNIP3: high, OPA1: high, YMEIL1: high, IMMT: high) had only a 60.26% (±21.61 S.D.) chance of survival, and the median survival was 42 months (Log-rank Test: p<0.0001; Chi square=93.45; df=6). When the expression levels of 5 genes matched the 6-gene signature (⅚), patients had on average a chance of 87.19% (±7.188 S.D.). When 4 genes matched the 6-gene signature ( 4/6), the chance was 79.59% (±9.399 S.D.). When 3 genes matched the 6-gene signature ( 3/6), the chance was 73.12% (±12.70 S.D.). When 2 genes matched the 6-gene signature ( 2/6), the chance was 69.15% (±13.49 S.D.). And when only 1 gene matched the 6-gene signature (⅙), there was a 60.72% (±17.04 S.D.) chance of survival with a median survival of 73 months.

FIG. 13 shows Kaplan-Meier curves showing the overall survival of 1764 patients with breast cancer (average; dark grey, solid line; GEO accession numbers: E-MTAB-365, GSE12276, GSE16391, GSE16446, GSE17907, GSE19615, GSE20685, GSE20711, GSE21653, GSE42568, GSE9195) stratified by a proprietary 6-gene signature based on OMA1, HIGD1A, OPA1, BNIP3, YMEIL1 and IMMT expression levels. Patients whose signature matched all 6 genes (positive; black, solid line) had a 100% chance of survival, while patients whose signature did not match any of the 6 genes (negative; light grey, dotted line) had only a 60% chance with a median survival of 42 months (Log-rank Test: p<0.0001; Chi square=93.45; df=6).

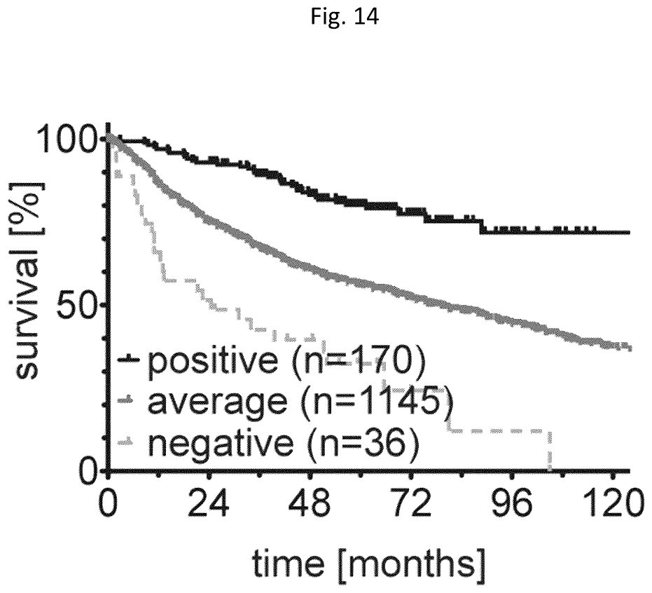

In another embodiment it was, inter alia, found that a proprietary 6-gene signature has proven useful for the prognosis of survival of patients with lung cancer (FIG. 14). The 6-gene signature comprises elevated levels of OMA1, HIGD1A, YMEIL1, PHB and SAMM50 compared to the median expression levels, and reduced PHB2 levels compared to the median expression levels (i.e., OMA1: high, HIGD1A: high, YMEIL1: high, PHB: high, SAMM50: high, PHB2: low). In a meta-study of 1145 patients with lung cancer, we were able to identify patients with an 81.73% (±7.114 S.D.) chance of survival provided that all 6 genes matched the 6-gene signature (FIG. 14, positive). Subjects whose gene expression profile did not match the 6-gene signature (negative; i.e., OMA1: low, HIGD1A: low, YMEIL1: low, PHB: low, SAMM50: low, PHB2: high) had only a 54.67% (±25.63 S.D.) chance of survival, and the median survival was 25 months (Log-rank Test: p<0.0001; Chi square=85.40; df=6). When expression levels of 5 genes matched the 6-gene signature (5/6), there was a 69.72% (±16.07 S.D.) chance and the median survival was 110 months. When 4 genes matched the 6-gene signature ( 4/6), the chance was 62.50% (±19.47 S.D.) and the median survival was 89 months. When 3 genes matched the 6-gene signature ( 3/6), there was a 57.84% (±20.73 S.D.) chance and the median survival was 55 months. When 2 genes matched the 6-gene signature ( 2/6), there was a 58.82% (±22.90 S.D.) chance and the median survival was 57 months. When only 1 gene matched the 6-gene signature (⅙), there was a 58.11% (±21.49 S.D.) chance and a subject's median survival was 45 months.

FIG. 14 shows Kaplan-Meier curves showing the overall survival of 1145 patients with lung cancer (average; dark grey, solid line; GEO accession numbers: GSE19188, GSE3141, GSE50081, GSE37745, GSE29013, GSE30219, GSE31210) stratified by a proprietary 6-gene signature based on OMA1, HIGD1A, YMEIL1, PHB, SAMM50 and PHB2 expression levels. Patients whose signature matched all 6 genes (positive; black, solid line) had an 82% chance of survival, while patients whose signature did not match any of the 6 genes (negative; light grey, dotted line) had only a 55% chance with a median survival of 25 months (Log-rank Test: p<0.0001; Chi square=85.40; df=6).

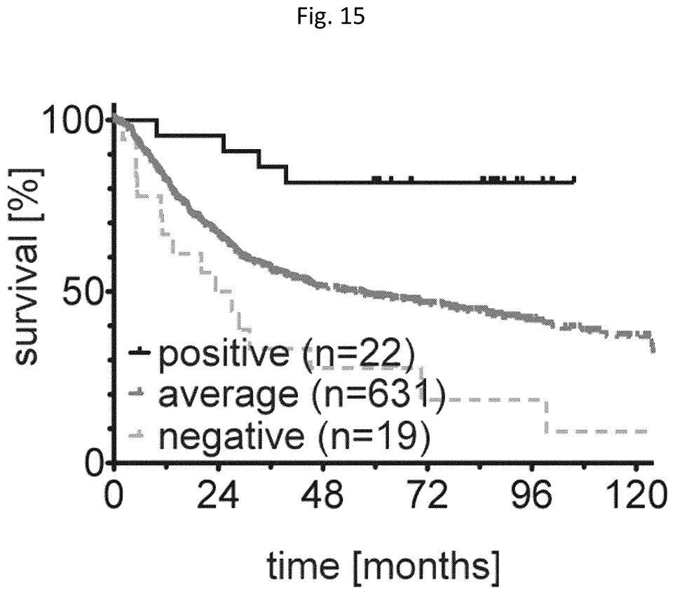

In yet another embodiment it was, inter alia, found that a proprietary 7-gene signature has proven useful for the prognosis of survival of patients with gastric cancer (FIG. 15). The 7-gene signature comprises OMA1, HIGD1A, OPA1, BNIP3, YMEIL1, SAMM50 and IMMT, wherein expression levels of BNIP3 are elevated while OMA1, HIGD1A, OPA1, YMEIL1, SAMM50 and IMMT levels are decreased compared to the median levels (i.e., OMA1: low, HIGD1A: low, OPA1: low, YMEIL1: low, SAMM50: low, IMMT: low, BNIP3: high). In a meta-study of 631 patients with gastric cancer, we were able to identify patients with an 83.79% (±4.908 S.D.) chance of survival provided that all 7 genes matched the 7-gene signature (FIG. 15, positive). Subjects whose gene expression profile did not match the 7-gene signature (negative; i.e., OMA1: high, HIGD1A: high, OPA1: high, YMEIL1: high, SAMM50: high, IMMT: high, BNIP3: low) had only a 52.53% (±29.71 S.D.) chance of survival, and the median survival was 25 months (Log-rank Test: p<0.0001; Chi square=50.81; df=7). When 6 genes matched the 7-gene signature ( 6/7), there was a 74.68% (±11.29 S.D.) chance of survival. When 5 genes matched the 7-gene signature (5/7), there was a 67.07% (±14.17 S.D.) chance of survival. When 4 genes matched the 7-gene signature ( 4/7), there was a 69.22% (±14.48 S.D.) chance of survival. When 3 genes matched the 7-gene signature ( 3/7), the likelihood to survive was 61.61% (±21.79 S.D.) and the median survival was 38 months. When 2 genes matched the 7-gene signature ( 2/7), there was a 58.66% (±25.76 S.D.) chance and the median survival was 31 months. When only 1 gene matched the 7-gene signature ( 1/7), there was a 57.70% (±23.51 S.D.) chance and the median survival was 29 months.

FIG. 15 shows Kaplan-Meier curves showing the overall survival of 631 patients with gastric cancer (average; dark grey, solid line; GEO accession numbers: GSE22377, GSE15459, GSE51105, GSE62254, GSE62254) stratified by a proprietary 7-gene signature based on OMA1, HIGD1A, YMEIL1, PHB, SAMM50 and PHB2 expression levels. Patients whose signature matched all 7 genes (positive; black, solid line) had an 84% chance of survival, while patients whose signature did not match any of the 7 genes (negative; light grey, dotted line) had only a 53% chance with a median survival of 25 months (Log-rank Test: p<0.0001; Chi square=50.81; df=7).

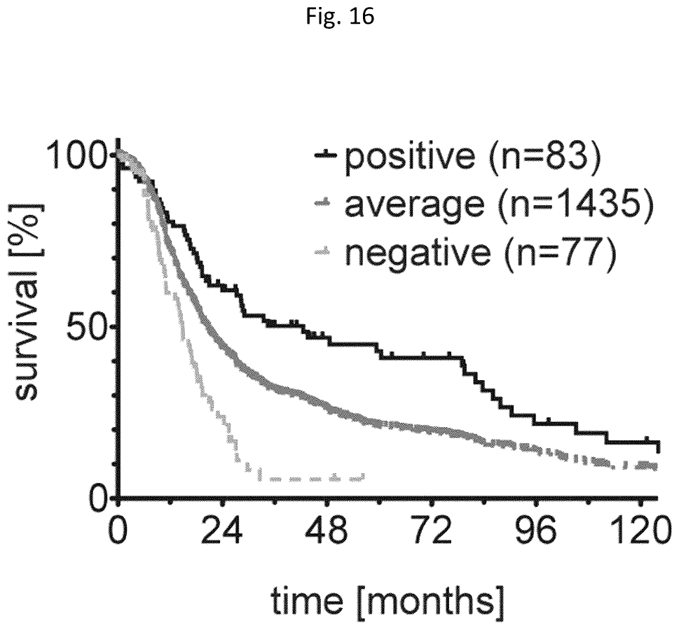

In yet another embodiment it was, inter alia, found that a proprietary 5-gene signature has proven useful for the prognosis of survival for patients with ovarian cancer (FIG. 16). The 5-gene expression signature comprises OPA1, BNIP3, YMEIL1 and IMMT, which are reduced in their gene expression levels, and PHB, which has elevated levels compared to the median expression levels (i.e., OPA1: low, BNIP3: low, YMEIL1: low, IMMT: low, PHB: high). In a meta-study of 1435 patients with ovarian cancer, we were able to identify patients with a 58.25% (±25.15 S.D) chance of survival provided that all 5 genes matched the 5-gene signature (FIG. 16, positive). These patients had a median survival of 43 months. When no gene matched the 5-gene signature (negative; i.e., OPA1: high, BNIP3: high, YMEIL1: high, IMMT: high, PHB: low), there was a 56.81% (±28.46 S.D.) chance of survival and subjects had a median survival of 15 months (Log-rank Test: p<0.0001; Chi square=34.92; df=5). Patients had a 55.13% (±25.97 S.D.) chance of survival with a median survival of 24 months provided that 4 genes matched the 5-gene signature (⅘). When 3 genes matched the 5-gene signature (⅗), there was a 53.06% (±27.76 S.D.) chance and the median survival was 21 months. When 2 genes matched the 5-gene signature (⅖), there was a 57.53% (±28.81 S.D.) chance and the median survival was 19 months. When only 1 gene matched the 5-gene signature (⅕), there was a 60.48% (±28.47 S.D.) chance of survival and a subject's median survival was 19 months.

FIG. 16 shows Kaplan-Meier curves showing the overall survival of 1435 patients with ovarian cancer (average; dark grey, solid line; GEO accession numbers: GSE51373, GSE9891, GSE15622, GSE26712, GSE26193, GSE63885, GSE65986, GSE30161, GSE14764, TCGA) stratified by a proprietary 5-gene signature based on OPA1, BNIP3, YMEIL1, IMMT, SAMM50 and PHB expression levels. Patients whose signature matched all 5 genes (positive; black, solid line) had a median survival of 43 months, while patients whose signature did not match any of the 5 genes (negative; light grey, dotted line) had only a median survival of 15 months (Log-rank Test: p<0.0001; Chi square=34.92; df=5).

It is known in the arts, and has been summarized above, that cancer also is a disorder correlated with mitochondrial dysfunction. In the context of the present invention, it was, inter alia, found that measurements of OMA1, HIGD1A, OPA1, BNIP3, YMEIL1, PHB, SAMM50, IMMT and/or PHB2 gene expression levels can be utilized for the prognosis of survival of a patient with cancer.

Cumulative evidence also exists for mitochondrial fusion/fission being necessary for normal cardiac function (Dorn 2013; Piquereau et al. 2013; Burke et al. 2015; Marin-Garcia and Akhmedov 2016; Ong et al. 2017). The first studies to investigate the role of OPA1 in the heart revealed reduced myocardial levels of OPA1 in ischemic heart failure patients and in a rat model of ischemic heart failure (Chen et al. 2009). OPA1 deficient mice show late onset cardiomyopathy with a decrease in cardiac output, reduced fractional shortening, and a blunted response to a B-adrenergic stimulus (Chen et al. 2012; Le Page et al. 2016). These findings were associated with mitochondrial fragmentation, impaired mitochondrial respiration, increased oxidative stress, attenuated calcium transients, and a reduction in mitochondrial DNA copy number (Chen et al. 2012). OPA1 deficient mice are also more susceptible to total aortic constriction, developing twice the extent of left ventricular hypertrophy, when compared to wild-type mice (Piquereau et al. 2012). These findings were associated with clustering of large mitochondria with abnormal cristae morphology, which were demonstrated to be resistant to calcium-induced mitochondrial permeability transition pore (MPTP) opening (Piquereau et al. 2012) Furthermore, cardiomyocytes from OPA1 deficient mice were more susceptible to cell death induced by ischemia/reperfusion injury (Chen et al. 2012; Le Page et al. 2016). Cardiac-specific ablation of YMEIL1 in mice activated OMA1 and triggered OPA1 proteolysis, leading to a dilated cardiomyopathy and heart failure with mitochondrial fragmentation and altered cardiac metabolism (Wai et al. 2015). Genetic OMA1 deletion prevented OPA1 cleavage and cardio myopathy (Wai et al. 2015).

It is envisioned that comparable gene signatures can be developed based on the activity and/or expression of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof.

### Example 4

The means and methods of the present disclosure can also be used to inform selection and/or assist in selecting appropriate courses of treatment and/or medical interventions for patients in need of such interventions.

We studied the OPA1 protein and its isoforms in fibroblast cell lines derived from patients that harbor a duplication of the exons 7 to 9 (c.678-984dup306) in the OPA1 gene (Fuhrmann et al. 2010). Exons 7 to 9 code for 102 amino acids in frame and one would expect a larger OPA1 protein. However, OPA1 protein levels were significantly reduced compared to healthy subjects and the ratios of the different OPA1 isoforms were altered. We could rule out nonsense mediated mRNA decay by qPCR analysis (Fuhrmann et al. 2010). Also mouse embryonic fibroblast cell lines with a splice-site mutation leading to skipping of Opal exon 10 (c.1065+5G>A) showed significantly reduced OPA1 protein levels despite no changes in the abundance of the mRNA (Alavi et al. 2007). These findings led us to the conclusion that OPA1 is regulated on protein levels and that only fully functional OPA1 is stable.

Co-culture experiments of mouse embryo fibroblasts from mutant OPA1 mice and wild-type control mice with epoxomicin, a specific inhibitor of the ubiquitin-proteasome degradation system, did not change OPA1 levels or the ratio of OPA1L to OPA1S isoforms, indicating that OPA1 is not regulated by the ubiquitin-proteasome pathway (Alavi et al. 2007). Phenanthroline is a chelator that sequesters divalent metal ions like zinc, which is crucial for the function of metallo-endopeptidases. When we cultured cells in the presence of phenanthroline, we found an increase in the OPA1L isoform indicating that OPA1 is regulated by proteolytic turnover (Alavi et al. 2007). Indeed, when we investigated the OPA1 protein in pulse-chase experiments we found a degradation of OPA1L to OPA1S and changes in the protein half-life depending on single amino acid substitutions.

Stress-induced OPA1 cleavage caused cell death in different experimental paradigms (Olichon et al. 2003; Duvezin-Caubet et al. 2006; Ishihara et al. 2006; Griparic et al. 2007; Song et al. 2007; Merkwirth et al. 2008; Ehses et al. 2009; Head et al. 2009), and expression of non-cleavable OPA1 isoforms could prevent cell death in these experiments (Ishihara et al. 2006; Griparic et al. 2007; Song et al. 2007; Merkwirth et al. 2008). OMA1 knock-down or knock-out also can prevent cell death while prolonged activation of OMA1 will cause cell death. OMA1 is activated upon stress in pre-clinical disease models for ischemic kidney injury (Xiao et al. 2014), myocardial infarct (Piquereau et al. 2012; Wai et al. 2015), cancer (Kong et al. 2014), and neurodegeneration (Merkwirth et al. 2008; Korwitz et al. 2016). Knock-down or knock-out of OMA1 could prevent cell death in these models (Ehses et al. 2009; Head et al. 2009; Wai et al. 2015; Korwitz et al. 2016). We therefore screened for compounds that can modify OMA1 protease activity in a way that reduces OMA1 activity (i.e., OMA1 antagonists).

HEK293T cells were cultured and maintained in Dulbecco's Modified Eagle's Medium supplemented with 10% fetal bovine serum (FBS) and 2 mM 1-glutamine in a 95% air-5% CO2 humidified atmosphere at 37° C. To test potential OMA1 antagonists, cells were seeded in 24-well plates and incubated over night to achieve around 90% confluency. For experiments the culture medium was replaced with Minimal Essential Medium and cells were pre-incubated with variable concentrations of the different test-compounds for 2 hours in a 95% air-5% CO2 humidified atmosphere at 37° C. After 2 hours, OMA1 protease was activated through the addition of carbonyl cyanide m-chlorophenyl hydrazone (CCCP;10 μM final concentration) for 30 min in the presence of different concentrations of compounds to be tested. After 30 minutes, cells were immediately placed on ice, the medium removed, and cells harvested through addition of 100 μl RIPA buffer supplemented with protease inhibitor cocktail. Samples were separated on 8% tris-glycine gels and transferred onto nitro-cellulose membranes by Wester blotting. Membranes were immunolabeled with anti-OPA1 Antibodies (1:1,000) and with goat-anti-mouse Alkaline Phosphatase conjugated secondary antibodies (1:5,000). OPA1 protein was visualized with NBT/BCIP between 5 and 15 minutes before the reaction was stopped with an excess of distilled water.

As illustrated in FIG. 1, lane 1, and described in more details above, 5 different OPA1 isoforms (i.e., OPA1-LI, OPA1-L2, OPA1-S3, OPA1-S4, OPA1-S5) can be detected in HEK293T cells under standard culture conditions. CCCP is a potent mitochondrial oxidative phosphorylation uncoupler that activates OMA1 and creates conditions that allow OPA1 cleavage to occur. As illustrated in FIG. 1, lane 2, 30 minutes of CCCP treatment resulted in cleavage of both large OPA1-LI and OPA1-L2 isoforms so that only the small OPA1-S3, OPA1-S4 and OPA1-S5 isoforms were detectable.

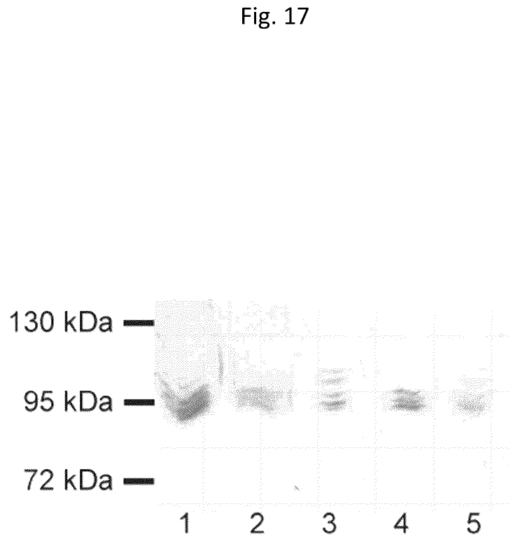

In a non-limiting example, it was, inter alia, found that Thiorphan (CAS #76721-89-6) can inhibit proteolytic cleavage of large OPA1 isoforms under conditions allowing OPA1 processing to occur (FIG. 17). As illustrated in FIG. 17, lanes 1, 2, 4 and 5, only the small OPA1 isoforms OPA1-S3, OPA1-S4 and OPA1-S5 were detectable by SDS-PAGE/Western-blotting upon treatment with 10 μM CCCP. In samples treated with 100 μM Thiorphan, however, all OPA1 isoforms including the two large OPA1-LI and OPA1-L2 isoforms were readily detectable (FIG. 17, lane 3), which demonstrates that Thiorphan can affect proteolytic cleavage of OPA1 by OMA1 upon dissipation of the mitochondrial membrane potential. Thiorphan thus represent an antagonist of OMA1 and/or an oligomeric complex comprising OMA1.

Referring to FIG. 17, thiorphan (CAS #76721-89-6) is a non-limiting example of an OMA1 antagonists. In HEK293T cells, 10 μM CCCP could activate OMA1, which resulted in the cleavage of the OPA1-LI and OPA1-L2 isoforms so that only the smaller three isoforms OPA1-S3, OPA1-S4 and OPA1-S5 were detectable by SDS-PAGE/Western-blot (lanes 1, 2, 4 and 5). The large OPA1-LI and OPA1-L2 isoforms were still readily detectable when cells were treated with 100 μM Thiorphan (lane 3) for 2 hours prior OMA1 activation.

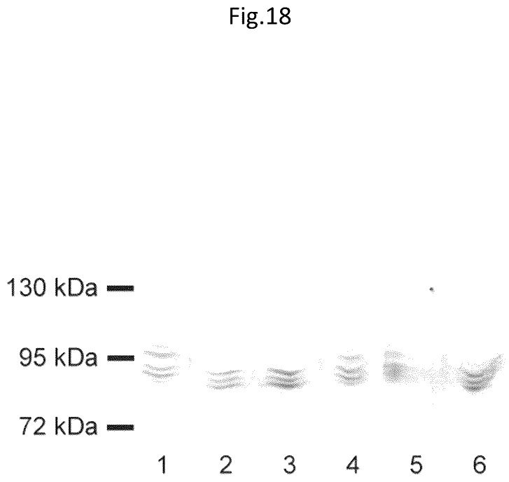

In another non-limiting example, it was, inter alia, found that ARP100 (CAS #704888-90-4) can inhibit proteolytic cleavage of large OPA1 isoforms under conditions allowing OPA1 processing to occur (FIG. 18). Again, only the small OPA1 isoforms OPA1—S3, OPA1-S4 and OPA1-S5 were detectable by SDS-PAGE/Western-blotting upon CCCP treatment (FIG. 18, lanes 2, 3 and 6,), while in samples treated with 70 M ARP100 all OPA1 isoforms including the large OPA1-LI and OPA1-L2 isoforms were readily detectable (FIG. 18, lane 4). This demonstrates that ARP100 can inhibit proteolytic cleavage of OPA1 by OMA1. ARP100 thus represent an antagonist of OMA1 and/or an oligomeric complex comprising OMA1.

Referring to FIG. 18, phenanthroline (CAS #66-71-7) and ARP100 (CAS #704888-90-4) are non-limiting examples of OMA1 antagonists. In HEK293T cells, 10 μM CCCP could activate OMA1, which resulted in the cleavage of the OPA1-LI and OPA1-L2 isoforms so that only the smaller three isoforms OPA1-S3, OPA1-S4 and OPA1-S5 were detectable by SDS-PAGE/Western-blot (lanes 2, 3 and 6). The large OPA1-LI and OPA1-L2 isoforms were still readily detectable when cells were treated with 500 μM phenanthroline (lane 1) or 70 μM ARP100 (lane 4) for 2 hours prior OMA1 activation.

We controlled the experiments with phenanthroline, a chelator with inhibitory effects on metallo-enzymes. As expected, 500 μM phenanthroline also inhibited proteolytic cleavage of OPA1 by OMA1 (FIG. 18, lane 1).

Thiorphan is a potent inhibitor of neprilysin, a membrane metallo-endopeptidase that cleaves peptide hormones, such as enkephalins, glucagon, and bradykinin (Eberlin et al. 2012). Thiorphan is known in the arts also for its neuroprotective activity against excitotoxic neuronal cell death (Medja et al. 2006), which supports the OMA1 protease as genuine target for neuroprotective therapies. Moreover, this validates our approach of developing means and methods for the development of therapies for patients with mitochondrial disease. ARP 100 is a biphenylsulfonamide that acts as a selective inhibitor of MMP-2 (Rossello et al. 2004; Tuccinardi et al. 2006). The antagonistic effects of ARP100 on OMA1 were not known and are non-obvious to a person skilled in the arts.

### Example 5

Cancer is a non-limiting example for a mitochondrial disorder or disease characterized by OPA1 alterations. As laid out in more details above, alterations in OMA1 and/or a heterooligomeric complex compromising OMA1 have a prognostic value for patients with various types of cancer. Certain cancer types were characterized, inter alia, by a 3-gene signature of increased OMA1 and HIGD1A levels and decreased BNIP3 levels. Patients with increased with this particular 3-gene signature had a significantly better prognosis of overall survival than patients without this signature. Moreover, patients that would have an inverted 3-gene signature of reduced OMA1 and HIGD1A levels and increased BNIP3 levels had a significantly worse prognosis of overall survival. In accordance with our results are findings of a study that compared cancer cells that are resistant against platin-based therapies with cancer cells that are amenable to these therapies. Chemoresistant cancer cells show significantly reduced OMA1 activity, which correlated with tumorigenesis, metastatic spread and overall survival of the cancer cells (Kong et al. 2015). Taken together, these data demonstrate that certain mitochondrial disorders or diseases are characterized by decreased OMA1 and that OMA1 agonists represent genuine medical interventions for these diseases. We therefore also screened for compounds that can modify OMA1 protease activity in a way that increases OMA1 activity (i.e., OMA1 agonists).

HEK293T cells were cultured and maintained in Dulbecco's Modified Eagle's Medium supplemented with 10% fetal bovine serum (FBS) and 2 mM 1-glutamine in a 95% air-5% CO₂ humidified atmosphere at 37° C. To test potential OMA1 antagonists, cells were seeded in 24-well plates and incubated over night to achieve around 90% confluency. For experiments the culture medium was replaced with Minimal Essential Medium and cells were pre-incubated with 100 μM compounds to be tested for their effects on OMA1 protease for 2 hours in a 95% air-5% CO₂ humidified atmosphere at 37° ° C. After 2 hours, cells were treated with increasing concentrations of 0 μM, 3 μM, 5 μM or 7 μM CCCP for 20 minutes to determine the minimum CCCP concentration that would lead to OMA1 activation and OPA1 cleavage, and whether this threshold level could be modified by the compound at question. After 20 minutes, cells were immediately placed on ice, the medium removed, and cells harvested through addition of 100 μl RIPA buffer supplemented with protease inhibitor cocktail. Samples were separated on 8% tris-glycine gels and transferred onto nitro-cellulose membranes by Wester blotting. Membranes were immunolabeled with anti-OPA1 Antibodies (1:1,000) and with goat-anti-mouse Alkaline Phosphatase conjugated secondary antibodies (1:5,000). OPA1 protein was visualized with NBT/BCIP between 5 and 15 minutes before the reaction was stopped with an excess of distilled water.

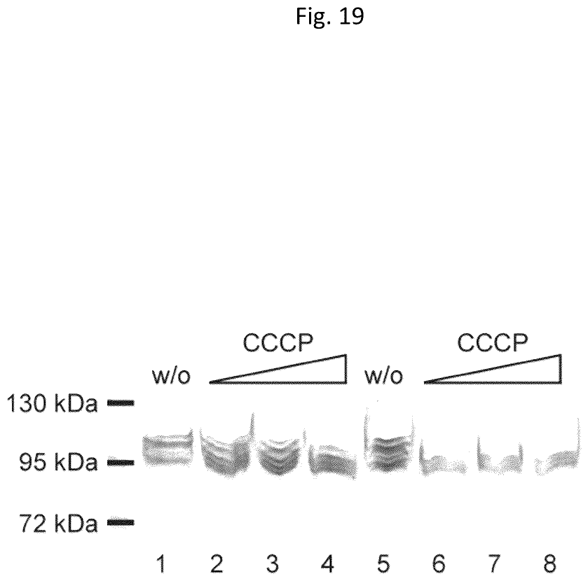

In one particular embodiment it was, inter alia, found that SB-3CT (CAS #292605-14-2) reduced the threshold levels of OMA1 activation and allowed for proteolytic cleavage of large OPA1 isoforms to occur under conditions that otherwise would not allow for OPA1 cleavage to occur. HEK293T cells were cultured in MEM only or in MEM supplemented with increasing concentrations of 0 μM, 3 μM, 5 M or 7 μM CCCP. As illustrated in FIG. 19, 0 μM, 3 μM and 5 μM CCCP had no effect on OPA1 processing in untreated cells (lanes 1, 2 and 3). In untreated cells OPA1 was cleaved only at the highest concentration of 7 μM CCCP (FIG. 19, lane 4). SB-3CT by itself had no effect on OPA1 under normal conditions that do not allow for OPA1 cleavage to occur, because cells that only were treated with 100 μM SB-3CT and that were not exposed to CCCP did not show OPA1 alterations (FIG. 19, lane 5). All 5 isoforms were readily detectable in these samples by SDS-PAGE/Western-blotting (FIG. 19, lane 5). To our surprise and against all expectations we found that 100 μM SB-3CT caused OPA1 cleavage to occur already at 3 μM and 5 μM CCCP (FIG. 19, lanes 6 and 7). As illustrated in FIG. 19, OPA1 cleavage in SB-3CT-treated cells, in particular at low CCCP concentrations, exceeded that of the control cells (FIG. 19, lanes 2 and 3, “vehicle-only”.)

Referring to FIG. 19, SB-3CT (CAS #292605-14-2) represents an OMA1 agonist because SB-3CT reduces the threshold levels of OMA1 activation and allows for proteolytic cleavage of large OPA1 isoforms to occur under conditions that otherwise would not allow for OPA1 cleavage to occur. HEK293T cells were cultured with increasing concentrations of 0 μM, 3 M, 5 UM and 7 μM CCCP (lanes 1˜4 & 5-8). In “vehicle-only” treated cells (lanes 1-4), OMA1 was activated at the highest concentration of 7 μM CCCP (lane 4). In cells treated with 100 μM SB-3CT (lanes 5-8), OMA1 was already activated at lower concentrations of 3 μM and 5 μM CCCP (lanes 6 & 7). 100 μM SB-3CT did not induce OPA1 cleavage without CCCP (lane 5) demonstrating that SB-3CT acts through the stress-dependent modulation of the OMA1 complex.

SB-3CT is known in the arts for its inhibitory effects on proteases (US 2009/0209615 and US 2013/0052184) and the above-mentioned finding that SB-3CT can activate the OMA1 protease under conditions that otherwise would not allow for OPA1 cleavage to occur was surprising and against all expectations. In context of the present invention SB-3CT thus represents an agonist of OMA1 and/or an oligomeric complex comprising OMA1 as defined above. Accordingly, SB-3CT represents a medical intervention in particular for the treatment, prevention and/or amelioration of a disorder or disease correlated with mitochondrial stress or dysfunction, a mitochondrial disorder or disease, or a disorder or disease characterized by OPA1 alterations.

### Example 6

An important aspect of the invention disclosed herein is that mitochondrial diseases or disorders or diseases can be characterized by alterations of OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof. Measurements of these alterations can support diagnosis and guide therapy selection. Moreover, compounds and/or interventions that can mitigate alterations in OMA1, HIGD1A, OPA1, BNIP3, YMEIL1, PHB, PHB2, SAMM50 or IMMT present suitable medical interventions for patients suffering from such a disorder or disease.

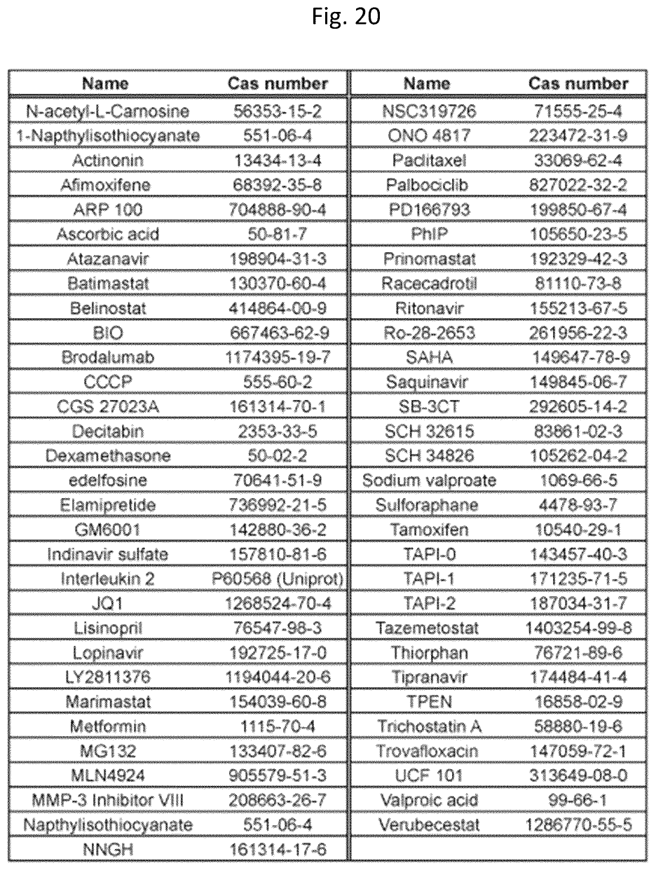

In an in silico approach we aimed to identify compounds and/or interventions that affect OMA1 and/or a heterooligomeric complex compromising OMA1. To this end, we mined publicly available data repositories for compounds that can modulate OMA1, HIGD1A, OPA1, BNIP3, YMEIL1, PHB, PHB2, SAMM50 or IMMT. The findings are summarized in the table depicted in FIG. 20, which provides a list of compounds and treatments that are suitable medical interventions for patients with a mitochondrial disease or disorder.

FIG. 20 shows a list of different drugs and compounds that can modify OMA1 and/or an oligomeric complex comprising OMA1 and/or HIGD1A and/or OPA1 and/or BNIP3 and/or YMEIL1 and/or PHB and/or PHB2 and/or SAMM50 and/or IMMT or a variant thereof. These drugs represent non-limiting examples for therapies and medical interventions for patients with mitochondrial disease or disorder.

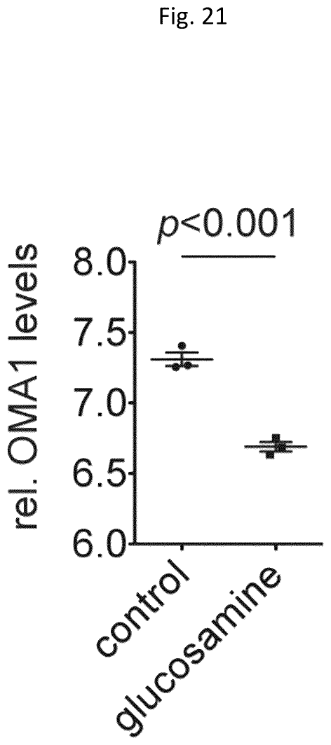

In one embodiment it was, inter alia, found that glucosamine (CAS #3416-24-8) significantly decreases the gene expression levels of OMA1 by 8.5% (FIG. 21; Student's T-test: p≤0.001). Human malignant lymphocytes (KMH2 cells) were cultured without or with 20 mM glucosamine (Carvalho et al. 2014). After 24 hours mRNA was isolated and analyzed by gene expression microarrays. Controls expressed OMA1 at 7.31 (±0.08 S.D.) and glucosamine treated cells expressed OMA1 at 6.69 (±0.06 S.D.). Glucosamine is a dietary supplement and its effects on mitochondria are known to a person skilled in the arts (Carvalho et al. 2014; Bond and Hanover 2015; Tan et al. 2017). However, the finding that glucosamine can significantly decrease OMA1 levels was unexpected and is non-obvious to a person skilled in the arts. In context of the present invention glucosamine thus represents an antagonist of OMA1 and/or an oligomeric complex comprising OMA1 as defined above. Accordingly, glucosamine represents a medical intervention in particular for the treatment, prevention and/or amelioration of a disorder or disease correlated with mitochondrial stress or dysfunction, a mitochondrial disorder or disease, or a disorder or disease characterized by OPA1 alterations.

Referring to FIG. 21, the dietary supplement glucosamine (CAS #3416-24-8) represents an antagonist of OMA1. Glucosamine can significantly decrease the gene expression levels of OMA1 in human malignant lymphocytes by 8.5% (Student's T-test: p≤0.001; GEO accession number: GDS5388). KMH2 cells were cultured for 24 hours without or with 20 mM glucosamine, after which mRNA was isolated and analyzed by gene expression microarrays. Controls expressed OMA1 at 7.31 (±0.08 S.D.) and glucosamine treated cells expressed OMA1 at 6.69 (±0.06 S.D.).

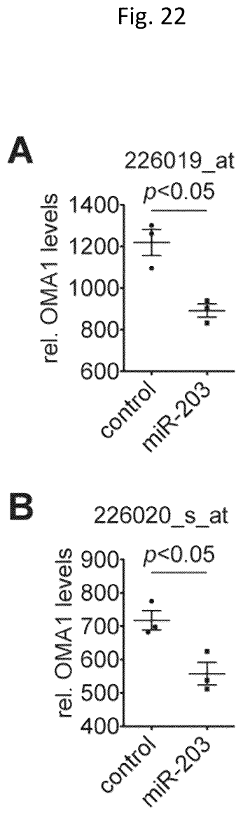

In another embodiment it was, inter alia, found that the micro-RNA miR-203 (NCBI Reference Sequence: NR_029620.1) significantly reduced the gene expression levels of OMA1 by 22% to 27% (FIGS. 22A and 22B); Student's T-test: p<0.05). miR-203 was overexpressed in the breast cancer cell line SUM159 using a retrovirus (Taube et al. 2013). SUM159 control cells expressed OMA1 at 1219.5 (±109.3 S.D.; Spot ID: 226019_at) and 718.4 (±50.0 S.D. (FIG. 22A); Spot ID: 226020_s_at), while SUM159 cells expressing miR-203 had significantly reduced OMA1 levels at 891.7 (±54.1 S.D.; Spot ID: 226019_at) and 557.8 (±59.1 S.D.; Spot ID: 226020_s_at) (FIG. 22B), respectively. A person skilled in the arts knows miR-203 for its role in the epithelial-mesenchymal transition (EMT). The finding that miR-203 can decrease OMA1 levels was unexpected and is non-obvious to a person skilled in the arts. In context of the present invention miR-203 thus represents an antagonist of OMA1 and/or an oligomeric complex comprising OMA1 as defined above. Accordingly, miR-203 represents a medical intervention in particular for the treatment, prevention and/or amelioration of a disorder or disease correlated with mitochondrial stress or dysfunction, a mitochondrial disorder or disease, or a disorder or disease characterized by OPA1 alterations.

Referring to FIGS. 22A and 22B, the micro-RNA miR-203 (NCBI Reference Sequence: NR_029620.1) represents an antagonist of OMA1. miR-203 can significantly decrease the expression levels of OMA1 in human SUM159 mesenchymal-like breast cancer cells by 22% to 27% (Student's T-test: p≤0.05; GEO accession number: GSE23031).

SUM159 control cells expressed OMA1 at 1219.5 (±109.3 S.D.; Spot ID: 226019_at) and 718.4 (±50.0 S.D. (FIG. 22A); Spot ID: 226020_s_at), while SUM159 cells expressing miR-203 had significantly reduced OMA1 levels at 891.7 (±54.1 S.D.; Spot ID: 226019_at) and 557.8 (±59.1 S.D.; Spot ID: 226020_s_at) (FIG. 22B), respectively.

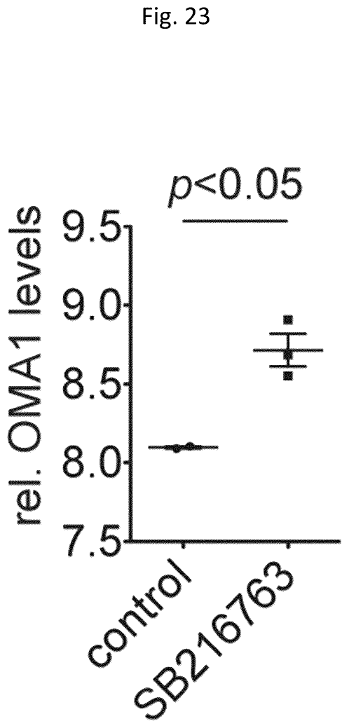

In another particular embodiment it was, inter alia, found that the GSK-3 inhibitor SB216763 (CAS #280744-09-4) significantly increased gene expression levels of OMA1 by 7.6% (FIG. 23; Student's T-test: p≤0.05). Human MLL leukemia RS4.11 cells were cultured without or with 10 μM SB216763 for 20 hours (Wang et al. 2010). Gene expression analyses by microarrays demonstrated that controls expressed OMA1 at 8.10 (±0.01 S.D.), while cells treated with SB216763 expressed OMA1 at 8.7(±0.18 S.D.). SB216763 is a kinase inhibitor and the finding that SB216763 can increase OMA1 levels was unexpected and is non-obvious to a person skilled in the arts. In context of the present invention SB216763 thus represents an agonist of OMA1 and/or an oligomeric complex comprising OMA1 as defined above. Accordingly, SB216763 represents a medical intervention in particular for the treatment, prevention and/or amelioration of a disorder or disease correlated with mitochondrial stress or dysfunction, a mitochondrial disorder or disease, or a disorder or disease characterized by OPA1 alterations.

Referring to FIG. 23, the GSK-3 inhibitor SB216763 (CAS #280744-09-4) represents an agonist of OMA1. SB216763 can significantly increase OMA1 gene expression levels in human MLL leukemia cells (RS4.11 cells) by 7.6% (Student's T-test: p≤0.05; GEO accession number: GDS4043). RS4.11 cells were cultured for 20 hours without or with 10 μM SB216763, after which mRNA was isolated and analyzed by gene expression microarrays. Controls expressed OMA1 at 8.10 (±0.01 S.D.) and SB216763 treated cells expressed OMA1 at 8.7(±0.18 S.D.).

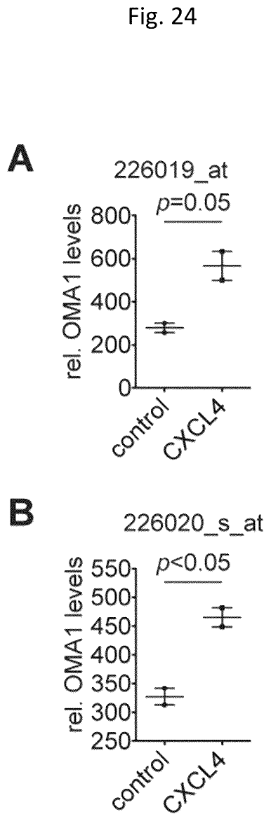

In one particular embodiment it was, inter alia, found that the cytokine CXCL4 (UniProt: P02776) significantly increased the gene expression levels of OMA1 in monocyte derived macrophages by 42% to 103% (FIGS. 24A and 24B; Student's T-test: p≤0.05). Monocytes were cultured for 6 days without or with 1 μM CXCL4 (Gleissner et al. 2010). Controls expressed OMA1 at 279.0 (±30.9 S.D.; Spot ID: 226019_at) and 327.3 (±20.8 S.D.; Spot ID: 226020_s_at), while CXCL4 treated cells had significantly increased OMA1 levels at 566.8 (±94.5 S.D. (FIG. 24A); Spot ID: 226019_at) and 465.3 (±23.3 S.D.; Spot ID: 226020_s_at) (FIG. 24B), respectively. CXCL4 is a small cytokine belonging to the CXC chemokine family that is also known in the arts as platelet factor 4 (PF4). The finding that CXCLA can increase OMA1 levels was unexpected and is non-obvious to a person skilled in the arts. In context of the present invention CXCLA thus represents an agonist of OMA1 and/or an oligomeric complex comprising OMA1 as defined above. Accordingly, CXCL4 represents a medical intervention in particular for the treatment, prevention and/or amelioration of a disorder or disease correlated with mitochondrial stress or dysfunction, a mitochondrial disorder or disease, or a disorder or disease characterized by OPA1 alterations.

Referring to FIGS. 24A and 24B, the small cytokine CXCL4 (UniProt: P02776) represents an agonist of OMA1. CXCL4 can significantly increase the gene expression levels of OMA1 in monocyte derived macrophages by 42% to 103% (Student's T-test: p≤0.05; GEO accession number: GDS3787). Controls expressed OMA1 at 279.0 (±30.9 S.D.; Spot ID: 226019_at) and 327.3 (±20.8 S.D. (FIG. 24A); Spot ID: 226020_s_at), while CXCL4 treated cells had significantly increased OMA1 levels at 566.8 (±94.5 S.D.; Spot ID: 226019_at) and 465.3 (±23.3 S.D.; Spot ID: 226020_s_at) (FIG. 24B), respectively.

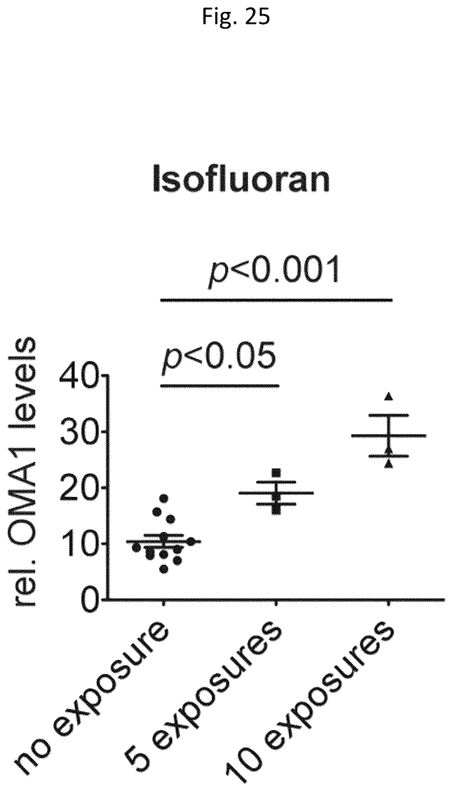

In another particular embodiment it was, inter alia, found that Isoflurane increased the gene expression levels of OMA1 in rat brains in a dose-dependent manner (FIG. 25). Rats were exposed to 1% Isoflurane for 90 minutes twice daily for a total of 5 or 10 exposures, after which animals were sacrificed and brain samples analyzed by gene expression micro-arrays (Pan et al. 2006). Control rats expressed OMA1 at 10.4 (±3.8 S.D.), while rats exposed to Isoflurane for 5-times expressed OMA1 at elevated levels of 19.1 (±3.4 S.D.). Rats exposed to Isoflurane for 10-times showed even higher OMA1 expression levels of 29.3 (±6.3 S.D.) The dose-dependent increase by 83% and 180%, respectively, was statistically significant (FIG. 25; Student's T-test: p≤0.05). Isoflurane belongs to the halogenated ether family of medication and was approved for medical use in the United States in 1979. Isoflurane is a general anesthetic administered in conjunction with air and/or pure oxygen through inhalation. The exact mechanism of the action has not been clearly delineated. Isoflurane likely binds to GABA, glutamate and glycine receptors. It also binds to the D subunit of ATP synthase and NADH dehydrogenase. The finding that Isoflurane can increase OMA1 levels was unexpected and is non-obvious to a person skilled in the arts. In context of the present invention Isoflurane thus represents an agonist of OMA1 and/or an oligomeric complex comprising OMA1 as defined above. Accordingly, Isoflurane represents a medical intervention in particular for the treatment, prevention and/or amelioration of a disorder or disease correlated with mitochondrial stress or dysfunction, a mitochondrial disorder or disease, or a disorder or disease characterized by OPA1 alterations.

Referring to FIG. 25, isoflurane represents an agonist of OMA1. Isoflurane exposure can increase the gene expression levels of OMA1 in rat brains in a dose-dependent manner (GEO accession number: GDS364). Control rats expressed OMA1 at 10.4 (±3.8 S.D.), while rats exposed to Isoflurane for 5-times expressed OMA1 at elevated levels of 19.1 (±3.4 S.D.). Rats exposed to Isoflurane for 10-times showed even higher OMA1 expression levels of 29.3 (±6.3 S.D.). The dose-dependent increase of 183% and 280%, respectively, was statistically significant (Student's T-test: p<0.05).

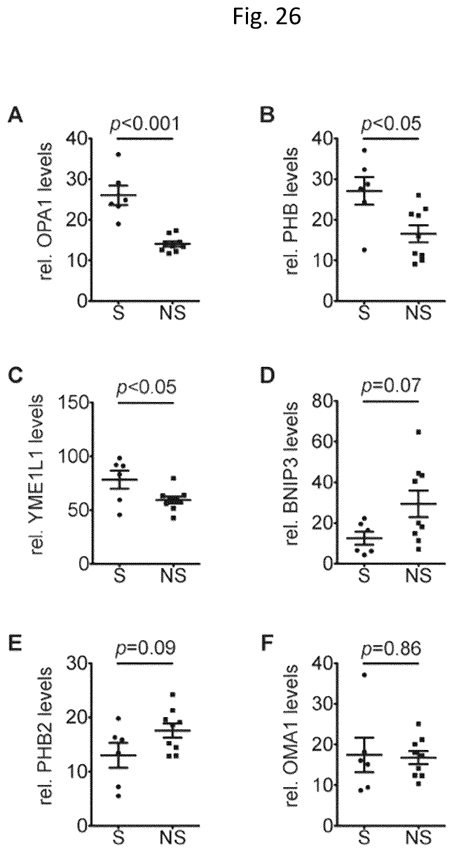

In another non-limiting and merely illustrative example it was, inter alia, found that OPA1, PHB and YMEIL1 gene expression levels were significantly elevated in tobacco smokers (FIGS. 26A-26F). Gene expression levels were determined in blood samples from tobacco smokers (n=6) and non-smokers (n=9) by micro array analysis (Philibert et al. 2007). OPA1 levels were highly significantly elevated by 85% in smokers (26.0+5.9 S.D.) compared to non-smokers (14.0+1.9 S.D.; FIG. 26A; Student's T-test: p<0.001). PHB levels were significantly elevated by 64% in smokers (27.1+8.3 S.D.) compared to non-smokers (16.6+6.3 S.D.; FIG. 26B; Student's T-test: p<0.05). And also YMEIL1 levels were significantly increased by 32% in smokers (78.3+20.0 S.D.) compared to non-smokers (59.4+10.0 S.D.; FIG. 26C; Student's T-test: p<0.05). BNIP3 levels and PHB2 levels appeared to be reduced in smokers (FIGS. 26D and E), while OMA1 expression was not changed (FIG. 26F). In view of the teaching provided herein, it is envisioned that smoking, and more preferably one or more pharmaceutically active compound(s) contained in tobacco smoke, will be administered to a patient in need of medical intervention for the treatment, prevention and/or amelioration of a disorder or disease correlated with mitochondrial stress or dysfunction, a mitochondrial disorder or disease, or a disorder or disease characterized by OPA1 alterations.

Referring to FIGS. 26A-26F, tobacco smoking alters OPA1, PHB and YMEIL1 gene expression. Smoking can increase the OPA1 (26A), PHB (26B) and YMEIL1 gene expression levels (C) in blood samples by 85%, 64% and 32%, respectively (Student's T-test: p≤0.05; GEO accession number: GDS2447). FIG. 26A: Non-smokers (NS) expressed OPA1 at 14.0 (±1.9 S.D.) and smokers (S) at 26.0 (±5.9 S.D.). FIG. 26B: Non-smokers expressed PHB at 16.6 (±6.3 S.D.) and smokers at 27.1 (±8.3 S.D.). FIG. 26C: And non-smokers expressed YMEIL1 at 59.4 (±10.0 S.D.) and smokers at 78.3 (±20.0 S.D.). FIG. 26D: BNIP3 levels appeared to be reduced in smokers (12.6+10.0 S.D.) compared to non-smokers (29.4+19.5 S.D.) though the difference did not reach statistical significance. FIG. 26E: PHB2 levels also appeared to be reduced in smokers (13.0+5.6 S.D.) compared to non-smokers (17.6+4.0 S.D.). FIG. 26F: OMA1 levels were not changed (smokers: 17.4+10.3 S.D.; non-smokers: 16.8+4.9 S.D.).

Preferentially the susceptibility for, predisposition for, and/or presence of, such a disorder or disease has been determined by measurements of OMA1, HIGD1A, OPA1, BNIP3, YMEIL1, PHB, SAMM50, IMMT and/or PHB2 levels as disclosed herein and the medical intervention has been selected based on these biomarkers. The skilled person is readily in the position to select the medical intervention for the patient in need of medical intervention based on these biomarkers.

### Example 7. Tipranavir can inhibit OMA1. OMA1 is a Zn2+-dependent

metalloendopeptidase located in the mitochondrial inner membrane with the OPA1 protein as major substrate. The protease is classified according to the MEROPS database as M48 family member of metalloendopeptidases. The M48 family also encompasses the metallopeptidase STE24 (ZMPSTE24, also known as farnesylated-protein converting enzyme 1, FACE1). ZMPSTE24 is an integral membrane protein which cleaves the nuclear envelope protein prelaminin A.

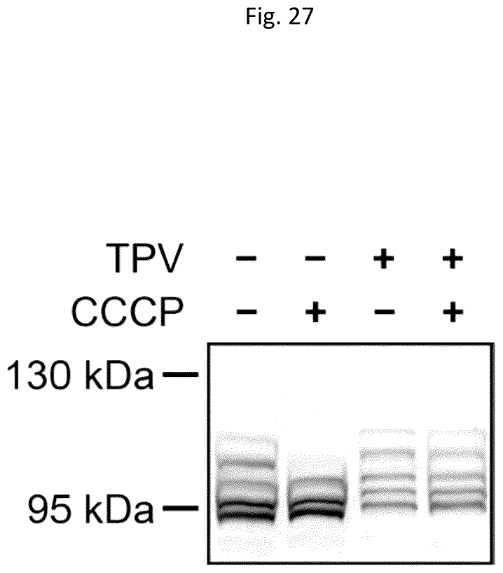

As explained in the manuscript entitled “OMA1—An integral membrane protease?” (Alavi MV. Biochim Biophys Acta Proteins Proteom. 2020 Oct. 29; 1869(2): 140558), it is known in the arts that HIV1 protease inhibitors can inhibit the ZMPSTE24 protease in the single-digit-micro molar range. We found—not unexpectedly and consistent with OMA1's homology with ZMPSTE24—that tipranavir, which is disclosed in present invention, appears to also inhibit OMA1 protease (see FIG. 27).

OMA1 shows little activity under physiological conditions. Experimentally, OMA1 can be activated in cellular assays by the mitochondrial uncoupler [(3-chlorophenyl)hydrazono] propanedinitrile (CCCP). This results in cleavage of the large L-OPA1 isoforms, which are a major OMA1 substrate. Referring to FIG. 27, all L-OPA1 isoforms are hydrolyzed upon CCCP treatment to the small, cleaved S-OPA1 isoforms. Tipranavir prevented this CCCP-dependent L-OPA 1 hydrolysis at a concentration of 100 μM, from which can be deduced that tipranavir can inhibit OMA1 protease.

Example 8. Tipranavir has unexpected effects on OMA1 activity. I further investigated tipranavir's effect on OPA1 as described in Example 7. To this end, I incubated Hek293T cells with different tipranavir concentrations.

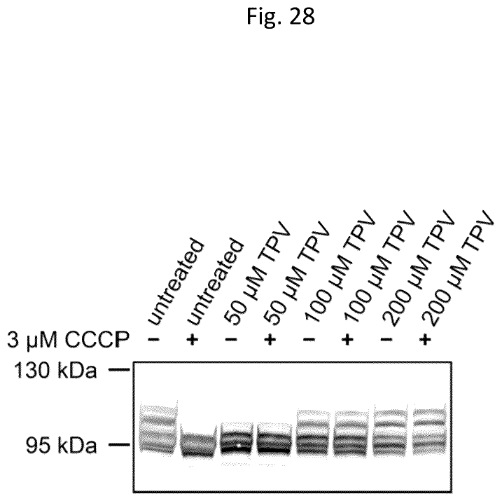

When I incubated Hek293T cells with 50 μM, 100 μM and 200 μM tipranavir for an hour, I found against all expectations and quite to my surprise that 50 μM tipranavir did not only not prevent OPA1 processing upon CCCP-treatment, which I observed at concentrations of 100 μM and 200 μM tipranavir, but resulted in OPA1 processing even in the absence of CCCP (see FIG. 28). This means tipranavir has an antagonistic effect on OMA1 depending on its dose: lower doses of about 50 μM can activate the enzyme and lead to OPA1 cleavage, while higher doses of about 100 μM inhibit the enzyme's activity and prevent OPA1 cleavage even in the presence of CCCP.

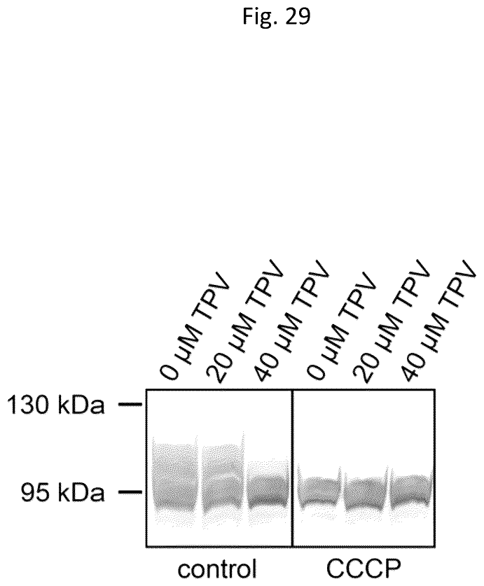

Example 9. Tipranavir can activate OMA1 at concentrations above 20 μM. To further determine tipranavir's effect on OPA1, I incubated Hek293T cells for an hour with low doses of 0 μM, 20 μM and 40 μM tipranavir (FIG. 29). I found OPA1 cleavage at 40 μM tipranavir without any CCCP, while 20 μM and 40 μM tipranavir were not sufficient to prevent CCCP-induced OPA1 cleavage.

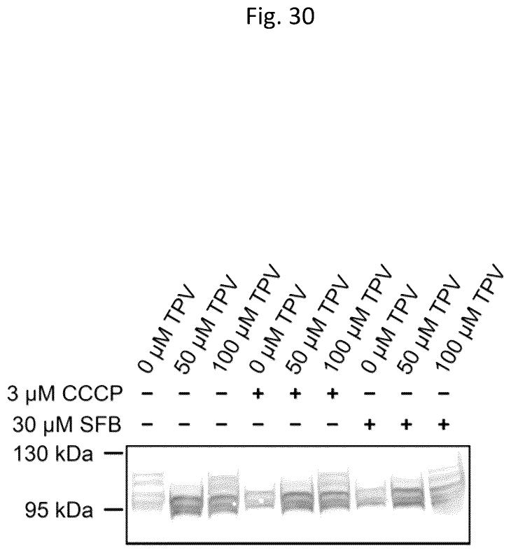

Example 10. 100 μM tipranavir can inhibit OPA1 cleavage upon CCCP-treatment and upon sorafenib treatment. 30 μM sorafenib (SFB) activate OMA1 and lead to OPA1 cleavage similar to CCCP. To investigate whether 100 μM tipranavir can inhibit CCCP-induced OPA1-cleavage or OPA1-cleavage induced by other agents as well, I incubated Hek293T cells for an hour with low doses of 0 μM, 50 UM or 100 μM tipranavir before activating OMA1 with either 3 μM CCCP or 30 μM sorafenib (see FIG. 30). I observed OPA1 hydrolysis at about 50 μM tipranavir irrespective of the presence or absence of CCCP or sorafenib. 100 μM tipranavir on the other hand prevented CCCP-induced OPA1 cleavage and sorafenib-induced OPA1 cleavage.

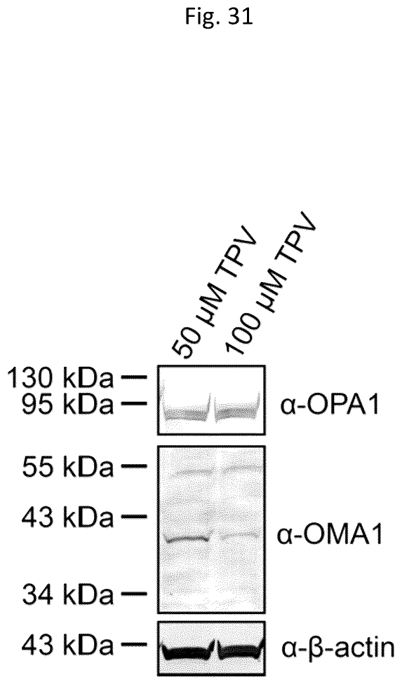

Example 11. Tipranavir has unexpected effects on the OMA1 protein itself. I examined the effects of tipranavir on the OMA1 protein. I incubated Hek293T cells for an hour with either 50 μM or 100 tipranavir (FIG. 31). I found OMA1 protein was reduced when cells were incubated with 100 μM tipranavir compared to 50 μM tipranavir. From this result the skilled artisan can deduce that tipranavir acts as a positive allosteric OMA1 modulator and that other compounds of the genus can act as OMA1 modulators as well.

It should be noted that there are alternative ways of implementing the embodiments disclosed herein. Accordingly, the present embodiments are to be considered as illustrative and not restrictive. Furthermore, the claims are not to be limited to the details given herein, and are entitled their full scope and equivalents thereof.

### PATENT CITATIONS

CN105769863B
CN110302199A
EP1712231A2
U.S. Pat. No. 5,852,195
U.S. Pat. No. 6,169,181
US20050020517A1
WO1995015972A1
WO2002036110A2
WO2004052831A2
WO2004087139A1
WO2008142090A1
WO2010033219A2
JP271816298
JP271819072
JP273710022
KR290283
KR513307
U.S. Ser. No. 15/296,2773
US224375049
U.S. Pat. No. 39,387,348
U.S. Pat. No. 39,444,655
U.S. Pat. No. 39,448,341
U.S. Pat. No. 39,464,492
U.S. Pat. No. 39,496,938
U.S. Pat. No. 39,508,105
U.S. Pat. No. 39,708,032
U.S. Pat. No. 39,828,430
U.S. Pat. No. 39,880,401
U.S. Pat. No. 39,914,181
U.S. Pat. No. 39,930,483
U.S. Pat. No. 39,977,358
U.S. Pat. No. 39,988,591
US40048676
US40076630
US40104645
US40108953
US40170483
US40244744
US40270458
US40274202
US40379080
US40454594
US40614434
US40680642
US40697127
US40923452
U.S. Pat. No. 41,144,813
U.S. Pat. No. 41,161,893
U.S. Pat. No. 41,207,728
U.S. Pat. No. 41,389,242
U.S. Pat. No. 41,412,787
U.S. Pat. No. 41,412,788
U.S. Pat. No. 41,429,131
U.S. Pat. No. 41,522,431
U.S. Pat. No. 41,646,456
U.S. Pat. No. 41,678,020
U.S. Pat. No. 41,682,212
U.S. Pat. No. 41,807,878
U.S. Pat. No. 41,838,863
U.S. Pat. No. 42,579,072
U.S. Pat. No. 42,983,588
U.S. Pat. No. 42,992,632
WO1995030670
WO1997021683
WO1997021685
WO1998022106
WO1999006024
WO1999006043
WO1999006044
WO2000055150
WO2001052821
WO2003090690
WO2004052831
WO2004052883
WO2004060370
WO2006055455
WO2006055660
WO2006060159
WO2006060175
WO2006060177
WO2007092802
WO2007103740
WO2007114978
WO2008112289
WO2008142090

### NON-PATENT CITATIONS

Akhtar, M. W., S. Sanz-Blasco, N. Dolatabadi, J. Parker, K. Chon, M. S. Lee, W. Soussou, S. R. McKercher, R. Ambasudhan, T. Nakamura and S. A. Lipton (2016). “Elevated glucose and oligomeric beta-amyloid disrupt synapses via a common pathway of aberrant protein S-nitrosylation.” Nat Commun 7: 10242.
Alavi, M. V., S. Bette, S. Schimpf, F. Schuettauf, U. Schraermeyer, H. F. Wehrl, L. Ruttiger, S. C. Beck, F. Tonagel, B. J. Pichler, M. Knipper, T. Peters, J. Laufs and B. Wissinger (2007). “A splice site mutation in the murine Opal gene features pathology of autosomal dominant optic atrophy.” Brain 130(Pt 4): 1029-1042.
Alavi, M. V. and N. Fuhrmann (2013). “Dominant optic atrophy, OPA1, and mitochondrial quality control: understanding mitochondrial network dynamics.” Mol Neurodegener 8(1): 32.
Alavi, M. V. (2019). “Targeted OMA1 therapies for cancer.” Int J Cancer 145(9): 2330-41.
Alavi M. V. (2020). “OMA1—An integral membrane protease” Biochim Biophys Acta Proteins Proteom. 1869(2):140558.
Alavi M. V. (2021). “Tau phosphorylation and OPA1 proteolysis are unrelated events: Implications for Alzheimer's Disease.” Biochim Biophys Acta Mol Cell Res 1868(12):119116.
Alavi M. V. (2021). “OMA1 High-Throughput Screen Reveals Protease Activation by Kinase Inhibitors.” ACS Chem Biol 16(11):2202-2211.
Alavi M. V. (2022) “Recent advances in, and challenges of, designing OMA1 drug screens.” Pharmacol Res 176:106063.
Alexander, C., M. Votruba, U. E. Pesch, D. L. Thiselton, S. Mayer, A. Moore, M. Rodriguez, U. Kellner, B. Leo-Kottler, G. Auburger, S. S. Bhattacharya and B. Wissinger (2000). “OPA1, encoding a dynamin-related GTPase, is mutated in autosomal dominant optic atrophy linked to chromosome 3q28.” Nat Genet 26(2): 211-215.
Aliev, G., D. Seyidova, B. T. Lamb, M. E. Obrenovich, S. L. Siedlak, H. V. Vinters, R. P. Friedland, J. C. LaManna, M. A. Smith and G. Perry (2003). “Mitochondria and vascular lesions as a central target for the development of Alzheimer's disease and Alzheimer disease-like pathology in transgenic mice.” Neurol Res 25(6): 665-674.
Alirol, E. and J. C. Martinou (2006). “Mitochondria and cancer: is there a morphological connection?” Oncogene 25(34): 4706-4716.
Ameri, K., A. Jahangiri, A. M. Rajah, K. V. Tormos, R. Nagarajan, M. Pekmezci, V. Nguyen, M. L. Wheeler, M. P. Murphy, T. A. Sanders, S. S. Jeffrey, Y. Yeghiazarians, P. F. Rinaudo, J. F. Costello, M. K. Aghi and E. Maltepe (2015). “HIGD1A Regulates Oxygen Consumption, ROS Production, and AMPK Activity during Glucose Deprivation to Modulate Cell Survival and Tumor Growth.” Cell Rep.
Ameri, K. and E. Maltepe (2015). “HIGD1A-mediated dormancy and tumor survival.” Mol Cell Oncol 2(4): e1030537.
Ameri, K., A. M. Rajah, V. Nguyen, T. A. Sanders, A. Jahangiri, M. Delay, M. Donne, H. J. Choi, K. V. Tormos, Y. Yeghiazarians, S. S. Jeffrey, P. F. Rinaudo, D. H. Rowitch, M. Aghi and E. Maltepe (2013). “Nuclear localization of the mitochondrial factor HIGD1A during metabolic stress.” PLOS One 8(4): e62758.
An, H. J., G. Cho, J. O. Lee, S. G. Paik, Y. S. Kim and H. Lee (2013). “Higd-la interacts with Opal and is required for the morphological and functional integrity of mitochondria.” Proc Natl Acad Sci USA 110(32): 13014-13019.
An, H. J., H. Shin, S. G. Jo, Y. J. Kim, J. O. Lee, S. G. Paik and H. Lee (2011). “The survival effect of mitochondrial Higd-la is associated with suppression of cytochrome C release and prevention of caspase activation.” Biochim Biophys Acta 1813(12): 2088-2098. APTIVUS (tipranavir) Label—FDA
Back, S. H., S. J. Park, J. I. Jeong, S. H. Kim, J. Han, J. W. Kyung, S. H. Baik, Y. Choi, B. Y. Choi, J. S. Park, G. Bahn, J. H. Shin, D. S. Jo, J. Y. Lec, C. G. Jang, T. V. Arumugam, J. Kim, J. W. Han, J. Y. Koh, D. H. Cho and D. G. Jo (2017). “Inhibition of Drp1 Ameliorates Synaptic Depression, Abeta Deposition, and Cognitive Impairment in an Alzheimer's Disease Model.” J Neurosci 37(20): 5099-5110.
Barrera, M., S. Koob, D. Dikov, F. Vogel and A. S. Reichert (2016). “OPA1 functionally interacts with MIC60 but is dispensable for crista junction formation.” FEBS Lett 590(19): 3309-3322.
Berge, S. M., et al. (1977). “Pharmaceutical Salts,” J. Pharm. Sci. 66: 1-19.
Bohovych, I., S. Kastora, S. Christianson, D. Topil, H. Kim, T. Fangman, Y. J. Zhou, A. Barrientos, J. Lee, A. J. Brown and O. Khalimonchuk (2016). “Omal Links Mitochondrial Protein Quality Control and TOR Signaling to Modulate Physiological Plasticity and Cellular Stress Responses.” Mol Cell Biol 36(17): 2300-2312.
Bond, M. R. and J. A. Hanover (2015). “A little sugar goes a long way: the cell biology of O-GlcNAc.” J Cell Biol 208(7): 869-880.
Bose, A. and M. F. Beal (2016). “Mitochondrial dysfunction in Parkinson's disease.” J Neurochem 139 Suppl 1: 216-231.
Burke, N., A. R. Hall and D. J. Hausenloy (2015). “OPA1 in Cardiovascular Health and Disease.” Curr Drug Targets 16(8): 912-920.
Burte, F., V. Carelli, P. F. Chinnery and P. Yu-Wai-Man (2015). “Disturbed mitochondrial dynamics and neurodegenerative disorders.” Nat Rev Neurol 11(1): 11-24.
Butterfield, D. A., J. Drake, C. Pocernich and A. Castegna (2001). “Evidence of oxidative damage in Alzheimer's disease brain: central role for amyloid beta-peptide.” Trends Mol Med 7(12): 548-554.
Cardoso, S. M., I. Santana, R. H. Swerdlow and C. R. Oliveira (2004). “Mitochondria dysfunction of Alzheimer's disease cybrids enhances Abeta toxicity.” J Neurochem 89(6): 1417-1426.
Carelli, V., F. N. Ross-Cisneros and A. A. Sadun (2002). “Optic nerve degeneration and mitochondrial dysfunction: genetic and acquired optic neuropathies.” Neurochem Int 40(6): 573-584.
Carelli, V., F. N. Ross-Cisneros and A. A. Sadun (2004). “Mitochondrial dysfunction as a cause of optic neuropathies.” Prog Retin Eye Res 23(1): 53-89.
Carvalho, A. S., H. Ribeiro, P. Voabil, D. Penque, O. N. Jensen, H. Molina and R. Matthiesen (2014). “Global mass spectrometry and transcriptomics array-based drug profiling provides novel insight into glucosamine induced endoplasmic reticulum stress.” Mol Cell Proteomics 13(12): 3294-3307.
Caspersen, C., N. Wang, J. Yao, A. Sosunov, X. Chen, J. W. Lustbader, H. W. Xu, D. Stern, G. McKhann and S. D. Yan (2005). “Mitochondrial Abeta: a potential focal point for neuronal metabolic dysfunction in Alzheimer's disease.” FASEB J 19(14): 2040-2041.
Chen, L., Q. Gong, J. P. Stice and A. A. Knowlton (2009). “Mitochondrial OPA1, apoptosis, and heart failure.” Cardiovasc Res 84(1): 91-99.
Chen, L., T. Liu, A. Tran, X. Lu, A. A. Tomilov, V. Davies, G. Cortopassi, N. Chiamvimonvat, D. M. Bers, M. Votruba and A. A. Knowlton (2012). “OPA1 mutation and late-onset cardiomyopathy: mitochondrial dysfunction and mtDNA instability.” J Am Heart Assoc 1(5): e003012.
Choubey, V., D. Safiulina, A. Vaarmann, M. Cagalinec, P. Wareski, M. Kuum, A. Zharkovsky and A. Kaasik (2011). “Mutant A53T alpha-synuclein induces neuronal death by increasing mitochondrial autophagy.” J Biol Chem 286(12): 10814-10824.
Chrysostomou, V., F. Rezania, I. A. Trounce and J. G. Crowston (2013). “Oxidative stress and mitochondrial dysfunction in glaucoma.” Curr Opin Pharmacol 13(1): 12-15.
Cipolat, S., T. Rudka, D. Hartmann, V. Costa, L. Serneels, K. Craessaerts, K. Metzger, C. Frezza, W. Annaert, L. D′Adamio, C. Derks, T. Dejaegere, L. Pellegrini, R. D′Hooge, L. Scorrano and B. De Strooper (2006). “Mitochondrial rhomboid PARL regulates cytochrome c release during apoptosis via OPA1-dependent cristae remodeling.” Cell 126(1): 163-175.
Cogliati, S., C. Frezza, M. E. Soriano, T. Varanita, R. Quintana-Cabrera, M. Corrado, S. Cipolat, V. Costa, A. Casarin, L. C. Gomes, E. Perales-Clemente, L. Salviati, P. Fernandez-Silva, J. A. Enriquez and L. Scorrano (2013). “Mitochondrial cristae shape determines respiratory chain supercomplexes assembly and respiratory efficiency.” Cell 155(1): 160-171.
Coughlin, L., R. S. Morrison, P. J. Horner and D. M. Inman (2015). “Mitochondrial morphology differences and mitophagy deficit in murine glaucomatous optic nerve.” Invest Ophthalmol Vis Sci 56(3): 1437-1446.
Daoud, H., P. N. Valdmanis, F. Gros-Louis, V. Belzil, D. Spiegelman, E. Henrion, O. Diallo, A. Desjarlais, J. Gauthier, W. Camu, P. A. Dion and G. A. Rouleau (2011). “Resequencing of 29 candidate genes in patients with familial and sporadic amyotrophic lateral sclerosis.” Arch Neurol 68(5): 587-593.
Delettre, C., G. Lenaers, J. M. Griffoin, N. Gigarel, C. Lorenzo, P. Belenguer, L. Pelloquin, J. Grosgeorge, C. Turc-Carel, E. Perret, C. Astarie-Dequeker, L. Lasquellec, B. Arnaud, B. Ducommun, J. Kaplan and C. P. Hamel (2000). “Nuclear gene OPA1, encoding a mitochondrial dynamin-related protein, is mutated in dominant optic atrophy.” Nat Genet 26(2): 207-210.
Devi, L., B. M. Prabhu, D. F. Galati, N. G. Avadhani and H. K. Anandatheerthavarada (2006). “Accumulation of amyloid precursor protein in the mitochondrial import channels of human Alzheimer's disease brain is associated with mitochondrial dysfunction.” J Neurosci 26(35): 9057-9068.
Diana, A., G. Simic, E. Sinforiani, N. Orru, G. Pichiri and G. Bono (2008). “Mitochondria morphology and DNA content upon sublethal exposure to beta-amyloid(1-42) peptide.” Coll Antropol 32 Suppl 1: 51-58.
Dolle, C., I. Flones, G. S. Nido, H. Miletic, N. Osuagwu, S. Kristoffersen, P. K. Lilleng, J. P. Larsen, O. B. Tysnes, K. Haugarvoll, L. A. Bindoff and C. Tzoulis (2016). “Defective mitochondrial DNA homeostasis in the substantia nigra in Parkinson disease.” Nat Commun 7: 13548.
Dorn, G. W., 2nd (2013). “Mitochondrial dynamics in heart disease.” Biochim Biophys Acta 1833(1): 233-241.
Duvezin-Caubet, S., R. Jagasia, J. Wagener, S. Hofmann, A. Trifunovic, A. Hansson, A. Chomyn, M. F. Bauer, G. Attardi, N. G. Larsson, W. Neupert and A. S. Reichert (2006). “Proteolytic processing of OPA1 links mitochondrial dysfunction to alterations in mitochondrial morphology.” J Biol Chem 281(49): 37972-37979.
Eberlin, M., T. Muck and M. C. Michel (2012). “A comprehensive review of the pharmacodynamics, pharmacokinetics, and clinical effects of the neutral endopeptidase inhibitor racecadotril.” Front Pharmacol 3: 93.
Eckert, A., S. Hauptmann, I. Scherping, V. Rhein, F. Muller-Spahn, J. Gotz and W. E. Muller (2008). “Soluble beta-amyloid leads to mitochondrial defects in amyloid precursor protein and tau transgenic mice.” Neurodegener Dis 5(3-4): 157-159.
Edgar, R., M. Domrachev and A. E. Lash (2002). “Gene Expression Omnibus: NCBI gene expression and hybridization array data repository.” Nucleic Acids Res 30(1): 207-210.
Ehses, S., I. Raschke, G. Mancuso, A. Bernacchia, S. Geimer, D. Tondera, J. C. Martinou, B. Westermann, E. I. Rugarli and T. Langer (2009). “Regulation of OPA1 processing and mitochondrial fusion by m-AAA protease isoenzymes and OMA1.” J Cell Biol 187(7): 1023-1036.
Faccenda, D., J. Nakamura, G. Gorini, G. K. Dhoot, M. Piacentini, M. Yoshida and M. Campanella (2017). “Control of Mitochondrial Remodeling by the ATPase Inhibitory Factor 1 Unveils a Pro-survival Relay via OPA1.” Cell Rep 18(8): 1869-1883.
Frezza, C., S. Cipolat, O. Martins de Brito, M. Micaroni, G. V. Beznoussenko, T. Rudka, D. Bartoli, R. S. Polishuck, N. N. Danial, B. De Strooper and L. Scorrano (2006). “OPA1 controls apoptotic cristae remodeling independently from mitochondrial fusion.” Cell 126(1): 177-189.
Frezza, C. and E. Gottlieb (2009). “Mitochondria in cancer: not just innocent bystanders.” Semin Cancer Biol 19(1): 4-11.
Fuhrmann, N., S. Schimpf, Y. Kamenisch, B. Leo-Kottler, C. Alexander, G. Auburger, E. Zrenner, B. Wissinger and M. V. Alavi (2010). “Solving a 50-year mystery of a missing OPA1 mutation: more insights from the first family diagnosed with autosomal dominant optic atrophy.” Mol Neurodegener 5(1): 25.
Ghio, S., F. Kamp, R. Cauchi, A. Giese and N. Vassallo (2016). “Interaction of alpha-synuclein with biomembranes in Parkinson's disease—role of cardiolipin.” Prog Lipid Res 61: 73-82.
Gibson, G. E., K. F. Sheu and J. P. Blass (1998). “Abnormalities of mitochondrial enzymes in Alzheimer disease.” J Neural Transm (Vienna) 105(8-9): 855-870.
Gleissner, C. A., I. Shaked, K. M. Little and K. Ley (2010). “CXC chemokine ligand 4 induces a unique transcriptome in monocyte-derived macrophages.” J Immunol 184(9): 4810-4818.
Glytsou, C., E. Calvo, S. Cogliati, A. Mehrotra, I. Anastasia, G. Rigoni, A. Raimondi, N. Shintani, M. Loureiro, J. Vazquez, L. Pellegrini, J. A. Enriquez, L. Scorrano and M. E. Soriano (2016). “Optic Atrophy 1 Is Epistatic to the Core MICOS Component MIC60 in Mitochondrial Cristae Shape Control.” Cell Rep 17(11): 3024-3034.
Griparic, L., T. Kanazawa and A. M. van der Blick (2007). “Regulation of the mitochondrial dynamin-like protein Opal by proteolytic cleavage.” J Cell Biol 178(5): 757-764.
Guardia-Laguarta, C., E. Area-Gomez, C. Rub, Y. Liu, J. Magrane, D. Becker, W. Voos, E. A. Schon and S. Przedborski (2014). “Alpha-Synuclein is localized to mitochondria-associated ER membranes.” J Neurosci 34(1): 249-259.
Guo, Y., X. Chen, H. Zhang, N. Li, X. Yang, W. Cheng and K. Zhao (2012). “Association of OPA1 polymorphisms with NTG and HTG: a meta-analysis.” PLOS One 7(8): c42387.
Hackenbrock, C. R. (1966). “Ultrastructural bases for metabolically linked mechanical activity in mitochondria. I. Reversible ultrastructural changes with change in metabolic steady state in isolated liver mitochondria.” J Cell Biol 30(2): 269-297.
Hanahan, D. and R. A. Weinberg (2011). “Hallmarks of cancer: the next generation.” Cell 144(5): 646-674.
Head, B., L. Griparic, M. Amiri, S. Gandre-Babbe and A. M. van der Blick (2009). “Inducible proteolytic inactivation of OPA1 mediated by the OMA1 protease in mammalian cells.” J Cell Biol 187(7): 959-966.
Hessenberger, M., R. M. Zerbes, H. Rampelt, S. Kunz, A. H. Xavier, B. Purfurst, H. Lilie, N. Pfanner, M. van der Laan and O. Daumke (2017). “Regulated membrane remodeling by Mic60 controls formation of mitochondrial crista junctions.” Nat Commun 8: 15258.
Hokama, M., S. Oka, J. Leon, T. Ninomiya, H. Honda, K. Sasaki, T. Iwaki, T. Ohara, T. Sasaki, F. M. LaFerla, Y. Kiyohara and Y. Nakabeppu (2014). “Altered expression of diabetes-related genes in Alzheimer's disease brains: the Hisayama study.” Cereb Cortex 24(9): 2476-2488.
Imaizumi, Y., Y. Okada, W. et al. (2012). “Mitochondrial dysfunction associated with increased oxidative stress and alpha-synuclein accumulation in PARK2 iPSC-derived neurons and postmortem brain tissue.” Mol Brain 5: 35.
Ishihara, N., Y. Fujita, T. Oka and K. Mihara (2006). “Regulation of mitochondrial morphology through proteolytic cleavage of OPA1.” EMBO J 25(13): 2966-2977.
Jakobs, S., N. Martini, A. C. Schauss, A. Egner, B. Westermann and S. W. Hell (2003). “Spatial and temporal dynamics of budding yeast mitochondria lacking the division component Fislp.” J Cell Sci 116(Pt 10): 2005-2014.
Jiang, X., H. Jiang, Z. Shen and X. Wang (2014). “Activation of mitochondrial protease OMA1 by Bax and Bak promotes cytochrome c release during apoptosis.” Proc Natl Acad Sci USA.
Ju, W. K., K. Y. Kim, J. D. Lindsey, M. Angert, K. X. Duong-Polk, R. T. Scott, J. J. Kim, I. Kukhmazov, M. H. Ellisman, G. A. Perkins and R. N. Weinreb (2008). “Intraocular pressure elevation induces mitochondrial fission and triggers OPA1 release in glaucomatous optic nerve.” Invest Ophthalmol Vis Sci 49(11): 4903-4911.
Kandimalla, R., M. Manczak, D. Fry, Y. Suneetha, H. Sesaki and P. H. Reddy (2016). “Reduced dynamin-related protein 1 protects against phosphorylated Tau-induced mitochondrial dysfunction and synaptic damage in Alzheimer's disease.” Hum Mol Genet 25(22): 4881-4897.
Kaser, M., M. Kambacheld, B. Kisters-Woike and T. Langer (2003). “Omal, a novel membrane-bound metallopeptidase in mitochondria with activities overlapping with the m-AAA protease.” J Biol Chem 278(47): 46414-46423.
Keeney, P. M., J. Xie, R. A. Capaldi and J. P. Bennett, Jr. (2006). “Parkinson's disease brain mitochondrial complex I has oxidatively damaged subunits and is functionally impaired and misassembled.” J Neurosci 26(19): 5256-5264.
Kim, K. Y., G. A. Perkins, et al. (2015). “DRP1 inhibition rescues retinal ganglion cells and their axons by preserving mitochondrial integrity in a mouse model of glaucoma.” Cell Death Dis 6: e1839.
Kong, B., H. Tsuyoshi, M. Orisaka, D. B. Shich, Y. Yoshida and B. K. Tsang (2015). “Mitochondrial dynamics regulating chemoresistance in gynecological cancers.” Ann N Y Acad Sci 1350: 1-16.
Kong, B., Q. Wang, E. Fung, K. Xue and B. K. Tsang (2014). “p53 is required for cisplatin-induced processing of the mitochondrial fusion protein L-Opal that is mediated by the mitochondrial metallopeptidase Omal in gynecologic cancers.” J Biol Chem 289(39): 27134-27145.
Kong, G. Y., N. J. Van Bergen, I. A. Trounce and J. G. Crowston (2009). “Mitochondrial dysfunction and glaucoma.” J Glaucoma 18(2): 93-100.
Koob, S., M. Barrera, R. Anand and A. S. Reichert (2015). “The non-glycosylated isoform of MIC26 is a constituent of the mammalian MICOS complex and promotes formation of crista junctions.” Biochim Biophys Acta 1853(7): 1551-1563.
Korwitz, A., C. Merkwirth, R. Richter-Dennerlein, S. E. Troder, H. G. Sprenger, P. M. Quiros, C. Lopez-Otin, E. I. Rugarli and T. Langer (2016). “Loss of OMA1 delays neurodegeneration by preventing stress-induced OPA1 processing in mitochondria.” J Cell Biol 212(2): 157-166.
Landes, T., L. J. Emorine, D. Courilleau, M. Rojo, P. Belenguer and L. Arnaune-Pelloquin (2010). “The BH3-only Bnip3 binds to the dynamin Opal to promote mitochondrial fragmentation and apoptosis by distinct mechanisms.” EMBO Rep 11(6): 459-465.
Le Page, S., M. Niro, J. Fauconnier, L. Cellier, S. Tamareille, A. Gharib, A. Chevrollier, L. Loufrani, C. Grenier, R. Kamel, E. Sarzi, A. Lacampagne, M. Ovize, D. Henrion, P. Reynier, G. Lenaers, D. Mirebeau-Prunier and F. Prunier (2016). “Increase in Cardiac Ischemia-Reperfusion Injuries in Opa1+/− Mouse Model.” PLOS One 11(10): c0164066.
Lee, S., N. J. Van Bergen, G. Y. Kong, V. Chrysostomou, H. S. Waugh, E. C. O'Neill, J. G. Crowston and I. A. Trounce (2011). “Mitochondrial dysfunction in glaucoma and emerging bioenergetic therapies.” Exp Eye Res 93(2): 204-212.
Lenaers, G., P. Reynier, G. Elachouri, C. Soukkarich, A. Olichon, P. Belenguer, L. Baricault, B. Ducommun, C. Hamel and C. Delettre (2009). “OPA1 functions in mitochondria and dysfunctions in optic nerve.” Int J Biochem Cell Biol 41(10): 1866-1874.
Lesnick, T. G., S. Papapetropoulos, D. C. Mash, J. Ffrench-Mullen, L. Shehadeh, M. de Andrade, J. R. Henley, W. A. Rocca, J. E. Ahlskog and D. M. Maraganore (2007). “A genomic pathway approach to a complex disease: axon guidance and Parkinson disease.” PLOS Genet 3(6): c98.
Li, F., N. Y. Calingasan, F. Yu, W. M. Mauck, M. Toidze, C. G. Almeida, R. H. Takahashi, G. A. Carlson, M. Flint Beal, M. T. Lin and G. K. Gouras (2004). “Increased plaque burden in brains of APP mutant MnSOD heterozygous knockout mice.” J Neurochem 89(5): 1308-1312.
Lustbader, J. W., M. Cirilli, C. Lin, H. W. Xu, K. Takuma, N. Wang, C. Caspersen, X. Chen, S. Pollak, M. Chaney, F. Trinchese, S. Liu, F. Gunn-Moore, L. F. Lue, D. G. Walker, P. Kuppusamy, Z. L. Zewier, O. Arancio, D. Stern, S. S. Yan and H. Wu (2004). “ABAD directly links Abeta to mitochondrial toxicity in Alzheimer's disease.” Science 304(5669): 448-452.
Lutz, A. K., N. Exner, M. E. Fett, J. S. Schlehe, K. Kloos, K. Lammermann, B. Brunner, A. Kurz-Drexler, F. Vogel, A. S. Reichert, L. Bouman, D. Vogt-Weisenhorn, W. Wurst, J. Tatzelt, C. Haass and K. F. Winklhofer (2009). “Loss of parkin or PINKI function increases Drp1-dependent mitochondrial fragmentation.” J Biol Chem 284(34): 22938-22951.
Manczak, M., T. S. Anekonda, E. Henson, B. S. Park, J. Quinn and P. H. Reddy (2006). “Mitochondria are a direct site of A beta accumulation in Alzheimer's disease neurons: implications for free radical generation and oxidative damage in disease progression.” Hum Mol Genet 15(9): 1437-1449.
Manczak, M., M. J. Calkins and P. H. Reddy (2011). “Impaired mitochondrial dynamics and abnormal interaction of amyloid beta with mitochondrial protein Drp1 in neurons from patients with Alzheimer's disease: implications for neuronal damage.” Hum Mol Genet 20(13): 2495-2509.
Manczak, M., R. Kandimalla, D. Fry, H. Sesaki and P. H. Reddy (2016). “Protective effects of reduced dynamin-related protein 1 against amyloid beta-induced mitochondrial dysfunction and synaptic damage in Alzheimer's disease.” Hum Mol Genet 25(23): 5148-5166.
Manczak, M. and P. H. Reddy (2012). “Abnormal interaction between the mitochondrial fission protein Drp1 and hyperphosphorylated tau in Alzheimer's disease neurons: implications for mitochondrial dysfunction and neuronal damage.” Hum Mol Genet 21(11): 2538-2547.
Maraganore, D. M., M. de Andrade, T. G. Lesnick, K. J. Strain, M. J. Farrer, W. A. Rocca, P. V. Pant, K. A. Frazer, D. R. Cox and D. G. Ballinger (2005). “High-resolution whole-genome association study of Parkinson disease.” Am J Hum Genet 77(5): 685-693.
Maresca, A., C. la Morgia, L. Caporali, M. L. Valentino and V. Carelli (2013). “The optic nerve: a “mito-window” on mitochondrial neurodegeneration.” Mol Cell Neurosci 55: 62-76.
Marin-Garcia, J. and A. T. Akhmedov (2016). “Mitochondrial dynamics and cell death in heart failure.” Heart Fail Rev 21(2): 123-136.
Maurer, I., S. Zierz and H. J. Moller (2000). “A selective defect of cytochrome c oxidase is present in brain of Alzheimer disease patients.” Neurobiol Aging 21(3): 455-462.
Medja, F., V. Lelievre, R. H. Fontaine, F. Lebas, P. Leroux, T. Ouimet, A. Saria, C. Rougeot, P. Dournaud and P. Gressens (2006). “Thiorphan, a neutral endopeptidase inhibitor used for diarrhoea, is neuroprotective in newborn mice.” Brain 129(Pt 12): 3209-3223.
Merkwirth, C., S. Dargazanli, T. Tatsuta, S. Geimer, B. Lower, F. T. Wunderlich, J. C. von Kleist-Retzow, A. Waisman, B. Westermann and T. Langer (2008). “Prohibitins control cell proliferation and apoptosis by regulating OPA1-dependent cristae morphogenesis in mitochondria.” Genes Dev 22(4): 476-488.
Miller, J. A., R. L. Woltjer, J. M. Goodenbour, S. Horvath and D. H. Geschwind (2013). “Genes and pathways underlying regional and cell type changes in Alzheimer's disease.” Genome Med 5(5): 48.
Mukherjee, U. A., S. B. Ong, S. G. Ong and D. J. Hausenloy (2015). “Parkinson's disease proteins: Novel mitochondrial targets for cardioprotection.” Pharmacol Ther 156: 34-43.
Nakamura, K. (2013). “Alpha-Synuclein and mitochondria: partners in crime?” Neurotherapeutics 10(3): 391-399.
Nakamura, K., V. M. Nemani, F. Azarbal, G. Skibinski, J. M. Levy, K. Egami, L. Munishkina, J. Zhang, B. Gardner, J. Wakabayashi, H. Sesaki, Y. Cheng, S. Finkbeiner, R. L. Nussbaum, E. Masliah and R. H. Edwards (2011). “Direct membrane association drives mitochondrial fission by the Parkinson disease-associated protein alpha-synuclein.” J Biol Chem 286(23): 20710-20726.
Niemann, A., M. Ruegg, V. La Padula, A. Schenone and U. Suter (2005). “Ganglioside-induced differentiation associated protein 1 is a regulator of the mitochondrial network: new implications for Charcot-Marie-Tooth disease.” J Cell Biol 170(7): 1067-1078.
Olichon, A., L. Baricault, N. Gas, E. Guillou, A. Valette, P. Belenguer and G. Lenaers (2003). “Loss of OPA1 perturbates the mitochondrial inner membrane structure and integrity, leading to cytochrome c release and apoptosis.” J Biol Chem 278(10): 7743-7746.
Ong, S. B., S. B. Kalkhoran, S. Hernandez-Resendiz, P. Samangouci, S. G. Ong and D. J. Hausenloy (2017). “Mitochondrial-Shaping Proteins in Cardiac Health and Disease—the Long and the Short of It!” Cardiovasc Drugs Ther 31(1): 87-107.
Osborne, N. N. (2010). “Mitochondria: Their role in ganglion cell death and survival in primary open angle glaucoma.” Exp Eye Res 90(6): 750-757.
Ott, C., E. Dorsch, M. Fraunholz, S. Straub and V. Kozjak-Pavlovic (2015). “Detailed analysis of the human mitochondrial contact site complex indicate a hierarchy of subunits.” PLOS One 10(3): c0120213.
Pal, H. C., et. al. (2017). “Cryptolepine inhibits melanoma cell growth through coordinated changes in mitochondrial biogenesis, dynamics and metabolic tumor suppressor AMPKalpha½-LKB1.” Sci Rep 7(1): 1498.
Pan, J. Z., H. Wei, J. G. Hecker, J. W. Tobias, R. G. Eckenhoff and M. F. Eckenhoff (2006). “Rat brain DNA transcript profile of halothane and isoflurane exposure.” Pharmacogenet Genomics 16(3): 171-182.
Parker, W. D., Jr., C. M. Filley and J. K. Parks (1990). “Cytochrome oxidase deficiency in Alzheimer's disease.” Neurology 40(8): 1302-1303.
Parker, W. D., Jr., J. K. Parks and R. H. Swerdlow (2008). “Complex I deficiency in Parkinson's disease frontal cortex.” Brain Res 1189: 215-218.
Philibert, R. A., G. Y. Ryu, J. G. Yoon, H. Sandhu, N. Hollenbeck, T. Gunter, A. Barkhurst, W. Adams and A. Madan (2007). “Transcriptional profiling of subjects from the Iowa adoption studies.” Am J Med Genet B Neuropsychiatr Genet 144B(5): 683-690.
Piquereau, J., F. Caffin, M. Novotova, C. Lemaire, V. Veksler, A. Garnier, R. Ventura-Clapier and F. Joubert (2013). “Mitochondrial dynamics in the adult cardiomyocytes: which roles for a highly specialized cell?” Front Physiol 4: 102.
Piquereau, J., F. Caffin, M. Novotova, A. Prola, A. Garnier, P. Mateo, D. Fortin, H. Huynh 1e, V. Nicolas, M. V. Alavi, C. Brenner, R. Ventura-Clapier, V. Veksler and F. Joubert (2012). “Down-regulation of OPA1 alters mouse mitochondrial morphology, PTP function, and cardiac adaptation to pressure overload.” Cardiovasc Res 94(3): 408-417.
Rainbolt, T. K., J. Lebeau, C. Puchades and R. L. Wiseman (2016). “Reciprocal Degradation of YMEIL and OMA1 Adapts Mitochondrial Proteolytic Activity during Stress.” Cell Rep 14(9): 2041-2049.
Rappold, P. M., M. Cui, J. C. Grima, R. Z. Fan, K. L. de Mesy-Bentley, L. Chen, X. Zhuang, W. J. Bowers and K. Ticu (2014). “Drp1 inhibition attenuates neurotoxicity and dopamine release deficits in vivo.” Nat Commun 5: 5244.
Rautio J, et al. (2008) “Prodrugs: design and clinical applications.” Nat Rev Drug Discov 7(3): 255-70.
Reddy, P. H., M. Manczak and X. Yin (2017). “Mitochondria-Division Inhibitor 1 Protects Against Amyloid-beta induced Mitochondrial Fragmentation and Synaptic Damage in Alzheimer's Disease.” J Alzheimers Dis 58(1): 147-162.
Reddy, P. H., S. McWeeney, B. S. Park, M. Manczak, R. V. Gutala, D. Partovi, Y. Jung, V. Yau, R. Searles, M. Mori and J. Quinn (2004). “Gene expression profiles of transcripts in amyloid precursor protein transgenic mice: up-regulation of mitochondrial metabolism and apoptotic genes is an early cellular change in Alzheimer's disease.” Hum Mol Genet 13(12): 1225-1240.
Remington's Pharmaceutical Sciences, 17th ed. (1985)
Richter, U., T. Lahtinen, P. Marttinen, F. Suomi and B. J. Battersby (2015). “Quality control of mitochondrial protein synthesis is required for membrane integrity and cell fitness.” J Cell Biol 211(2): 373-389.
Rossello, A., E. Nuti, E. Orlandini, P. Carelli, S. Rapposelli, M. Macchia, F. Minutolo, L. Carbonaro, A. Albini, R. Benelli, G. Cercignani, G. Murphy and A. Balsamo (2004). “New N-arylsulfonyl-N-alkoxyaminoacetohydroxamic acids as selective inhibitors of gelatinase A (MMP-2).” Bioorg Med Chem 12(9): 2441-2450.
Sadun, A. A. (2002). “Mitochondrial optic neuropathies.” J Neurol Neurosurg Psychiatry 72(4): 423-425.
Salminen, A., A. Haapasalo, A. Kauppinen, K. Kaarniranta, H. Soininen and M. Hiltunen (2015). “Impaired mitochondrial energy metabolism in Alzheimer's disease: Impact on pathogenesis via disturbed epigenetic regulation of chromatin landscape.” Prog Neurobiol 131: 1-20.
Santin, G., et. al. (2013). “Mitochondrial fusion: a mechanism of cisplatin-induced resistance in neuroblastoma cells?” Neurotoxicology 34: 51-60.
Santos, D., A. R. Esteves, D. F. Silva, C. Januario and S. M. Cardoso (2015). “The Impact of Mitochondrial Fusion and Fission Modulation in Sporadic Parkinson's Disease.” Mol Neurobiol 52(1): 573-586.
Schapira, A. H., J. M. Cooper, D. Dexter, J. B. Clark, P. Jenner and C. D. Marsden (1990). “Mitochondrial complex I deficiency in Parkinson's disease.” J Neurochem 54(3): 823-827.
Schmidt, C., E. Lepsverdize, S. L. Chi, A. M. Das, S. V. Pizzo, A. Dityatev and M. Schachner (2008). “Amyloid precursor protein and amyloid beta-peptide bind to ATP synthase and regulate its activity at the surface of neural cells.” Mol Psychiatry 13(10): 953-969.
Shields, L. Y., H. Kim, L. Zhu, D. Haddad, A. Berthet, D. Pathak, M. Lam, R. Ponnusamy, L. G. Diaz-Ramirez, T. M. Gill, H. Sesaki, L. Mucke and K. Nakamura (2015). “Dynamin-related protein 1 is required for normal mitochondrial bioenergetic and synaptic function in CAI hippocampal neurons.” Cell Death Dis 6: e1725.
Sit, A. J. (2014). “Intraocular pressure variations: causes and clinical significance.” Can J Ophthalmol 49(6): 484-488.
Smith, M. A., G. Perry, P. L. Richey, L. M. Sayre, V. E. Anderson, M. F. Beal and N. Kowall (1996). “Oxidative damage in Alzheimer's.” Nature 382(6587): 120-121.
Song, Z., H. Chen, M. Fiket, C. Alexander and D. C. Chan (2007). “OPA1 processing controls mitochondrial fusion and is regulated by mRNA splicing, membrane potential, and YmelL.” J Cell Biol 178(5): 749-755.
Stafa, K., E. Tsika, R. Moser, A. Musso, L. Glauser, A. Jones, S. Biskup, Y. Xiong, R. Bandopadhyay, V. L. Dawson, T. M. Dawson and D. J. Moore (2014). “Functional interaction of Parkinson's disease-associated LRRK2 with members of the dynamin GTPase superfamily.” Hum Mol Genet 23(8): 2055-2077.
Takihara, Y., M. Inatani, K. Eto, T. Inouc, A. Kreymerman, S. Miyake, S. Ueno, M. Nagaya, A. Nakanishi, K. Iwao, Y. Takamura, H. Sakamoto, K. Satoh, M. Kondo, T. Sakamoto, J. L. Goldberg, J. Nabekura and H. Tanihara (2015). “In vivo imaging of axonal transport of mitochondria in the diseased and aged mammalian CNS.” Proc Natl Acad Sci U SA 112(33): 10515-10520.
Tan, E. P., S. R. McGreal, S. Graw, R. Tessman, S. J. Koppel, P. Dhakal, Z. Zhang, M. Machacek, N. E. Zachara, D. C. Koestler, K. R. Peterson, J. P. Thyfault, R. H. Swerdlow, P. Krishnamurthy, L. DiTacchio, U. Apte and C. Slawson (2017). “Sustained O-GlcNAcylation reprograms mitochondrial function to regulate energy metabolism.” J Biol Chem 292(36): 14940-14962.
Tang, Q., et. al. (2018). “Dynamin-related protein 1-mediated mitochondrial fission contributes to IR-783-induced apoptosis in human breast cancer cells.” J Cell Mol Med 22(9): 4474-4485.
Taube, J. H., G. G. Malouf, E. Lu, N. Sphyris, V. Vijay, P. P. Ramachandran, K. R. Ueno, S. Gaur, M. S. Nicoloso, S. Rossi, J. I. Herschkowitz, J. M. Rosen, J. P. Issa, G. A. Calin, J. T. Chang and S. A. Mani (2013). “Epigenetic silencing of microRNA-203 is required for EMT and cancer stem cell properties.” Sci Rep 3: 2687.
Taylor, J. P., J. Hardy and K. H. Fischbeck (2002). “Toxic proteins in neurodegenerative disease.” Science 296(5575): 1991-1995.
Thomas, E., J. J. Feld, Q. Li, Z. Hu, M. W. Fried and T. J. Liang (2011). “Ribavirin potentiates interferon action by augmenting interferon-stimulated gene induction in hepatitis C virus cell culture models.” Hepatology 53(1): 32-41.
Tuccinardi, T., A. Martinelli, E. Nuti, P. Carelli, F. Balzano, G. Uccello-Barretta, G. Murphy and A. Rossello (2006). “Amber force field implementation, molecular modelling study, synthesis and MMP-1/MMP-2 inhibition profile of (R)- and (S)-N-hydroxy-2-(N-isopropoxybiphenyl-4-ylsulfonamido)-3-methylbutanamides.” Bioorg Med Chem 14(12): 4260-4276.
Voigt, A., L. A. Berlemann and K. F. Winklhofer (2016). “The mitochondrial kinase PINK1: functions beyond mitophagy.” J Neurochem 139 Suppl 1: 232-239.
Vyas, S., E. Zaganjor and M. C. Haigis (2016). “Mitochondria and Cancer.” Cell 166(3): 555-566.
Wai, T., J. Garcia-Pricto, M. J. Baker, C. Merkwirth, P. Benit, P. Rustin, F. J. Ruperez, C. Barbas, B. Ibanez and T. Langer (2015). “Imbalanced OPA1 processing and mitochondrial fragmentation cause heart failure in mice.” Science 350(6265): aad0116.
Wai, T., S. Saita, H. Nolte, S. Muller, T. Konig, R. Richter-Dennerlein, H. G. Sprenger, J. Madrenas, M. Muhlmeister, U. Brandt, M. Kruger and T. Langer (2016). “The membrane scaffold SLP2 anchors a proteolytic hub in mitochondria containing PARL and the i-AAA protease YMEIL.” EMBO Rep 17(12): 1844-1856.
Wallace, D. C. (2012). “Mitochondria and cancer.” Nat Rev Cancer 12(10): 685-698.
Wang, H., P. Song, L. Du, W. Tian, W. Yuc, M. Liu, D. Li, B. Wang, Y. Zhu, C. Cao, J. Zhou and Q. Chen (2011). “Parkin ubiquitinates Drp1 for proteasome-dependent degradation: implication of dysregulated mitochondrial dynamics in Parkinson disease.” J Biol Chem 286(13): 11649-11658.
Wang, X., B. Su, S. L. Siedlak, P. I. Moreira, H. Fujioka, Y. Wang, G. Casadesus and X. Zhu (2008). “Amyloid-beta overproduction causes abnormal mitochondrial dynamics via differential modulation of mitochondrial fission/fusion proteins.” Proc Natl Acad Sci USA 105(49): 19318-19323.
Wang, Z., M. Iwasaki, F. Ficara, C. Lin, C. Matheny, S. H. Wong, K. S. Smith and M. L. Cleary (2010). “GSK-3 promotes conditional association of CREB and its coactivators with MEISI to facilitate HOX-mediated transcription and oncogenesis.” Cancer Cell 17(6): 597-608.
Warburg, O. (1956). “On the origin of cancer cells.” Science 123(3191): 309-314.
Xiao, X., Y. Hu, P. M. Quiros, Q. Wei, C. Lopez-Otin and Z. Dong (2014). “OMA1 mediates OPA1 proteolysis and mitochondrial fragmentation in experimental models of ischemic kidney injury.” Am J Physiol Renal Physiol 306(11): F1318-1326.
Yan, J., X. H. Liu, M. Z. Han, Y. M. Wang, X. L. Sun, N. Yu, T. Li, B. Su and Z. Y. Chen (2015). “Blockage of GSK3beta-mediated Drp1 phosphorylation provides neuroprotection in neuronal and mouse models of Alzheimer's disease.” Neurobiol Aging 36(1): 211-227.
Yang, X., Q. Shi, J. Sun, Y. Lv, Y. Ma, C. Chen, K. Xiao, W. Zhou and X. P. Dong (2017). “Aberrant Alterations of Mitochondrial Factors Drp1 and Opal in the Brains of Scrapie Experiment Rodents.” J Mol Neurosci 61(3): 368-378.
Yang, Y., Y. Ouyang, L. Yang, M. F. Beal, A. McQuibban, H. Vogel and B. Lu (2008). “Pink 1 regulates mitochondrial dynamics through interaction with the fission/fusion machinery.” Proc Natl Acad Sci USA 105(19): 7070-7075.
Yao, J., R. W. Irwin, L. Zhao, J. Nilsen, R. T. Hamilton and R. D. Brinton (2009). “Mitochondrial bioenergetic deficit precedes Alzheimer's pathology in female mouse model of Alzheimer's disease.” Proc Natl Acad Sci USA 106(34): 14670-14675.
Zhang, T., L. Xue, L. Li, C. Tang, Z. Wan, R. Wang, J. Tan, Y. Tan, H. Han, R. Tian, T. R. Billiar, W. A. Tao and Z. Zhang (2016). “BNIP3 Protein Suppresses PINK1 Kinase Proteolytic Cleavage to Promote Mitophagy.” J Biol Chem 291(41): 21616-21629.
Zhang, Y., M. James, F. A. Middleton and R. L. Davis (2005). “Transcriptional analysis of multiple brain regions in Parkinson's disease supports the involvement of specific protein processing, energy metabolism, and signaling pathways, and suggests novel disease mechanisms.” Am J Med Genet B Neuropsychiatr Genet 137B(1): 5-16.
Zhao, X., C. Tian, W. M. Puszyk, O. O. Ogunwobi, M. Cao, T. Wang, R. Cabrera, D. R. Nelson and C. Liu (2013). “OPA1 downregulation is involved in sorafenib-induced apoptosis in hepatocellular carcinoma.” Lab Invest 93(1): 8-19.
Zuchner, S., I. V. Mersiyanova, M. Muglia, N. Bissar-Tadmouri, J. Rochelle, E. L. Dadali, M. Zappia, E. Nelis, A. Patitucci, J. Senderek, Y. Parman, O. Evgrafov, P. D. Jonghe, Y. Takahashi, S. Tsuji, M. A. Pericak-Vance, A. Quattrone, E. Battaloglu, A. V. Polyakov, V. Timmerman, J. M. Schroder and J. M. Vance (2004). “Mutations in the mitochondrial GTPase mitofusin 2 cause Charcot-Marie-Tooth neuropathy type 2A.” Nat Genet 36(5): 449-451.

What is claimed is:

1. A method of treating a subject with an abnormal activity or an abnormal gene expression level of OMA1 or an oligomeric complex comprising OMA1 and in need of medical intervention comprising:
(a) obtaining a sample from the subject;
(b) performing a reaction to detect the activity or the gene expression level of OMA1 or the oligomeric complex comprising OMA1 in the sample; and
(c) administering to the subject a pharmaceutically effective amount of tipranavir or an analog thereof based on the activity or the gene expression level of OMA1 or the oligomeric complex comprising OMA1,
whereby the pharmaceutically effective amount of tipranavir or the analog thereof is appropriate to normalize the abnormal activity or the abnormal gene expression level of OMA1 or the oligomeric complex comprising OMA1 in the subject.

2. The method of claim 1, wherein the appropriate pharmaceutically effective amount of tipranavir or the analog thereof can reduce the activity or the gene expression level of OMA1 or the oligomeric complex comprising OMA1, if the reaction to detect the activity or the gene expression level of OMA1 or the oligomeric complex comprising OMA1 in the sample indicates increased activity or gene expression level of OMA1 or the oligomeric complex comprising OMA1 in the subject.

3. The method of claim 2, wherein the appropriate pharmaceutically effective amount is about 100 to 300 micromolar.

4. The method of claim 1, wherein the appropriate pharmaceutically effective amount of tipranavir or the analog thereof can increase the activity or the gene expression level of OMA1 or the oligomeric complex comprising OMA1 if the reaction to detect the activity or the gene expression level of OMA1 or the oligomeric complex comprising OMA1 in the sample indicates reduced activity or gene expression level of OMA1 or the oligomeric complex comprising OMA1 in the subject.

5. The method of claim 4, wherein the appropriate pharmaceutically effective amount is about 0.001 to 50 micromolar.

6. The method of claim 1, wherein the appropriate pharmaceutically effective amount of tipranavir or the analog thereof is administered in a composition comprising a pharmaceutically acceptable excipient.

7. The method of claim 1, wherein said reaction to detect the activity or the gene expression level of OMA1 or the oligomeric complex comprising OMA1 uses an apparatus for gene expression analysis.

8. The method of claim 1, wherein said reaction to detect the activity or the gene expression level of OMA1 or the oligomeric complex comprising OMA1 uses a reagent suitable for the detection of different OPA1 isoforms.

9. The method of claim 1, wherein the appropriate pharmaceutically effective amount of tipranavir or the analog thereof is administered by an apparatus so that the subject can inhale tipranavir or the analog thereof.

10. Tipranavir or a stereochemically isomeric form thereof or a pharmaceutically acceptable salt thereof for the treatment of a mitochondrial disease characterized by OPA1 alterations, wherein an effective amount of tipranavir or the stereochemically isomeric form thereof or the pharmaceutically acceptable salt thereof is selected after detecting altered OMA1 protease activity and/or altered OPA1 cleavage in a patient with the mitochondrial disease.

11. Tipranavir or the stereochemically isomeric form thereof or the pharmaceutically acceptable salt thereof of claim 10, wherein the effective amount is about 100 to 300 micromolar, and wherein the effective amount is selected after detecting elevated OMA1 protease activity and/or elevated OPA1 cleavage in the patient with the mitochondrial disease.

12. Tipranavir or the stereochemically isomeric form thereof or the pharmaceutically acceptable salt thereof of claim 10, wherein the effective amount is about 0.001 to 50 micromolar, and wherein the effective amount is selected after detecting decreased OMA1 protease activity and/or decreased OPA1 cleavage in the patient with the mitochondrial disease.

13. Tipranavir or the stereochemically isomeric form thereof or the pharmaceutically acceptable salt thereof of claim 10, wherein tipranavir or the stereochemically isomeric form thereof or the pharmaceutically acceptable salt thereof is present in a composition comprising a pharmaceutically acceptable excipient.

14. A pharmaceutical composition for the treatment of a mitochondrial disease comprising a compound of the formula V 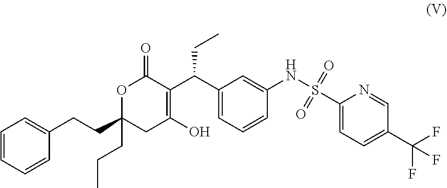 [SMILES: CCC[C@@]1(CCc2ccccc2)CC(O)=C([C@H](CC)c2cccc(NS(=O)(=O)c3ccc(C(F)(F)F)cn3)c2)C(=O)O1]
or a stereochemically isomeric form thereof or a pharmaceutically acceptable salt thereof, wherein said pharmaceutical composition is useful for the dose-dependent modulation of OPA1, OMA1, or an oligomeric complex comprising OPA1 and/or OMA1.

15. The pharmaceutical composition of claim 14, wherein the mitochondrial disease is characterized by amplified OMA1 protease activity and/or amplified OPA1 cleavage.

16. The pharmaceutical composition of claim 15, comprising a pharmaceutically effective amount of about 100 to 300 micromolar of the compound or the stereochemically isomeric form thereof or the pharmaceutically acceptable salt thereof.

17. The pharmaceutical composition of claim 14, wherein the mitochondrial disease is characterized by diminished OMA1 protease activity and/or diminished OPA1 cleavage.

18. The pharmaceutical composition of claim 17, comprising a pharmaceutically effective amount of about 0.001 to 50 micromolar of the compound or the stereochemically isomeric form thereof or the pharmaceutically acceptable salt thereof.

19. The pharmaceutical composition of claim 14, further comprising a pharmaceutically acceptable excipient.

20. The pharmaceutical composition of claim 14, further comprising a pharmaceutically acceptable carrier for intranasal or intrabronchial administration.


SEQUENCE LISTING (63 sequences)

SEQ ID NO: 1 - DNA, 6345 bp, Homo sapiens
  source 1..6345: mol_type = other DNA
gtgctgcccgcctagaaagggtgaagtggttgtttccgtgacggactgagtacgggtgcctgtcaggctcttgcggaagtccatgcgccattgggagggcctcggccgcggctctgtgcccttgctgctgagggccacttcctgggtcattcctggaccgggagccgggctggggctcacacgggggctcccgcgtggccgtctcggcgcctgcgtgacctccccgccggcgggatgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttgagaaaattagaaaagcccttcctagttcagaagaccttgtaaagttagcaccagactttgacaagattgttgaaagccttagcttattgaaggacttttttacctcaggttctccggaagaaacggcgtttagagcaacagatcgtggatctgaaagtgacaagcattttagaaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataaattaaaatcgtactcataatcagctctgcatacatctgaagaacaaaaacatcaacgtcttttgtccagcctctttttcttctgctgttccacctttctaaacatacaataaagtcatgggataaaaataatcgatgtatgttacgggcgctttaaccatcagctgcctctcgaatggaagaacagtggtaatggattaacatcctattttgttgtactaaagtgacaaatcggaataatataattggtatggccattaggttcagtccttgaagataagaaacttgttctctgtttgttgtcttatttgtggtggcactcgtttaatggattaactgaggttgctcaatgttcagtttcttttccagaaatacaatgctaggtgttttgaaataaaacttatatagcaattgtttaaagttatcaattgtatataaaatcacagtagcctgctaaatcattgtatgtgtctgtagtattctattcccagaaactatttgaccatgataattcagtttatattcaccacatgaaagaaaaatgggtaacagaagaacccttaaaacaggttaatttggattgtaacgttcagtgaaagaaatttcaacccttcatagccagcgaagaaatttgccttggaagccaagtcagtaccagcttacctatttgattcagttgctgttttctcactctctatatccatttgaaattgatttattttagatgttgtatacttacgttaggctttctgttaatagtggtttttctcctgttgacagagccaccggattatgacacaggatgaggaagattaaggataatcaattgactaatttcatttagaatattatcaaacatttcaactaggtatcagaaaaaggctttctttcataagactattttaaatagaaattatttcaacaattaaagtaatgttgaccatccccctctcagctgaataaagaaaaatttagttcaatttattgcaatttaattacaatactaccttcacaacattttcatgtgttttaaataaatattttttaattggctaaaggacattcaagcaaagaaatgctttctttacttaaaatgtctatctcatttgctgccttttcactaagcctttactttgttaataaaagtgtccattgtgtgatgtttttgattttacagtttgctaaatcttattttcttggagttgctttttggtaacagccccattgctactccccattttattgttttacatcaatgcatgcttcgttgtgatccctcaagatgtaacacttggtatgctcggttgaggatatgaaaaaatacttccgaaaccaggaattcaatgtatgtttgttttatactgtttgataagaaaagtaggtccagccttaagcagcacagatgcgctggtagatgcatagtcaggaactttttttatttcttttaggtctagggacaggagtgaatagaaagggaggagagctctattatgttctatacacagattaggagatgaccttactgggtacacccctctaaccagtgcttacaggttaatgcatgttaatgaatatttttgcagttgtaaagcataacaattacaactacacatctatttctaaagaataaaacaggaccatatttatttacttctgtcaactatagaaagaaagaccttcagctgtatttccacagatttctcccaaggaaaaggctaatattagtcactactgttatcacatccctttgtataagttttaaaaagagatggagggagatcttcatttctttgaggagatcagtattgtaacgtatgtgaatagatgataacaattaatattactaaaagtcccacatgagagtcctgacgccctctccatgccccacagtaatgtggcttctttcatgggtttttttttcttctttttagctgatctcatcctaagcatgctttatttttccttgaaagctaggtatttatcaactgcagatgttattgaaagaaaataaaattcagtctcaagagtaaaccctgtgtcttgtgtctgtagttcaaaagtcagaaatgattctaatttaaacaaaaagatactaaatatacagaagttaaattcgaactagccacagaatcatttgtttttatgtcagaatttgcaaagagtggagtggacaaagctctgtatggaagactgaacaactgtaaatagatgatatccaaacttaatttggctaggacttcaattttaaaaatcagtgtacctaggcagtgcacagcacgaaataagtggcccttgcagcttccccgtttaacccactgtgctatagttgcgggtggaacagtcaacctttctagtagtttatgatattgccctctttgtattcccattttctacagttttttccgcagacttctttctgcaaattattcagcctccaaatgcaaatgaatgatataaaaataagtagggaacatggcagagagtggtgcttcccagcctcacaatgtgggaatttgacataggatgagagtcagagtataggtttaaaagataaaatctttagttaataattttgtatttatttattctagatgtatgtatctgaggaaagaaatctggtatttttgctttccaataaaggggatcaaagtaatggtttttctctcagttctctaagctggtctatgttatagctctagcagtatggaaatgtgctttaaaatatgcttaccttttgaatgatcatggctatatgttgttgagatatttgaaacttaccttgttttcacttgtgcactgtgaatgaactttgtattatttttttaaaaccttcacattacgtgtagatattattgcaacttatattttgcctgagcttgatcaaaggtcatttgtgtagatgagtaattaaaaaatatttaaatcacattataattctattattggagagcatcttttaaatttttttctgttttaacgagggaaagagaaacctgtatacctagggtcattatttgaccccatagtataaccagattcatggtctaacaagctctcagtgtggcttttctctgaatgcttgaatttcacatgccttgcatttcacagttgtactccatggtcaaccggtgctttttttcacatcgtggtacttgtcaaaacattttgttattttccttggtaaaatatataaaaaaggttttctaatttcaaaaaaaaaaaaaaaaaa

SEQ ID NO: 2 - protein, 960 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFEKIRKALPSSEDLVKLAPDFDKIVESLSLLKDFFTSGSPEETAFRATDRGSESDKHFRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 3 - DNA, 6237 bp, Homo sapiens
  source 1..6237: mol_type = other DNA
gtgctgcccgcctagaaagggtgaagtggttgtttccgtgacggactgagtacgggtgcctgtcaggctcttgcggaagtccatgcgccattgggagggcctcggccgcggctctgtgcccttgctgctgagggccacttcctgggtcattcctggaccgggagccgggctggggctcacacgggggctcccgcgtggccgtctcggcgcctgcgtgacctccccgccggcgggatgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttggttctccggaagaaacggcgtttagagcaacagatcgtggatctgaaagtgacaagcattttagaaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataaattaaaatcgtactcataatcagctctgcatacatctgaagaacaaaaacatcaacgtcttttgtccagcctctttttcttctgctgttccacctttctaaacatacaataaagtcatgggataaaaataatcgatgtatgttacgggcgctttaaccatcagctgcctctcgaatggaagaacagtggtaatggattaacatcctattttgttgtactaaagtgacaaatcggaataatataattggtatggccattaggttcagtccttgaagataagaaacttgttctctgtttgttgtcttatttgtggtggcactcgtttaatggattaactgaggttgctcaatgttcagtttcttttccagaaatacaatgctaggtgttttgaaataaaacttatatagcaattgtttaaagttatcaattgtatataaaatcacagtagcctgctaaatcattgtatgtgtctgtagtattctattcccagaaactatttgaccatgataattcagtttatattcaccacatgaaagaaaaatgggtaacagaagaacccttaaaacaggttaatttggattgtaacgttcagtgaaagaaatttcaacccttcatagccagcgaagaaatttgccttggaagccaagtcagtaccagcttacctatttgattcagttgctgttttctcactctctatatccatttgaaattgatttattttagatgttgtatacttacgttaggctttctgttaatagtggtttttctcctgttgacagagccaccggattatgacacaggatgaggaagattaaggataatcaattgactaatttcatttagaatattatcaaacatttcaactaggtatcagaaaaaggctttctttcataagactattttaaatagaaattatttcaacaattaaagtaatgttgaccatccccctctcagctgaataaagaaaaatttagttcaatttattgcaatttaattacaatactaccttcacaacattttcatgtgttttaaataaatattttttaattggctaaaggacattcaagcaaagaaatgctttctttacttaaaatgtctatctcatttgctgccttttcactaagcctttactttgttaataaaagtgtccattgtgtgatgtttttgattttacagtttgctaaatcttattttcttggagttgctttttggtaacagccccattgctactccccattttattgttttacatcaatgcatgcttcgttgtgatccctcaagatgtaacacttggtatgctcggttgaggatatgaaaaaatacttccgaaaccaggaattcaatgtatgtttgttttatactgtttgataagaaaagtaggtccagccttaagcagcacagatgcgctggtagatgcatagtcaggaactttttttatttcttttaggtctagggacaggagtgaatagaaagggaggagagctctattatgttctatacacagattaggagatgaccttactgggtacacccctctaaccagtgcttacaggttaatgcatgttaatgaatatttttgcagttgtaaagcataacaattacaactacacatctatttctaaagaataaaacaggaccatatttatttacttctgtcaactatagaaagaaagaccttcagctgtatttccacagatttctcccaaggaaaaggctaatattagtcactactgttatcacatccctttgtataagttttaaaaagagatggagggagatcttcatttctttgaggagatcagtattgtaacgtatgtgaatagatgataacaattaatattactaaaagtcccacatgagagtcctgacgccctctccatgccccacagtaatgtggcttctttcatgggtttttttttcttctttttagctgatctcatcctaagcatgctttatttttccttgaaagctaggtatttatcaactgcagatgttattgaaagaaaataaaattcagtctcaagagtaaaccctgtgtcttgtgtctgtagttcaaaagtcagaaatgattctaatttaaacaaaaagatactaaatatacagaagttaaattcgaactagccacagaatcatttgtttttatgtcagaatttgcaaagagtggagtggacaaagctctgtatggaagactgaacaactgtaaatagatgatatccaaacttaatttggctaggacttcaattttaaaaatcagtgtacctaggcagtgcacagcacgaaataagtggcccttgcagcttccccgtttaacccactgtgctatagttgcgggtggaacagtcaacctttctagtagtttatgatattgccctctttgtattcccattttctacagttttttccgcagacttctttctgcaaattattcagcctccaaatgcaaatgaatgatataaaaataagtagggaacatggcagagagtggtgcttcccagcctcacaatgtgggaatttgacataggatgagagtcagagtataggtttaaaagataaaatctttagttaataattttgtatttatttattctagatgtatgtatctgaggaaagaaatctggtatttttgctttccaataaaggggatcaaagtaatggtttttctctcagttctctaagctggtctatgttatagctctagcagtatggaaatgtgctttaaaatatgcttaccttttgaatgatcatggctatatgttgttgagatatttgaaacttaccttgttttcacttgtgcactgtgaatgaactttgtattatttttttaaaaccttcacattacgtgtagatattattgcaacttatattttgcctgagcttgatcaaaggtcatttgtgtagatgagtaattaaaaaatatttaaatcacattataattctattattggagagcatcttttaaatttttttctgttttaacgagggaaagagaaacctgtatacctagggtcattatttgaccccatagtataaccagattcatggtctaacaagctctcagtgtggcttttctctgaatgcttgaatttcacatgccttgcatttcacagttgtactccatggtcaaccggtgctttttttcacatcgtggtacttgtcaaaacattttgttattttccttggtaaaatatataaaaaaggttttctaatttcaaaaaaaaaaaaaaaaaa

SEQ ID NO: 4 - protein, 924 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFGSPEETAFRATDRGSESDKHFRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 5 - DNA, 6291 bp, Homo sapiens
  source 1..6291: mol_type = other DNA
gtgctgcccgcctagaaagggtgaagtggttgtttccgtgacggactgagtacgggtgcctgtcaggctcttgcggaagtccatgcgccattgggagggcctcggccgcggctctgtgcccttgctgctgagggccacttcctgggtcattcctggaccgggagccgggctggggctcacacgggggctcccgcgtggccgtctcggcgcctgcgtgacctccccgccggcgggatgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttggtcacaaattggttagtgaagtcataggagcttctgacctacttctcttgttaggttctccggaagaaacggcgtttagagcaacagatcgtggatctgaaagtgacaagcattttagaaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataaattaaaatcgtactcataatcagctctgcatacatctgaagaacaaaaacatcaacgtcttttgtccagcctctttttcttctgctgttccacctttctaaacatacaataaagtcatgggataaaaataatcgatgtatgttacgggcgctttaaccatcagctgcctctcgaatggaagaacagtggtaatggattaacatcctattttgttgtactaaagtgacaaatcggaataatataattggtatggccattaggttcagtccttgaagataagaaacttgttctctgtttgttgtcttatttgtggtggcactcgtttaatggattaactgaggttgctcaatgttcagtttcttttccagaaatacaatgctaggtgttttgaaataaaacttatatagcaattgtttaaagttatcaattgtatataaaatcacagtagcctgctaaatcattgtatgtgtctgtagtattctattcccagaaactatttgaccatgataattcagtttatattcaccacatgaaagaaaaatgggtaacagaagaacccttaaaacaggttaatttggattgtaacgttcagtgaaagaaatttcaacccttcatagccagcgaagaaatttgccttggaagccaagtcagtaccagcttacctatttgattcagttgctgttttctcactctctatatccatttgaaattgatttattttagatgttgtatacttacgttaggctttctgttaatagtggtttttctcctgttgacagagccaccggattatgacacaggatgaggaagattaaggataatcaattgactaatttcatttagaatattatcaaacatttcaactaggtatcagaaaaaggctttctttcataagactattttaaatagaaattatttcaacaattaaagtaatgttgaccatccccctctcagctgaataaagaaaaatttagttcaatttattgcaatttaattacaatactaccttcacaacattttcatgtgttttaaataaatattttttaattggctaaaggacattcaagcaaagaaatgctttctttacttaaaatgtctatctcatttgctgccttttcactaagcctttactttgttaataaaagtgtccattgtgtgatgtttttgattttacagtttgctaaatcttattttcttggagttgctttttggtaacagccccattgctactccccattttattgttttacatcaatgcatgcttcgttgtgatccctcaagatgtaacacttggtatgctcggttgaggatatgaaaaaatacttccgaaaccaggaattcaatgtatgtttgttttatactgtttgataagaaaagtaggtccagccttaagcagcacagatgcgctggtagatgcatagtcaggaactttttttatttcttttaggtctagggacaggagtgaatagaaagggaggagagctctattatgttctatacacagattaggagatgaccttactgggtacacccctctaaccagtgcttacaggttaatgcatgttaatgaatatttttgcagttgtaaagcataacaattacaactacacatctatttctaaagaataaaacaggaccatatttatttacttctgtcaactatagaaagaaagaccttcagctgtatttccacagatttctcccaaggaaaaggctaatattagtcactactgttatcacatccctttgtataagttttaaaaagagatggagggagatcttcatttctttgaggagatcagtattgtaacgtatgtgaatagatgataacaattaatattactaaaagtcccacatgagagtcctgacgccctctccatgccccacagtaatgtggcttctttcatgggtttttttttcttctttttagctgatctcatcctaagcatgctttatttttccttgaaagctaggtatttatcaactgcagatgttattgaaagaaaataaaattcagtctcaagagtaaaccctgtgtcttgtgtctgtagttcaaaagtcagaaatgattctaatttaaacaaaaagatactaaatatacagaagttaaattcgaactagccacagaatcatttgtttttatgtcagaatttgcaaagagtggagtggacaaagctctgtatggaagactgaacaactgtaaatagatgatatccaaacttaatttggctaggacttcaattttaaaaatcagtgtacctaggcagtgcacagcacgaaataagtggcccttgcagcttccccgtttaacccactgtgctatagttgcgggtggaacagtcaacctttctagtagtttatgatattgccctctttgtattcccattttctacagttttttccgcagacttctttctgcaaattattcagcctccaaatgcaaatgaatgatataaaaataagtagggaacatggcagagagtggtgcttcccagcctcacaatgtgggaatttgacataggatgagagtcagagtataggtttaaaagataaaatctttagttaataattttgtatttatttattctagatgtatgtatctgaggaaagaaatctggtatttttgctttccaataaaggggatcaaagtaatggtttttctctcagttctctaagctggtctatgttatagctctagcagtatggaaatgtgctttaaaatatgcttaccttttgaatgatcatggctatatgttgttgagatatttgaaacttaccttgttttcacttgtgcactgtgaatgaactttgtattatttttttaaaaccttcacattacgtgtagatattattgcaacttatattttgcctgagcttgatcaaaggtcatttgtgtagatgagtaattaaaaaatatttaaatcacattataattctattattggagagcatcttttaaatttttttctgttttaacgagggaaagagaaacctgtatacctagggtcattatttgaccccatagtataaccagattcatggtctaacaagctctcagtgtggcttttctctgaatgcttgaatttcacatgccttgcatttcacagttgtactccatggtcaaccggtgctttttttcacatcgtggtacttgtcaaaacattttgttattttccttggtaaaatatataaaaaaggttttctaatttcaaaaaaaaaaaaaaaaaa

SEQ ID NO: 6 - protein, 942 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFGHKLVSEVIGASDLLLLLGSPEETAFRATDRGSESDKHFRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 7 - DNA, 6348 bp, Homo sapiens
  source 1..6348: mol_type = other DNA
gtgctgcccgcctagaaagggtgaagtggttgtttccgtgacggactgagtacgggtgcctgtcaggctcttgcggaagtccatgcgccattgggagggcctcggccgcggctctgtgcccttgctgctgagggccacttcctgggtcattcctggaccgggagccgggctggggctcacacgggggctcccgcgtggccgtctcggcgcctgcgtgacctccccgccggcgggatgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttggttctccggaagaaacggcgtttagagcaacagatcgtggatctgaaagtgacaagcattttagaaagggtctgcttggtgagctcattctcttacaacaacaaattcaagagcatgaagaggaagcgcgcagagccgctggccaatatagcacgagctatgcccaacagaagcgcaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataaattaaaatcgtactcataatcagctctgcatacatctgaagaacaaaaacatcaacgtcttttgtccagcctctttttcttctgctgttccacctttctaaacatacaataaagtcatgggataaaaataatcgatgtatgttacgggcgctttaaccatcagctgcctctcgaatggaagaacagtggtaatggattaacatcctattttgttgtactaaagtgacaaatcggaataatataattggtatggccattaggttcagtccttgaagataagaaacttgttctctgtttgttgtcttatttgtggtggcactcgtttaatggattaactgaggttgctcaatgttcagtttcttttccagaaatacaatgctaggtgttttgaaataaaacttatatagcaattgtttaaagttatcaattgtatataaaatcacagtagcctgctaaatcattgtatgtgtctgtagtattctattcccagaaactatttgaccatgataattcagtttatattcaccacatgaaagaaaaatgggtaacagaagaacccttaaaacaggttaatttggattgtaacgttcagtgaaagaaatttcaacccttcatagccagcgaagaaatttgccttggaagccaagtcagtaccagcttacctatttgattcagttgctgttttctcactctctatatccatttgaaattgatttattttagatgttgtatacttacgttaggctttctgttaatagtggtttttctcctgttgacagagccaccggattatgacacaggatgaggaagattaaggataatcaattgactaatttcatttagaatattatcaaacatttcaactaggtatcagaaaaaggctttctttcataagactattttaaatagaaattatttcaacaattaaagtaatgttgaccatccccctctcagctgaataaagaaaaatttagttcaatttattgcaatttaattacaatactaccttcacaacattttcatgtgttttaaataaatattttttaattggctaaaggacattcaagcaaagaaatgctttctttacttaaaatgtctatctcatttgctgccttttcactaagcctttactttgttaataaaagtgtccattgtgtgatgtttttgattttacagtttgctaaatcttattttcttggagttgctttttggtaacagccccattgctactccccattttattgttttacatcaatgcatgcttcgttgtgatccctcaagatgtaacacttggtatgctcggttgaggatatgaaaaaatacttccgaaaccaggaattcaatgtatgtttgttttatactgtttgataagaaaagtaggtccagccttaagcagcacagatgcgctggtagatgcatagtcaggaactttttttatttcttttaggtctagggacaggagtgaatagaaagggaggagagctctattatgttctatacacagattaggagatgaccttactgggtacacccctctaaccagtgcttacaggttaatgcatgttaatgaatatttttgcagttgtaaagcataacaattacaactacacatctatttctaaagaataaaacaggaccatatttatttacttctgtcaactatagaaagaaagaccttcagctgtatttccacagatttctcccaaggaaaaggctaatattagtcactactgttatcacatccctttgtataagttttaaaaagagatggagggagatcttcatttctttgaggagatcagtattgtaacgtatgtgaatagatgataacaattaatattactaaaagtcccacatgagagtcctgacgccctctccatgccccacagtaatgtggcttctttcatgggtttttttttcttctttttagctgatctcatcctaagcatgctttatttttccttgaaagctaggtatttatcaactgcagatgttattgaaagaaaataaaattcagtctcaagagtaaaccctgtgtcttgtgtctgtagttcaaaagtcagaaatgattctaatttaaacaaaaagatactaaatatacagaagttaaattcgaactagccacagaatcatttgtttttatgtcagaatttgcaaagagtggagtggacaaagctctgtatggaagactgaacaactgtaaatagatgatatccaaacttaatttggctaggacttcaattttaaaaatcagtgtacctaggcagtgcacagcacgaaataagtggcccttgcagcttccccgtttaacccactgtgctatagttgcgggtggaacagtcaacctttctagtagtttatgatattgccctctttgtattcccattttctacagttttttccgcagacttctttctgcaaattattcagcctccaaatgcaaatgaatgatataaaaataagtagggaacatggcagagagtggtgcttcccagcctcacaatgtgggaatttgacataggatgagagtcagagtataggtttaaaagataaaatctttagttaataattttgtatttatttattctagatgtatgtatctgaggaaagaaatctggtatttttgctttccaataaaggggatcaaagtaatggtttttctctcagttctctaagctggtctatgttatagctctagcagtatggaaatgtgctttaaaatatgcttaccttttgaatgatcatggctatatgttgttgagatatttgaaacttaccttgttttcacttgtgcactgtgaatgaactttgtattatttttttaaaaccttcacattacgtgtagatattattgcaacttatattttgcctgagcttgatcaaaggtcatttgtgtagatgagtaattaaaaaatatttaaatcacattataattctattattggagagcatcttttaaatttttttctgttttaacgagggaaagagaaacctgtatacctagggtcattatttgaccccatagtataaccagattcatggtctaacaagctctcagtgtggcttttctctgaatgcttgaatttcacatgccttgcatttcacagttgtactccatggtcaaccggtgctttttttcacatcgtggtacttgtcaaaacattttgttattttccttggtaaaatatataaaaaaggttttctaatttcaaaaaaaaaaaaaaaaaa

SEQ ID NO: 8 - protein, 961 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFGSPEETAFRATDRGSESDKHFRKGLLGELILLQQQIQEHEEEARRAAGQYSTSYAQQKRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 9 - DNA, 6399 bp, Homo sapiens
  source 1..6399: mol_type = other DNA
gtgctgcccgcctagaaagggtgaagtggttgtttccgtgacggactgagtacgggtgcctgtcaggctcttgcggaagtccatgcgccattgggagggcctcggccgcggctctgtgcccttgctgctgagggccacttcctgggtcattcctggaccgggagccgggctggggctcacacgggggctcccgcgtggccgtctcggcgcctgcgtgacctccccgccggcgggatgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttgagaaaattagaaaagcccttcctagttcagaagaccttgtaaagttagcaccagactttgacaagattgttgaaagccttagcttattgaaggacttttttacctcaggtcacaaattggttagtgaagtcataggagcttctgacctacttctcttgttaggttctccggaagaaacggcgtttagagcaacagatcgtggatctgaaagtgacaagcattttagaaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataaattaaaatcgtactcataatcagctctgcatacatctgaagaacaaaaacatcaacgtcttttgtccagcctctttttcttctgctgttccacctttctaaacatacaataaagtcatgggataaaaataatcgatgtatgttacgggcgctttaaccatcagctgcctctcgaatggaagaacagtggtaatggattaacatcctattttgttgtactaaagtgacaaatcggaataatataattggtatggccattaggttcagtccttgaagataagaaacttgttctctgtttgttgtcttatttgtggtggcactcgtttaatggattaactgaggttgctcaatgttcagtttcttttccagaaatacaatgctaggtgttttgaaataaaacttatatagcaattgtttaaagttatcaattgtatataaaatcacagtagcctgctaaatcattgtatgtgtctgtagtattctattcccagaaactatttgaccatgataattcagtttatattcaccacatgaaagaaaaatgggtaacagaagaacccttaaaacaggttaatttggattgtaacgttcagtgaaagaaatttcaacccttcatagccagcgaagaaatttgccttggaagccaagtcagtaccagcttacctatttgattcagttgctgttttctcactctctatatccatttgaaattgatttattttagatgttgtatacttacgttaggctttctgttaatagtggtttttctcctgttgacagagccaccggattatgacacaggatgaggaagattaaggataatcaattgactaatttcatttagaatattatcaaacatttcaactaggtatcagaaaaaggctttctttcataagactattttaaatagaaattatttcaacaattaaagtaatgttgaccatccccctctcagctgaataaagaaaaatttagttcaatttattgcaatttaattacaatactaccttcacaacattttcatgtgttttaaataaatattttttaattggctaaaggacattcaagcaaagaaatgctttctttacttaaaatgtctatctcatttgctgccttttcactaagcctttactttgttaataaaagtgtccattgtgtgatgtttttgattttacagtttgctaaatcttattttcttggagttgctttttggtaacagccccattgctactccccattttattgttttacatcaatgcatgcttcgttgtgatccctcaagatgtaacacttggtatgctcggttgaggatatgaaaaaatacttccgaaaccaggaattcaatgtatgtttgttttatactgtttgataagaaaagtaggtccagccttaagcagcacagatgcgctggtagatgcatagtcaggaactttttttatttcttttaggtctagggacaggagtgaatagaaagggaggagagctctattatgttctatacacagattaggagatgaccttactgggtacacccctctaaccagtgcttacaggttaatgcatgttaatgaatatttttgcagttgtaaagcataacaattacaactacacatctatttctaaagaataaaacaggaccatatttatttacttctgtcaactatagaaagaaagaccttcagctgtatttccacagatttctcccaaggaaaaggctaatattagtcactactgttatcacatccctttgtataagttttaaaaagagatggagggagatcttcatttctttgaggagatcagtattgtaacgtatgtgaatagatgataacaattaatattactaaaagtcccacatgagagtcctgacgccctctccatgccccacagtaatgtggcttctttcatgggtttttttttcttctttttagctgatctcatcctaagcatgctttatttttccttgaaagctaggtatttatcaactgcagatgttattgaaagaaaataaaattcagtctcaagagtaaaccctgtgtcttgtgtctgtagttcaaaagtcagaaatgattctaatttaaacaaaaagatactaaatatacagaagttaaattcgaactagccacagaatcatttgtttttatgtcagaatttgcaaagagtggagtggacaaagctctgtatggaagactgaacaactgtaaatagatgatatccaaacttaatttggctaggacttcaattttaaaaatcagtgtacctaggcagtgcacagcacgaaataagtggcccttgcagcttccccgtttaacccactgtgctatagttgcgggtggaacagtcaacctttctagtagtttatgatattgccctctttgtattcccattttctacagttttttccgcagacttctttctgcaaattattcagcctccaaatgcaaatgaatgatataaaaataagtagggaacatggcagagagtggtgcttcccagcctcacaatgtgggaatttgacataggatgagagtcagagtataggtttaaaagataaaatctttagttaataattttgtatttatttattctagatgtatgtatctgaggaaagaaatctggtatttttgctttccaataaaggggatcaaagtaatggtttttctctcagttctctaagctggtctatgttatagctctagcagtatggaaatgtgctttaaaatatgcttaccttttgaatgatcatggctatatgttgttgagatatttgaaacttaccttgttttcacttgtgcactgtgaatgaactttgtattatttttttaaaaccttcacattacgtgtagatattattgcaacttatattttgcctgagcttgatcaaaggtcatttgtgtagatgagtaattaaaaaatatttaaatcacattataattctattattggagagcatcttttaaatttttttctgttttaacgagggaaagagaaacctgtatacctagggtcattatttgaccccatagtataaccagattcatggtctaacaagctctcagtgtggcttttctctgaatgcttgaatttcacatgccttgcatttcacagttgtactccatggtcaaccggtgctttttttcacatcgtggtacttgtcaaaacattttgttattttccttggtaaaatatataaaaaaggttttctaatttcaaaaaaaaaaaaaaaaaa

SEQ ID NO: 10 - protein, 978 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFEKIRKALPSSEDLVKLAPDFDKIVESLSLLKDFFTSGHKLVSEVIGASDLLLLLGSPEETAFRATDRGSESDKHFRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 11 - DNA, 6402 bp, Homo sapiens
  source 1..6402: mol_type = other DNA
gtgctgcccgcctagaaagggtgaagtggttgtttccgtgacggactgagtacgggtgcctgtcaggctcttgcggaagtccatgcgccattgggagggcctcggccgcggctctgtgcccttgctgctgagggccacttcctgggtcattcctggaccgggagccgggctggggctcacacgggggctcccgcgtggccgtctcggcgcctgcgtgacctccccgccggcgggatgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttggtcacaaattggttagtgaagtcataggagcttctgacctacttctcttgttaggttctccggaagaaacggcgtttagagcaacagatcgtggatctgaaagtgacaagcattttagaaagggtctgcttggtgagctcattctcttacaacaacaaattcaagagcatgaagaggaagcgcgcagagccgctggccaatatagcacgagctatgcccaacagaagcgcaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataaattaaaatcgtactcataatcagctctgcatacatctgaagaacaaaaacatcaacgtcttttgtccagcctctttttcttctgctgttccacctttctaaacatacaataaagtcatgggataaaaataatcgatgtatgttacgggcgctttaaccatcagctgcctctcgaatggaagaacagtggtaatggattaacatcctattttgttgtactaaagtgacaaatcggaataatataattggtatggccattaggttcagtccttgaagataagaaacttgttctctgtttgttgtcttatttgtggtggcactcgtttaatggattaactgaggttgctcaatgttcagtttcttttccagaaatacaatgctaggtgttttgaaataaaacttatatagcaattgtttaaagttatcaattgtatataaaatcacagtagcctgctaaatcattgtatgtgtctgtagtattctattcccagaaactatttgaccatgataattcagtttatattcaccacatgaaagaaaaatgggtaacagaagaacccttaaaacaggttaatttggattgtaacgttcagtgaaagaaatttcaacccttcatagccagcgaagaaatttgccttggaagccaagtcagtaccagcttacctatttgattcagttgctgttttctcactctctatatccatttgaaattgatttattttagatgttgtatacttacgttaggctttctgttaatagtggtttttctcctgttgacagagccaccggattatgacacaggatgaggaagattaaggataatcaattgactaatttcatttagaatattatcaaacatttcaactaggtatcagaaaaaggctttctttcataagactattttaaatagaaattatttcaacaattaaagtaatgttgaccatccccctctcagctgaataaagaaaaatttagttcaatttattgcaatttaattacaatactaccttcacaacattttcatgtgttttaaataaatattttttaattggctaaaggacattcaagcaaagaaatgctttctttacttaaaatgtctatctcatttgctgccttttcactaagcctttactttgttaataaaagtgtccattgtgtgatgtttttgattttacagtttgctaaatcttattttcttggagttgctttttggtaacagccccattgctactccccattttattgttttacatcaatgcatgcttcgttgtgatccctcaagatgtaacacttggtatgctcggttgaggatatgaaaaaatacttccgaaaccaggaattcaatgtatgtttgttttatactgtttgataagaaaagtaggtccagccttaagcagcacagatgcgctggtagatgcatagtcaggaactttttttatttcttttaggtctagggacaggagtgaatagaaagggaggagagctctattatgttctatacacagattaggagatgaccttactgggtacacccctctaaccagtgcttacaggttaatgcatgttaatgaatatttttgcagttgtaaagcataacaattacaactacacatctatttctaaagaataaaacaggaccatatttatttacttctgtcaactatagaaagaaagaccttcagctgtatttccacagatttctcccaaggaaaaggctaatattagtcactactgttatcacatccctttgtataagttttaaaaagagatggagggagatcttcatttctttgaggagatcagtattgtaacgtatgtgaatagatgataacaattaatattactaaaagtcccacatgagagtcctgacgccctctccatgccccacagtaatgtggcttctttcatgggtttttttttcttctttttagctgatctcatcctaagcatgctttatttttccttgaaagctaggtatttatcaactgcagatgttattgaaagaaaataaaattcagtctcaagagtaaaccctgtgtcttgtgtctgtagttcaaaagtcagaaatgattctaatttaaacaaaaagatactaaatatacagaagttaaattcgaactagccacagaatcatttgtttttatgtcagaatttgcaaagagtggagtggacaaagctctgtatggaagactgaacaactgtaaatagatgatatccaaacttaatttggctaggacttcaattttaaaaatcagtgtacctaggcagtgcacagcacgaaataagtggcccttgcagcttccccgtttaacccactgtgctatagttgcgggtggaacagtcaacctttctagtagtttatgatattgccctctttgtattcccattttctacagttttttccgcagacttctttctgcaaattattcagcctccaaatgcaaatgaatgatataaaaataagtagggaacatggcagagagtggtgcttcccagcctcacaatgtgggaatttgacataggatgagagtcagagtataggtttaaaagataaaatctttagttaataattttgtatttatttattctagatgtatgtatctgaggaaagaaatctggtatttttgctttccaataaaggggatcaaagtaatggtttttctctcagttctctaagctggtctatgttatagctctagcagtatggaaatgtgctttaaaatatgcttaccttttgaatgatcatggctatatgttgttgagatatttgaaacttaccttgttttcacttgtgcactgtgaatgaactttgtattatttttttaaaaccttcacattacgtgtagatattattgcaacttatattttgcctgagcttgatcaaaggtcatttgtgtagatgagtaattaaaaaatatttaaatcacattataattctattattggagagcatcttttaaatttttttctgttttaacgagggaaagagaaacctgtatacctagggtcattatttgaccccatagtataaccagattcatggtctaacaagctctcagtgtggcttttctctgaatgcttgaatttcacatgccttgcatttcacagttgtactccatggtcaaccggtgctttttttcacatcgtggtacttgtcaaaacattttgttattttccttggtaaaatatataaaaaaggttttctaatttcaaaaaaaaaaaaaaaaaa

SEQ ID NO: 12 - protein, 979 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFGHKLVSEVIGASDLLLLLGSPEETAFRATDRGSESDKHFRKGLLGELILLQQQIQEHEEEARRAAGQYSTSYAQQKRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 13 - DNA, 6456 bp, Homo sapiens
  source 1..6456: mol_type = other DNA
gtgctgcccgcctagaaagggtgaagtggttgtttccgtgacggactgagtacgggtgcctgtcaggctcttgcggaagtccatgcgccattgggagggcctcggccgcggctctgtgcccttgctgctgagggccacttcctgggtcattcctggaccgggagccgggctggggctcacacgggggctcccgcgtggccgtctcggcgcctgcgtgacctccccgccggcgggatgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttgagaaaattagaaaagcccttcctagttcagaagaccttgtaaagttagcaccagactttgacaagattgttgaaagccttagcttattgaaggacttttttacctcaggttctccggaagaaacggcgtttagagcaacagatcgtggatctgaaagtgacaagcattttagaaagggtctgcttggtgagctcattctcttacaacaacaaattcaagagcatgaagaggaagcgcgcagagccgctggccaatatagcacgagctatgcccaacagaagcgcaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataaattaaaatcgtactcataatcagctctgcatacatctgaagaacaaaaacatcaacgtcttttgtccagcctctttttcttctgctgttccacctttctaaacatacaataaagtcatgggataaaaataatcgatgtatgttacgggcgctttaaccatcagctgcctctcgaatggaagaacagtggtaatggattaacatcctattttgttgtactaaagtgacaaatcggaataatataattggtatggccattaggttcagtccttgaagataagaaacttgttctctgtttgttgtcttatttgtggtggcactcgtttaatggattaactgaggttgctcaatgttcagtttcttttccagaaatacaatgctaggtgttttgaaataaaacttatatagcaattgtttaaagttatcaattgtatataaaatcacagtagcctgctaaatcattgtatgtgtctgtagtattctattcccagaaactatttgaccatgataattcagtttatattcaccacatgaaagaaaaatgggtaacagaagaacccttaaaacaggttaatttggattgtaacgttcagtgaaagaaatttcaacccttcatagccagcgaagaaatttgccttggaagccaagtcagtaccagcttacctatttgattcagttgctgttttctcactctctatatccatttgaaattgatttattttagatgttgtatacttacgttaggctttctgttaatagtggtttttctcctgttgacagagccaccggattatgacacaggatgaggaagattaaggataatcaattgactaatttcatttagaatattatcaaacatttcaactaggtatcagaaaaaggctttctttcataagactattttaaatagaaattatttcaacaattaaagtaatgttgaccatccccctctcagctgaataaagaaaaatttagttcaatttattgcaatttaattacaatactaccttcacaacattttcatgtgttttaaataaatattttttaattggctaaaggacattcaagcaaagaaatgctttctttacttaaaatgtctatctcatttgctgccttttcactaagcctttactttgttaataaaagtgtccattgtgtgatgtttttgattttacagtttgctaaatcttattttcttggagttgctttttggtaacagccccattgctactccccattttattgttttacatcaatgcatgcttcgttgtgatccctcaagatgtaacacttggtatgctcggttgaggatatgaaaaaatacttccgaaaccaggaattcaatgtatgtttgttttatactgtttgataagaaaagtaggtccagccttaagcagcacagatgcgctggtagatgcatagtcaggaactttttttatttcttttaggtctagggacaggagtgaatagaaagggaggagagctctattatgttctatacacagattaggagatgaccttactgggtacacccctctaaccagtgcttacaggttaatgcatgttaatgaatatttttgcagttgtaaagcataacaattacaactacacatctatttctaaagaataaaacaggaccatatttatttacttctgtcaactatagaaagaaagaccttcagctgtatttccacagatttctcccaaggaaaaggctaatattagtcactactgttatcacatccctttgtataagttttaaaaagagatggagggagatcttcatttctttgaggagatcagtattgtaacgtatgtgaatagatgataacaattaatattactaaaagtcccacatgagagtcctgacgccctctccatgccccacagtaatgtggcttctttcatgggtttttttttcttctttttagctgatctcatcctaagcatgctttatttttccttgaaagctaggtatttatcaactgcagatgttattgaaagaaaataaaattcagtctcaagagtaaaccctgtgtcttgtgtctgtagttcaaaagtcagaaatgattctaatttaaacaaaaagatactaaatatacagaagttaaattcgaactagccacagaatcatttgtttttatgtcagaatttgcaaagagtggagtggacaaagctctgtatggaagactgaacaactgtaaatagatgatatccaaacttaatttggctaggacttcaattttaaaaatcagtgtacctaggcagtgcacagcacgaaataagtggcccttgcagcttccccgtttaacccactgtgctatagttgcgggtggaacagtcaacctttctagtagtttatgatattgccctctttgtattcccattttctacagttttttccgcagacttctttctgcaaattattcagcctccaaatgcaaatgaatgatataaaaataagtagggaacatggcagagagtggtgcttcccagcctcacaatgtgggaatttgacataggatgagagtcagagtataggtttaaaagataaaatctttagttaataattttgtatttatttattctagatgtatgtatctgaggaaagaaatctggtatttttgctttccaataaaggggatcaaagtaatggtttttctctcagttctctaagctggtctatgttatagctctagcagtatggaaatgtgctttaaaatatgcttaccttttgaatgatcatggctatatgttgttgagatatttgaaacttaccttgttttcacttgtgcactgtgaatgaactttgtattatttttttaaaaccttcacattacgtgtagatattattgcaacttatattttgcctgagcttgatcaaaggtcatttgtgtagatgagtaattaaaaaatatttaaatcacattataattctattattggagagcatcttttaaatttttttctgttttaacgagggaaagagaaacctgtatacctagggtcattatttgaccccatagtataaccagattcatggtctaacaagctctcagtgtggcttttctctgaatgcttgaatttcacatgccttgcatttcacagttgtactccatggtcaaccggtgctttttttcacatcgtggtacttgtcaaaacattttgttattttccttggtaaaatatataaaaaaggttttctaatttcaaaaaaaaaaaaaaaaaa

SEQ ID NO: 14 - protein, 997 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFEKIRKALPSSEDLVKLAPDFDKIVESLSLLKDFFTSGSPEETAFRATDRGSESDKHFRKGLLGELILLQQQIQEHEEEARRAAGQYSTSYAQQKRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 15 - DNA, 6510 bp, Homo sapiens
  source 1..6510: mol_type = other DNA
gtgctgcccgcctagaaagggtgaagtggttgtttccgtgacggactgagtacgggtgcctgtcaggctcttgcggaagtccatgcgccattgggagggcctcggccgcggctctgtgcccttgctgctgagggccacttcctgggtcattcctggaccgggagccgggctggggctcacacgggggctcccgcgtggccgtctcggcgcctgcgtgacctccccgccggcgggatgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttgagaaaattagaaaagcccttcctagttcagaagaccttgtaaagttagcaccagactttgacaagattgttgaaagccttagcttattgaaggacttttttacctcaggtcacaaattggttagtgaagtcataggagcttctgacctacttctcttgttaggttctccggaagaaacggcgtttagagcaacagatcgtggatctgaaagtgacaagcattttagaaagggtctgcttggtgagctcattctcttacaacaacaaattcaagagcatgaagaggaagcgcgcagagccgctggccaatatagcacgagctatgcccaacagaagcgcaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggctgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataaattaaaatcgtactcataatcagctctgcatacatctgaagaacaaaaacatcaacgtcttttgtccagcctctttttcttctgctgttccacctttctaaacatacaataaagtcatgggataaaaataatcgatgtatgttacgggcgctttaaccatcagctgcctctcgaatggaagaacagtggtaatggattaacatcctattttgttgtactaaagtgacaaatcggaataatataattggtatggccattaggttcagtccttgaagataagaaacttgttctctgtttgttgtcttatttgtggtggcactcgtttaatggattaactgaggttgctcaatgttcagtttcttttccagaaatacaatgctaggtgttttgaaataaaacttatatagcaattgtttaaagttatcaattgtatataaaatcacagtagcctgctaaatcattgtatgtgtctgtagtattctattcccagaaactatttgaccatgataattcagtttatattcaccacatgaaagaaaaatgggtaacagaagaacccttaaaacaggttaatttggattgtaacgttcagtgaaagaaatttcaacccttcatagccagcgaagaaatttgccttggaagccaagtcagtaccagcttacctatttgattcagttgctgttttctcactctctatatccatttgaaattgatttattttagatgttgtatacttacgttaggctttctgttaatagtggtttttctcctgttgacagagccaccggattatgacacaggatgaggaagattaaggataatcaattgactaatttcatttagaatattatcaaacatttcaactaggtatcagaaaaaggctttctttcataagactattttaaatagaaattatttcaacaattaaagtaatgttgaccatccccctctcagctgaataaagaaaaatttagttcaatttattgcaatttaattacaatactaccttcacaacattttcatgtgttttaaataaatattttttaattggctaaaggacattcaagcaaagaaatgctttctttacttaaaatgtctatctcatttgctgccttttcactaagcctttactttgttaataaaagtgtccattgtgtgatgtttttgattttacagtttgctaaatcttattttcttggagttgctttttggtaacagccccattgctactccccattttattgttttacatcaatgcatgcttcgttgtgatccctcaagatgtaacacttggtatgctcggttgaggatatgaaaaaatacttccgaaaccaggaattcaatgtatgtttgttttatactgtttgataagaaaagtaggtccagccttaagcagcacagatgcgctggtagatgcatagtcaggaactttttttatttcttttaggtctagggacaggagtgaatagaaagggaggagagctctattatgttctatacacagattaggagatgaccttactgggtacacccctctaaccagtgcttacaggttaatgcatgttaatgaatatttttgcagttgtaaagcataacaattacaactacacatctatttctaaagaataaaacaggaccatatttatttacttctgtcaactatagaaagaaagaccttcagctgtatttccacagatttctcccaaggaaaaggctaatattagtcactactgttatcacatccctttgtataagttttaaaaagagatggagggagatcttcatttctttgaggagatcagtattgtaacgtatgtgaatagatgataacaattaatattactaaaagtcccacatgagagtcctgacgccctctccatgccccacagtaatgtggcttctttcatgggtttttttttcttctttttagctgatctcatcctaagcatgctttatttttccttgaaagctaggtatttatcaactgcagatgttattgaaagaaaataaaattcagtctcaagagtaaaccctgtgtcttgtgtctgtagttcaaaagtcagaaatgattctaatttaaacaaaaagatactaaatatacagaagttaaattcgaactagccacagaatcatttgtttttatgtcagaatttgcaaagagtggagtggacaaagctctgtatggaagactgaacaactgtaaatagatgatatccaaacttaatttggctaggacttcaattttaaaaatcagtgtacctaggcagtgcacagcacgaaataagtggcccttgcagcttccccgtttaacccactgtgctatagttgcgggtggaacagtcaacctttctagtagtttatgatattgccctctttgtattcccattttctacagttttttccgcagacttctttctgcaaattattcagcctccaaatgcaaatgaatgatataaaaataagtagggaacatggcagagagtggtgcttcccagcctcacaatgtgggaatttgacataggatgagagtcagagtataggtttaaaagataaaatctttagttaataattttgtatttatttattctagatgtatgtatctgaggaaagaaatctggtatttttgctttccaataaaggggatcaaagtaatggtttttctctcagttctctaagctggtctatgttatagctctagcagtatggaaatgtgctttaaaatatgcttaccttttgaatgatcatggctatatgttgttgagatatttgaaacttaccttgttttcacttgtgcactgtgaatgaactttgtattatttttttaaaaccttcacattacgtgtagatattattgcaacttatattttgcctgagcttgatcaaaggtcatttgtgtagatgagtaattaaaaaatatttaaatcacattataattctattattggagagcatcttttaaatttttttctgttttaacgagggaaagagaaacctgtatacctagggtcattatttgaccccatagtataaccagattcatggtctaacaagctctcagtgtggcttttctctgaatgcttgaatttcacatgccttgcatttcacagttgtactccatggtcaaccggtgctttttttcacatcgtggtacttgtcaaaacattttgttattttccttggtaaaatatataaaaaaggttttctaatttcaaaaaaaaaaaaaaaaaa

SEQ ID NO: 16 - protein, 1015 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFEKIRKALPSSEDLVKLAPDFDKIVESLSLLKDFFTSGHKLVSEVIGASDLLLLLGSPEETAFRATDRGSESDKHFRKGLLGELILLQQQIQEHEEEARRAAGQYSTSYAQQKRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 17 - DNA, 1953 bp, Homo sapiens
  source 1..1953: mol_type = other DNA
gcgaagggcgccccggccggtatggcgggcaggaagtgcgggtgcgcgcctgcgcataggtcgggtctgcggtgtcaccgctttcgcttctgcttgagtaatcaagtgaaaaaatgagcttcatctgtggattgcagtctgctgctagaaaccatgttttcttccgatttaattcactgtctaactggagaaaatgtaacacattagcatccacctcacggggctgtcatcaagtacaagttaaccatatagtaaataagtatcagggactgggagtaaatcagtgtgacaggtggagttttctgcctggaaactttcatttttatagtacttttaacaacaaaagaacaggaggcctctcaagtaccaaaagtaaggaaatttggaggattaccagcaaatgtactgtatggaatgatgctttttcaagacagctgctaataaaagaagttacagcagtccctagtctgtcagtattgcatcctctaagccctgcttccataagagctattaggaatttccatacttctccacggtttcaagctgctccggttcctctcttgttgatgattcttaaaccagtacagaagttatttgcaatcattgtaggcaggggcataaggaaatggtggcaggcacttcctcctaacaagaaggaagtagttaaagaaaatataaggaagaataaatggaagctattccttggtttgagtagttttggattgctctttgtggtgttttattttactcacctggaagtaagtccaatcacaggaaggagcaagctactattattggggaaagaacagttcagacttttatcggaactggaatatgaagcatggatggaagaatttaaaaatgatatgctaactgagaaagatgcccgatacctggctgttaaagaagtgctttgtcatctaattgaatgcaataaagatgttccagggatctctcagatcaattgggttattcatgtggttgattccccaattattaatgccttcgtgcttccaaatggacaaatgtttgttttcactggatttttaaatagtgtaaccgatattcatcaactttctttccttctgggccatgaaatagcacatgcagtacttgggcatgctgcagaaaaggctggcatggttcatttgttggatttcctaggtatgattttcctcacaatgatttgggccatttgtcctcgagatagcttggcacttttgtgccagtggatacagtctaaattgcaggagtatatgtttaatagaccatacagcagaaaattggaggccgaagctgacaaaattggactactgcttgctgcaaaggcttgtgcagacataagagccagttcagtgttttggcagcaaatggagttcgttgatagcctgcatggccaacccaagatgccagaatggttatctacacacccttctcatggcaatcgagttgagtacttggatagacttatacctcaggctctcaaaattagagagatgtgtaattgtccaccactgtctaatccagaccctcgattactattcaaactcagcacgaagcattttcttgaagaatcagagaaagaagacctaaatatcacgaagaaacagaaaatggatactcttcctattcaaaaacaggagcaaataccattaacatacatagttgagaaaagaacgggcagttgaattaaaatttatgagacacaagatatatgaagaatgttgcagtccttatcattttatgttactttttaaaaaatgatgtttgaagtgaaaaaaaaaaggatattcagggtcaaatcatgtacattacagatattatctaaattcttctagaatttatttttcatgaaatattgatgtattttaatctatgttaaaatatcttcaatgaggaaaatgtcacagaataaatttatattacacattttaaaaaaaaaaaaaaaaaaaa

SEQ ID NO: 18 - protein, 524 aa, Homo sapiens
MSFICGLQSAARNHVFFRFNSLSNWRKCNTLASTSRGCHQVQVNHIVNKYQGLGVNQCDRWSFLPGNFHFYSTFNNKRTGGLSSTKSKEIWRITSKCTVWNDAFSRQLLIKEVTAVPSLSVLHPLSPASIRAIRNFHTSPRFQAAPVPLLLMILKPVQKLFAIIVGRGIRKWWQALPPNKKEVVKENIRKNKWKLFLGLSSFGLLFVVFYFTHLEVSPITGRSKLLLLGKEQFRLLSELEYEAWMEEFKNDMLTEKDARYLAVKEVLCHLIECNKDVPGISQINWVIHVVDSPIINAFVLPNGQMFVFTGFLNSVTDIHQLSFLLGHEIAHAVLGHAAEKAGMVHLLDFLGMIFLTMIWAICPRDSLALLCQWIQSKLQEYMFNRPYSRKLEAEADKIGLLLAAKACADIRASSVFWQQMEFVDSLHGQPKMPEWLSTHPSHGNRVEYLDRLIPQALKIREMCNCPPLSNPDPRLLFKLSTKHFLEESEKEDLNITKKQKMDTLPIQKQEQIPLTYIVEKRTGS

SEQ ID NO: 19 - DNA, 2952 bp, Homo sapiens
  source 1..2952: mol_type = other DNA
gctctaggtaagcgaaacaagccgggggactgcgagccagggactcgggccgcggggcgggaagaagtggggcagcgcttggccaggccgaaaggactttgggggtgggggctgggagtccgtgtctcgaatgagggaggagaggtggagttgccggggctcaggcccggcctcgagcatgggcggatgagaggagtcgggagccgaggcctagggtccttcgggtgaggggagacggagccagcgaggagatggagcagaagcttgtggaggagattcttcaagcaatcactatgtcaacagacacaggtgtttcccttccttcatatgaggaagatcagggatcaaaactcattcgaaaagctaaagaggcaccattcgtacccgttggaatagcgggttttgcagcaattgttgcatatggattatataaactgaagagcaggggaaatactaaaatgtccattcatctgatccacatgcgtgtggcagcccaaggctttgttgtaggagcaatgactgttggtatgggctattccatgtatcgggaattctgggcaaaacctaagccttagaagaagagatgctgtcttggtcttgttggaggagcttgctttagttagatgtcttattattaaagttacctattattgttggaaataaactaatttgtatgggtttagatggtaacatggcattttgaatattggcttcctttcttgcaggcttgatttgcttggtgaccgaattactagtgactagtttactaactaggtcattcaaggaagtcaagttaacttaaacatgtcacctaaatgcacttgatggtgttgaaatgtccaccttcttaaatttttaagatgaacttagttctaaagaagataacaggccaatcctgaaggtactccctgtttgctgcagaatgtcagatattttggatgttgcataagagtcctatttgccccagttaattcaacttttgtctgcctgttttgtggactggctggctctgttagaactctgtccaaaaagtgcatggaatataacttgtaaagcttcccacaattgacaatatatatgcatgtgtttaaaccaaatccagaaagcttaaacaatagagctgcataatagtatttattaaagaatcacaactgtaaacatgagaataatttaaggattctagtttagttttttgtaattgcaaattatatttttgctgctgatatattagaataatttttaaatgtcatcttgaaatagaaatatgtattttaagcactcacgcaaaggtaaatgaacacgttttaaatgtgtgtgttgctaattttttccataagaattgtaaacattgaactgaacaaattacctataatggatttggttaatgacttatgagcaagctggtttggccagacagtatacccaaacttttatataatatacagaaggctatcacacttgtgaaattctcttgtctaatctgaatttgcattccatggtgttaacatggtatatgtattgttattaaagtaagtgacccatgtcaaatgtcttttatttatttcatgaggaaaagctttctgtagaggaacaaatttgagaacaagtttcaggaaattgtcttttttgttgttgttctctaatcatgttctcctttctgtttcaacgattttaaaaatattttactttatggtatgttttattttttttccttttgtgggtaatttttgttctattgattatccatataaattttttttttttttttttgagacggagttattctctgtcatcttggctggcgtgcagtggcacgatcttggctcactgcaacttccatcccccaggttcaagtgattctcttgcctcagcctcctgagtagctgtgattacaggcatgcaccaacatgcctggctaatttttgtatatttagtagagacgaggtttcaccatgttggccaggctgatcttgaattcctgacctcaggttatccacctgcctcggcctcccaaagtgctaggattacaggcgtgagccaccacgcccagctgattatccatataattataacactcttctatttattttcagtcaccaataattccttttgagcaatatttaagcctagcatatttcttccttccctcctcctctactaaccagttctggtcgatatattattggattttactcttatactgtttgtttgtttgtttgtttgtttattttgagacagagtcttgctctgttgctgaggctggagtacagtggtgtgatctgagctcacggcaccctccacctcctagcttcgtgcgattctgatgcttcaaccttccgagtagctgggattacaggcatgcgccaccatgtcgggctaatttttgtgtttttagtaaaggcgaggtttcaccatgttggatgatcttgaactcctggcttcaagagatctatctgcctcagcctcccaaagtgctgggattacaagcataatccaccacacctgacctacttttgtatgttaaatgtgattatacttctctttgacttgtcagcttagctttagctgatacactctggtgcccaactattattgtatcagtgaacttccactttcttttccttttctctcaatttttgttgtatcattcctaccttgtgaggacatataatatttacattctgttgtcatcctcacatttcttagttccacagtttaaatgtatttgaaactcaaaacattcccattaatctcttggtcagctgaaattaatgatttaatagtttccttaaaaaagactcatggaacaatttccctaaatttttgccatgtcaaatatgtttatctgtagcctttacacagtaaaaacaatttggctagataatacaattctcagttcatatttttctttggaatattaaagtattgctatagagaaaaaaaaaaaaaaaa

SEQ ID NO: 20 - protein, 107 aa, Homo sapiens
MEQKLVEEILQAITMSTDTGVSLPSYEEDQGSKLIRKAKEAPFVPVGIAGFAAIVAYGLYKLKSRGNTKMSIHLIHMRVAAQGFVVGAMTVGMGYSMYREFWAKPKP

SEQ ID NO: 21 - DNA, 2805 bp, Homo sapiens
  source 1..2805: mol_type = other DNA
gattggctgggacggctgtgggtggggagaagccgggaggactgggtgcgcctgcagggatcggaagccggttggggtgtgagaggttttctcgctctagaacctcatagaaattctttctcagggagattcttcaagcaatcactatgtcaacagacacaggtgtttcccttccttcatatgaggaagatcagggatcaaaactcattcgaaaagctaaagaggcaccattcgtacccgttggaatagcgggttttgcagcaattgttgcatatggattatataaactgaagagcaggggaaatactaaaatgtccattcatctgatccacatgcgtgtggcagcccaaggctttgttgtaggagcaatgactgttggtatgggctattccatgtatcgggaattctgggcaaaacctaagccttagaagaagagatgctgtcttggtcttgttggaggagcttgctttagttagatgtcttattattaaagttacctattattgttggaaataaactaatttgtatgggtttagatggtaacatggcattttgaatattggcttcctttcttgcaggcttgatttgcttggtgaccgaattactagtgactagtttactaactaggtcattcaaggaagtcaagttaacttaaacatgtcacctaaatgcacttgatggtgttgaaatgtccaccttcttaaatttttaagatgaacttagttctaaagaagataacaggccaatcctgaaggtactccctgtttgctgcagaatgtcagatattttggatgttgcataagagtcctatttgccccagttaattcaacttttgtctgcctgttttgtggactggctggctctgttagaactctgtccaaaaagtgcatggaatataacttgtaaagcttcccacaattgacaatatatatgcatgtgtttaaaccaaatccagaaagcttaaacaatagagctgcataatagtatttattaaagaatcacaactgtaaacatgagaataatttaaggattctagtttagttttttgtaattgcaaattatatttttgctgctgatatattagaataatttttaaatgtcatcttgaaatagaaatatgtattttaagcactcacgcaaaggtaaatgaacacgttttaaatgtgtgtgttgctaattttttccataagaattgtaaacattgaactgaacaaattacctataatggatttggttaatgacttatgagcaagctggtttggccagacagtatacccaaacttttatataatatacagaaggctatcacacttgtgaaattctcttgtctaatctgaatttgcattccatggtgttaacatggtatatgtattgttattaaagtaagtgacccatgtcaaatgtcttttatttatttcatgaggaaaagctttctgtagaggaacaaatttgagaacaagtttcaggaaattgtcttttttgttgttgttctctaatcatgttctcctttctgtttcaacgattttaaaaatattttactttatggtatgttttattttttttccttttgtgggtaatttttgttctattgattatccatataaattttttttttttttttttgagacggagttattctctgtcatcttggctggcgtgcagtggcacgatcttggctcactgcaacttccatcccccaggttcaagtgattctcttgcctcagcctcctgagtagctgtgattacaggcatgcaccaacatgcctggctaatttttgtatatttagtagagacgaggtttcaccatgttggccaggctgatcttgaattcctgacctcaggttatccacctgcctcggcctcccaaagtgctaggattacaggcgtgagccaccacgcccagctgattatccatataattataacactcttctatttattttcagtcaccaataattccttttgagcaatatttaagcctagcatatttcttccttccctcctcctctactaaccagttctggtcgatatattattggattttactcttatactgtttgtttgtttgtttgtttgtttattttgagacagagtcttgctctgttgctgaggctggagtacagtggtgtgatctgagctcacggcaccctccacctcctagcttcgtgcgattctgatgcttcaaccttccgagtagctgggattacaggcatgcgccaccatgtcgggctaatttttgtgtttttagtaaaggcgaggtttcaccatgttggatgatcttgaactcctggcttcaagagatctatctgcctcagcctcccaaagtgctgggattacaagcataatccaccacacctgacctacttttgtatgttaaatgtgattatacttctctttgacttgtcagcttagctttagctgatacactctggtgcccaactattattgtatcagtgaacttccactttcttttccttttctctcaatttttgttgtatcattcctaccttgtgaggacatataatatttacattctgttgtcatcctcacatttcttagttccacagtttaaatgtatttgaaactcaaaacattcccattaatctcttggtcagctgaaattaatgatttaatagtttccttaaaaaagactcatggaacaatttccctaaatttttgccatgtcaaatatgtttatctgtagcctttacacagtaaaaacaatttggctagataatacaattctcagttcatatttttctttggaatattaaagtattgctatagagaaaaaaaaaaaaaaaa

SEQ ID NO: 22 - protein, 93 aa, Homo sapiens [sequence omitted]

SEQ ID NO: 23 - DNA, 1661 bp, Homo sapiens
  source 1..1661: mol_type = other DNA
aagtcgcggccaatgggcgacgcggccgcagatccgcccggccccgccctgccctgtgagttcctccggccgggctgcggggctccgctcagtccgggagcgcagctgggccgcggcgctccgacctccgctttcccaccgcccgcagctgaagcacatcccgcagcccggcgcggactccgatcgccgcagttgccctctggcgccatgtcgcagaacggagcgcccgggatgcaggaggagagcctgcagggctcctgggtagaactgcacttcagcaataatgggaacgggggcagcgttccagcctcggtttctatttataatggagacatggaaaaaatactgctggacgcacagcatgagtctggacggagtagctccaagagctctcactgtgacagcccacctcgctcgcagacaccacaagataccaacagagcttctgaaacagatacccatagcattggagagaaaaacagctcacagtctgaggaagatgatattgaaagaaggaaagaagttgaaagcatcttgaagaaaaactcagattggatatgggattggtcaagtcggccggaaaatattccccccaaggagttcctctttaaacacccgaagcgcacggccaccctcagcatgaggaacacgagcgtcatgaagaaagggggcatattctctgcagaatttctgaaagttttccttccatctctgctgctctctcatttgctggccatcggattggggatctatattggaaggcgtctgacaacctccaccagcaccttttgatgaagaactggagtctgacttggttcgttagtggattacttctgagcttgcaacatagctcactgaagagctgttagatcctggggtggccacgtcacttgtgtttatttgttctgtaaatgctgcgttcctaatttagtaaaataaaagaatagacactaaaatcatgttgatctataattacacctatgggatcaataagcatgtcagactgattaatgtctactgtgaaaatttggtagtaaattttcatttgatattagatataaatatctgaatataaataattttaatatactagtcatgatgtgtgttgtattttaaaaattatctgcaaccttaattcagctgaagtactttatatttcaaaagaatgaataacattgataataaaatcgctactttaaggggtttgtccaaaataaatattgtggccttatatatcacactattgtagaaagtattatttaatttaaatggatgcaggttgtctactaaagaaagattatatataactatgctaattgttcataatcaacagaaaccaagatagagctacaaactcagctgtacagttcgtacactaaactcttcttgcttttgcattataaggaattaagtctccgattattaggtgatcaccctggatgatcagttttctgctgaaggcacctactcagtatcttttcctctttatcactctgcattggtgaatttaatcctctcctttgtgttcaacttttgtgtgcttttaaaatcagctttattctaagcaaatctgtgtctactttaaaaaactggaaatggaaaaaaaaataaatctttgccaaatccttcagataaaaaaaaaaaaaaaaaaaaaaaaaaaa

SEQ ID NO: 24 - protein, 259 aa, Homo sapiens
MGDAAADPPGPALPCEFLRPGCGAPLSPGAQLGRGAPTSAFPPPAAEAHPAARRGLRSPQLPSGAMSQNGAPGMQEESLQGSWVELHFSNNGNGGSVPASVSIYNGDMEKILLDAQHESGRSSSKSSHCDSPPRSQTPQDTNRASETDTHSIGEKNSSQSEEDDIERRKEVESILKKNSDWIWDWSSRPENIPPKEFLFKHPKRTATLSMRNTSVMKKGGIFSAEFLKVFLPSLLLSHLLAIGLGIYIGRRLTTSTSTF

SEQ ID NO: 25 - DNA, 4403 bp, Homo sapiens
  source 1..4403: mol_type = other DNA
actgttgagttagcgcctcgccttccggggcggattgtctgtcgttgcagtagctgtaggaaggggaggccattttccgtttctgggaggagtgaggggcaacgggtcggagaaaaaggaaaaaagaagggctcagcgcctccccgccgggccgtggacagaggggcacagtttcggcaggcgggtgaggtcgctgagggcccgccggagatgttttccttgtcgagcacggtgcaaccccaggttacagttcctctgagtcatctcatcaatgccttccatacaccaaaaaacacttctgtttctctcagtggagtgtcagtttctcaaaaccagcatcgagatgtagttcctgagcatgaggctcccagcagtgagtgtatgttcagtgacttcctgacgaagcttaacattgtttcaattggcaaaggaaaaatattcgaagggtacagatccatgttcatggagccagcaaaaaggatgaagaagagcttggacacaaccgataactggcacatccgtccagaacccttctccctctcaatccctccttcacttaacttaagggaccttggattatctgaactaaaaattggacagattgatcagctggtagaaaatctacttcctggattttgtaaaggcaaaaacatttcttcccattggcatacatcccatgtctctgcacaatccttctttgaaaataaatatggtaacttagatatatttagtacattacgttcctcttgcttgtatcgacatcattcaagagctcttcaaagcatttgttcagatcttcagtactggccagttttcatacagtctcggggttttaaaactttgaaatcaaggacacgacgtctccagtctacctccgagagattagctgaaacacagaatatagcgccatcattcgtgaaggggtttcttttgcgggacagaggatcagatgttgagagtttggacaaactcatgaaaaccaaaaatatacctgaagctcaccaagatgcatttaaaactggttttgcggaaggttttctgaaagctcaagcactcacacaaaaaaccaatgattccctaaggcgaacccgtctgattctcttcgttctgctgctattcggcatttatggacttctaaaaaacccatttttatctgtccgcttccggacaacaacagggcttgattctgcagtagatcctgtccagatgaaaaatgtcacctttgaacatgttaaaggggtggaggaagctaaacaagaattacaggaagttgttgaattcttgaaaaatccacaaaaatttactattcttggaggtaaacttccaaaaggaattcttttagttggacccccagggactggaaagacacttcttgcccgagctgtggcgggagaagctgatgttcctttttattatgcttctggatccgaatttgatgagatgtttgtgggtgtgggagccagccgtatcagaaatctttttagggaagcaaaggcgaatgctccttgtgttatatttattgatgaattagattctgttggtgggaagagaattgaatctccaatgcatccatattcaaggcagaccataaatcaacttcttgctgaaatggatggttttaaacccaatgaaggagttatcataataggagccacaaacttcccagaggcattagataatgccttaatacgtcctggtcgttttgacatgcaagttacagttccaaggccagatgtaaaaggtcgaacagaaattttgaaatggtatctcaataaaataaagtttgatcaatccgttgatccagaaattatagctcgaggtactgttggcttttccggagcagagttggagaatcttgtgaaccaggctgcattaaaagcagctgttgatggaaaagaaatggttaccatgaaggagctggagttttccaaagacaaaattctaatggggcctgaaagaagaagtgtggaaattgataacaaaaacaaaaccatcacagcatatcatgaatctggtcatgccattattgcatattacacaaaagatgcaatgcctatcaacaaagctacaatcatgccacgggggccaacacttggacatgtgtccctgttacctgagaatgacagatggaatgaaactagagcccagctgcttgcacaaatggatgttagtatgggaggaagagtggcagaggagcttatatttggaaccgaccatattacaacaggtgcttccagtgattttgataatgccactaaaatagcaaagcggatggttaccaaatttggaatgagtgaaaagcttggagttatgacctacagtgatacagggaaactaagtccagaaacccaatctgccatcgaacaagaaataagaatccttctaagggactcatatgaacgagcaaaacatatcttgaaaactcatgcaaaggagcataagaatctcgcagaagctttattgacctatgagactttggatgccaaagagattcaaattgttcttgaggggaaaaagttggaagtgagatgataactctcttgatatggatgcttgctggttttattgcaagaatacaagtagcattgcagtagtctacttttacaacgctttcccctcattcttgatgtggtgtaattgaagggtgtgaaatgctttgtcaatcatttgtcacatttatccagtttgggttattctcattatgacacctattgcaaattagcatcccatggcaaatatattttgaaaaaataaagaactatcaggattgaaaacagctcttttgaggaatgtcaattagttattaagttgaaagtaattaatgattttatgtttggttactctactagatttgataaaaattgtgcctttagccttctatatacatcagtggaaacttaagatgcagtaattatgttccagattgaccatgaataaaatattttttaatctaaatgtagagaagttgggattaaaagcagtctcggaaacacagagccaggaatatagccttttggcatggtgccatggctcacatctgtaatcccagcacttttggaggctgaggcgggtggattgcttgaggccaggagttcgagaccagcctggccaacgtggtgaaacgctgtctctactaaaatacaaaaaaatagggctgggcgcggttgctcacgcctgtaatcccagcacttttcagaggccaaggcgggcaaatcacctgaggtcaagagtttgagaccagcctggccaacatggtgaaaccccatctctactaaacatgcaaaaattacctgggcatggtggcaggtgcttataatcccagctactctgggggccaaggcaggagaattgcttgagcctgggagatggaggttgcagtgagctgagatcatgccactgcactccagcctgggcaacagagcaagactctgcctcaaaaaaaaattaaaataaatttaaatacaaaaaaaaatagccaggtgtggggtgcatgcctggaatcccagctacttgagaggctgaggcacgagaattgcttgaacccaggaggtggaggttgcagtgagccaagatcacaggagccactgcactccagcctgggtgacagagtgagactctgtctcaaaaaaaaattaaataaattattataacctttcagaaatgctgtgtgcattttcatgttcttttttttagcattactgtcactctccctaatgaaatgtacttcagagaagcagtattttgttaaataaatacataacctcattctgaataatgtccctcattttgactataactgtgcttggtttcaaaagcaaaattaaacaaaaatctcagtcccctccgaagtgaactttgtgttaccctgcgtcagaaatgccaagttgtgtttacttttcattcagattttgtgaatatgaacatgctgttataggatctacagatgaatatttaactcaatagaaaaattattttagaacacattgtattggtattacaaccagattatattcttgacgttgacttcattaaaattatctacaatttcctaataatttaagctgtatatggtcttcattgaaaaaagatagatattgttacaggaagcttgttacattatattcttgaccttttggttgataatcttaaatcttaatgtaatttcaaactggcagaaatgttgccagcataatacatggatgtctcatataccctgcatccagatttaccagttgttatcattctgcccgttttttattgccccaaacctgttctgtctccctctctgtatgtacatacatacacgtataaaatattgataaagtcttatctgtcttaaatttttttacatatttgttgaggtataatttacatatgataaaattcattttaaatgtaaaaaa

SEQ ID NO: 26 - protein, 773 aa, Homo sapiens
MFSLSSTVQPQVTVPLSHLINAFHTPKNTSVSLSGVSVSQNQHRDVVPEHEAPSSECMFSDFLTKLNIVSIGKGKIFEGYRSMFMEPAKRMKKSLDTTDNWHIRPEPFSLSIPPSLNLRDLGLSELKIGQIDQLVENLLPGFCKGKNISSHWHTSHVSAQSFFENKYGNLDIFSTLRSSCLYRHHSRALQSICSDLQYWPVFIQSRGFKTLKSRTRRLQSTSERLAETQNIAPSFVKGFLLRDRGSDVESLDKLMKTKNIPEAHQDAFKTGFAEGFLKAQALTQKTNDSLRRTRLILFVLLLFGIYGLLKNPFLSVRFRTTTGLDSAVDPVQMKNVTFEHVKGVEEAKQELQEVVEFLKNPQKFTILGGKLPKGILLVGPPGTGKTLLARAVAGEADVPFYYASGSEFDEMFVGVGASRIRNLFREAKANAPCVIFIDELDSVGGKRIESPMHPYSRQTINQLLAEMDGFKPNEGVIIIGATNFPEALDNALIRPGRFDMQVTVPRPDVKGRTEILKWYLNKIKFDQSVDPEIIARGTVGFSGAELENLVNQAALKAAVDGKEMVTMKELEFSKDKILMGPERRSVEIDNKNKTITAYHESGHAIIAYYTKDAMPINKATIMPRGPTLGHVSLLPENDRWNETRAQLLAQMDVSMGGRVAEELIFGTDHITTGASSDFDNATKIAKRMVTKFGMSEKLGVMTYSDTGKLSPETQSAIEQEIRILLRDSYERAKHILKTHAKEHKNLAEALLTYETLDAKEIQIVLEGKKLEVR

SEQ ID NO: 27 - DNA, 4232 bp, Homo sapiens
  source 1..4232: mol_type = other DNA
actgttgagttagcgcctcgccttccggggcggattgtctgtcgttgcagtagctgtaggaaggggaggccattttccgtttctgggaggagtgaggggcaacgggtcggagaaaaaggaaaaaagaagggctcagcgcctccccgccgggccgtggacagaggggcacagtttcggcaggcgggtgaggtcgctgagggcccgccggagatgttttccttgtcgagcacggtgcaaccccaggttacagttcctctgagtcatctcatcaatgccttccatacaccaaaaaacacttctgtttctctcagtggagtgtcagtttctcaaaaccagcatcgagatgtagttcctgagcatgaggctcccagcagtgagccttcacttaacttaagggaccttggattatctgaactaaaaattggacagattgatcagctggtagaaaatctacttcctggattttgtaaaggcaaaaacatttcttcccattggcatacatcccatgtctctgcacaatccttctttgaaaataaatatggtaacttagatatatttagtacattacgttcctcttgcttgtatcgacatcattcaagagctcttcaaagcatttgttcagatcttcagtactggccagttttcatacagtctcggggttttaaaactttgaaatcaaggacacgacgtctccagtctacctccgagagattagctgaaacacagaatatagcgccatcattcgtgaaggggtttcttttgcgggacagaggatcagatgttgagagtttggacaaactcatgaaaaccaaaaatatacctgaagctcaccaagatgcatttaaaactggttttgcggaaggttttctgaaagctcaagcactcacacaaaaaaccaatgattccctaaggcgaacccgtctgattctcttcgttctgctgctattcggcatttatggacttctaaaaaacccatttttatctgtccgcttccggacaacaacagggcttgattctgcagtagatcctgtccagatgaaaaatgtcacctttgaacatgttaaaggggtggaggaagctaaacaagaattacaggaagttgttgaattcttgaaaaatccacaaaaatttactattcttggaggtaaacttccaaaaggaattcttttagttggacccccagggactggaaagacacttcttgcccgagctgtggcgggagaagctgatgttcctttttattatgcttctggatccgaatttgatgagatgtttgtgggtgtgggagccagccgtatcagaaatctttttagggaagcaaaggcgaatgctccttgtgttatatttattgatgaattagattctgttggtgggaagagaattgaatctccaatgcatccatattcaaggcagaccataaatcaacttcttgctgaaatggatggttttaaacccaatgaaggagttatcataataggagccacaaacttcccagaggcattagataatgccttaatacgtcctggtcgttttgacatgcaagttacagttccaaggccagatgtaaaaggtcgaacagaaattttgaaatggtatctcaataaaataaagtttgatcaatccgttgatccagaaattatagctcgaggtactgttggcttttccggagcagagttggagaatcttgtgaaccaggctgcattaaaagcagctgttgatggaaaagaaatggttaccatgaaggagctggagttttccaaagacaaaattctaatggggcctgaaagaagaagtgtggaaattgataacaaaaacaaaaccatcacagcatatcatgaatctggtcatgccattattgcatattacacaaaagatgcaatgcctatcaacaaagctacaatcatgccacgggggccaacacttggacatgtgtccctgttacctgagaatgacagatggaatgaaactagagcccagctgcttgcacaaatggatgttagtatgggaggaagagtggcagaggagcttatatttggaaccgaccatattacaacaggtgcttccagtgattttgataatgccactaaaatagcaaagcggatggttaccaaatttggaatgagtgaaaagcttggagttatgacctacagtgatacagggaaactaagtccagaaacccaatctgccatcgaacaagaaataagaatccttctaagggactcatatgaacgagcaaaacatatcttgaaaactcatgcaaaggagcataagaatctcgcagaagctttattgacctatgagactttggatgccaaagagattcaaattgttcttgaggggaaaaagttggaagtgagatgataactctcttgatatggatgcttgctggttttattgcaagaatacaagtagcattgcagtagtctacttttacaacgctttcccctcattcttgatgtggtgtaattgaagggtgtgaaatgctttgtcaatcatttgtcacatttatccagtttgggttattctcattatgacacctattgcaaattagcatcccatggcaaatatattttgaaaaaataaagaactatcaggattgaaaacagctcttttgaggaatgtcaattagttattaagttgaaagtaattaatgattttatgtttggttactctactagatttgataaaaattgtgcctttagccttctatatacatcagtggaaacttaagatgcagtaattatgttccagattgaccatgaataaaatattttttaatctaaatgtagagaagttgggattaaaagcagtctcggaaacacagagccaggaatatagccttttggcatggtgccatggctcacatctgtaatcccagcacttttggaggctgaggcgggtggattgcttgaggccaggagttcgagaccagcctggccaacgtggtgaaacgctgtctctactaaaatacaaaaaaatagggctgggcgcggttgctcacgcctgtaatcccagcacttttcagaggccaaggcgggcaaatcacctgaggtcaagagtttgagaccagcctggccaacatggtgaaaccccatctctactaaacatgcaaaaattacctgggcatggtggcaggtgcttataatcccagctactctgggggccaaggcaggagaattgcttgagcctgggagatggaggttgcagtgagctgagatcatgccactgcactccagcctgggcaacagagcaagactctgcctcaaaaaaaaattaaaataaatttaaatacaaaaaaaaatagccaggtgtggggtgcatgcctggaatcccagctacttgagaggctgaggcacgagaattgcttgaacccaggaggtggaggttgcagtgagccaagatcacaggagccactgcactccagcctgggtgacagagtgagactctgtctcaaaaaaaaattaaataaattattataacctttcagaaatgctgtgtgcattttcatgttcttttttttagcattactgtcactctccctaatgaaatgtacttcagagaagcagtattttgttaaataaatacataacctcattctgaataatgtccctcattttgactataactgtgcttggtttcaaaagcaaaattaaacaaaaatctcagtcccctccgaagtgaactttgtgttaccctgcgtcagaaatgccaagttgtgtttacttttcattcagattttgtgaatatgaacatgctgttataggatctacagatgaatatttaactcaatagaaaaattattttagaacacattgtattggtattacaaccagattatattcttgacgttgacttcattaaaattatctacaatttcctaataatttaagctgtatatggtcttcattgaaaaaagatagatattgttacaggaagcttgttacattatattcttgaccttttggttgataatcttaaatcttaatgtaatttcaaactggcagaaatgttgccagcataatacatggatgtctcatataccctgcatccagatttaccagttgttatcattctgcccgttttttattgccccaaacctgttctgtctccctctctgtatgtacatacatacacgtataaaatattgataaagtcttatctgtcttaaatttttttacatatttgttgaggtataatttacatatgataaaattcattttaaatgtaaaaaa

SEQ ID NO: 28 - protein, 716 aa, Homo sapiens
MFSLSSTVQPQVTVPLSHLINAFHTPKNTSVSLSGVSVSQNQHRDVVPEHEAPSSEPSLNLRDLGLSELKIGQIDQLVENLLPGFCKGKNISSHWHTSHVSAQSFFENKYGNLDIFSTLRSSCLYRHHSRALQSICSDLQYWPVFIQSRGFKTLKSRTRRLQSTSERLAETQNIAPSFVKGFLLRDRGSDVESLDKLMKTKNIPEAHQDAFKTGFAEGFLKAQALTQKTNDSLRRTRLILFVLLLFGIYGLLKNPFLSVRFRTTTGLDSAVDPVQMKNVTFEHVKGVEEAKQELQEVVEFLKNPQKFTILGGKLPKGILLVGPPGTGKTLLARAVAGEADVPFYYASGSEFDEMFVGVGASRIRNLFREAKANAPCVIFIDELDSVGGKRIESPMHPYSRQTINQLLAEMDGFKPNEGVIIIGATNFPEALDNALIRPGRFDMQVTVPRPDVKGRTEILKWYLNKIKFDQSVDPEIIARGTVGFSGAELENLVNQAALKAAVDGKEMVTMKELEFSKDKILMGPERRSVEIDNKNKTITAYHESGHAIIAYYTKDAMPINKATIMPRGPTLGHVSLLPENDRWNETRAQLLAQMDVSMGGRVAEELIFGTDHITTGASSDFDNATKIAKRMVTKFGMSEKLGVMTYSDTGKLSPETQSAIEQEIRILLRDSYERAKHILKTHAKEHKNLAEALLTYETLDAKEIQIVLEGKKLEVR

SEQ ID NO: 29 - DNA, 4133 bp, Homo sapiens
  source 1..4133: mol_type = other DNA
actgttgagttagcgcctcgccttccggggcggattgtctgtcgttgcagtagctgtaggaaggggaggccattttccgtttctgggaggagtgaggggcaacgggtcggagaaaaaggaaaaaagaagggctcagcgcctccccgccgggccgtggacagaggggcacagtttcggcaggcgggtgaggtcgctgagggcccgccggagatgttttccttgtcgagcacggtgcaaccccaggttacagttcctctgagtcatctcatcaatgccttccatacaccaaaaaacacttctgtttctctcagtggagtgtcagtttctcaaaaccagcatcgagatgtagttcctgagcatgaggctcccagcagtgagccttcacttaacttaagggaccttggattatctgaactaaaaattggacagattgatcagctggtagaaaatctacttcctggattttgtaaaggcaaaaacatttcttcccattggcatacatcccatgtctctgcacaatccttctttgaaaataaatatgttttcatacagtctcggggttttaaaactttgaaatcaaggacacgacgtctccagtctacctccgagagattagctgaaacacagaatatagcgccatcattcgtgaaggggtttcttttgcgggacagaggatcagatgttgagagtttggacaaactcatgaaaaccaaaaatatacctgaagctcaccaagatgcatttaaaactggttttgcggaaggttttctgaaagctcaagcactcacacaaaaaaccaatgattccctaaggcgaacccgtctgattctcttcgttctgctgctattcggcatttatggacttctaaaaaacccatttttatctgtccgcttccggacaacaacagggcttgattctgcagtagatcctgtccagatgaaaaatgtcacctttgaacatgttaaaggggtggaggaagctaaacaagaattacaggaagttgttgaattcttgaaaaatccacaaaaatttactattcttggaggtaaacttccaaaaggaattcttttagttggacccccagggactggaaagacacttcttgcccgagctgtggcgggagaagctgatgttcctttttattatgcttctggatccgaatttgatgagatgtttgtgggtgtgggagccagccgtatcagaaatctttttagggaagcaaaggcgaatgctccttgtgttatatttattgatgaattagattctgttggtgggaagagaattgaatctccaatgcatccatattcaaggcagaccataaatcaacttcttgctgaaatggatggttttaaacccaatgaaggagttatcataataggagccacaaacttcccagaggcattagataatgccttaatacgtcctggtcgttttgacatgcaagttacagttccaaggccagatgtaaaaggtcgaacagaaattttgaaatggtatctcaataaaataaagtttgatcaatccgttgatccagaaattatagctcgaggtactgttggcttttccggagcagagttggagaatcttgtgaaccaggctgcattaaaagcagctgttgatggaaaagaaatggttaccatgaaggagctggagttttccaaagacaaaattctaatggggcctgaaagaagaagtgtggaaattgataacaaaaacaaaaccatcacagcatatcatgaatctggtcatgccattattgcatattacacaaaagatgcaatgcctatcaacaaagctacaatcatgccacgggggccaacacttggacatgtgtccctgttacctgagaatgacagatggaatgaaactagagcccagctgcttgcacaaatggatgttagtatgggaggaagagtggcagaggagcttatatttggaaccgaccatattacaacaggtgcttccagtgattttgataatgccactaaaatagcaaagcggatggttaccaaatttggaatgagtgaaaagcttggagttatgacctacagtgatacagggaaactaagtccagaaacccaatctgccatcgaacaagaaataagaatccttctaagggactcatatgaacgagcaaaacatatcttgaaaactcatgcaaaggagcataagaatctcgcagaagctttattgacctatgagactttggatgccaaagagattcaaattgttcttgaggggaaaaagttggaagtgagatgataactctcttgatatggatgcttgctggttttattgcaagaatacaagtagcattgcagtagtctacttttacaacgctttcccctcattcttgatgtggtgtaattgaagggtgtgaaatgctttgtcaatcatttgtcacatttatccagtttgggttattctcattatgacacctattgcaaattagcatcccatggcaaatatattttgaaaaaataaagaactatcaggattgaaaacagctcttttgaggaatgtcaattagttattaagttgaaagtaattaatgattttatgtttggttactctactagatttgataaaaattgtgcctttagccttctatatacatcagtggaaacttaagatgcagtaattatgttccagattgaccatgaataaaatattttttaatctaaatgtagagaagttgggattaaaagcagtctcggaaacacagagccaggaatatagccttttggcatggtgccatggctcacatctgtaatcccagcacttttggaggctgaggcgggtggattgcttgaggccaggagttcgagaccagcctggccaacgtggtgaaacgctgtctctactaaaatacaaaaaaatagggctgggcgcggttgctcacgcctgtaatcccagcacttttcagaggccaaggcgggcaaatcacctgaggtcaagagtttgagaccagcctggccaacatggtgaaaccccatctctactaaacatgcaaaaattacctgggcatggtggcaggtgcttataatcccagctactctgggggccaaggcaggagaattgcttgagcctgggagatggaggttgcagtgagctgagatcatgccactgcactccagcctgggcaacagagcaagactctgcctcaaaaaaaaattaaaataaatttaaatacaaaaaaaaatagccaggtgtggggtgcatgcctggaatcccagctacttgagaggctgaggcacgagaattgcttgaacccaggaggtggaggttgcagtgagccaagatcacaggagccactgcactccagcctgggtgacagagtgagactctgtctcaaaaaaaaattaaataaattattataacctttcagaaatgctgtgtgcattttcatgttcttttttttagcattactgtcactctccctaatgaaatgtacttcagagaagcagtattttgttaaataaatacataacctcattctgaataatgtccctcattttgactataactgtgcttggtttcaaaagcaaaattaaacaaaaatctcagtcccctccgaagtgaactttgtgttaccctgcgtcagaaatgccaagttgtgtttacttttcattcagattttgtgaatatgaacatgctgttataggatctacagatgaatatttaactcaatagaaaaattattttagaacacattgtattggtattacaaccagattatattcttgacgttgacttcattaaaattatctacaatttcctaataatttaagctgtatatggtcttcattgaaaaaagatagatattgttacaggaagcttgttacattatattcttgaccttttggttgataatcttaaatcttaatgtaatttcaaactggcagaaatgttgccagcataatacatggatgtctcatataccctgcatccagatttaccagttgttatcattctgcccgttttttattgccccaaacctgttctgtctccctctctgtatgtacatacatacacgtataaaatattgataaagtcttatctgtcttaaatttttttacatatttgttgaggtataatttacatatgataaaattcattttaaatgtaaaaaa

SEQ ID NO: 30 - protein, 683 aa, Homo sapiens
MFSLSSTVQPQVTVPLSHLINAFHTPKNTSVSLSGVSVSQNQHRDVVPEHEAPSSEPSLNLRDLGLSELKIGQIDQLVENLLPGFCKGKNISSHWHTSHVSAQSFFENKYVFIQSRGFKTLKSRTRRLQSTSERLAETQNIAPSFVKGFLLRDRGSDVESLDKLMKTKNIPEAHQDAFKTGFAEGFLKAQALTQKTNDSLRRTRLILFVLLLFGIYGLLKNPFLSVRFRTTTGLDSAVDPVQMKNVTFEHVKGVEEAKQELQEVVEFLKNPQKFTILGGKLPKGILLVGPPGTGKTLLARAVAGEADVPFYYASGSEFDEMFVGVGASRIRNLFREAKANAPCVIFIDELDSVGGKRIESPMHPYSRQTINQLLAEMDGFKPNEGVIIIGATNFPEALDNALIRPGRFDMQVTVPRPDVKGRTEILKWYLNKIKFDQSVDPEIIARGTVGFSGAELENLVNQAALKAAVDGKEMVTMKELEFSKDKILMGPERRSVEIDNKNKTITAYHESGHAIIAYYTKDAMPINKATIMPRGPTLGHVSLLPENDRWNETRAQLLAQMDVSMGGRVAEELIFGTDHITTGASSDFDNATKIAKRMVTKFGMSEKLGVMTYSDTGKLSPETQSAIEQEIRILLRDSYERAKHILKTHAKEHKNLAEALLTYETLDAKEIQIVLEGKKLEVR

SEQ ID NO: 31 - DNA, 1919 bp, Homo sapiens
  source 1..1919: mol_type = other DNA
atgtggaggtcagagtggaagcaggtgagaatggagggggcggcaaaggctcgtttctgggcatctctgcagtcctcctctgctccatgatgtgcactttgggcgaggagagtgcgtgcgtgtgagagggtccagcagaaggaaacatggctgccaaagtgtttgagtccattggcaagtttggcctggccttagctgttgcaggaggcgtggtgaactctgccttatataatgtggatgctgggcacagagctgtcatctttgaccgattccgtggagtgcaggacattgtggtaggggaagggactcattttctcatcccgtgggtacagaaaccaattatctttgactgccgttctcgaccacgtaatgtgccagtcatcactggtagcaaagatttacagaatgtcaacatcacactgcgcatcctcttccggcctgtcgccagccagcttcctcgcatcttcaccagcatcggagaggactatgatgagcgtgtgctgccgtccatcacaactgagatcctcaagtcagtggtggctcgctttgatgctggagaactaatcacccagagagagctggtctccaggcaggtgagcgacgaccttacagagcgagccgccacctttgggctcatcctggatgacgtgtccttgacacatctgaccttcgggaaggagttcacagaagcggtggaagccaaacaggtggctcagcaggaagcagagagggccagatttgtggtggaaaaggctgagcaacagaaaaaggcggccatcatctctgctgagggcgactccaaggcagctgagctgattgccaactcactggccactgcaggggatggcctgatcgagctgcgcaagctggaagctgcagaggacatcgcgtaccagctctcacgctctcggaacatcacctacctgccagcggggcagtccgtgctcctccagctgccccagtgagggcccaccctgcctgcacctccgcgggctgactgggccacagccccgatgattcttaacacagccttccttctgctcccaccccagaaatcactgtgaaatttcatgattggcttaaagtgaaggaaataaaggtaaaatcacttcagatctctaattagtctatcaaatgaaactctttcattcttctcacatccatctacttttttatccacctccctaccaaaaattgccaagtgcctatgcaaaccagctttaggtcccaattcggggcctgctggagttccggcctgggcaccagcatttggcagcacgcaggcggggcagtatgtgatggactggggagcacaggtgtctgcctagatccacgtgtggcctccgtcctgtcactgatggaaggtttgcggatgagggcatgtgcggctgaactgagaaggcaggcctccgtcttcccagcggttcctgtgcagatgctgctgaagagaggtgccggggaggggcagagaggaagtggtctgtctgttaccataagtctgattctctttaactgtgtgaccagcggaaacaggtgtgtgtgaactgggcacagattgaagaatctgcccctgttgaggtgggtgggcctgactgttgccccccagggtcctaaaacttggatggacttgtatagtgagagaggaggcctggaccgagatgtgagtcctgttgaagacttcctctctaccccccaccttggtccctctcagatacccagtggaattccaacttgaaggattgcatcctgctggggctgaacatgcctgccaaagacgtgtccgacctacgttcctggccccctcgttcagagactgcccttctcacgggctctatgcctgcactgggaaggaaacaaatgtgtataaactgctgtcaataaatgacacccagaccttccggctcagccaaaaaaaaaaa

SEQ ID NO: 32 - protein, 272 aa, Homo sapiens
MAAKVFESIGKFGLALAVAGGVVNSALYNVDAGHRAVIFDRFRGVQDIVVGEGTHFLIPWVQKPIIFDCRSRPRNVPVITGSKDLQNVNITLRILFRPVASQLPRIFTSIGEDYDERVLPSITTEILKSVVARFDAGELITQRELVSRQVSDDLTERAATFGLILDDVSLTHLTFGKEFTEAVEAKQVAQQEAERARFVVEKAEQQKKAAIISAEGDSKAAELIANSLATAGDGLIELRKLEAAEDIAYQLSRSRNITYLPAGQSVLLQLPQ

SEQ ID NO: 33 - DNA, 1520 bp, Homo sapiens
  source 1..1520: mol_type = other DNA
gggcggggcagaggagctcatgcgcagtatgtgtggttggggaattcatgtggaggtcagagtggaagcaggtgtgagagggtccagcagaaggaaacatggctgccaaagtgtttgagtccattggcaagtttggcctggccttagctgttgcaggaggcgtggtgaactctgccttatataatgtggatgctgggcacagagctgtcatctttgaccgattccgtggagtgcaggacattgtggtaggggaagggactcattttctcatcccgtgggtacagaaaccaattatctttgactgccgttctcgaccacgtaatgtgccagtcatcactggtagcaaagatttacagaatgtcaacatcacactgcgcatcctcttccggcctgtcgccagccagcttcctcgcatcttcaccagcatcggagaggactatgatgagcgtgtgctgccgtccatcacaactgagatcctcaagtcagtggtggctcgctttgatgctggagaactaatcacctacctgccagcggggcagtccgtgctcctccagctgccccagtgagggcccaccctgcctgcacctccgcgggctgactgggccacagccccgatgattcttaacacagccttccttctgctcccaccccagaaatcactgtgaaatttcatgattggcttaaagtgaaggaaataaaggtaaaatcacttcagatctctaattagtctatcaaatgaaactctttcattcttctcacatccatctacttttttatccacctccctaccaaaaattgccaagtgcctatgcaaaccagctttaggtcccaattcggggcctgctggagttccggcctgggcaccagcatttggcagcacgcaggcggggcagtatgtgatggactggggagcacaggtgtctgcctagatccacgtgtggcctccgtcctgtcactgatggaaggtttgcggatgagggcatgtgcggctgaactgagaaggcaggcctccgtcttcccagcggttcctgtgcagatgctgctgaagagaggtgccggggaggggcagagaggaagtggtctgtctgttaccataagtctgattctctttaactgtgtgaccagcggaaacaggtgtgtgtgaactgggcacagattgaagaatctgcccctgttgaggtgggtgggcctgactgttgccccccagggtcctaaaacttggatggacttgtatagtgagagaggaggcctggaccgagatgtgagtcctgttgaagacttcctctctaccccccaccttggtccctctcagatacccagtggaattccaacttgaaggattgcatcctgctggggctgaacatgcctgccaaagacgtgtccgacctacgttcctggccccctcgttcagagactgcccttctcacgggctctatgcctgcactgggaaggaaacaaatgtgtataaactgctgtcaataaatgacacccagaccttccggctcagccaaaaaaaaaaa

SEQ ID NO: 34 - protein, 155 aa, Homo sapiens
MAAKVFESIGKFGLALAVAGGVVNSALYNVDAGHRAVIFDRFRGVQDIVVGEGTHFLIPWVQKPIIFDCRSRPRNVPVITGSKDLQNVNITLRILFRPVASQLPRIFTSIGEDYDERVLPSITTEILKSVVARFDAGELITYLPAGQSVLLQLPQ

SEQ ID NO: 35 - DNA, 1457 bp, Homo sapiens
  source 1..1457: mol_type = other DNA
tccgtatgcgcgattcctgtgcgcgaagttcgggtccgtagtgggctaagggggagggtttcaaagggagcgcacttccgctgccctttctttcgccagccttacgggcccgaaccctcgtgtgaagggtgcagtacctaagccggagcggggtagaggcgggccggcacccccttctgacctccagtgccgccggcctcaagatcagacatggcccagaacttgaaggacttggcgggacggctgcccgccgggccccggggcatgggcacggccctgaagctgttgctgggggccggcgccgtggcctacggtgtgcgcgaatctgtgttcaccgtggaaggcgggcacagagccatcttcttcaatcggatcggtggagtgcagcaggacactatcctggccgagggccttcacttcaggatcccttggttccagtaccccattatctatgacattcgggccagacctcgaaaaatctcctcccctacaggctccaaagacctacagatggtgaatatctccctgcgagtgttgtctcgacccaatgctcaggagcttcctagcatgtaccagcgcctagggctggactacgaggaacgagtgttgccgtccattgtcaacgaggtgctcaagagtgtggtggccaagttcaatgcctcacagctgatcacccagcgggcccaggtatccctgttgatccgccgggagctgacagagagggccaaggacttcagcctcatcctggatgatgtggccatcacagagctgagctttagccgagagtacacagctgctgtagaagccaaacaagtggcccagcaggaggcccagcgggcccaattcttggtagaaaaagcaaagcaggaacagcggcagaaaattgtgcaggccgagggtgaggccgaggctgccaagatgcttggagaagcactgagcaagaaccctggctacatcaaacttcgcaagattcgagcagcccagaatatctccaagacgatcgccacatcacagaatcgtatctatctcacagctgacaaccttgtgctgaacctacaggatgaaagtttcaccaggggaagtgacagcctcatcaagggtaagaaatgagcctagtcaccaagaactccacccccagaggaagtggatctgcttctccagtttttgaggagccagccaggggtccagcacagccctaccccgccccagtatcatgcgatggtcccccacaccggttccctgaacccctcttggattaaggaagactgaagactagccccttttctggggaattactttcctcctccctgtgttaactggggctgttggggacagtgcgtgatttctcagtgatttcctacagtgttgttccctccctcaaggctgggaggagataaacaccaacccaggaattctcaataaatttttattacttaacctgaaaaaaaaaaaaaaaa

SEQ ID NO: 36 - protein, 299 aa, Homo sapiens
MAQNLKDLAGRLPAGPRGMGTALKLLLGAGAVAYGVRESVFTVEGGHRAIFFNRIGGVQQDTILAEGLHFRIPWFQYPIIYDIRARPRKISSPTGSKDLQMVNISLRVLSRPNAQELPSMYQRLGLDYEERVLPSIVNEVLKSVVAKFNASQLITQRAQVSLLIRRELTERAKDFSLILDDVAITELSFSREYTAAVEAKQVAQQEAQRAQFLVEKAKQEQRQKIVQAEGEAEAAKMLGEALSKNPGYIKLRKIRAAQNISKTIATSQNRIYLTADNLVLNLQDESFTRGSDSLIKGKK

SEQ ID NO: 37 - DNA, 1343 bp, Homo sapiens
  source 1..1343: mol_type = other DNA
tccgtatgcgcgattcctgtgcgcgaagttcgggtccgtagtgggctaagggggagggtttcaaagggagcgcacttccgctgccctttctttcgccagccttacgggcccgaaccctcgtgtgaagggtgcagtacctaagccggagcggggtagaggcgggccggcacccccttctgacctccagtgccgccggcctcaagatcagacatggcccagaacttgaaggacttggcgggacggctgcccgccgggccccggggcatgggcacggccctgaagctgttgctgggggccggcgccgtggcctacggtgtgcgcgaatctgtgttcaccgtggaaggcgggcacagagccatcttcttcaatcggatcggtggagtgcagcaggacactatcctggccgagggccttcacttcaggatcccttggttccagtaccccattatctatgacattcgggccagacctcgaaaaatctcctcccctacaggctccaaagacctacagatggtgaatatctccctgcgagtgttgtctcgacccaatgctcaggagcttcctagcatgtaccagcgcctagggctggactacgaggaacgagtgttgccgtccattgtcaacgaggtgctcaagagtgtggtggccaagttcaatgcctcacagctgatcacccagcgggcccaggtatccctgttgatccgccgggagctgacagagagggccaaggacttcagcctcatcctggatgatgtggccatcacagagctgagctttagccgagagtacacagctgctgtagaagccaaacaagtggcactgagcaagaaccctggctacatcaaacttcgcaagattcgagcagcccagaatatctccaagacgatcgccacatcacagaatcgtatctatctcacagctgacaaccttgtgctgaacctacaggatgaaagtttcaccaggggaagtgacagcctcatcaagggtaagaaatgagcctagtcaccaagaactccacccccagaggaagtggatctgcttctccagtttttgaggagccagccaggggtccagcacagccctaccccgccccagtatcatgcgatggtcccccacaccggttccctgaacccctcttggattaaggaagactgaagactagccccttttctggggaattactttcctcctccctgtgttaactggggctgttggggacagtgcgtgatttctcagtgatttcctacagtgttgttccctccctcaaggctgggaggagataaacaccaacccaggaattctcaataaatttttattacttaacctgaaaaaaaaaaaaaaaa

SEQ ID NO: 38 - protein, 261 aa, Homo sapiens
MAQNLKDLAGRLPAGPRGMGTALKLLLGAGAVAYGVRESVFTVEGGHRAIFFNRIGGVQQDTILAEGLHFRIPWFQYPIIYDIRARPRKISSPTGSKDLQMVNISLRVLSRPNAQELPSMYQRLGLDYEERVLPSIVNEVLKSVVAKFNASQLITQRAQVSLLIRRELTERAKDFSLILDDVAITELSFSREYTAAVEAKQVALSKNPGYIKLRKIRAAQNISKTIATSQNRIYLTADNLVLNLQDESFTRGSDSLIKGKK

SEQ ID NO: 39 - DNA, 1773 bp, Homo sapiens
  source 1..1773: mol_type = other DNA
aatgagcgaaggcatcgcgagccagggggcgcggagaaggcggggaatcatggccgcccccagtgttccgcgtccgggggtttgtgggagttgccttgacctgcagctccgccaccgcggacccgccttctgccctcagcagcagacgctctgtcccgcccgggcagctctgcgaggcagcggctggagagggaaccatggggactgtgcacgcccggagtttggagcctcttccatcaagtggacctgattttggaggattaggagaagaagctgaatttgttgaagttgagcctgaagctaaacaggaaattcttgaaaacaaagatgtggttgttcaacatgttcattttgatggacttggaaggactaaagatgatatcatcatttgtgaaattggagatgttttcaaggccaaaaacctaattgaggtaatgcggaaatctcatgaagcccgtgaaaaattgctccgtcttggaatttttagacaagtggatgttttgattgacacatgtcaaggtgatgacgcacttccaaatgggttagacgttacctttgaagtaactgaattgaggagattaacgggcagttataacaccatggttggaaacaatgaaggcagtatggtacttggcctcaagcttcctaatcttcttggtcgtgcagaaaaggtgacctttcagttttcctatggaacaaaagaaacttcgtatggcctgtccttcttcaaaccacggcccggaaacttcgaaagaaatttctctgtaaacttatataaagttactggacagttcccttggagctcactgcgggagacggacagaggaatgtcagctgagtacagttttcccatatggaagaccagccacactgtcaagtgggaaggcgtatggcgagaactgggctgcctctcaaggacggcgtcatttgctgttcgaaaagaaagcggacattcactgaaatcatctctttcgcacgccatggtcatcgattctcggaattcttccatcttaccaaggagaggtgctttgctgaaagttaaccaggaactggcaggctacactggcggggatgtgagcttcatcaaagaagattttgaacttcagttgaacaagcaactcatatttgattcagttttttcagcgtctttctggggcggaatgttggtacccattggtgataagccgtcaagcattgctgataggttttaccttgggggacccacaagcatccgcggattcagcatgcacagcatcgggccacagagcgaaggagactacctaggtggagaagcgtactgggccggcggcctgcacctctacaccccattacctttccggccaggccagggtggctttggagaacttttccgaacacacttctttctcaacgcaggaaacctctgcaacctcaactatggggagggccccaaagctcatattcgtaagctggctgagtgcatccgctggtcgtacggggccgggattgtcctcaggcttggcaacatcgctcggttggaacttaattactgcgtccccatgggagtacagacaggcgacaggatatgtgatggcgtccagtttggagctgggataaggttcctgtagccgacacccctacaggagaagctctgggactggggcagcagcaaggcgcccatgccacacaccgtctctcgaggaaacgcggttcagcgattctttgactgcggaccctgtgggaaaccccgtcaataaatgttaaagacacactccgaaaaaaaaaaaaaaaaaa

SEQ ID NO: 40 - protein, 469 aa, Homo sapiens
MGTVHARSLEPLPSSGPDFGGLGEEAEFVEVEPEAKQEILENKDVVVQHVHFDGLGRTKDDIIICEIGDVFKAKNLIEVMRKSHEAREKLLRLGIFRQVDVLIDTCQGDDALPNGLDVTFEVTELRRLTGSYNTMVGNNEGSMVLGLKLPNLLGRAEKVTFQFSYGTKETSYGLSFFKPRPGNFERNFSVNLYKVTGQFPWSSLRETDRGMSAEYSFPIWKTSHTVKWEGVWRELGCLSRTASFAVRKESGHSLKSSLSHAMVIDSRNSSILPRRGALLKVNQELAGYTGGDVSFIKEDFELQLNKQLIFDSVFSASFWGGMLVPIGDKPSSIADRFYLGGPTSIRGFSMHSIGPQSEGDYLGGEAYWAGGLHLYTPLPFRPGQGGFGELFRTHFFLNAGNLCNLNYGEGPKAHIRKLAECIRWSYGAGIVLRLGNIARLELNYCVPMGVQTGDRICDGVQFGAGIRFL

SEQ ID NO: 41 - DNA, 3018 bp, Homo sapiens
  source 1..3018: mol_type = other DNA
aattctctttaagagtcacagctgtccattttccacgtgcggctgaagaatggatttcagagcgctctccctccggcagtgttcacctagtaaccccttccggataaggacctgcagcccgggcgcgacccggaaagggaatccgcccttttcgcctccttcgcgccaatcgcctggagttggcgtttccgcagccgggacacagcccaccctctaagagccgcggagtcggggacggtagaaggggccgcgcgtgcgcagtggcgtccgctgtgcttccggtgcgccgggcgcggacgcgggcacgcacacacgcaagcacgcctccacttaactcgcgccgccgcggcagctcgagtccaccagcagcgccgtccgcttgaccgagatgctgcgggcctgtcagttatcgggtgtgaccgccgccgcccagagttgtctctgtgggaagtttgtcctccgtccattgcgaccatgccgcagatactctacttcaggcagctctgggttgactactggcaaaattgctggagctggccttttgtttgttggtggaggtattggtggcactatcctatatgccaaatgggattcccatttccgggaaagtgtagagaaaaccataccttactcagacaaactcttcgagatggttcttggtcctgcagcttataatgttccattgccaaagaaatcgattcagtcgggtccactaaaaatctctagtgtatcagaagtaatgaaagaatctaaacagcctgcctcacaactccaaaaacaaaagggagatactccagcttcagcaacagcacctacagaagcggctcaaattatttctgcagcaggtgataccctgtcggtcccagcccctgcagttcagcctgaggaatctttaaaaactgatcaccctgaaattggtgaaggaaaacccacacctgcactttcagaagaagcatcctcatcttctataagggagcgaccacctgaagaagttgcagctcgccttgcacaacaggaaaaacaagaacaagttaaaattgagtctctagccaagagcttagaagatgctctgaggcaaactgcaagtgtcactctgcaggctattgcagctcagaatgctgcggtccaggctgtcaatgcacactccaacatattgaaagccgccatggacaattctgagattgcaggcgagaagaaatctgctcagtggcgcacagtggagggtgcattgaaggaacgcagaaaggcagtagatgaagctgccgatgcccttctcaaagccaaagaagagttagagaagatgaaaagtgtgattgaaaatgcaaagaaaaaagaggttgctggggccaagcctcatataactgctgcagagggtaaacttcacaacatgatagttgatctggataatgtggtcaaaaaggtccaagcagctcagtctgaggctaaggttgtatctcagtatcatgagctggtggtccaagctcgggatgactttaaacgagagctggacagtattactccagaagtccttcctgggtggaaaggaatgagtgtttcagacttagctgacaagctctctactgatgatctgaactccctcattgctcatgcacatcgtcgtattgatcagctgaacagagagctggcagaacagaaggccaccgaaaagcagcacatcacgttagccttggagaaacaaaagctggaagaaaagcgggcatttgactctgcagtagcaaaagcattagaacatcacagaagtgaaatacaggctgaacaggacagaaagatagaagaagtcagagatgccatggaaaatgaaatgagaacccagcttcgccgacaggcagctgcccacactgatcacttgcgagatgtccttagggtacaagaacaggaattgaagtctgaatttgagcagaacctgtctgagaaactctctgaacaagaattacaatttcgtcgtctcagtcaagagcaagttgacaactttactctggatataaatactgcctatgccagactcagaggaatcgaacaggctgttcagagccatgcagttgctgaagaggaagccagaaaagcccaccaactctggctttcagtggaggcattaaagtacagcatgaagacctcatctgcagaaacacctactatcccgctgggtagtgcagttgaggccatcaaagccaactgttctgataatgaattcacccaagctttaaccgcagctatccctccagagtccctgacccgtggggtgtacagtgaagagacccttagagcccgtttctatgctgttcaaaaactggcccgaagggtagcaatgattgatgaaaccagaaatagcttgtaccagtacttcctctcctacctacagtccctgctcctattcccacctcagcaactgaagccgcccccagagctctgccctgaggatataaacacatttaaattactgtcatatgcttcctattgcattgagcatggtgatctggagctagcagcaaagtttgtcaatcagctgaagggggaatccagacgagtggcacaggactggctgaaggaagcccgaatgaccctagaaacgaaacagatagtggaaatcctgacagcatatgccagcgccgtaggaataggaaccactcaggtgcagccagagtgaggtttaggaagattttcataaagtcatatttcatgtcaaaggaaatcagcagtgatagatgaagggttcgcagcgagagtcccggacttgtctagaaatgagcaggtttacaagtactgttctaaatgttaacacctgttgcatttatattctttccatttgctatcatgtcagtgaacgccaggagtgctttctttgcaacttgtgtaacattttctgttttttcaggttttactgatgaggcttgtgaggccaatcaaaataatgtttgtgatctctactactgttgattttgccctcggagcaaactgaataaagcaacaagatgaaaactgaaaaaaaaaaaaaaaaaa

SEQ ID NO: 42 - protein, 758 aa, Homo sapiens
MLRACQLSGVTAAAQSCLCGKFVLRPLRPCRRYSTSGSSGLTTGKIAGAGLLFVGGGIGGTILYAKWDSHFRESVEKTIPYSDKLFEMVLGPAAYNVPLPKKSIQSGPLKISSVSEVMKESKQPASQLQKQKGDTPASATAPTEAAQIISAAGDTLSVPAPAVQPEESLKTDHPEIGEGKPTPALSEEASSSSIRERPPEEVAARLAQQEKQEQVKIESLAKSLEDALRQTASVTLQAIAAQNAAVQAVNAHSNILKAAMDNSEIAGEKKSAQWRTVEGALKERRKAVDEAADALLKAKEELEKMKSVIENAKKKEVAGAKPHITAAEGKLHNMIVDLDNVVKKVQAAQSEAKVVSQYHELVVQARDDFKRELDSITPEVLPGWKGMSVSDLADKLSTDDLNSLIAHAHRRIDQLNRELAEQKATEKQHITLALEKQKLEEKRAFDSAVAKALEHHRSEIQAEQDRKIEEVRDAMENEMRTQLRRQAAAHTDHLRDVLRVQEQELKSEFEQNLSEKLSEQELQFRRLSQEQVDNFTLDINTAYARLRGIEQAVQSHAVAEEEARKAHQLWLSVEALKYSMKTSSAETPTIPLGSAVEAIKANCSDNEFTQALTAAIPPESLTRGVYSEETLRARFYAVQKLARRVAMIDETRNSLYQYFLSYLQSLLLFPPQQLKPPPELCPEDINTFKLLSYASYCIEHGDLELAAKFVNQLKGESRRVAQDWLKEARMTLETKQIVEILTAYASAVGIGTTQVQPE

SEQ ID NO: 43 - DNA, 3015 bp, Homo sapiens
  source 1..3015: mol_type = other DNA
aattctctttaagagtcacagctgtccattttccacgtgcggctgaagaatggatttcagagcgctctccctccggcagtgttcacctagtaaccccttccggataaggacctgcagcccgggcgcgacccggaaagggaatccgcccttttcgcctccttcgcgccaatcgcctggagttggcgtttccgcagccgggacacagcccaccctctaagagccgcggagtcggggacggtagaaggggccgcgcgtgcgcagtggcgtccgctgtgcttccggtgcgccgggcgcggacgcgggcacgcacacacgcaagcacgcctccacttaactcgcgccgccgcggcagctcgagtccaccagcagcgccgtccgcttgaccgagatgctgcgggcctgtcagttatcgggtgtgaccgccgccgcccagagttgtctctgtgggaagtttgtcctccgtccattgcgaccatgccgcagatactctacttcaggcagctctgggttgactactggcaaaattgctggagctggccttttgtttgttggtggaggtattggtggcactatcctatatgccaaatgggattcccatttccgggaaagtgtagagaaaaccataccttactcagacaaactcttcgagatggttcttggtcctgcagcttataatgttccattgccaaagaaatcgattcagtcgggtccactaaaaatctctagtgtatcagaagtaatgaaagaatctaaacagcctgcctcacaactccaaaaacaaaagggagatactccagcttcagcaacagcacctacagaagcggctcaaattatttctgcagcaggtgataccctgtcggtcccagcccctgcagttcagcctgaggaatctttaaaaactgatcaccctgaaattggtgaaggaaaacccacacctgcactttcagaagcatcctcatcttctataagggagcgaccacctgaagaagttgcagctcgccttgcacaacaggaaaaacaagaacaagttaaaattgagtctctagccaagagcttagaagatgctctgaggcaaactgcaagtgtcactctgcaggctattgcagctcagaatgctgcggtccaggctgtcaatgcacactccaacatattgaaagccgccatggacaattctgagattgcaggcgagaagaaatctgctcagtggcgcacagtggagggtgcattgaaggaacgcagaaaggcagtagatgaagctgccgatgcccttctcaaagccaaagaagagttagagaagatgaaaagtgtgattgaaaatgcaaagaaaaaagaggttgctggggccaagcctcatataactgctgcagagggtaaacttcacaacatgatagttgatctggataatgtggtcaaaaaggtccaagcagctcagtctgaggctaaggttgtatctcagtatcatgagctggtggtccaagctcgggatgactttaaacgagagctggacagtattactccagaagtccttcctgggtggaaaggaatgagtgtttcagacttagctgacaagctctctactgatgatctgaactccctcattgctcatgcacatcgtcgtattgatcagctgaacagagagctggcagaacagaaggccaccgaaaagcagcacatcacgttagccttggagaaacaaaagctggaagaaaagcgggcatttgactctgcagtagcaaaagcattagaacatcacagaagtgaaatacaggctgaacaggacagaaagatagaagaagtcagagatgccatggaaaatgaaatgagaacccagcttcgccgacaggcagctgcccacactgatcacttgcgagatgtccttagggtacaagaacaggaattgaagtctgaatttgagcagaacctgtctgagaaactctctgaacaagaattacaatttcgtcgtctcagtcaagagcaagttgacaactttactctggatataaatactgcctatgccagactcagaggaatcgaacaggctgttcagagccatgcagttgctgaagaggaagccagaaaagcccaccaactctggctttcagtggaggcattaaagtacagcatgaagacctcatctgcagaaacacctactatcccgctgggtagtgcagttgaggccatcaaagccaactgttctgataatgaattcacccaagctttaaccgcagctatccctccagagtccctgacccgtggggtgtacagtgaagagacccttagagcccgtttctatgctgttcaaaaactggcccgaagggtagcaatgattgatgaaaccagaaatagcttgtaccagtacttcctctcctacctacagtccctgctcctattcccacctcagcaactgaagccgcccccagagctctgccctgaggatataaacacatttaaattactgtcatatgcttcctattgcattgagcatggtgatctggagctagcagcaaagtttgtcaatcagctgaagggggaatccagacgagtggcacaggactggctgaaggaagcccgaatgaccctagaaacgaaacagatagtggaaatcctgacagcatatgccagcgccgtaggaataggaaccactcaggtgcagccagagtgaggtttaggaagattttcataaagtcatatttcatgtcaaaggaaatcagcagtgatagatgaagggttcgcagcgagagtcccggacttgtctagaaatgagcaggtttacaagtactgttctaaatgttaacacctgttgcatttatattctttccatttgctatcatgtcagtgaacgccaggagtgctttctttgcaacttgtgtaacattttctgttttttcaggttttactgatgaggcttgtgaggccaatcaaaataatgtttgtgatctctactactgttgattttgccctcggagcaaactgaataaagcaacaagatgaaaactgaaaaaaaaaaaaaaaaaa

SEQ ID NO: 44 - protein, 757 aa, Homo sapiens
MLRACQLSGVTAAAQSCLCGKFVLRPLRPCRRYSTSGSSGLTTGKIAGAGLLFVGGGIGGTILYAKWDSHFRESVEKTIPYSDKLFEMVLGPAAYNVPLPKKSIQSGPLKISSVSEVMKESKQPASQLQKQKGDTPASATAPTEAAQIISAAGDTLSVPAPAVQPEESLKTDHPEIGEGKPTPALSEASSSSIRERPPEEVAARLAQQEKQEQVKIESLAKSLEDALRQTASVTLQAIAAQNAAVQAVNAHSNILKAAMDNSEIAGEKKSAQWRTVEGALKERRKAVDEAADALLKAKEELEKMKSVIENAKKKEVAGAKPHITAAEGKLHNMIVDLDNVVKKVQAAQSEAKVVSQYHELVVQARDDFKRELDSITPEVLPGWKGMSVSDLADKLSTDDLNSLIAHAHRRIDQLNRELAEQKATEKQHITLALEKQKLEEKRAFDSAVAKALEHHRSEIQAEQDRKIEEVRDAMENEMRTQLRRQAAAHTDHLRDVLRVQEQELKSEFEQNLSEKLSEQELQFRRLSQEQVDNFTLDINTAYARLRGIEQAVQSHAVAEEEARKAHQLWLSVEALKYSMKTSSAETPTIPLGSAVEAIKANCSDNEFTQALTAAIPPESLTRGVYSEETLRARFYAVQKLARRVAMIDETRNSLYQYFLSYLQSLLLFPPQQLKPPPELCPEDINTFKLLSYASYCIEHGDLELAAKFVNQLKGESRRVAQDWLKEARMTLETKQIVEILTAYASAVGIGTTQVQPE

SEQ ID NO: 45 - DNA, 2985 bp, Homo sapiens
  source 1..2985: mol_type = other DNA
aattctctttaagagtcacagctgtccattttccacgtgcggctgaagaatggatttcagagcgctctccctccggcagtgttcacctagtaaccccttccggataaggacctgcagcccgggcgcgacccggaaagggaatccgcccttttcgcctccttcgcgccaatcgcctggagttggcgtttccgcagccgggacacagcccaccctctaagagccgcggagtcggggacggtagaaggggccgcgcgtgcgcagtggcgtccgctgtgcttccggtgcgccgggcgcggacgcgggcacgcacacacgcaagcacgcctccacttaactcgcgccgccgcggcagctcgagtccaccagcagcgccgtccgcttgaccgagatgctgcgggcctgtcagttatcgggtgtgaccgccgccgcccagagttgtctctgtgggaagtttgtcctccgtccattgcgaccatgccgcagatactctacttcaggcagctctgggttgactactggcaaaattgctggagctggccttttgtttgttggtggaggtattggtggcactatcctatatgccaaatgggattcccatttccgggaaagtgtagagaaaaccataccttactcagacaaactcttcgagatggttcttggtcctgcagcttataatgttccattgccaaagaaatcgattcagtcgggtccactaaaaatctctagtgtatcagaagtaatgaaagaatctaaacagcctgcctcacaactccaaaaacaaaagggagatactccagcttcagcaacagcaggtgataccctgtcggtcccagcccctgcagttcagcctgaggaatctttaaaaactgatcaccctgaaattggtgaaggaaaacccacacctgcactttcagaagaagcatcctcatcttctataagggagcgaccacctgaagaagttgcagctcgccttgcacaacaggaaaaacaagaacaagttaaaattgagtctctagccaagagcttagaagatgctctgaggcaaactgcaagtgtcactctgcaggctattgcagctcagaatgctgcggtccaggctgtcaatgcacactccaacatattgaaagccgccatggacaattctgagattgcaggcgagaagaaatctgctcagtggcgcacagtggagggtgcattgaaggaacgcagaaaggcagtagatgaagctgccgatgcccttctcaaagccaaagaagagttagagaagatgaaaagtgtgattgaaaatgcaaagaaaaaagaggttgctggggccaagcctcatataactgctgcagagggtaaacttcacaacatgatagttgatctggataatgtggtcaaaaaggtccaagcagctcagtctgaggctaaggttgtatctcagtatcatgagctggtggtccaagctcgggatgactttaaacgagagctggacagtattactccagaagtccttcctgggtggaaaggaatgagtgtttcagacttagctgacaagctctctactgatgatctgaactccctcattgctcatgcacatcgtcgtattgatcagctgaacagagagctggcagaacagaaggccaccgaaaagcagcacatcacgttagccttggagaaacaaaagctggaagaaaagcgggcatttgactctgcagtagcaaaagcattagaacatcacagaagtgaaatacaggctgaacaggacagaaagatagaagaagtcagagatgccatggaaaatgaaatgagaacccagcttcgccgacaggcagctgcccacactgatcacttgcgagatgtccttagggtacaagaacaggaattgaagtctgaatttgagcagaacctgtctgagaaactctctgaacaagaattacaatttcgtcgtctcagtcaagagcaagttgacaactttactctggatataaatactgcctatgccagactcagaggaatcgaacaggctgttcagagccatgcagttgctgaagaggaagccagaaaagcccaccaactctggctttcagtggaggcattaaagtacagcatgaagacctcatctgcagaaacacctactatcccgctgggtagtgcagttgaggccatcaaagccaactgttctgataatgaattcacccaagctttaaccgcagctatccctccagagtccctgacccgtggggtgtacagtgaagagacccttagagcccgtttctatgctgttcaaaaactggcccgaagggtagcaatgattgatgaaaccagaaatagcttgtaccagtacttcctctcctacctacagtccctgctcctattcccacctcagcaactgaagccgcccccagagctctgccctgaggatataaacacatttaaattactgtcatatgcttcctattgcattgagcatggtgatctggagctagcagcaaagtttgtcaatcagctgaagggggaatccagacgagtggcacaggactggctgaaggaagcccgaatgaccctagaaacgaaacagatagtggaaatcctgacagcatatgccagcgccgtaggaataggaaccactcaggtgcagccagagtgaggtttaggaagattttcataaagtcatatttcatgtcaaaggaaatcagcagtgatagatgaagggttcgcagcgagagtcccggacttgtctagaaatgagcaggtttacaagtactgttctaaatgttaacacctgttgcatttatattctttccatttgctatcatgtcagtgaacgccaggagtgctttctttgcaacttgtgtaacattttctgttttttcaggttttactgatgaggcttgtgaggccaatcaaaataatgtttgtgatctctactactgttgattttgccctcggagcaaactgaataaagcaacaagatgaaaactgaaaaaaaaaaaaaaaaaa

SEQ ID NO: 46 - protein, 747 aa, Homo sapiens
MLRACQLSGVTAAAQSCLCGKFVLRPLRPCRRYSTSGSSGLTTGKIAGAGLLFVGGGIGGTILYAKWDSHFRESVEKTIPYSDKLFEMVLGPAAYNVPLPKKSIQSGPLKISSVSEVMKESKQPASQLQKQKGDTPASATAGDTLSVPAPAVQPEESLKTDHPEIGEGKPTPALSEEASSSSIRERPPEEVAARLAQQEKQEQVKIESLAKSLEDALRQTASVTLQAIAAQNAAVQAVNAHSNILKAAMDNSEIAGEKKSAQWRTVEGALKERRKAVDEAADALLKAKEELEKMKSVIENAKKKEVAGAKPHITAAEGKLHNMIVDLDNVVKKVQAAQSEAKVVSQYHELVVQARDDFKRELDSITPEVLPGWKGMSVSDLADKLSTDDLNSLIAHAHRRIDQLNRELAEQKATEKQHITLALEKQKLEEKRAFDSAVAKALEHHRSEIQAEQDRKIEEVRDAMENEMRTQLRRQAAAHTDHLRDVLRVQEQELKSEFEQNLSEKLSEQELQFRRLSQEQVDNFTLDINTAYARLRGIEQAVQSHAVAEEEARKAHQLWLSVEALKYSMKTSSAETPTIPLGSAVEAIKANCSDNEFTQALTAAIPPESLTRGVYSEETLRARFYAVQKLARRVAMIDETRNSLYQYFLSYLQSLLLFPPQQLKPPPELCPEDINTFKLLSYASYCIEHGDLELAAKFVNQLKGESRRVAQDWLKEARMTLETKQIVEILTAYASAVGIGTTQVQPE

SEQ ID NO: 47 - protein, 15 aa, Homo sapiens
YLILGSAVGGGYTAK

SEQ ID NO: 48 - protein, 6 aa, Homo sapiens
TFDQWK

SEQ ID NO: 49 - protein, 10 aa, Homo sapiens
DMIPDLSEYK

SEQ ID NO: 50 - protein, 18 aa, Homo sapiens
WIVPDIVWEIDEYIDFEK

SEQ ID NO: 51 - protein, 7 aa, Homo sapiens
LAPDFDK

SEQ ID NO: 52 - protein, 9 aa, Homo sapiens
IVESLSLLK

SEQ ID NO: 53 - protein, 10 aa, Homo sapiens
ALPNSEDLVK

SEQ ID NO: 54 - protein, 14 aa, Homo sapiens
DFFTSGSPEETAFR

SEQ ID NO: 55 - protein, 13 aa, Homo sapiens
TRLLKLRYLILGS

SEQ ID NO: 56 - protein, 20 aa, Homo sapiens
FWPARLATRLLKLRYLILGS

SEQ ID NO: 57 - protein, 21 aa, Homo sapiens
GLLGELILLQQQIQEHEEEAR

SEQ ID NO: 58 - protein, 13 aa, Homo sapiens
AAGQYSTSYAQQK

SEQ ID NO: 59 - protein, 14 aa, Homo sapiens
IDQLQEELLHTQLK

SEQ ID NO: 60 - protein, 37 aa, Homo sapiens
EYKWIVPDIVWEIDEYIDFGHKLVSEVIGASDLLLLL

SEQ ID NO: 61 - protein, 42 aa, Homo sapiens
EYKWIVPDIVWEIDEYIDFGSPEETAFRATDRGSESDKHFRK

SEQ ID NO: 62 - protein, 59 aa, Homo sapiens
EKIRKALPNSEDLVKLAPDFDKIVESLSLLKDFFTSGSPEETAFRATDRGSESDKHFRK

SEQ ID NO: 63 - protein, 60 aa, Homo sapiens
GSPEETAFRATDRGSESDKHFRKVSDKEKIDQLQEELLHTQLKYQRILERLEKENKELRK